(19) United States
(12) Patent Application Publication
(10) Pub. No.: US 2007/0129334 A1
(43) Pub. Date: Jun. 7, 2007

(54) Orally Active Purine-Based Inhibitors of Heat Shock Protein 90

(75) Inventors: Srinivas R. Kasibhatla, San Diego, CA (US); Lin Zhang, San Diego, CA (US); Marco Antonio Biamonte, San Diego, CA (US); Marcus F. Boehm, San Diego, CA (US); Junhua Fan, San Diego, CA (US); Jiandong Shi, San Diego, CA (US); Kevin D. Hong, San Diego, CA (US)

Correspondence Address: WILSON SONSINI GOODRICH & ROSATI, 650 PAGE MILL ROAD, PALO ALTO, CA, 94304-1050, US

(73) Assignee: CONFORMA THERAPEUTICS CORPORATION, San Diego, CA (US)

(21) Appl. No.: 11/612,418

(22) Filed: Dec. 18, 2006

Related U.S. Application Data
(63) Continuation-in-part of application No. 10/494,414, filed on Oct. 4, 2004.
(60) Provisional application No. 60/335,391, filed on Oct. 30, 2001.
(60) Provisional application No. 60/753,636, filed on Dec. 22, 2005.
(60) Provisional application No. 60/753,448, filed on Dec. 22, 2005.
(60) Provisional application No. 60/753,698, filed on Dec. 22, 2005.

(51) Int. Cl. A61K 31/675; C07D 473/00; C07D 473/12

(52) U.S. Cl. 514/81; 514/263.2; 514/263.3; 544/244; 544/276

(57) ABSTRACT

Novel purine compounds and tautomers and pharmaceutically acceptable salts thereof are described, as are pharmaceutical compositions comprising the same, complexes comprising the same, e.g., HSP90 complexes, and methods of using the same. Methods of using the novel purine compounds of the invention, and tautomers and pharmaceutically acceptable salts thereof, include their use in inhibiting heat shock protein 90's (HSP90's) to thereby treat or prevent HSP90-dependent diseases, e.g., proliferative disorders such as breast cancer.

### CROSS-REFERENCE

This application is a continuation-in-part application of Ser. No. 10/494,414, filed Apr. 30, 2004 which is a US national phase application of International Application PCTUS02/35069 filed Oct. 30, 2002, which in turn claims priority to U.S. provisional application 60/335,391 filed Oct. 30, 2001. This application also claims the benefit of U.S. Provisional Application No. 60/753,636, filed Dec. 22, 2005, Provisional Application No. 60/753,448, filed Dec. 22, 2005 and Provisional Application No. 60/753,698, also filed Dec. 22, 2005. The contents of all the above applications are incorporated herein by reference in their entirety.

### FIELD OF THE INVENTION

The invention relates in general to purine analogs and their use in inhibiting heat shock protein 90's (HSP90's) to thereby treat or prevent HSP90-dependent diseases, e.g., proliferative disorders such as breast cancer.

### BACKGROUND OF THE INVENTION

The following description includes information that may be useful in understanding the present invention. It is not an admission that any of the information provided herein is prior art or relevant to the presently claimed inventions, or that any publication specifically or implicitly referenced is prior art.

Heat Shock Protein 90's (Hsp90s) are ubiquitous chaperone proteins that maintain the proper conformation of many “client” proteins (see Kamal et. al. Trends Mol. Med. 2004, 10, 283-290; Dymock et. al. Expert Opin. Ther. Patents 2004, 14, 837-847; Isaacs et. al. Cancer Cell, 2003, 3, 213; Maloney et. al. Expert Opin. Biol. Ther. 2002, 2, 3-24 and Richter et. al. J. Cell. Physiol. 2001, 188, 281-290), and are involved in folding, activation and assembly of a wide range of proteins, including key proteins involved in signal transduction, cell cycle control and transcriptional regulation. Researchers have reported that HSP90 chaperone proteins are associated with important signaling proteins, such as steroid hormone receptors and protein kinases, including, e.g., Raf-1, EGFR, v-Src family kinases, Cdk4, and ErbB-2 (Buchner, TIBS, 1999, 24, 136-141; Stepanova et. al., Genes Dev. 1996, 10, 1491-502; Dai et. al., J. Biol. Chem. 1996, 271, 22030-4). Studies further indicate that certain co-chaperones, e.g., Hsp70, p60/Hop/Sti1, Hip, Bag1, HSP40/Hdj2/Hsj1, immunophilins, p23, and p50, may assist HSP90 in its function (see for example Caplan, Trends in Cell Biol., 1999, 9, 262-268). Inhibition of Hsp90 causes these client proteins to adopt aberrant conformations, and these abnormally folded proteins are rapidly eliminated by the cell via ubiquitinylation and proteasome degradation. Interestingly, the list of Hsp90 client proteins includes a series of notorious oncogenes. Four of them are clinically validated cancer targets: HER-2/neu (Herceptin® (trastuzumab)), Bcr-Abl (Gleevec® (imatinib mesylate)), the estrogen receptor (tamoxifen), and the androgen receptor (Casodex® (bicalutamide)), while the others play a critical role in the development of cancer. Some of the most sensitive Hsp90 clients are involved in growth signalling (Raf-1, Akt, cdk4, Src, Bcr-Abl, etc). In contrast, few tumor suppressor genes, if any, seem to be clients of Hsp90 (for lists of client proteins see Pratt et. al. Exp. Biol. Med. 2003, 228, 111-133; Workman et. al. Cancer Lett. 2004, 206, 149-157 and Zhang et. al. J. Mol. Med. 2004, 82, 488-499.), and consequently, inhibition of Hsp90 has an overall anti-proliferative effect. In addition, some client proteins are involved in other fundamental processes of tumorigenesis, namely apoptosis evasion (e.g. Apaf-1, RIP, Akt), immortality (e.g. hTert), angiogenesis (e.g. VEGFR, Flt-3, FAK, HIF-1), and metastasis (c-Met).

The various client proteins are not equally responsive to Hsp90 inhibitors, and some undergo degradation at lower concentrations of the inhibitor, or with faster kinetics, depending on the cell line. The more sensitive clients are usually those involved in growth signaling, but some mutated proteins found in tumor cells (mutant p53, Gleevec-resistant Bcr-Abl, see Gorre et. al. Blood, 2002, 100, 3041-3044) are particularly dependent on Hsp90 to preserve their conformation and function. This unique feature sensitizes tumor cells to Hsp90 inhibitors, and when these factors converge, they confer on Hsp90 inhibitors notable anti-cancer properties in vitro and in vivo.

A remarkable advantage of targeting Hsp90 lies in the simultaneous depletion of multiple oncogenic proteins, thereby attacking several pathways necessary for cancer development, and reducing the likelihood of the tumor acquiring resistance to the Hsp90 inhibitor. Another striking feature of Hsp90 is that it occurs in an activated form in cancer cells, and in a latent form in normal cells (Kamal et. al. Nature, 2003, 425, 407-410 and Workman et. al. Trends Mol. Med. 2004, 10, 47-51.) This provides an opportunity to specifically target cancer cells with inhibitors selective for the activated form. What distinguishes the activated and latent forms of Hsp90 at a molecular level is not well understood. It is clear, however, that the activity of Hsp90 is regulated by a highly sophisticated process involving at a minimum (1) Hsp90 dimerization, (2) formation of multi-protein complexes with numerous co-chaperones, and (3) ATP/ADP binding, ATP hydrolysis being essential for the chaperone cycle and function.

The chaperoning function of Hsp90 can be “switched off” by inhibiting its ATP-ase activity. The nucleotides ADP and ATP can bind to two sites, one located close to the N-terminal, the other close to the C-terminal. Geldanamycin, isolated from the microorganism Streptomyces hygroscopicus, was originally identified for its antiprotozoal, herbicidal and antifungal activities. Ansamycin antibiotics, such as geldanamycin (GM), herbimycin A (HA), and 17-AAG are thought to exert their anticancerous effects by tight binding of the N-terminus pocket of HSP90, (while for example novobiocin binds to the C-terminal domain, see Yun et. al. Biochemistry, 2004, 43, 8217-8229), thereby destabilizing substrates that normally interact with HSP90 (Stebbins et al. Cell, 1997, 89, 239-250). This pocket is highly conserved and has weak homology to the ATP-binding site of DNA gyrase (Stebbins, C. et al., supra; Grenert, J. P. et al., 1997, J. Biol. Chem., 272:23843-50). Further, ATP and ADP have both been shown to bind this pocket with low affinity and to have weak ATPase activity (Proromou, et. al., Cell, 1997, 90, 65-75 and Panaretou, et. al., EMBO J., 1998, 17, 4829-36). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T., H. et al., 1999, Proc. Natl. Acad. Sci. USA 96:1297-302; Schulte, T. W. et al., 1995, J. Biol. Chem. 270:24585-8; Whitesell, L., et al., 1994, Proc. Natl. Acad. Sci. USA 91:8324-8328). Ansamycins have also been demonstrated to inhibit the ATP-dependent release of chaperone-associated protein substrates (Schneider, C., L. et al., 1996, Proc. Natl. Acad. Sci. USA, 93:14536-41; Sepp-Lorenzino et al., 1995, J. Biol. Chem. 270:16580-16587). In either event, the substrates are degraded by a ubiquitin-dependent process in the proteasome (Schneider, C., L., supra; Sepp-Lorenzino, L., et al., 1995, J. Biol. Chem., 270:16580-16587; Whitesell, L. et al., 1994, Proc. Natl. Acad. Sci. USA, 91: 8324-8328).

HSP90 substrate destabilization occurs in tumor and non-transformed cells alike and has been shown to be especially effective on a subset of signaling regulators, e.g., Raf (Schulte, T. W. et al., 1997, Biochem. Biophys. Res. Commun. 239:655-9; Schulte, T. W., et al., 1995, J. Biol. Chem. 270:24585-8), nuclear steroid receptors (Segnitz, B., and U. Gehring. 1997, J. Biol. Chem. 272:18694-18701; Smith, D. F. et al., 1995, Mol. Cell. Biol. 15:6804-12), v-src (Whitesell, L., et al., 1994, Proc. Natl. Acad. Sci. USA 91:8324-8328) and certain transmembrane tyrosine kinases (Sepp-Lorenzino, L. et al., 1995, J. Biol. Chem. 270:16580-16587) such as EGF receptor (EGFR) and Her2/Neu (Hartmann, F., et al., 1997, Int. J. Cancer 70:221-9; Miller, P. et al., 1994, Cancer Res. 54:2724-2730; Mimnaugh, E. G., et al., 1996, J. Biol. Chem. 271:22796-801; Schnur, R. et al., 1995, J. Med. Chem. 38:3806-3812), CDK4, and mutant p53. Erlichman et al., Proc. AACR (2001), 42, abstract 4474. The ansamycin-induced loss of these proteins leads to the selective disruption of certain regulatory pathways and results in growth arrest at specific phases of the cell cycle (Muise-Heimericks, R. C. et al., 1998, J. Biol. Chem. 273:29864-72), and apoptsosis, and/or differentiation of cells so treated (Vasilevskaya, A. et al., 1999, Cancer Res., 59:3935-40).

In addition to anti-cancer and antitumorgenic activity, HSP90 inhibitors have also been implicated in a wide variety of other utilities, including use as anti-inflammation agents, anti-infectious disease agents, agents for treating autoimmunity, agents for treating ischemia, and agents useful in promoting nerve regeneration (See, e.g., Rosen et al., WO 02/09696; PCT/US01/23640; Degranco et al., WO 99/51223; PCT/US99/07242; Gold, U.S. Pat. No. 6,210,974 B1). There are reports in the literature that fibrogenetic disorders including but not limited to scleroderma, polymyositis, systemic lupus, rheumatoid arthritis, liver cirrhosis, keloid formation, interstitial nephritis, and pulmonary fibrosis may be treatable. (Strehlow, WO 02/02123; PCT/US01/20578).

Ansamycins and other HSP90 inhibitors thus hold great promise for the treatment and/or prevention of many types of disorders. However, many of the natural-product derived Hsp90 inhibitors exhibit pharmaceutical deficiencies; their relative insolubility makes them difficult to formulate and administer, and they are not easily synthesized and currently must, at least in part, be generated through fermentation. Further, the dose limiting toxicity of ansamyins is hepatic. For example, the semi-synthetic inhibitor 17-allylamino,17-desmethoxy-geldanamycin (17-AAG), currently in phase II clinical trials, is expensive to manufacture, difficult to formulate (the NCI clinical protocol consists of injecting a DMSO solution of 17-AAG) and at present administered only parenterally. Although the 17-dimethylaminoethylamino analog (17-DMAG) is more soluble, it exhibits all of the side effects of 17-AAG as well as gastrointestinal hemorrhaging in preclinical toxicity studies (Glaze et. al. Proc. Am. Assoc. Cancer. Res. 2003, 44, 162-162 and Eiseman et. al. Cancer Chemother. Pharmacol. 2005, 55, 21-32). Radicicol (RC), another natural product Hsp90 inhibitor, is poorly water-soluble and is inactive in tumor xenograft models. Semi-synthetic oxime derivatives of radicicol provide better solubility and substantially improved the pharmacological profile in murine models, but are still limited to intravenous administration (Ikuina et. al. J. Med. Chem. 2003, 46, 2534-2541. Furthermore, radicicol and its oximes contain an oxirane ring which has been viewed as a liability for stability and toxicity, prompting the synthesis of cycloproparadicicol: Yang et. al. J. Am. Chem. Soc. 2004, 126, 7881 and 2003, 125, 9602-9603.) Despite the potential of ansamycins, alternative HSP90 inhibitors are therefore needed.

Fully synthetic, orally active inhibitors of Hsp90 have been sought in order to provide more flexible dosing schedule options, and to possibly avoid the side-effects of the natural product inhibitors. Chiosis et al. described the design and synthesis of purine analogs that mimic geldanamycin and other ansamycins in their ability to bind the ATP binding pocket of, and thus inhibit, HSP90. See International Patent Application PCT/US01/46303 (WO 02/36075; Chemistry & Biology 8:289-299 (2001). The specific compounds that Chiosis et al. described included a trimethoxybenzyl entity substituted at positions 3, 4, and 5. Using gel-binding assays, these were shown to bind HSP90 approximately 20-fold less avidly than 17-AAG. Chiosis et al. did not attempt a quinone mimic for the methoxybenzyl entity, speculating that to do so would lead to hepatoxicity. Id., pg. 290, col. 1, ¶4. Nor did Chiosis et al. teach, suggest, or otherwise report the use of sulfides, sulfoxides, and sulfones as described herein.

More recently, other novel non-natural product Hsp90 inhibitors have been reported (e.g. PU3 and CCT018159; see Chiosis et. al. Bioorg. Med. Chem. Lett. 2002, 10, 3555-3564; Vilenchik et. al. Chem. Biol. 2004, 11, 787-797; Chiosis et. al. WO 0236075, 2002; Drysdale et. al. WO 03/055860 A1, 2003; Wright et. al. Chem. Biol. 2004, 11, 775-785; Dymock et. al. Bioorg. Med. Chem. Lett. 2004, 14, 325-328; Dymock et. al. J. Med. Chem. 2005, 48, 4212-4215. Structure of Hsp90 in complex with PU3 pdb code 1UY6, and with PU24FCl: pdb code 1UYF and Clevenger et. al. Org. Lett. 2004, 6, 4459-4462). The structures of these inhibitors were designed using the crystal structures of Hsp90 in complex with ATP, geldanamycin, or radicicol. The 8-benzyladenines such as PU3 were designed to adopt the same C-shaped conformation as geldanamycin (Chiosis et. al. Current Cancer Drug Targets, 2003, 3, 371-376) with the adenine ring pointing to the adenine-binding site (hinge region), and the trimethoxybenzene ring emulating the H-bond accepting nature of the quinone ring of geldanamycin. (The benzene ring of PU3 was not designed to have exactly the same orientation as the quinone ring of geldanamycin. Rather, the trimethoxybenzene moiety was designed to point in the same general direction and form a hydrogen bond with Lys112, an amino acid which forms a hydrogen bond with the quinone ring of geldanamycin.) The recently obtained crystal structure of Hsp90 in complex with PU3 confirmed that the purine ring occupies the position normally occupied by ADP/ATP, but the benzene ring points in a direction opposite to the predicted one, to form a r-stacking interaction with Phe138. Nevertheless, PU3 inhibits Hsp90 (HER-2 degradation assay, HER-2 IC₅₀=40 μM) and afforded a valuable starting point for further optimization. Structure-activity studies based on PU3 led to the more active PU24FCl (HER-2 IC₅₀=1.7 μM) which was subsequently also co-crystallized with Hsp90. When PU24FCl was formulated in DMSO/EtOH/phosphate-buffered saline 1:1:1 and administered intraperitoneally to mice bearing MCF-7 xenograft tumors, it induced at 100-300 mg/kg down-regulation of HER-2 and Raf-1, a pharmacodynamic response consistent with Hsp90 inhibition, and at 200 mg/kg it significantly repressed tumor growth. Very high doses (500-1000 mg/kg) of PU24FCl were required to observe a similar pharmacodynamic response upon oral administration, and no 8-benzyladenine has been reported to inhibit tumor growth by the oral route. In our hands, PU24FCl proved to be too insoluble to be effectively formulated and delivered orally. So far, despite extensive SAR studies to improve potency and pharmaceutics properties, Hsp90 inhibitors have not demonstrated activity in animal models of human cancer (xenografts) when administered orally.

The discovery of the 8-benzyladenines led to the design of 8-sulfanyladenines (Kasibhatla et. al. WO 3037860, 2003 and Llauger et. al. J. Med. Chem. 2005, 48, 2892-2905), exemplified by 8-(2-iodo-5-methoxy-phenylsulfanyl)-9-pent-4-ynyl-9H-purin-6-ylamine, which exhibited excellent potency in several cell-based assays, but was poorly soluble in water and did not have sufficient oral bioavailability in clinically acceptable formulations.

The present invention provides water-soluble, orally bioavailable purine analogs, and their use in inhibiting heat shock protein 90's to thereby treat or prevent Hsp90-dependent diseases as demonstrated by their oral efficacy in tumor xenograft models.

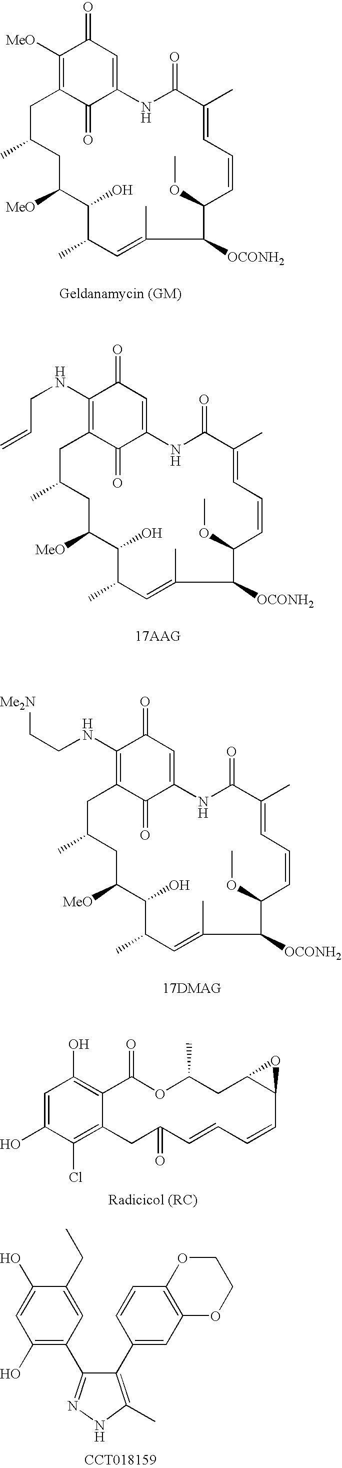

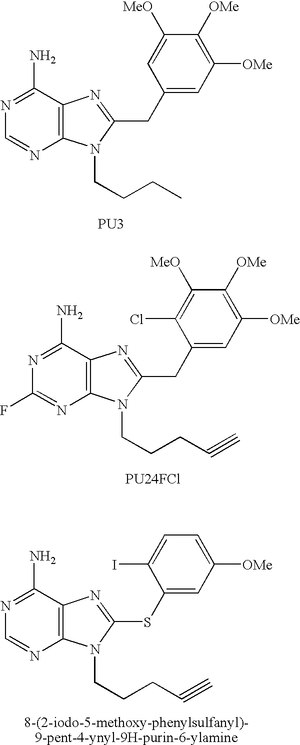

### SUMMARY OF THE INVENTION

In one embodiment, the invention provides a compound of Formula I:

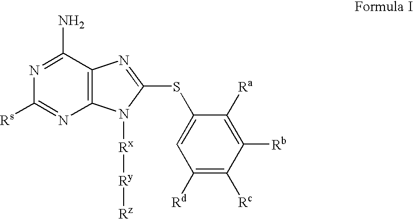

or tautomer or pharmaceutically acceptable salt thereof, wherein

R^{s} is independently selected from H and F;

each R^{a}, R^{b}, R^{c}, and R^{d} is independently selected from H, halo, lower alkyl, OR³, SR³, C(O)N(R⁴)₂, NR⁴R⁴, C(O)R², and —C(O)OR⁴;

R^{x} is independently selected from optionally substituted C₁-C₆ alkyl, optionally substituted C₂-C₆ alkenyl and optionally substituted C₂-C₆ alkynyl;

R^{y} is independently selected from O, NR¹ and a bond;

R^{z} is independently selected from H, C₁-C₆ alkyl, C₂-C₆ alkenyl, C₂-C₆ alkynyl, —P(O)(OR⁴)₂ and C(O)R²;

R¹ is independently selected from H, optionally substituted alkyl, optionally substituted alkenyl, optionally substituted alkynyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;

R² is independently selected from optionally substituted alkyl, optionally substituted cycloalkyl, optionally substituted heteroalkyl, optionally substituted aryl, optionally substituted heterocyclyl and optionally substituted heteroaryl;

R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²; and

R⁴ is independently selected from H, optionally substituted alkyl, optionally substituted cycloalkyl, optionally substituted heteroalkyl, optionally substituted aryl, and optionally substituted heterocyclyl. The invention also provides the following embodiments:
compounds of Formula I wherein R^{a}, R^{b}, R^{c}, and R^{d} are independently selected from halo and OR³;
compounds of Formula I wherein at least two of R^{a}, R^{b}, R^{c}, and R^{d} are independently selected from halo and methoxy;
compounds of Formula I wherein at least three of R^{a}, R^{b}, R^{c}, and R^{d} are independently selected from halo and OR³;
compounds of Formula I wherein at least three of R^{a}, R^{b}, R^{c}, and R^{d} are independently selected from halo and methoxy;
compounds of Formula I wherein R^{a} is halo, and R^{d} is OR³;
compounds of Formula I wherein R^{x} is optionally substituted C₂-C₃ alkyl, optionally substituted C₂-C₃ alkenyl or optionally substituted C₂-C₃ alkynyl, R^{y} is NR¹; and R^{z} is C₁-C₆ alkyl;
compounds of Formula I wherein R^{x} is optionally substituted C₂-C₃ alkyl, optionally substituted C₂-C₃ alkenyl or optionally substituted C₂-C₃ alkynyl; R^{y} is a bond; and R^{z} is H;
compounds of Formula I wherein R^{x} is optionally substituted C₂-C₃ alkyl, optionally substituted C₂-C₃ alkenyl or optionally substituted C₂-C₃ alkynyl; R^{y} is NR¹; and R^{z} is C(O)R².
compounds of Formula I wherein R^{x} is optionally substituted C₂-C₃ alkyl, optionally substituted C₂-C₃ alkenyl or optionally substituted C₂-C₃ alkynyl; R^{y} is NH; and R^{z} is H.
compounds of Formula I wherein R^{x} is optionally substituted C₂-C₃ alkyl, optionally substituted C₂-C₃ alkenyl or optionally substituted C₂-C₃ alkynyl; R^{y} is NH; and R^{z} is C₁-C₆ alkyl; and
compounds of Formula I wherein R^{x} is optionally substituted C₂-C₃ alkyl; R^{y} is NH; and R^{z} is C₁-C₆ alkyl.

In a further embodiment, the invention provides a compound of formula II:

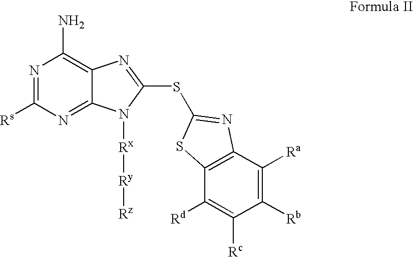

or tautomer or pharmaceutically acceptable salt thereof, wherein

R^{s} is independently selected from H and F;

each R^{a}, R^{b}, R^{c}, and R^{d} is independently selected from H, halo, lower alkyl, OR³, SR³, C(O)N(R⁴)₂, NR⁴R⁴, C(O)R², and —C(O)OR⁴;

R^{x} is independently selected from optionally substituted C₂-C₆ alkyl, optionally substituted C₂-C₆ alkenyl and optionally substituted C₂-C₆ alkynyl;

R^{y} is independently selected from O, NR¹ or a bond;

R^{z} is independently selected from H, C₁-C₆ alkyl, C₂-C₆ alkenyl, C₂-C₆ alkynyl, —P(O)(OR⁴)₂ and C(O)R²;

R¹ is independently selected from H, optionally substituted alkyl, optionally substituted alkenyl, optionally substituted alkynyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;

R² is independently selected from optionally substituted alkyl, optionally substituted cycloalkyl, optionally substituted heteroalkyl, optionally substituted aryl, optionally substituted heterocyclyl and optionally substituted heteroaryl;

R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²; and

R⁴ is independently selected from H, optionally substituted alkyl, optionally substituted cycloalkyl, optionally substituted heteroalkyl, optionally substituted aryl, and optionally substituted heterocyclyl.

The invention also provides the following embodiments:
compounds of Formula II wherein at least one of R^{a}, R^{b}, R^{c}, and R^{d} is halo; R^{x} is optionally substituted C₂-C₃ alkyl; R^{y} is a bond; and R^{z} is H;
compounds of Formula II wherein at least one of R^{a}, R^{b}, R^{c}, and R^{d} is halo; R^{x} is optionally substituted C₂-C₃ alkyl; R^{y} is NR¹; and R^{z} is H;
compounds of Formula II wherein at least one of R^{a}, R^{b}, R^{c}, and R^{d} is halo; R^{x} is optionally substituted C₂-C₃ alkyl; R^{y} is NR¹; and R^{z} is C₁-C₆ alkyl;
compounds of Formula II wherein at least one of R^{a}, R^{b}, R^{c}, and R^{d} is halo; R^{x} is optionally substituted C₂-C₃ alkyl; R^{y} is a bond; and R^{y} is —P(O)(OR⁴)₂; and
compounds of Formula II wherein at least one of R^{a}, R^{b}, R^{c}, and R^{d} is methoxy; R^{x} is optionally substituted C₂-C₃ alkyl; R^{y} is a bond; and R^{z} is H.

In yet another embodiment, the invention provides a compound of formula III:

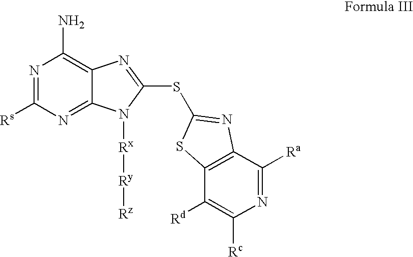

or tautomer or pharmaceutically acceptable salt thereof, wherein

R^{s} is independently selected from H and F;

each R^{a}, R^{c} and R^{d} is independently selected from H, halo, lower alkyl, OR³, SR³, C(O)N(R⁴)₂, NR⁴R⁴, C(O)R², and —C(O)OR⁴;

R^{x} is independently selected from optionally substituted C₂-C₄ alkyl, optionally substituted C₂-C₄ alkenyl and optionally substituted C₂-C₄ alkynyl;

R^{y} is independently selected from O, NR¹ and a bond; and

R^{z} is independently selected from H, C₁-C₆ alkyl, C₂-C₆ alkenyl, C₂-C₆ alkynyl, —P(O)(OR⁴)₂ and C(O)R²;

R¹ is independently selected from H, optionally substituted alkyl, optionally substituted alkenyl, optionally substituted alkynyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;

R² is independently selected from optionally substituted alkyl, optionally substituted cycloalkyl, optionally substituted heteroalkyl, optionally substituted aryl, optionally substituted heterocyclyl and optionally substituted heteroaryl;

R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²; and

R⁴ is independently selected from H, optionally substituted alkyl, optionally substituted cycloalkyl, optionally substituted heteroalkyl, optionally substituted aryl, and optionally substituted heterocyclyl.

The invention also provides the following embodiments:
compounds of Formula III wherein at least one of R^{a}, R^{c}, and R^{d} is halo; R^{x} is optionally substituted C₂-C₃ alkyl; R^{y} is a bond; and R^{z} is H;
compounds of Formula III wherein at least one of R^{a}, R^{c}, and R^{d} is halo; R^{x} is optionally substituted C₂-C₃ alkyl; R^{y} is NR¹; and R^{z} is H;
compounds of Formula III wherein at least one of R^{a}, R^{c}, and R^{d} is halo; R^{x} is optionally substituted C₂-C₃ alkyl; R^{y} is a bond; and R^{z} is C₁-C₆ alkyl; and
compounds of Formula III wherein at least one of R^{a}, R^{c}, and R^{d} is halo;

R^{x} is optionally substituted C₂-C₃ alkyl; R^{y} is a bond; and R^{z} is —P(O)(OR⁴)₂.

In yet a further embodiment, the invention provides a compound of formula IV:

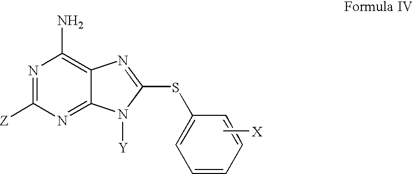

or tautomer or pharmaceutically acceptable salt thereof, wherein

X is independently selected from H, halo, CN, N₃, N(R¹)₂, NR¹S(O)₂R², OR³, SR³, lower alkyl, C(O)N(R⁴)₂, perhaloalkyl, C(O)R², and —C(O)OR⁴;

Y is independently selected from optionally substituted alkyl, optionally substituted alkenyl, optionally substituted alkynyl, optionally substituted aryl, optionally substituted alicyclic, optionally substituted araalkyl, optionally substituted aryloxyalkyl, optionally substituted alkoxyalkyl, optionally substituted heterocyclyl, optionally substituted alkylaminoalkyl (—(CH₂)ₙ—NHR²), optionally substituted alkylaminodialkyl (—(CH₂)ₙ—NR²R²), optionally substituted alkylcarbonylaminoalkyl, (—(CH₂)ₙ—C(O)—NR⁴R⁴), optionally substituted alkylcarbonyloxylalkyl (—(CH₂)ₙ—C(O)—O—R⁴), hydroxyalkyl (—(CH₂)ₙ—OH), haloalkyl (—(CH₂)ₙ-halo), perhaloalkyl, aminoalkyl (—(CH₂)ₙ—NH₂), C(O)R², S(O)₂R², C(O)NR⁴₂, and C(O)OR²;

Z is independently selected from H and halogen;

R¹ is independently selected from H, optionally substituted alkyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;

R² is independently selected from optionally substituted alkyl, optionally substituted cycloalkyl, optionally substituted heteroalkyl, optionally substituted aryl, optionally substituted heterocyclyl and optionally substituted heteroaryl;

R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²;

R⁴ is independently selected from H, optionally substituted alkyl, optionally substituted cycloalkyl, optionally substituted heteroalkyl, optionally substituted aryl, and optionally substituted heterocyclyl; and

n is from 1 to 3.

In still a further embodiment, the invention provides a compound of formula V:

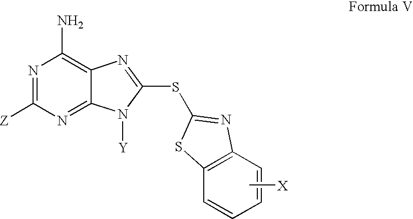

or tautomer or pharmaceutically acceptable salt thereof, wherein

X is independently selected from H, halo, CN, N₃, N(R¹)₂, NR¹S(O)₂R², OR³, SR³, lower alkyl, C(O)N(R⁴)₂, perhaloalkyl, C(O)R², and —C(O)OR⁴;

Y is independently selected from optionally substituted alkyl, optionally substituted alkenyl, optionally substituted alkynyl, optionally substituted aryl, optionally substituted alicyclic, optionally substituted araalkyl, optionally substituted aryloxyalkyl, optionally substituted alkoxyalkyl, optionally substituted heterocyclyl, optionally substituted alkylaminoalkyl (—(CH₂)ₙ—NHR²), optionally substituted alkylaminodialkyl (—(CH₂)ₙ—NR²R²), optionally substituted alkylcarbonylaminoalkyl, (—(CH₂)ₙ—C(O)—NR⁴R⁴), optionally substituted alkylcarbonyloxylalkyl (—(CH₂)ₙ—C(O)—O—R⁴), hydroxyalkyl (—(CH₂)ₙ—OH), haloalkyl (—(CH₂)ₙ-halo), perhaloalkyl, aminoalkyl (—(CH₂)ₙ—NH₂), C(O)R², S(O)₂R², C(O)NR⁴₂, and C(O)OR²;

Z is independently selected from H and halogen;

R¹ is independently selected from H, optionally substituted alkyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;

R² is independently selected from optionally substituted alkyl, optionally substituted cycloalkyl, optionally substituted heteroalkyl, optionally substituted aryl, optionally substituted heterocyclyl and optionally substituted heteroaryl;

R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²;

R⁴ is independently selected from H, optionally substituted alkyl, optionally substituted cycloalkyl, optionally substituted heteroalkyl, optionally substituted aryl, and optionally substituted heterocyclyl; and

n is from 1 to 3.

In a further embodiment, the invention provides a compound of formula VI:

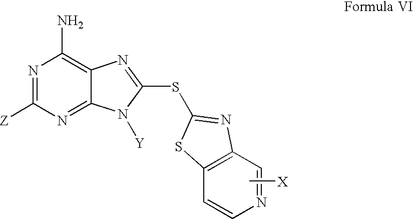

or tautomer or pharmaceutically acceptable salt thereof, wherein

X is independently selected from H, halo, CN, N₃, N(R¹)₂, NR¹S(O)₂R², OR³, SR³, lower alkyl, C(O)N(R⁴)₂, perhaloalkyl, C(O)R², and —C(O)OR⁴;

Y is independently selected from optionally substituted alkyl, optionally substituted alkenyl, optionally substituted alkynyl, optionally substituted aryl, optionally substituted alicyclic, optionally substituted araalkyl, optionally substituted aryloxyalkyl, optionally substituted alkoxyalkyl, optionally substituted heterocyclyl, optionally substituted alkylaminoalkyl (—(CH₂)ₙ—NHR²), optionally substituted alkylaminodialkyl (—(CH₂)ₙ—NR²R²), optionally substituted alkylcarbonylaminoalkyl, (—(CH₂)ₙ—C(O)—NR⁴R⁴), optionally substituted alkylcarbonyloxylalkyl (—(CH₂)ₙ—C(O)—O—R⁴), hydroxyalkyl (—(CH₂)ₙ—OH), haloalkyl (—(CH₂)ₙ-halo), perhaloalkyl, aminoalkyl (—(CH₂)ₙ—NH₂), C(O)R², S(O)₂R², C(O)NR⁴₂, and C(O)OR²;

Z is independently selected from H and halogen;

R¹ is independently selected from H, optionally substituted alkyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;

R² is independently selected from optionally substituted alkyl, optionally substituted cycloalkyl, optionally substituted heteroalkyl, optionally substituted aryl, optionally substituted heterocyclyl and optionally substituted heteroaryl;

R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²;

R⁴ is independently selected from H, optionally substituted alkyl, optionally substituted cycloalkyl, optionally substituted heteroalkyl, optionally substituted aryl, and optionally substituted heterocyclyl; and

n is from 1 to 3.

In yet another embodiment, the invention provides a compound selected from the group consisting of:
9-(tert-Butyl-dimethyl-silanyloxymethyl)-8-(2-iodo-5-methoxy-phenylsulfanyl)-9H-purin-6-ylamine; 9-(2-Chloro-ethyl)-8-(2-iodo-5-methoxy-phenylsulfanyl)-9H-purin-6-ylamine; 9-(3-chloro-propyl)-8-(2-iodo-5-methoxy-phenylsulfanyl)-9H-purin-6-ylamine; 9-(4-Chloro-butyl)-8-(2-iodo-5-methoxy-phenylsulfanyl)-9H-purin-6-ylamine; 8-(2-Iodo-5-methoxy-phenylsulfanyl)-9-[3-(4-methyl-piperazin-1-yl)-propyl]-9H-purin-6-ylamine; 9-(3-Dimethylamino-propyl)-8-(2-Iodo-5-methoxy-phenylsulfanyl)-9H-purin-6-ylamine; 8-(2-Iodo-5-methoxy-phenylsulfanyl)-9-(3-piperidin-1-yl-propyl)-9H-purin-6-ylamine; 9-(3-Cyclopropylamino-propyl)-8-(2-iodo-5-methoxy-phenylsulfanyl)-9H-purin-6-ylamine; 8-(2-Iodo-5-methoxy-phenylsulfanyl)-9-(3-morpholin-4-yl-propyl)-9H-purin-6-ylamine; 8-(2-Iodo-5-methoxy-phenylsulfanyl)-9-(3-methylamino-propyl)-9H-purin-6-ylamine; 9-(3-Ethylamino-propyl)-8-(2-iodo-5-methoxy-phenylsulfanyl)-9H-purin-6-ylamine; 8-(2-Iodo-5-methoxy-phenylsulfanyl)-9-[2-(4-methyl-piperazin-1-yl)-ethyl]-9H-purin-6-ylamine; 8-(2-Iodo-5-methoxy-phenylsulfanyl)-9-(2-piperidin-1-yl-ethyl)-9H-purin-6-ylamine; 8-(2-Iodo-5-methoxy-phenylsulfanyl)-9-(2-propylamino-ethyl)-9H-purin-6-ylamine; 8-(2,5-Dimethoxy-phenylsulfanyl)-9-(3-dimethylamino-propyl)-9H-purin-6-ylamine; 8-(2-Iodo-5-methoxy-phenylsulfanyl)-9-(2-isopropylamino-ethyl)-9H-purin-6-ylamine; 9-(2-Butylamino-ethyl)-8-(2-iodo-5-methoxy-phenylsulfanyl)-9H-purin-6-ylamine; 9-(2-sec-Butylamino-ethyl)-8-(2-iodo-5-methoxy-phenylsulfanyl)-9H-purin-6-ylamine; 9-[2-(1-Ethyl-propylamino)-ethyl]-8-(2-iodo-5-methoxy-phenylsulfanyl)-9H-purin-6-ylamine; 9-(2-Cyclopropylamino-ethyl)-8-(2-iodo-5-methoxy-phenylsulfanyl)-9H-purin-6-ylamine; 8-(2-Iodo-5-methoxy-phenylsulfanyl)-9-[2-(3-methyl-butylamino)-ethyl]-9H-purin-6-ylamine; 9-[2-(3,3-Dimethyl-butylamino)-ethyl]-8-(2-iodo-5-methoxy-phenylsulfanyl)-9H-purin-6-ylamine; {2-[6-Amino-8-(2-iodo-5-methoxy-phenylsulfanyl)-purin-9-yl]-ethylamino}-acetonitrile; 2-{2-[6-Amino-8-(2-iodo-5-methoxy-phenylsulfanyl)-purin-9-yl]-ethylamino}-ethanol; 8-(2-Iodo-5-methoxy-phenylsulfanyl)-9-[2-(2-methoxy-ethylamino)-ethyl]-9H-purin-6-ylamine; 9-(2-Cyclopentylamino-ethyl)-8-(2-iodo-5-methoxy-phenylsulfanyl)-9H-purin-6-ylamine; 9-(2-Cyclohexylamino-ethyl)-8-(2-iodo-5-methoxy-phenylsulfanyl)-9H-purin-6-ylamine; 9-(2-Cycloheptylamino-ethyl)-8-(2-iodo-5-methoxy-phenylsulfanyl)-9H-purin-6-ylamine; 9-(2-Cyclooctylamino-ethyl)-8-(2-iodo-5-methoxy-phenylsulfanyl)-9H-purin-6-ylamine; 9-[2-(Cyclopropylmethyl-amino)-ethyl]-8-(2-iodo-5-methoxy-phenylsulfanyl)-9H-purin-6-ylamine; 8-(2-Iodo-5-methoxy-phenylsulfanyl)-9-[2-(2-methyl-allylamino)-ethyl]-9H-purin-6-ylamine; 9-(2-tert-Butylamino-ethyl)-8-(2-iodo-5-methoxy-phenylsulfanyl)-9H-purin-6-ylamine; 9-(3-Amino-propyl)-8-(2-iodo-5-methoxy-phenylsulfanyl)-9H-purin-6-ylamine; 9-(2-Cyclopropylamino-ethyl)-8-(2-iodo-5-methoxy-phenylsulfanyl)-9H-purin-6-ylamine; 9-(2-Allylamino-ethyl)-8-(2-iodo-5-methoxy-phenylsulfanyl)-9H-purin-6-ylamine; 8-(2-Iodo-5-methoxy-phenylsulfanyl)-9-(2-morpholin-4-yl-ethyl)-9H-purin-6-ylamine; 8-(2-Iodo-5-methoxy-phenylsulfanyl)-9-(3-propylamino-propyl)-9H-purin-6-ylamine; 9-(3-Heptylamino-propyl)-8-(2-iodo-5-methoxy-phenylsulfanyl)-9H-purin-6-ylamine; 9-(3-Cyclopentylamino-propyl)-8-(2-iodo-5-methoxy-phenylsulfanyl)-9H-purin-6-ylamine; 9-(3-Cyclooctylamino-propyl)-8-(2-iodo-5-methoxy-phenylsulfanyl)-9H-purin-6-ylamine; 8-(2-Iodo-5-methoxy-phenylsulfanyl)-9-(3-isobutylamino-propyl)-9H-purin-6-ylamine; 8-(2-Iodo-5-methoxy-phenylsulfanyl)-9-[3-(1,2,2-trimethyl-propylamino)-propyl]-9H-purin-6-ylamine; 4-{3-[6-Amino-8-(2-iodo-5-methoxy-phenylsulfanyl)-purin-9-yl]-propylamino}-piperidine-1-carboxylic acid tert-butyl ester; 9-(2-Benzylamino-ethyl)-8-(2-iodo-5-methoxy-phenylsulfanyl)-9H-purin-6-ylamine; 9-[3-(1,1-Dimethyl-propylamino)-propyl]-8-(2-iodo-5-methoxy-phenylsulfanyl)-9H-purin-6-ylamine; 9-(3-Cyclobutylamino-propyl)-8-(2-Iodo-5-methoxy-phenylsulfanyl)-9H-purin-6-ylamine; 9-(3-Amino-propyl)-8-(2-iodo-5-methoxy-phenylsulfanyl)-9H-purin-6-ylamine; {2-[6-Amino-8-(2-iodo-5-methoxy-phenylsulfanyl)-purin-9-yl]-ethyl}-carbamic acid tert-butyl ester; 9-(2-Amino-ethyl)-8-(2-iodo-5-methoxy-phenylsulfanyl)-9H-purin-6-ylamine; 2-[6-Amino-8-(2-iodo-5-methoxy-phenylsulfanyl)-purin-9-yl]-acetamide; 1-[6-Amino-8-(2-iodo-5-methoxy-phenylsulfanyl)-purin-9-yl]-propan-2-one; N-{2-[6-Amino-8-(2-iodo-5-methoxy-phenylsulfanyl)-purin-9-yl]-ethyl}-acetamide; N-{2-[6-Amino-8-(2-iodo-5-methoxy-phenylsulfanyl)-purin-9-yl]-ethyl}-methanesulfonamide; N-{2-[6-Amino-8-(2-iodo-5-methoxy-phenylsulfanyl)-purin-9-yl]-ethyl}-N-isobutyl-acetamide; N-{2-[6-Amino-8-(2-iodo-5-methoxy-phenylsulfanyl)-purin-9-yl]-ethyl}-N-isobutyl-methanesulfonamide; 8-(3-Bromo-2,5-dimethoxy-phenylsulfanyl)-9-(4-methyl-pent-3-enyl)-9H-purin-6-ylamine; 8-(3-Bromo-2,5-dimethoxy-phenylsulfanyl)-9-pent-4-ynyl-9H-purin-6-ylamine; 8-(2,5-Dimethoxy-biphenyl-3-ylsulfanyl)-9-pent-4-ynyl-9H-purin-6-ylamine; 9-(4-Methyl-pent-3-enyl)-8-(thiazol-2-ylsulfanyl)-9H-purin-6-ylamine; 8-(Benzothiazol-2-ylsulfanyl)-9-(4-methyl-pent-3-enyl)-9H-purin-6-ylamine; 8-(1H-Benzoimidazol-2-ylsulfanyl)-9-(4-methyl-pent-3-enyl)-9H-purin-6-ylamine; Acetic acid 2-[6-amino-8-(naphthalen-2-ylsulfanyl)-purin-9-yl]-ethyl ester; Acetic acid 2-[6-amino-8-(naphthalen-1-ylsulfanyl)-purin-9-yl]-ethyl ester; Acetic acid 2-[6-amino-8-(quinolin-8-ylsulfanyl)-purin-9-yl]-ethyl ester; Acetic acid 2-[6-amino-8-(1H-indol-2-ylsulfanyl)-purin-9-yl]-ethyl ester; Acetic acid 2-[6-amino-8-(2,5-dimethoxy-phenylsulfanyl)-purin-9-yl]-ethyl ester; Acetic acid 2-[6-amino-8-(benzo[b]thiophen-2-ylsulfanyl)-purin-9-yl]-ethyl ester; 8-(Benzothiazol-2-ylsulfanyl)-9-pent-4-ynyl-9H-purin-6-ylamine; 9-Pent-4-ynyl-8-(quinolin-2-ylsulfanyl)-9H-purin-6-ylamine; 8-(1-Allyl-1H-benzoimidazol-2-ylsulfanyl)-9-(4-methyl-pent-3-enyl)-9H-purin-6-ylamine; 8-(1-Methyl-1H-benzoimidazol-2-ylsulfanyl)-9-(4-methyl-pent-3-enyl)-9H-purin-6-ylamine; 2-[6-Amino-8-(naphthalen-2-ylsulfanyl)-purin-9-yl]-ethanol; 2-[6-Amino-8-(naphthalen-1-ylsulfanyl)-purin-9-yl]-ethanol; 2-[6-Amino-8-(quinolin-8-ylsulfanyl)-purin-9-yl]-ethanol; Acetic acid 2-[6-amino-8-(3-chloro-1H-indol-2-ylsulfanyl)-purin-9-yl]-ethyl ester; Acetic acid 2-[6-amino-8-(3-bromo-1H-indol-2-ylsulfanyl)-purin-9-yl]-ethyl ester; Acetic acid 2-[6-amino-8-(3-iodo-1H-indol-2-ylsulfanyl)-purin-9-yl]-ethyl ester; Acetic acid 2-[6-amino-8-(1-propyl-1H-indol-2-ylsulfanyl)-purin-9-yl]-ethyl ester; Acetic acid 2-[6-amino-8-(3-iodo-1-propyl-1H-indol-2-ylsulfanyl)-purin-9-yl]-ethyl ester; Acetic acid 2-[6-amino-8-(1,4-dimethoxy-naphthalen-2-ylsulfanyl)-purin-9-yl]-ethyl ester; 3-[6-Amino-8-(benzo[1,3]dioxol-5-ylsulfanyl)-purin-9-yl]-propan-1-ol; 3-[6-Amino-8-(2,3-dihydro-benzo[1,4]dioxin-6-ylsulfanyl)-purin-9-yl]-propan-1-ol; 9-Butyl-8-(7-chloro-benzothiazol-2-ylsulfanyl)-9H-purine-6-ylamine; 9-ethyl-8-(7-chloro-benzothiazol-2-ylsulfanyl)-9H-purine-6-ylamine; 9-Propyl-8-(7-chloro-benzothiazol-2-ylsulfanyl)-9H-purine-6-ylamine; 9-Pentyl-8-(7-chloro-benzothiazol-2-ylsulfanyl)-9H-purine-6-ylamine; 8-(7-Bromo-benzothiazol-2-ylsulfanyl)-9-butyl-9H-purine-6-ylamine; 9-Butyl-8-(7-methyl-benzothiazol-2-ylsulfanyl)-9H-purine-6-ylamine; 9-Butyl-8-(7-methoxy-benzothiazol-2-ylsulfanyl)-9H-purine-6-ylamine; 9-Butyl-8-(7-ethoxy-benzothiazol-2-ylsulfanyl)-9H-purine-6-ylamine; 9-Butyl-8-(7-fluoro-benzothiazol-2-ylsulfanyl)-9H-purine-6-ylamine; 9-Butyl-8-(7-trifluoromethyl-benzothiazol-2-ylsulfanyl)-9H-purine-6-ylamine; 8-(Benzothiazol-2-ylsulfanyl)-9-butyl-9H-purine-6-ylamine; 9-Butyl-8-(6-chloro-benzothiazol-2-ylsulfanyl)-9H-purine-6-ylamine; 9-Butyl-8-(5-chloro-benzothiazol-2-ylsulfanyl)-9H-purine-6-ylamine; 9-Butyl-8-(4-chloro-benzothiazol-2-ylsulfanyl)-9H-purine-6-ylamine; 9-Butyl-8-(4-chloro-benzothiazol-2-ylsulfanyl)-9H-purine-6-ylamine; 8-(7-Chloro-benzothiazol-2-ylsulfanyl)-9-pent-4-ynyl-9H-purin-6-ylamine; 8-(7-Chloro-benzothiazol-2-ylsulfanyl)-9-(2-methoxy-ethyl)-9H-purin-6-ylamine; 8-(7-Chloro-benzothiazol-2-ylsulfanyl)-9-(2-vinyloxy-ethyl)-9H-purin-6-ylamine; 9-Butyl-8-(thiazolo[5,4-b]pyridin-2-ylsulfanyl)-9H-purine-6-ylamine; Acetic acid 4-[6-amino-8-(7-fluoro-benzothiazol-2-ylsulfanyl)-purin-9-yl]-butyl ester; 8-(4-Bromo-6,7-difluoro-benzothiazol-2-ylsulfanyl)-9-butyl-9H-purine-6-ylamine; 9-Butyl-8-(6,7-dichloro-benzothiazol-2-ylsulfanyl)-9H-purine-6-ylamine; 9-Butyl-8-(6,7-difluoro-benzothiazol-2-ylsulfanyl)-9H-purine-6-ylamine; 8-(6,7-Dichloro-benzothiazol-2-ylsulfanyl)-9-pent-4-ynyl-9H-purin-6-ylamine; Acetic acid 3-[6-amino-8-(6,7-dichloro-benzothiazol-2-ylsulfanyl)-purin-9-yl]-propyl ester; 9-But-3-enyl-8-(7-chloro-benzothoazol-2-ylsulfanyl)-9H-purine-6-ylamine; 8-(7-Chloro-benzothiazol-2-ylsulfanyl)-9-pent-4-ynyl-9H-purin-6-ylamine; 8-(7-Methoxy-benzothiazol-2-ylsulfanyl)-9-pent-4-ynyl-9H-purin-6-ylamine; 8-(7-Methyl-benzothiazol-2-ylsulfanyl)-9-pent-4-ynyl-9H-purin-6-ylamine; 9-Butyl-8-(7-methoxymethoxymethyl-benzothiazol-2-ylsulfanyl)-9H-purine-6-ylamine; Acetic acid 3-[6-amino-8-(7-methoxy-benzothiazol-2-ylsulfanyl)-purin-9-yl]-propyl ester; Acetic acid 3-[6-amino-8-(7-methyl-benzothiazol-2-ylsulfanyl)-purin-9-yl]-propyl ester; 8-(4-Amino-7-fluorol-benzothiazol-2-ylsulfanyl)-9-pent-4-ynyl-9H-purin-6-ylamine; 8-(7-Ethoxy-benzothiazol-2-ylsulfanyl)-9-pent-4-ynyl-9H-purin-6-ylamine; Acetic acid 2-[6-amino-8-(7-methoxy-benzothiazol-2-ylsulfanyl)-purin-9-yl]-ethyl ester; 8-(7-Chloro-benzothiazol-2-ylsulfanyl)-9-ethyl-9H-purine-6-ylamine; 2-Chloro-8-(7-chloro-benzothiazol-2-ylsulfanyl)-9-methyl-9H-purine-6-ylamine; 8-(7-Bromo-thiazolo[5,4-b]pyridin-2-ylsulfanyl)-9-butyl-9H-purine-6-ylamine; 8-(7-Bromo-thiazole[4,5-c]pyridin-2-ylsulfanyl)-2-chloro-9-methyl-9H-purine-6-ylamine; Acetic acid 2-[6-amino-8-(7-bromo-thiazole[4,5-c]pyridin-2-ylsulfanyl)-purin-9-yl]-ethyl ester; 8-(7-Bromo-thiazolo[5,4-b]pyridin-2-ylsulfanyl)-9-butyl-9H-purine-6-ylamine; Acetic acid 3-[6-amino-8-(7-bromo-thiazole[4,5-c]pyridin-2-ylsulfanyl)-purin-9-yl]-propyl ester; 8-(7-Bromo-thiazole[4,5-c]pyridin-2-ylsulfanyl)-9-(4-methyl-pent-3-enyl)-9H-purin-6-ylamine; Acetic acid 2-[6-amino-8-(7-bromo-thiazole[4,5-c]pyridin-2-ylsulfanyl)-purin-9-yl]-ethyl ester; 8-(7-Bromo-thiazolo[5,4-b]pyridin-2-ylsulfanyl)-2-chloro-9-methyl-9H-purine-6-ylamine; Acetic acid 3-[6-amino-8-(7-chloro-thiazolo[4,5-c]pyridin-2-ylsulfanyl)-purin-9-yl]-propyl ester; 9-Butyl-8-(7-chloro-benzooxazol-2-ylsulfanyl)-9H-purine-6-ylamine; Acetic acid 2-[6-amino-8-(7-chloro-benzooxazol-2-ylsulfanyl)-purin-9-yl]-ethyl ester; Acetic acid 3-[6-amino-8-(7-chloro-benzooxazol-2-ylsulfanyl)-purin-9-yl]-propyl ester; 9-Butyl-8-(7-fluoro-benzooxazol-2-ylsulfanyl)-9H-purine-6-ylamine; Acetic acid 2-[6-amino-8-(7-chloro-benzothiazol-2-ylsulfanyl)-purin-9-yl]-ethyl ester; Acetic acid 2-[6-amino-8-(7-fluoro-benzothiazol-2-ylsulfanyl)-purin-9-yl]-ethyl ester; Acetic acid 4-[6-amino-8-(7-chloro-benzothiazol-2-ylsulfanyl)-purin-9-yl]-butyl ester; Acetic acid 3-[6-amino-8-(7-fluoro-benzothiazol-2-ylsulfanyl)-purin-9-yl]-propyl ester; Acetic acid 3-[6-amino-8-(7-bromo-benzothiazol-2-ylsulfanyl)-purin-9-yl]-propyl ester; 2-[6-Amino-8-(7-chloro-benzothiazol-2-ylsulfanyl)-purin-9-yl]-ethanol; 2-[6-Amino-8-(7-fluoro-benzothiazol-2-ylsulfanyl)-purin-9-yl]-ethanol; 2-[6-Amino-8-(7-chloro-benzothiazol-2-ylsulfanyl)-purin-9-yl]-propan-1-ol; 4-[6-Amino-8-(7-chloro-benzothiazol-2-ylsulfanyl)-purin-9-yl]-butan-1-ol; 4-[6-amino-8-(7-fluoro-benzothiazol-2-ylsulfanyl)-purin-9-yl]-butan-1-ol; 3-[6-amino-8-(7-fluoro-benzothiazol-2-ylsulfanyl)-purin-9-yl]-propan-ol; 3-[6-Amino-8-(6,7-dichloro-benzothiazol-2-ylsulfanyl)-purin-9-yl]-propan-1-ol; 3-[6-Amino-8-(7-bromo-benzothiazol-2-ylsulfanyl)-purin-9-yl]-propan-ol; 3-[6-Amino-8-(7-methoxy-benzothiazol-2-ylsulfanyl)-purin-9-yl]-propan-ol; 2-[6-Amino-8-(7-methoxy-benzothiazol-2-ylsulfanyl)-purin-9-yl]-ethanol; 3-[6-Amino-8-(7-methyl-benzothiazol-2-ylsulfanyl)-purin-9-yl]-propan-ol; 2-[6-amino-8-(7-bromo-thiazolo[4,5-c]pyridin-2-ylsulfanyl)-purin-9-yl]-ethanol; 2-[6-Amino-8-(7-chloro-thiazolo[5,4-b]pyridin-2-ylsulfanyl)-purin-9-yl]-ethanol; 8-(7-Chloro-benzothiazol-2-ylsulfanyl)-9-[3-(1-ethyl-propylamino)-propyl]-9H-purin-6-yl amine; 9-(3-tert-Butylamino-propyl)-8-(7-chloro-benzothiazol-2-ylsulfanyl)-9H-purin-6-yl amine; 8-(7-Chloro-benzothiazol-2-ylsulfanyl)-9-(3-isobutylamino-propyl)-9H-purin-6-yl amine; 9-(3-sec-Butylamino-propyl)-8-(7-Chloro-benzothiazol-2-ylsulfanyl)-9H-purin-6-yl amine; 9-[2-(2,2-Dimethyl-propylamino)-ethyl]-8-(7-Chloro-benzothiazol-2-ylsulfanyl)-9H-purin-6-yl amine; 9-[2-Isopropylamino-ethyl]-8-(7-methoxy-benzothiazol-2-ylsulfanyl)-9H-purin-6-yl amine; 9-[2-tert-Butylamino-ethyl]-8-(7-methoxy-benzothiazol-2-ylsulfanyl)-9H-purin-6-yl amine; 9-(2-Isobutylamino-ethyl)-8-(7-methoxy-benzothiazol-2-ylsulfanyl)-9H-purin-6-yl amine; 8-(7-Chloro-benzothiazol-2-ylsulfanyl)-9-[3-(2,2-dimethyl-propylamino)-propyl]-9H-purin-6-yl amine; 8-(7-Chloro-benzothiazol-2-ylsulfanyl)-9-(2-prop-2-ynylamino-ethyl)-9H-purin-6-yl amine; 8-(7-Chloro-benzothiazol-2-ylsulfanyl)-9-(2-cyclopentylamino-ethyl)-9H-purin-6-yl amine; 8-(7-Chloro-benzothiazol-2-ylsulfanyl)-9-[2-(3-methyl-butylamino)-ethyl]-9H-purin-6-yl amine; 8-(7-Chloro-benzothiazol-2-ylsulfanyl)-9-[2-(1,1-dimethyl-propylamino)-ethyl]-9H-purin-6-yl amine; 9-(2-Allylamino-ethyl)-8-(7-chloro-benzothiazol-2-ylsulfanyl)-9H-purin-6-ylamine; 8-(7-Chloro-benzothiazol-2-ylsulfanyl)-9-(3-isopropylamino-propyl) 9H-purin-6-yl amine; 8-(7-Chlorol-benzothiazol-2-ylsulfanyl)-9-(3-pyrrol-1-yl-propyl)-9H-purine-6-ylamine; 8-(7-Chloro-benzothiazol-2-ylsulfanyl)-9-[2-(3,3-dimethyl-butylamino)-ethyl]-9H-purin-6-yl amine; 8-(7-Chloro-benzothiazol-2-ylsulfanyl)-9-(3-morpholin-4-yl-propyl)-9H-purin-6-ylamine; 8-(7-Chloro-benzothiazol-2-ylsulfanyl)-9-(2-morpholin-4-yl-ethyl)-9H-purin-6-ylamine; 9-(2-Bromo-ethyl)-8-(7-chloro-benzothiazol-2-ylsulfanyl)-9H-purine-6-ylamine; 8-(7-Fluoro-benzothiazol-2-ylsulfanyl)-9-(2-vinyloxy-ethyl)-9H-purin-6-ylamine; 8-(7-Fluoro-benzothiazol-2-ylsulfanyl)-9-pent-4-ynyl-9H-purin-6-ylamine; 8-(7-Chloro-benzothiazol-2-ylsulfanyl)-9-(2-chloro-ethyl)-9H-purine-6-ylamine; 9-(3-Bromo-propyl)-8-(7-chloro-benzothiazol-2-ylsulfanyl)-9H-purine-6-ylamine; 8-(7-Chloro-benzothoazol-2-ylsulfanyl)-9-pent-4-enyl-9H-purine-6-ylamine; 8-(7-Chloro-benzothoazol-2-ylsulfanyl)-9-hex-5-enyl-9H-purine-6-ylamine; 8-(7-Chloro-benzothiazol-2-ylsulfanyl)-9-[2,(2,5-dimethoxy-phenyl)-ethyl]-9H-purine-6-ylamine; 9-But-2-ynyl-8-(7-chloro-benzothiazol-2-ylsulfanyl)-9H-purine-6-ylamine; 8-(7-Chloro-benzothiazol-2-ylsulfanyl)-9-(3,4,4-trifluoro-but-3-enyl)-9H-purin-6-ylamine; 6-[6-Amino-8-(7-chloro-benzothiazol-2-ylsulfanyl)-purin-9-yl]-hexanenitrile; 8-(7-Chloro-benzothiazol-2-ylsulfanyl)-9-(3-methyl-but-3-enyl)-9H-purin-6-ylamine; 4-[6-Amino-8-(7-chloro-benzothiazol-2-ylsulfanyl)-purin-9-yl]-butyronitrile; 8-(7-Chloro-benzothiazol-2-ylsulfanyl)-9-hex-5-ynyl-9H-purin-6-ylamine; 8-(7-Chloro-benzothiazol-2-ylsulfanyl)-9-[3-(tetrahydro-furan-2-yl)-propyl]-9H-purin-6-ylamine; 8-(7-Chloro-benzothiazol-2-ylsulfanyl)-9-(tetrahydro-furan-2-ylmethyl)-9H-purin-6-ylamine; 8-(7-Chloro-benzothiazol-2-ylsulfanyl)-9-[2-(2-ethoxy-ethoxy)-ethyl]-9H-purin-6-ylamine; 5-[6-Amino-8-(7-chloro-benzothiazol-2-ylsulfanyl)-purin-9-yl]-pentanenitrile; 8-(7-Chlorol-benzothiazol-2-ylsulfanyl)-9-(4-methoxy-3,5-dimethyl-pyridin-2-ylmethyl)-9H-purine-6-ylamine; 8-(7-Chloro-benzothiazol-2-ylsulfanyl)-9-prop-2-ynyl-9H-purine-6-ylamine; 8-(7-Chloro-benzothiazol-2-ylsulfanyl)-9-(2-piperidin-1-yl-ethyl]-9H-purin-6-yl amine; 8-(7-Chloro-benzothiazol-2-ylsulfanyl)-9-(2-methylsulfanyl-ethyl)-9H-purin-6-ylamine; 8-(7-Chloro-benzothiazol-2-ylsulfanyl)-9-(2-ethylsulfanyl-ethyl)-9H-purin-6-ylamine; Phosphoric acid 3-[6-amino-8-(7-chloro-benzothiazol-2-ylsulfanyl)-9-yl]-propyl ester diethyl ester; Phosphoric acid 2-[6-amino-8-(7-chloro-benzothiazol-2-ylsulfanyl)-purin-9-yl]-ethyl bis-(2-chloro-ethyl)ester; {3-[6-Amino-8-(7-chloro-benzothiazol-2-ylsulfanyl)-purin-9-yl]-propenyl}-phosphonic acid diethyl ester; Phosphoric acid 2-[6-amino-8-(7-chloro-benzothiazol-2-ylsulfanyl)-9-yl]-ethyl ester diethyl ester; 8-(7-Chloro-benzothiazol-2-ylsulfanyl)-9-methyl-9H-purine-6-ylamine; 4-[6-Amino-8-(7-chloro-benzothiazol-2-ylsulfanyl)-purin-9-yl]-butan-2-one; 8-(7-Chloro-benzothiazol-2-ylsulfanyl)-9-(2-ethylsulfinyl-ethyl)-9H-purin-6-ylamine; 4-[6-Amino-8-(7-chloro-benzothiazol-2-ylsulfanyl)-purin-9-yl]-butan-2-thione; 8-(7-Chloro-benzothiazol-2-ylsulfanyl)-9-(2-ethanesulfonyl-ethyl)-9H-purin-6-ylamine; 8-(7-Chloro-benzothiazol-2-ylsulfanyl)-9-(2-methanesulfonyl-ethyl)-9H-purin-6-ylamine; (6-Amino-9-butyl-9H-purin-8-ylsulfanyl)-benzothiazol-7-yl]-methanol; 9-(2-Dimethylamino-ethyl)-8-(2-iodo-5-methoxy-phenylsulfanyl)-9H-purin-6-ylamine; 9-(2-Diethylamino-ethyl)-8-(2-iodo-5-methoxy-phenylsulfanyl)-9H-purin-6-ylamine; 8-(2-Iodo-5-methoxy-phenylsulfanyl)-9-(2-pyrrolidin-1-yl-ethyl)-9H-purin-6-ylamine; 8-(2-Iodo-5-methoxy-phenylsulfanyl)-9-(2-vinyloxy-ethyl)-9H-purin-6-ylamine; 8-(2-Iodo-5-methoxy-phenylsulfanyl)-9-(2-isopropoxy-ethyl)-9H-purin-6-ylamine; {3-[6-Amino-8-(2-iodo-5-methoxy-phenylsulfanyl)-purin-9-yl]-propyl}-methyl-carbamic acid tert-butyl ester; 8-(2-Iodo-5-methoxy-phenylsulfanyl)-9-(3-pyrrol-1-yl-propyl)-9H-purin-6-ylamine; (2,4-Diiodo-5-methoxy-phenylsulfanyl)-9-pent-4-ynyl-9H-purin-6-ylamine; {3-[6-Amino-8-(2-iodo-5-methoxy-phenylsulfanyl)-purin-9-yl]-propyl}-carbamic acid tert-butyl ester; 8-(2-Iodo-5-trifluoromethoxy-phenylsulfanyl)-9-pent-4-ynyl-9H-purin-6-ylamine and 8-(2-Iodo-5-trifluoromethyl-phenylsulfanyl)-9-pent-4-ynyl-9H-purin-6-ylamine.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 (a) represents levels of Hsp90 clients, Hsp70, and PI-3K p85 in murine A549 tumor xenografts following a single oral administration of 89.H₃PO₄ at 200 mg/kg, and (b) Levels of Hsp90 clients and PI-3K in murine N87 tumor xenografts 24 h after a three-day course of 17-AAG (intraperitoneally, 1×90 mg/kg/day) or 126.H₃PO₄ (orally, 2×200 or 2×100 mg/kg/day).

FIG. 2 represents tumor growth inhibition in murine N87 xenografts models induced by (a) inhibitors 109.H₃PO₄ and 126.H₃PO₄ delivered orally (1×200 mg/kg/day, 5 days/week) or (b) inhibitor 132.H₃PO₄ delivered orally (2×100 mg/kg/day, 5 days/week). Error bars=SEM

FIG. 3 represents a pharmacokinetic study of 264 delivered at 100 mg/kg via oral gavage

FIG. 4 represents a tumor growth inhibition study of 264 in the N87 xenograft model

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Definitions

A “pharmaceutically acceptable salt” may be prepared for any compound of the invention having a functionality capable of forming a salt, for example an acid or base functionality. Pharmaceutically acceptable salts may be derived from organic or inorganic acids and bases.

Compounds of the invention that contain one or more basic functional groups, e.g., amino or alkylamino, are capable of forming pharmaceutically acceptable salts with pharmaceutically acceptable organic and inorganic acids. These salts can be prepared in situ during the final isolation and purification of the compounds of the invention, or by separately reacting a purified compound of the invention in its free base form with a suitable organic or inorganic acid, and isolating the salt thus formed. Examples of suitable acids include hydrochloric, hydrobromic, sulfuric, nitric, perchloric, fumaric, maleic, phosphoric, glycolic, gluconic, lactic, salicylic, succinic, toluene-p-sulfonic, tartaric, acetic, citric, methanesulfonic, formic, benzoic, malonic, naphthalene-2-sulfonic, benzenesulfonic, 1,2 ethanesulfonic acid (edisylate), galactosyl-d-gluconic acid, and the like. Other acids, such as oxalic acid, while not themselves pharmaceutically acceptable, may be employed in the preparation of salts useful as intermediates in obtaining the compounds of this invention and their pharmaceutically acceptable acid addition salts. See, e.g., Berge et al. “Pharmaceutical Salts”, J. Pharm. Sci. 66:1-19 (1977).

Compounds of the present invention that contain one or more acidic functional groups are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Illustrative examples of some of the bases that can be used include sodium hydroxide, potassium hydroxide, choline hydroxide, sodium carbonate, and the like. Representative organic amines useful for the formation of base addition salts include ethylamine, diethylamine, ethylenediamine, ethanolamine, diethanolamine, piperazine and the like. See, for example, Berge et al., supra.

“Prodrugs” are derivative compounds derivatized by the addition of a group that endows greater solubility to the compound desired to be delivered. Once in the body, the prodrug is typically acted upon by an enzyme, e.g., an esterase, amidase, or phosphatase, to generate the active compound. Suitable positions for derivatization of the compounds of the invention to create “prodrugs” include but are not limited to the Y group, the phenyl ring of the purines, and the Q group. Those of ordinary skill in the art have the knowledge and means to accomplish this without undue experimentation. Examples of prodrugs of contemplated by the present application, without limitation, include:

Alcohols prodrugs,

Drug-OH,

Drug-OX, X=prodrug moiety, 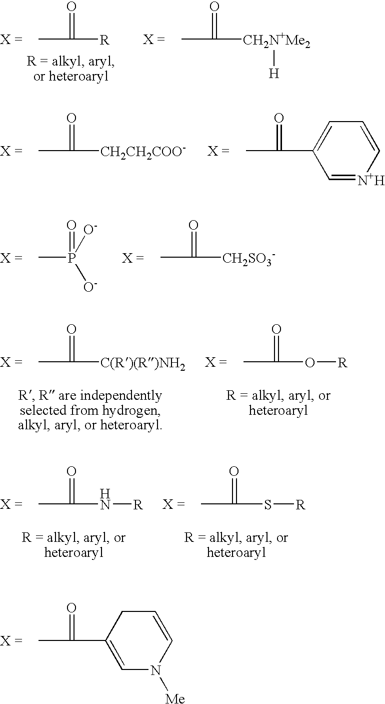
Amine prodrugs
Drug-NHX 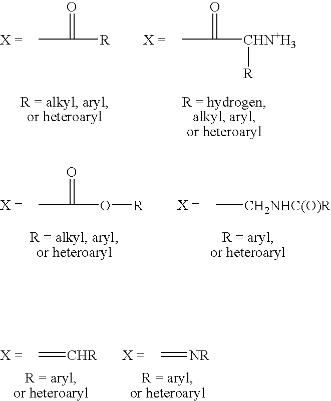
Carboxylic prodrugs
Drug-COOX
X=alkyl, aryl or heteroaryl

“Tautomers” are compounds whose structures differ in arrangements of atoms, but which exist in equilibrium. By way of example, the structure shown below and designated T is in equilibrium with a second tautomeric form designated T′.

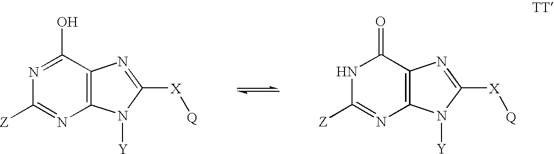

The predominance of one tautomer versus another is controlled by factors which include but are not limited to the nature of the solvent, temperature, pressure, the presence or absence of other molecules, and the nature of substituents on the molecule having tautomeric forms.

The term “alkyl,” alone or in combination, refers to an optionally substituted straight-chain, optionally substituted branched-chain, or optionally substituted cyclic alkyl radical having from 1 to about 30 carbons, more preferably 1 to 12 carbons. Examples of alkyl radicals include methyl, ethyl, n-propyl, isopropyl, n-butyl, isobutyl, sec-butyl, tert-butyl, tert-amyl, pentyl, hexyl, heptyl, octyl and the like. The term “cycloalkyl” embraces cyclic configurations, is subsumed within the definition of alkyl and specifically refers to a monocyclic, bicyclic, tricyclic, and higher multicyclic alkyl radicals wherein each cyclic moiety has from 3 to about 8 carbon atoms. Examples of cycloalkyl radicals include cyclopropyl, cyclobutyl, cyclopentyl, cyclohexyl and the like. A “lower alkyl” is a shorter alkyl, e.g., one containing from 1 to about 6 carbon atoms.

The term “alkenyl,” alone or in combination, refers to an optionally substituted straight-chain, optionally substituted branched-chain, or optionally substituted cyclic alkenyl hydrocarbon radical having one or more carbon-carbon double-bonds and having from 2 to about 30 carbon atoms, more preferably 2 to about 18 carbons. Examples of alkenyl radicals include ethenyl, propenyl, butenyl, 1,4-butadienyl and the like. The term can also embrace cyclic alkenyl structures. A “lower alkenyl” refers to an alkenyl having from 2 to about 6 carbons.

The term “alkynyl,” alone or in combination, refers to an optionally substituted straight-chain, optionally substituted branched-chain, or cyclic alkynyl hydrocarbon radical having one or more carbon-carbon triple-bonds and having from 2 to about 30 carbon atoms, more preferably 2 to about 12 carbon atoms. The term also includes optionally substituted straight-chain or optionally substituted branched-chain hydrocarbon radicals having one or more carbon-carbon triple bonds and having from 2 to about 6 carbon atoms as well as those having from 2 to about 4 carbon atoms. Examples of alkynyl radicals include ethynyl, propynyl, butynyl and the like.

The terms heteroalkyl, heteroalkenyl and heteroalkynyl include optionally substituted alkyl, alkenyl and alkynyl structures, as described above, and which have one or more skeletal chain atoms selected from an atom other that carbon, e.g., oxygen, nitrogen, sulfur, phosphorous or combinations thereof.

The term “carbon chain” may embrace any alkyl, alkenyl, alkynyl, or heteroalkyl, heteroalkenyl, or heteroalkynyl group, and may be linear, cyclic, or any combination thereof. If part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining chain length. If the chain contains heteroatoms as part of the backbone, those atoms are not calculated as part of the carbon chain length.

The term “alkoxy,” alone or in combination, refers to an alkyl ether radical, alkyl-O—, wherein the term alkyl is defined as above. Examples of alkoxy radicals include methoxy, ethoxy, n-propoxy, isopropoxy, n-butoxy, iso-butoxy, sec-butoxy, tert-butoxy and the like.

The term “aryloxy,” alone or in combination, refers to an aryl ether radical wherein the term aryl is defined as below. Examples of aryloxy radicals include phenoxy, benzyloxy and the like.

The term “alkylthio,” alone or in combination, refers to an alkyl thio radical, alkyl-S—, wherein the term alkyl is defined as above.

The term “arylthio,” alone or in combination, refers to an aryl thio radical, aryl-S—, wherein the term aryl is defined as below.

The term “oxo” refers to ═O.

The term “aryl,” alone or in combination, refers to an optionally substituted aromatic ring system. The term aryl includes monocyclic aromatic rings, polyaromatic rings and polycyclic aromatic ring systems containing from six to about twenty carbon atoms. The term aryl also includes monocyclic aromatic rings, polyaromatic rings and polycyclic ring systems containing from 6 to about 12 carbon atoms, as well as those containing from 6 to about 10 carbon atoms. The polyaromatic and polycyclic aromatic rings systems may contain from two to four rings. Examples of aryl groups include, without limitation, phenyl, biphenyl, naphthyl and anthryl ring systems.

The term “heteroaryl” refers to optionally substituted aromatic ring systems containing from about five to about 20 skeletal ring atoms and having one or more heteroatoms such as, for example, oxygen, nitrogen, sulfur, and phosphorus. The term heteroaryl also includes optionally substituted aromatic ring systems having from 5 to about 12 skeletal ring atoms, as well as those having from 5 to about 10 skeletal ring atoms. The term heteroaryl may include five- or six-membered heterocyclic rings, polycyclic heteroaromatic ring systems and polyheteroaromatic ring systems where the ring system has two, three or four rings. The terms heterocyclic, polycyclic heteroaromatic and polyheteroaromatic include ring systems containing optionally substituted heteroaromatic rings having more than one heteroatom as described above (e.g., a six membered ring with two nitrogens), including polyheterocyclic ring systems of from two to four rings. The term heteroaryl includes ring systems such as, for example, furanyl, benzofuranyl, chromenyl, pyridyl, pyrrolyl, indolyl, quinolinyl, N-alkyl pyrrolyl, pyridyl-N-oxide, pyrimidoyl, pyrazinyl, imidazolyl, pyrazolyl, oxazolyl, benzothiophenyl, purinyl, indolizinyl, thienyl and the like.

The term “heteroarylalkyl” refers to a C₁-C₄ alkyl group containing a heteroaryl group, each of which may be optionally substituted.

The term “heteroarylthio” refers to the group —S-heteroaryl.

The term “acyloxy” refers to the ester group —OC(O)—R, where R is H, alkyl, alkenyl, alkynyl, aryl, or arylalkyl, wherein the alkyl, alkenyl, alkynyl and arylalkyl groups may be optionally substituted.

The term “carboxy esters” refers to —C(O)OR where R is alkyl, aryl or arylalkyl, wherein the alkyl, aryl and arylalkyl groups may be optionally substituted.

The term “carboxamido” refers to

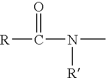

where each of R and R′ are independently selected from the group consisting of H, alkyl, aryl and arylalkyl, wherein the alkyl, aryl and arylalkyl groups may be optionally substituted.

The term “arylalkyl,” alone or in combination, refers to an alkyl radical as defined above in which one H atom is replaced by an aryl radical as defined above, such as, for example, benzyl, 2-phenylethyl and the like.

The term “alkylaryl,” alone or in combination, refers to an aryl radical as defined above in which one H atom is replaced by an alkyl radical as defined above, such as, for example, tolyl, xylyl and the like.

The terms haloalkyl, haloalkenyl, haloalkynyl and haloalkoxy include alkyl, alkenyl, alkynyl and alkoxy structures, as described above, that are substituted with one or more fluorines, chlorines, bromines or iodines, or with combinations thereof.

The terms cycloalkyl, aryl, arylalkyl, heteroaryl, alkyl, alkynyl, alkenyl, haloalkyl and heteroalkyl include optionally substituted cycloalkyl, aryl, arylalkyl, heteroaryl, alkyl, alkynyl, alkenyl, haloalkyl and heteroalkyl groups.

The term “carbocycle” includes optionally substituted, saturated or unsaturated, three- to eight-membered cyclic structures in which all of the skeletal atoms are carbon.

The term “heterocycle” includes optionally substituted, saturated or unsaturated, three- to eight-membered cyclic structures in which one or more skeletal atoms is oxygen, nitrogen, sulfur, phosphorus or combinations thereof. Illustrative examples include pyridine, pyran, thiophan, pyrrole, furan, thiophen, pentatomic and hexatomic lactam rings, and the like.

The term “membered ring” can embrace any cyclic structure, including carbocycles and heterocycles as described above. The term “membered” is meant to denote the number of skeletal atoms that constitute the ring. Thus, for example, pyridine, pyran, and thiophan are 6 membered rings and pyrrole, furan, and thiophen are 5 membered rings.

The term “acyl” includes alkyl, aryl, heteroaryl, arylalkyl or heteroarylalkyl substituents attached to a compound via a carbonyl functionality (e.g., —CO-alkyl, —CO-aryl, —CO-arylalkyl or —CO-heteroarylalkyl, etc.).

“Optionally substituted” groups may be substituted or unsubstituted. The substituents of an “optionally substituted” group may include, without limitation, one or more substituents independently selected from the following groups or designated subsets thereof: alkyl, alkenyl, alkynyl, heteroalkyl, haloalkyl, haloalkenyl, haloalkynyl, cycloalkyl, aryl, heteroaryl, arylalkyl, heteroarylalkyl, alkoxy, aryloxy, haloalkoxy, amino, alkylamino, dialkylamino, alkylthio, arylthio, heteroarylthio, oxo, carboxyesters (C(O)OR^{y}), carboxamido (C(O)NR^{y}₂), acyloxy, H; halo, CN, NO₂, N₃, OH, C(O)R^{y}, pyridinyl, thiophenyl, furanyl, indolyl, indazolyl, phosphonates (—P(O)(OR^{y})₂), phosphates (—O—P(O)(OR^{y})₂), phosphoramides (—NR^{x}—P(O)(OR^{y})₂), sulfonates (—S(O)₂—O—), sulfates (—O—S(O)₂—O—R^{y}), sulfonamides (—NR^{x}—S(O)₂—O—R^{y}), carbamates (—NH—C(O)—O—R^{y}), ureayl, thioureyl, thioamidyl, thioalkyl. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The term “halogen” includes F, Cl, Br and I.

The term sulfide refers to a sulfur atom covalently linked to two atoms; the formal oxidation state of said sulfur is (II). The term “thioether” may used interchangebly with the term “sulfide”.

The term “sulfoxide” refers to a sulfur atom covalently linked to three atoms, at least one of which is an oxygen atom; the formal oxidation state of said sulfur atom is (IV).

The term “sulfone” refers to a sulfur atom covalently linked to four atoms, at least two of which are oxygen atoms; the formal oxidation state of said sulfur atom is (VI).

Some of the compounds of the present invention may contain one or more chiral centers and therefore may exist in enantiomeric and diastereomeric forms. The scope of the present invention is intended to cover all isomers per se, as well as mixtures of cis and trans isomers, mixtures of diastereomers and racemic mixtures of enantiomers (optical isomers) as well. Further, it is possible using well known techniques to separate the various forms, and some embodiments of the invention may feature purified or enriched species of a given enantiomer or diasteriomer.

A “pharmacological composition” refers to a mixture of one or more of the compounds described herein, or pharmaceutically acceptable salts thereof, with other chemical components, such as pharmaceutically acceptable carriers and/or excipients. The purpose of a pharmacological composition is to facilitate administration of a compound to an organism.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. A physiologically acceptable carrier should not cause significant irritation to an organism and does not abrogate the biological activity and properties of the administered compound.

An “excipient” refers to an inert substance added to a pharmacological composition to further facilitate administration of a compound. Examples of excipients include but are not limited to calcium carbonate, calcium phosphate, various sugars and types of starch, cellulose derivatives, gelatin, vegetable oils and polyethylene glycols.

Assays to Determine HSP90 Binding and Downstream Effect

A variety of in vitro and in vivo assays are available to test the effect of the compounds of the invention on HSP90. HSP90 competitive binding assays and functional assays can be performed as known in the art substituting in the compounds of the invention. Chiosis et al., Chemistry & Biology 8:289-299 (2001), describe some of the known ways in which this can be done. For example, competition binding assays using, e.g., geldanamycin or 17-AAG as a competitive binding inhibitor of HSP90 can be used to determine relative HSP90 affinity of the compounds of the invention by immobilizing the compound of interest or other competitive inhibitor on a gel or solid matrix, preincubating HSP90 with the other inhibitor, passing the preincubated mix over the gel or matrix, and then measuring the amount of HSP90 that sticks or does not stick to the gel or matrix.

Downstream effects can also be evaluated based on the known effect of HSP90 inhibition on function and stability of various steroid receptors and signaling proteins including, e.g., Raf1 and Her2. Compounds of the present invention induce dose-dependent degradation of these molecules, which can be measured using standard techniques. Inhibition of HSP90 also results in up-regulation of HSP90 and related chaperone proteins that can similarly be measured. Antiproliferative activity on various cancer cell lines can also be measured, as can morphological and functional differentiation related to HSP90 inhibition. For example, the

Many different types of methods are known in the art for determining protein concentrations and measuring or predicting the level of proteins within cells and in fluid samples. Indirect techniques include nucleic acid hybridization and amplification using, e.g., polymerase chain reaction (PCR). These techniques are known to the person of skill and are discussed, e.g., in Sambrook, Fritsch & Maniatis, Molecular Cloning: A Laboratory Manual, Second Edition (1989) Cold Spring Harbor Laboratory Press, Cold Spring Harbor, N.Y., Ausubel, et al., Current Protocols in Molecular Biology, John Wiley & Sons, NY, 1994, and, as specifically applied to the quantification, detection, and relative activity of Her-2/neu in patient samples, e.g., in U.S. Pat. Nos. 4,699,877, 4,918,162, 4,968,603, and 5,846,749. A brief discussion of two generic techniques that can be used follows.

The determination of whether cells overexpress or contain elevated levels of HER-2 can be determined using well known antibody techniques such as immunoblotting, radioimmunoassays, western blotting, immunoprecipitation, enzyme-linked immunosorbant assays (ELISA), and derivative techniques that make use of antibodies directed against HER-2. As an example, HER-2 expression in breast cancer cells can be determined with the use of an immunohistochemical assay, such as the Dako Hercep™ test (Dako Corp., Carpinteria, Calif.). The Hercep™ test is an antibody staining assay designed to detect HER-2 overexpression in tumor tissue specimens. This particular assay grades HER-2 expression into four levels: 0, 1, 2, and 3, with level 3 representing the highest level of HER-2 expression. Accurate quantitation can be enhanced by employing an Automated Cellular Imaging System (ACIS) as described, e.g., by Press, M, et al, (2000), Modern Pathology 13:225A.

Antibodies, polyclonal or monoclonal, can be purchased from a variety of commercial suppliers, or may be manufactured using well-known methods, e.g., as described in Harlow et al., Antibodies: A Laboratory Manual, 2nd Ed; Cold Spring Harbor Laboratory Press, Cold Spring Harbor, N.Y. (1988).

HER-2 overexpression can also be determined at the nucleic acid level since there is a reported high correlation between overexpression of the HER-2 protein and amplification of the gene that codes for it. One way to test this is by using RT-PCR. The genomic and cDNA sequences for HER-2 are known. Specific DNA primers can be generated using standard, well-known techniques, and can then be used to amplify template already present in the cell. An example of this is described in Kurokawa, H et al, Cancer Res. 60: 5887-5894 (2000). PCR can be standardized such that quantitative differences are observed as between normal and abnormal cells, e.g., cancerous and noncancerous cells. Well known methods employing, e.g., densitometry, can be used to quantitate and/or compare nucleic acid levels amplified using PCR.

Similarly, fluorescent in situ hybridization (FISH) assays and other assays can be used, e.g., Northern and/or Southern blotting. These rely on nucleic acid hybridization between the HER-2 gene or mRNA and a corresponding nucleic acid probe that can be designed in the same or a similar way as for PCR primers, above. See, e.g., Mitchell M S, and Press M F., 1999, Semin. Oncol., Suppl. 12:108-16. For FISH, this nucleic acid probe can be conjugated to a fluorescent molecule, e.g., fluorescein and/or rhodamine, that preferably does not interfere with hybridization, and which fluorescence can later be measured following hybridization. See, e.g., Kurokawa, H et al, Cancer Res. 60: 5887-5894 (2000) (describing a specific nucleic acid probe having sequence 5′-FAM-NucleicAcid-TAMRA-p-3′ sequence). ACIS-based approaches as described above can be employed to make the assay more quantitative (de la Torre-Bueno, J, et al, 2000, Modern Pathology 13:221A).

Immuno and nucleic acid detection can also be directed against proteins other than HSP90 and Her-2, which proteins are nevertheless affected in response to HSP90 inhibition.

### Synthesis of the Compounds of the Invention

The following synthetic schemes are applicable to various compounds, compositions, methods, and formulations of the invention:

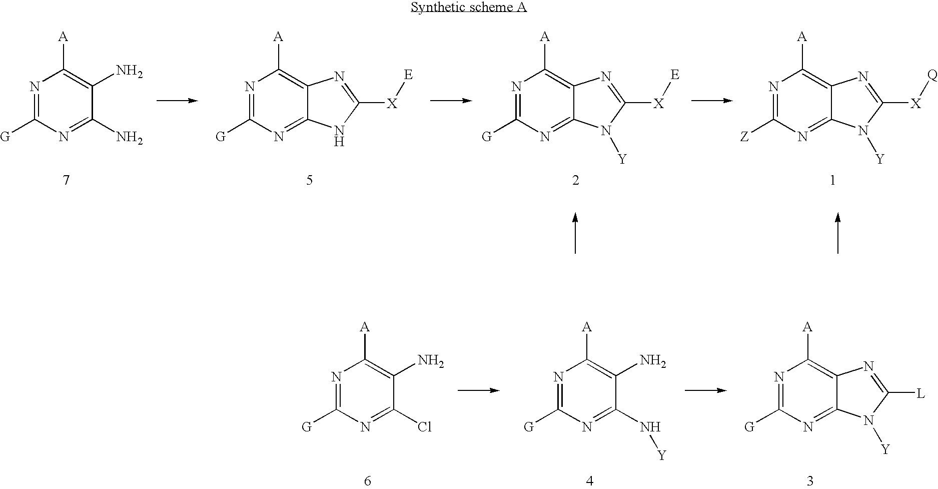

### Synthesis of Compounds of Formula 1 [X═C]

Synthesis of compounds of formula 1 (when X═C) in synthetic scheme A may include some or all of the following general steps. The 8-substituted purine analogs of formula 5 or 2 can be prepared from 4,5-diaminopyrimidines and the carboxylates or their derivatives, such as amides, esters, nitriles, orthoesters, imidates etc (see, e.g., Townsend Chemistry of Nucleosides and Nucleotides, Vol. 1; Plenum Press, New York and London, page 148-158; Tetrahedron Lett. 36, 4249, 1995). Substituted 4,5-diaminopyrimidines can be obtained commercially or from substituted 2-chloro-3-amino pyrimidine or 2-chloro-3-nitropyrimidines as known in the art. See, e.g., Tetrahedron, 40, 1433 (1984); J. Am. Chem. Soc., 118, 135 (1975); Synthesis 135 (1975); J. Med. Chem. 39, 4099 (1996).

Compounds of formula 5 can be converted to compounds of formula 2 by simple alkylation with alkylhalides, alkyltosylates, mesolates or triflates in polar solvents like THF, DMF or DMSO using bases like NaH, Cs₂CO₃ or K₂CO₃, or by the well-known Mitsunobu alkylation method.

Compounds of formula 2 can be further modified to give compounds of formula 1 or the intermediates to prepare compounds of formula 1, e.g., substitution of 6-chloropurine by ammonia or alkylamines. C-2 substitution of purines, e.g., halogenation with F, Cl or Br can be introduced via 2-aminopurines as described by Eaton et al., J. Org. Chem. 34(3), 747-8 (1969) or by nucleophilic substitution as described, e.g., in. J. Med. Chem. 36, 2938 (1993) and Heterocycles, 30, 435, (1990). These C-2 substitutions also can be introduced via metalation as described, e.g., in J. Org. Chem. 62(20), 6833 (1997), followed by addition of desired electrophile. General purine substitution can be accomplished as described in J. Med. Chem. 42, 2064 (1999).

Alternatively, intermediates of formula 2 can be prepared from chloroaminopyrimidines such as formula 6 by the following two steps: (1) treatment of the compounds of formula 6 with corresponding amine (Y—NH₂), e.g., butylamine, in presence of base such as triethyl amine or N,N-diisopropyl amine in polar solvents such as n-BuOH to give the substituted diamine compounds of formula 4; (2) treatment of the compounds of formula 4 using the same methods as described earlier going from formula 7 to formula 5. Similar methods as described earlier can be used to introduce the C-2 substitution (point at which Z or G moiety attaches).

Compounds of formula 1 where A is other than NH₂, e.g., halogen, methoxy, alkyl, or trifluoro alkyl, can be prepared starting with the corresponding substituent in place (if it can withstand the transformations), or, for halogen or substituted amines, these can be prepared from the 6-amine.

The compounds of formula I can also be prepared from formula 3, where L is halogen, using Negishi-type couplings (e.g., as described in J. Org. Chem. 2001, 66, 7522; J. Org. Chem. 1991, 56, 1445).

### Synthesis of Compounds of Formula 1 [X=Heteroatom e.g. S, O, N]

Compounds of formula 1 wherein X is a heteroatom such as S, O or N can be prepared by scheme B. In general, these compounds are linked via their C-8 to one of the heteroatoms X═S, O, or N and can be prepared from the corresponding 8-halo (e.g., bromo, iodo or chloro) compounds such as formula 10 using nucleophiles such as sulfides, alkyl or arylthiols, amines, azides, and alcohols.

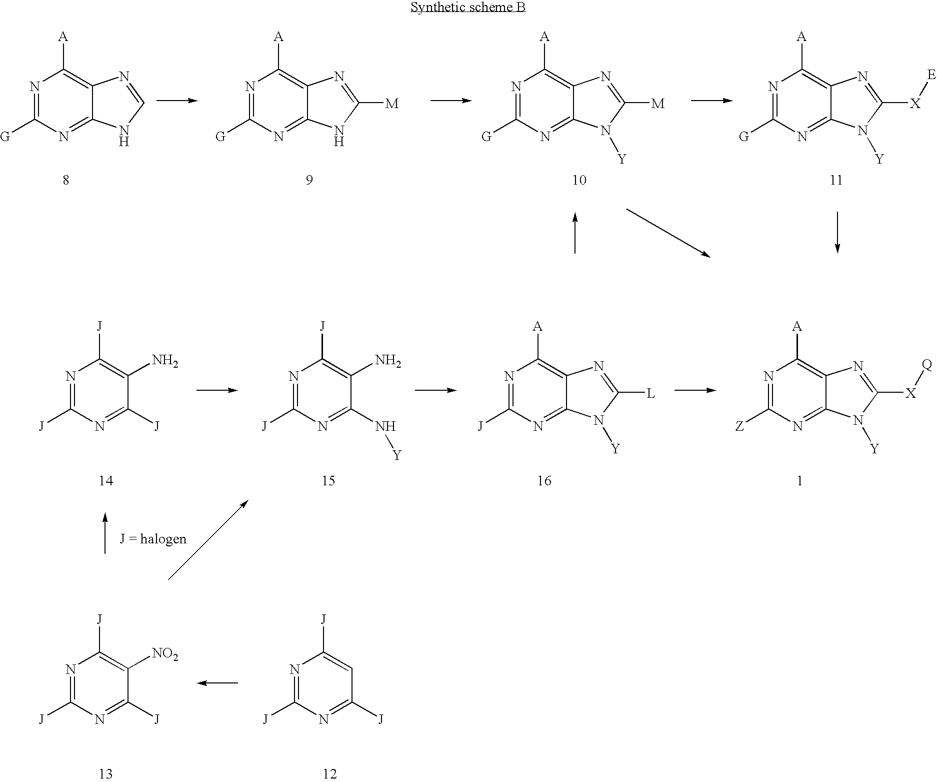

With reference to scheme B, substituted adenines or purines of formula 8 can be treated with halogenating agents such as bromine or iodine, followed by alkylation at N-9 to give compounds of formula 10, wherein M is halogen such as bromine or iodine (Dang et.al. PCT, WO 98/39344). Compounds of formula 16 can be prepared from trihalopyrimidines such as those of formula 12 by nitration to give compounds of formula 13. Subsequent displacement of the halogen with amine (YNH₂) and reduction of the nitrogroup gives the diamines of formula 15. Alternatively, reduction of the nitrogroup may precede halogen displacement. Diamines of formula 15 can be readily cyclized to the imidazole ring of the compounds of formula 16, wherein L is H, SH, OH or NH₂ (Org. Syn. Collective Vol. 2, 65; Org. Syn. Collective Vol. 4, 569). The compounds of formula 1 can also be synthesized from the compounds of formula 16, wherein L is SH, OH, or NH₂, by reacting with aromatic halides, boronic acids, triflates, or their equivalents in presence of a catalyst such as palladium or copper (Buchwald, S. L. et. al. J. Am. Chem. Soc., 1998, 120, 213-214; Buchwald, S. L. et. al. Acc. Chem. Res. 1998, 31, 805; Buchwald, S. L. et. al Org. Lett., 2002, 4, 3517-3520).

Alternately, compounds of formulae 1 and 11 (wherein X═S or O) can be synthesized by coupling of the diazonium salts of the compounds of formulae 10 or 16 (wherein M or L is N₂.BF₄, N₂.HCl, N₂.H₂SO₄ etc.) with HXE or HXQ (wherein X═S or O) in the presence of base such as t-BuOK, NaH, etc. in solvents such as DMF, MeOH, etc.

Z-groups of formula 1 can be introduced by modifying existing 2-substituents such as G. For example, 2-halopurines of formula 1 can be prepared from 2-aminopurines (G=NH₂) via chemistry well described in the literature. Other substitutions such as S-alkyl or aryl, O-alkyl can be made from nucleophilic substitution reactions; metal-catalysed reactions, etc. (see, e.g., Aerschot et. al., J. Med. Chem. 36:2938 (1993); Buchwald, S. L. et. al., Heterocycles, 30: 435 (1990).

The E component (aromatic or heteroaromatic or alkyl) of the compounds of formula 11 can be further modified as needed using well known procedures including, e.g., nucleophilic additions, electrophilic additions, halogenations, etc. to give Q (see, e.g., Advanced Organic Chemistry, March. J. Wiley Interscience).

Compounds of formula 1, wherein X is S(O) or S(O)₂ can be prepared by the oxidation of the compounds of formula 1, wherein X═S, using reagents such as MCPBA, H₂O₂, NaIO₄, Oxone, etc. in solvents such as CHCl₃, CH₂Cl₂ etc. Also, these sulfone compounds can be made by coupling of sulfonyl salts such as Li, Na, K (ArS(O)₂Li) and compounds of formulae 10 or 16 (wherein M or L is halogen such as Br or I) in polar solvents such as DMF. (Chem. Abstr. 1952, 4549). With controlled reduction of these sulfones, one can make compounds of formula 1 where X is S(O) and S(O)₂.

### Synthesis of 8-(sulfanyl)adenines from 8-haloadenines

8-Haloadenines can be coupled to thiophenols under basic conditions. A wide array of bases is available, as for instance, LiOH, NaOH, t-BuONa, K₂CO₃, KOH, t-BuOK, Cs₂CO₃, or CsOH. The thiophenol can already carry all the substituents necessary for biological activity or can be modified after coupling (Scheme C).

Preparation of Thiophenol, then Coupling

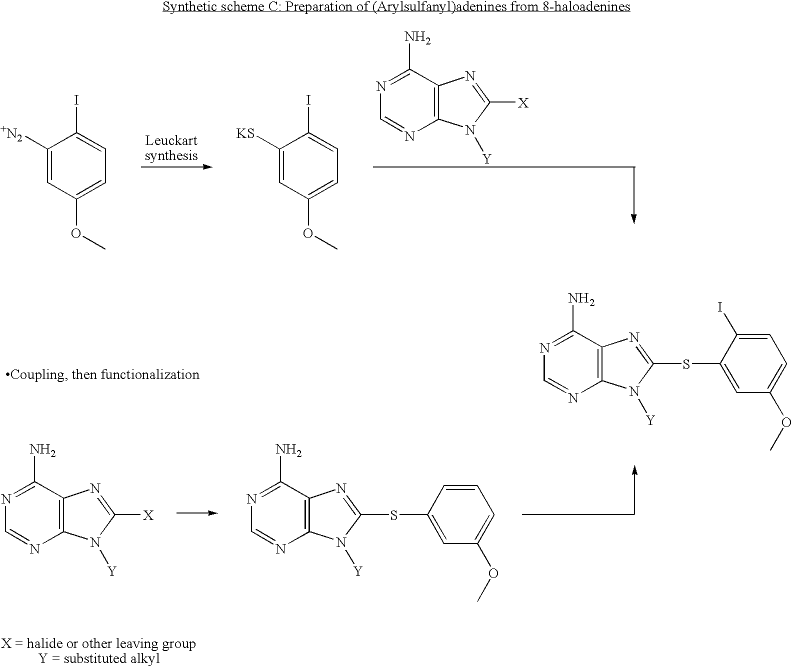

Following the first route, the thiophenol is first prepared using one of the many known methods. These methods have been extensively reviewed (Wardell, J. L. Preparation of Thiols. In The Chemistry of the Thio Group, Part 1. Patai S. Ed. John Wiley & Sons. London, 1974, pp 163-263.). The most popular of them is perhaps the Leuckart synthesis, in which an aryl diazonium salt is treated with a sulfur nucleophile, typically EtOCS₂K, to give a xanthate which is hydrolyzed with a base. For instance, the 2-iodo-5-(methoxy)benzenethiophenol indicated scheme C was prepared in this manner. (Ma, C. J. Org. Chem. 2001, 66, 4525. Flynn, B. L. Org. Lett. 2001, 3, 651). The thiophenol is then deprotonated (e.g. K₂CO₃) and coupled to a 8-haloadenine. The coupling can be catalyzed by transition metals, e.g. Ni(acac)₂.

The second route of scheme C entails coupling of 8-haloadenine to a thiophenol, and subsequent treatment with an electrophilic species (Cl⁺, Br⁺, I⁺, NO⁺ etc.) using standard reagents for electrophilic aromatic substitutions.

### Synthesis of 8-(sulfanyl)adenines from 8-mercaptoadenines

8-(Arylsulfanyl)adenines can be prepared from 8-mercaptoadenines with electrophilic species, as illustrated in scheme D. The mercaptoadenine is reacted with a diazonium salt, in a polar solvent such as DMF or DMSO, in the presence or absence of base (Biamonte, M. A., J. Org. Chem., 2005, 70, 717), or a radical cation is generated with PhI(OCOCF₃)₂ and is trapped with a 8-mercaptoadenine (Kita, Y., J. Org. Chem., 1995, 60, 7144).

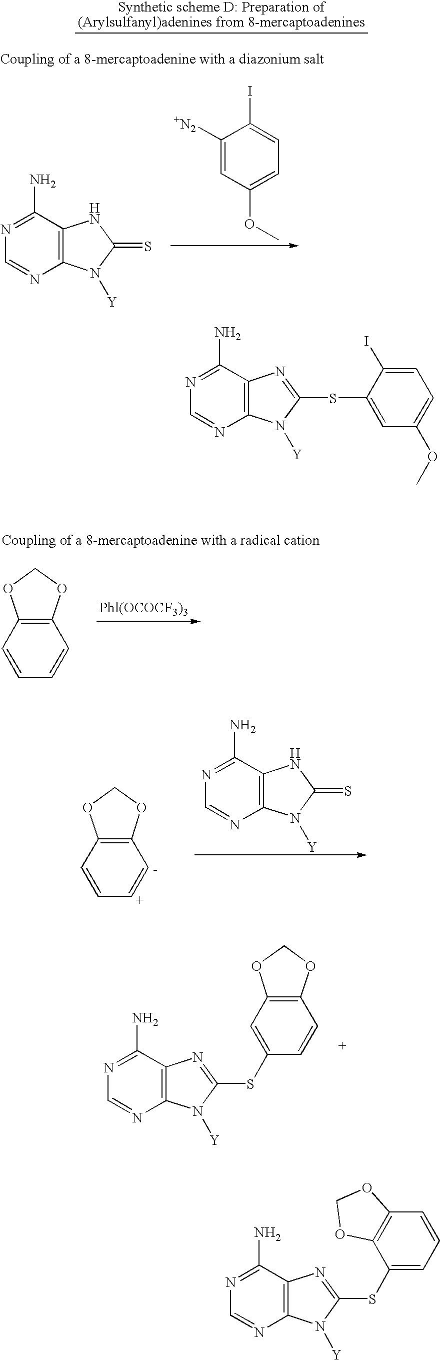

### Synthesis of Benzothi(ox)azolopurines or Pyridothiazolopurines

These compounds can be prepared from the intermediate 10, in scheme B. Compounds of formula 3, wherein L is halogen, Y is H or a substituent that can be modified if necessary (for example (CH₂)ₙO(CO)CH₃, n=2-4) G is H or halogen, A is NH₂ can be treated with substituted benzothi(ox)azole-2-thiol or pyridothi(ox)azole-2-thiol in presence of base for example t-BuOK, NaH, or K₂CO₃ in polar solvents such as DMF, THF or DMSO to give the formula 17, wherein T is ‘O’ or ‘S’, V is ‘C’ or ‘N’, R is a substituent such as halogen, alkyl, aryl, alkoxy, CN etc., scheme E.

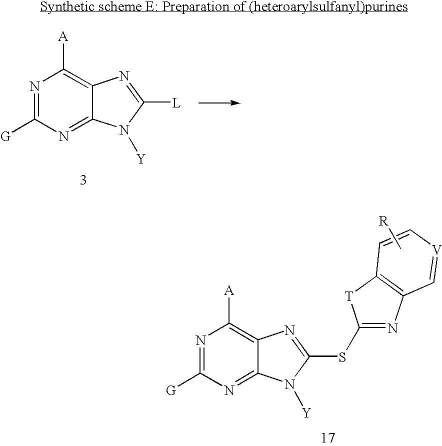

Substituted benzothiazole-2-thiols and benzoxazole-2-thiols were prepared from the condensation O-ethylxanthic acid, potassium salt or any suitable salts and 2-haloanilines or 2-hydroxyanilines respectively, scheme F. These compounds can also be prepared from 2-aminobenzothiazoles (ref. Kasibhatla et. al. U.S. Pat. No. 6,489,476 B1) by diazotization followed by displacement with SH using thiourea or O-ethylxanthic acid, potassium salt (see ref. The Chemistry of the Thio Group, Part 1, Patai S. Ed. John Wiley & Sons. London, 1974, pp 163-263 and Ma, C. J. Org. Chem. 2001, 66, 4525.).

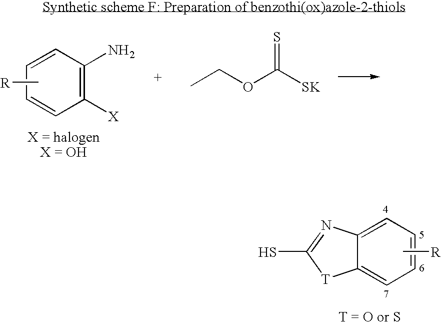

Similarly, condensation of 2-amino-3-halopyridines or 4-amino-3-halopyridines or 3-amino-2-halopyridines with O-ethylxanthic acid, potassium salt can give the other pyridylthiazole-2-thiols isomers, scheme G. These can also be prepared from 2-amino pyridothiazoles.

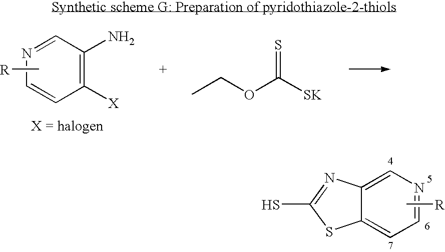

### Synthesis of 8-benzyladenines

The 8-benzyladenines were synthesized by either of the two methods illustrated in Scheme H. The first method followed a sequence closely related to the one described by Drysdale et al., and started from commercial 4,6-dichloro-5-aminopyrimidine, which was treated with butylamine, acylated with the appropriate phenacyl chloride, and cyclized to afford the desired 9-butyl-8-(2,5-dimethoxybenzyl)-9H-purin-6-ylamine. The second method was similar to the one of Chiosis et al. and started with 4,5,6-triaminopyrimidine, which was acylated and cyclized to give 8-(3-methoxy-benzyl)-9H-purin-6-ylamine. The final alkylation gave predominantly the desired N(9)-alkyl isomer, together with a minor regioisomer which was removed by chromatography (regioselectivity=5:1 by ¹H NMR analysis of the crude product). The anisole was halogenated using standard reagents (SO₂Cl₂, Br₂, NIS/AcOH). One improvement in the synthetic sequence pertained to the acylation step. The published method involves acylation of 4,5,6-triaminopyrimidine hemisulfate in aqueous solution (4,5,6-triaminopyrimidine is soluble only in water at pH ≧7), and required, in our hands, several equivalents of the appropriate acyl fluoride to compensate for the accompanying hydrolysis of the reagent. We found that the free base of 4,5,6-triaminopyrimidine was readily isolated as needles by neutralizing and cooling to 0-5° C. an aqueous solution of the commercial hemisulfate. The free base proved to be soluble in N-methyl-2-pyrrolidone (NMP), and could be efficiently acylated in this solvent with a single equivalent of acyl chloride to give the amide. The use of DMF as a solvent was less satisfactory, since it gave rise to a competitive formylation of the 5-NH₂ group via a Vilsmeier-Haack type of reaction. Bases such as Et₃N were best avoided, to prevent over-acylation, and the desired amide precipitated as its HCl salt. The symmetry of the ¹H-NMR spectrum of the acyated product indicated that the acylation had occurred selectively at the 5-position. Finally, the cyclization of amide to the desired purine was carried out with MeONa in refluxing n-BuOH, a minor deviation from the original MeOH, but which provided more forceful and generally applicable conditions.

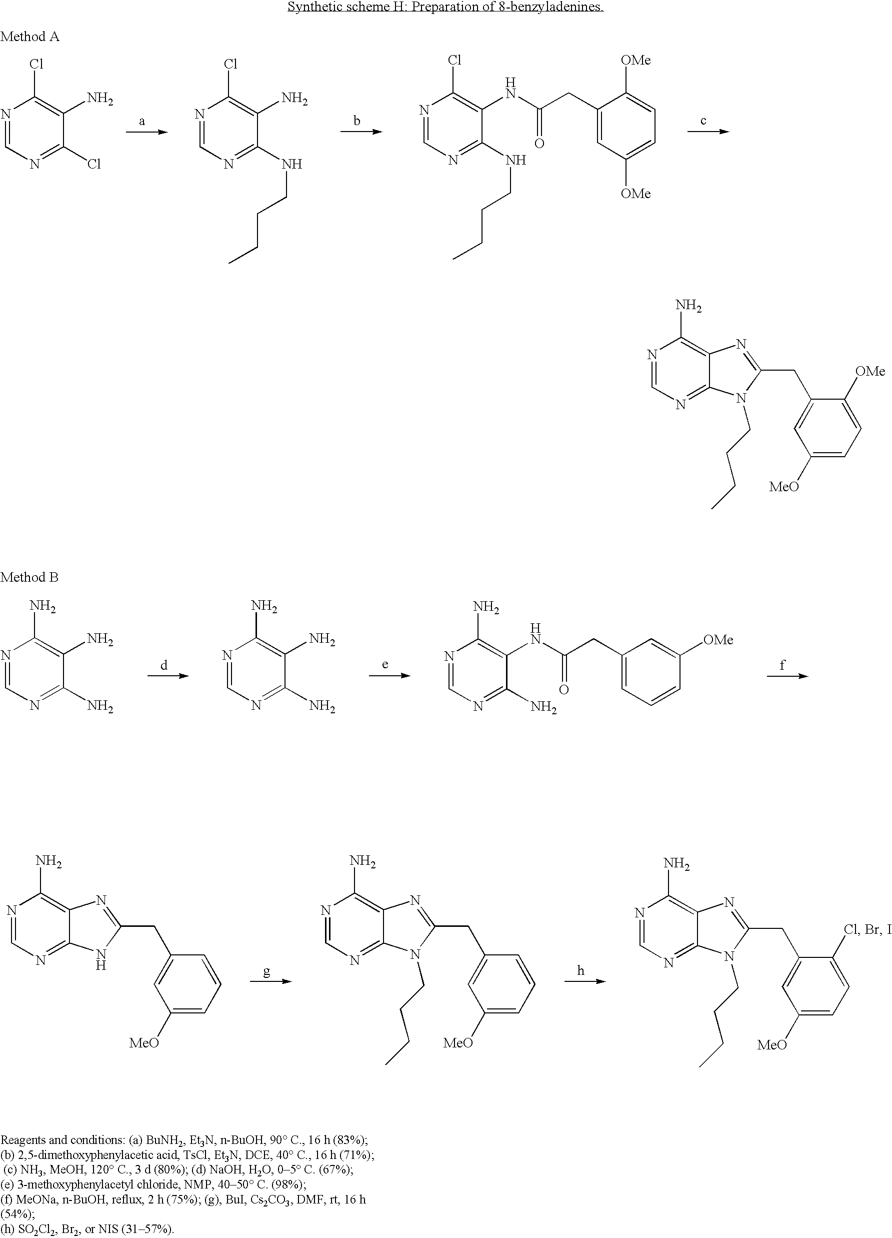

### Synthesis of 8-arylsulfanyladenines

A different approach was necessary to investigate the effect of the linker between the purine and the benzene ring. The compounds with a sulfur atom as a linker were prepared according to the example shown in Scheme I. 8-Bromoadenine was alkylated to give a 2:1 mixture of the N(9)- and N(3)-alkylated isomers, from which the desired N(9)-butyl isomer could be isolated by chromatography. Displacement of the bromine atom with the desired thiophenolate gave the desired 8-sulfanyladenine.

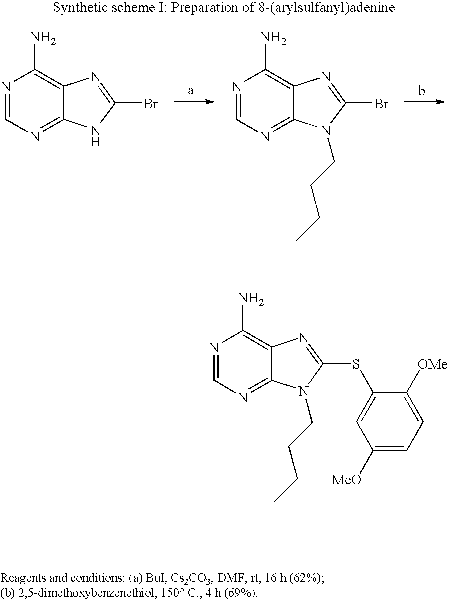

### Synthesis of 8-(Iodo-substituted-phenylsulfanyl)-adenines

A similar approach was used to generate 8-(benzenesulfanyl)-adenines carrying an iodo substituent on the benzene ring via the convergent synthesis shown in Scheme J. The methoxy-nitroaniline was converted in three known steps (1. NaNO₂, KI. 2. H₂N—NH₂/Fe. 3. NaNO₂, HBF₄) to the diazonium salt, which gave after a two-step Leuckart synthesis, (4. EtOCS₂K, 5. KOH) the potassium salt of 2-iodo-5-methoxy-benzenethiophenol. Purification of this compound proved to be challenging. Dissolution in MeOH and precipitation with EtOAc gave a low recovery (<20%) of the desired thiophenolate, while neutralization and chromatography was complicated by the odor of the thiophenol and its tendency to oxidize to the corresponding disulfide. In the event, the highest overall yield for the conversion to phenylsulfanyl adenine was achieved by hydrolyzing the intermediate xanthate with 2 equivalents of KOH in MeOH, concentrating the reaction mixture, and using it without removing the excess of potassium salts. Alkylation of 8-bromoadenine with homoprenyl bromide gave a 2:1 mixture of the desired N(9)-vs. N(3)-alkylated products, and the desired isomer was isolated by chromatography. Coupling the thiophenolate with 8-bromoadenine gave the desired 8-(2-Iodo-5-methoxy-phenylsulfanyl)-9-(4-methyl-pent-3-enyl)-9H-purin-6-ylamine. Similarly, 8-bromoadenine could be alkylated with 5-chloro-1-pentyne to give 8-Bromo-9-pent-4-ynyl-9H-purin-6-ylamine, and coupled to the thiophenolate to provide the pentyne analog 8-(2-Iodo-5-methoxy-phenylsulfanyl)-9-pent-4-ynyl-9H-purin-6-ylamine. 2-Fluoro-8-(2-iodo-5-methoxy-phenylsulfanyl)-9-pent-4-ynyl-9H-purin-6-ylamine was prepared by a conceptually similar route starting from 2,6-diamino-8-bromopurine, using a Balz-Schiemann reaction (iso-AmONO/HBF₄) to replace the 2-NH₂ group with a fluorine.

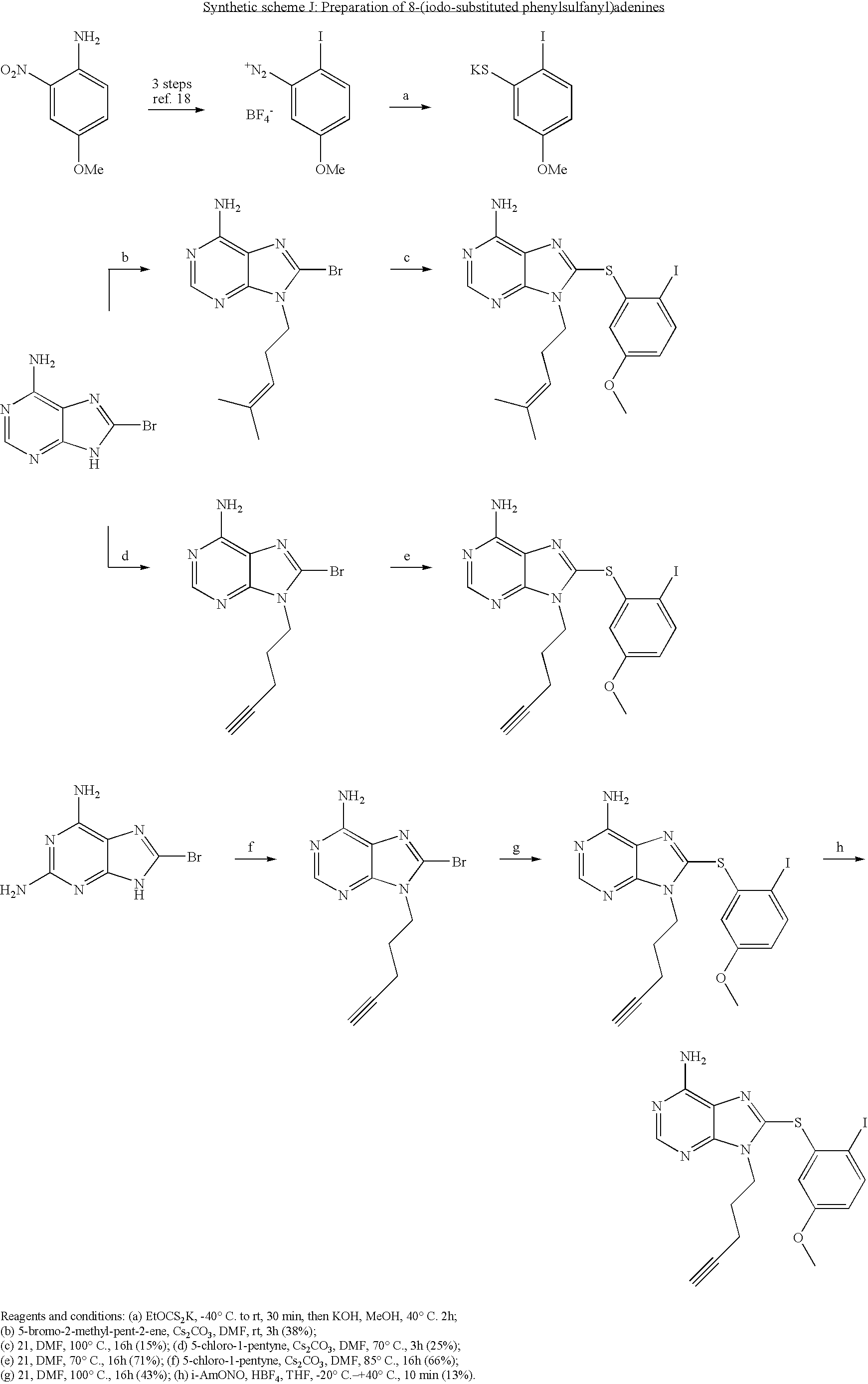

However, these routes suffered from two drawbacks. First, the alkylation was not regioselective and required a tedious chromatographic separation. Second, the thiophenolate was malodorous, difficult to purify, and if used without chromatographic purification gave results which were very sensitive to its purity. One improvement (Scheme K, route A) consisted of starting from pure adenine, which underwent alkylation solely at N(9). Br—(CH₂)₂—OAc or Cl—(CH₂)₃—OAc were selected as alkylating agents, in which the masked hydroxyl group provided a handle for further functionalization. Unlike the unsubstituted adenine, the alkylated product was easily brominated at C(8). The bromine atom was then displaced with the potassium thiophenolate, and the acetyl protecting group cleaved in situ to give the 8-sulfanyladenine. The hydroxy group was mesylated and displaced with amines to give the corresponding N-alkylamines of generic structure A (Scheme K).

This route, however, still required the disagreeable preparation of the thiophenolate. We therefore investigated the direct treatment of the diazonium salt with the anion of 8-thionoadenine, which already contained the desired sulfur atom (Scheme K, route B), thus avoiding the preparation of the thiophenolate. Condensation of 4,5,6-triaminopyrimidine with thiourea, followed by treatment with the diazonium salt gave the desired 8-(2-Iodo-5-methoxy-phenylsulfanyl)-9H-purin-6-ylamine. The yield (un-optimized) was low and the subsequent alkylation with Br—(CH₂)₃—Cl still gave a 2:1 mixture of N(9)- and N(3)-regioisomers. In spite of these problems, the route was effective in generating small amounts of amines of formula A. However, an improved route was needed to prepare gram amounts of A. A third route was selected (Scheme K, route C) which eliminated the use of the thiophenolate and gave only one regioisomer. The alkylated bromoadenine from route A, scheme K was converted with thiourea to the 8-thionoadenine, which was coupled directly with the diazonium salt to give the desired, known adduct. Finally, the same standard manipulations of the side-chain (deacetylation, mesylation, amination) gave the desired amines of generic formula A. Routes A and C gave similar overall yields and were used interchangeably, but the latter was consistently reproducible, and avoided the use of the thiophenylate.

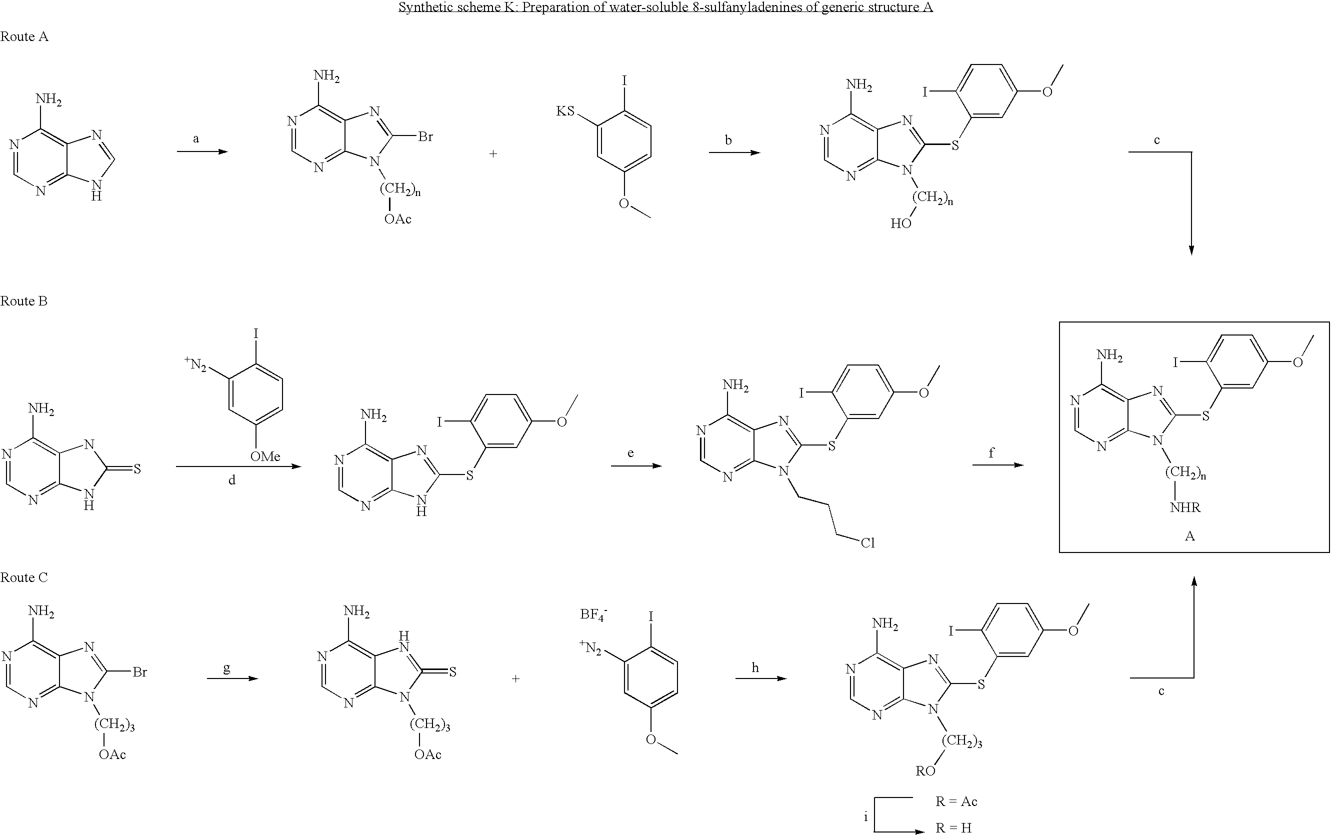

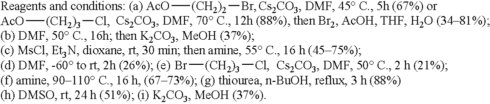

### Synthesis of 7′-substituted benzolothio and pyridothiazole purines

Hsp90 inhibitor analogs could also be prepared using the three-step general procedure outlined in Scheme L. Adenine alkylation with the appropriate alkyl halide in the presence of Cs₂CO₃ in DMF gave predominantly the N-9 substituted isomers. Bromination of the purines followed by coupling with substituted benzothiazole-2-thiols in the presence of t-BuOK in DMF at elevated temperature provided the final products.

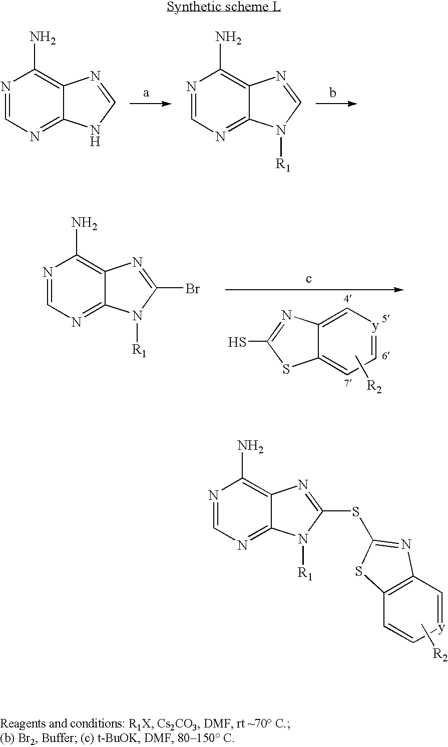

Adenine alkylation to give the N-9 regiosisomer was unambiguously assigned by preparing a representative compound via a different synthetic route (Scheme M). Following the described synthesis (Howson et al. J. Med. Chem. 1988, 23, 433-439), 5-amino-4,6-dichloropyrimidine was treated with aminopropylalcohol to give the diaminosubstituted pyrimidine. Cyclization with triethylorthoformate in acetic anhydride gave the 6-chloropurine derivative which was further reacted with ammonia in MeOH to give, without purification, the 9-substituted adenine. Acylation with acetic anhydride in the presence of DMAP and pyridine afforded the protected alcohol whose NMR spectrum was identical to the compound obtained via the scheme outlined above. This unequivocally established that the alkylation of the purine in this step occurred predominantly at the 9-position.

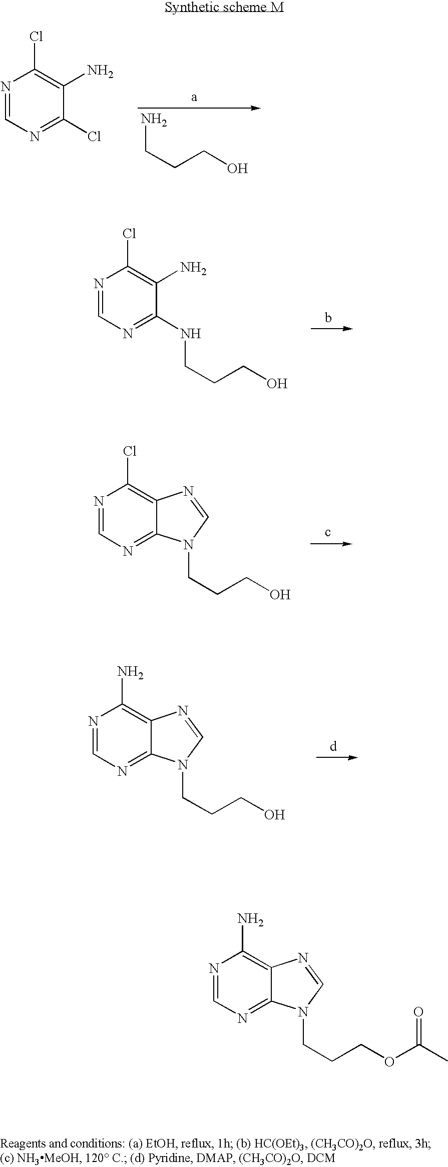

### Synthesis of 7′-substituted benzolothio-2-thiols

Benzothiazole-2-thiols were obtained by four different synthetic approaches as depicted in Schemes N, O, P, Q. The key reaction in all four routes involved the condensation of 2-haloanilines with the potassium salt of ethylxanthic acid to give benzothiazole-2-thiols. The substituted 2-haloanilines were prepared via three different routes: 1) Reduction of 2-nitrobromobenzene with Fe in EtOH to give the substituted 2-haloaniline, 2) bromination of 2-nitroaniline through Sandmeyer reaction followed by reduction with Fe in EtOH (Scheme O) and, 3) nitrolation of 1,2-dibromobenzene with HNO₃ in H₂SO₄ (Scheme P) to give a mixture of the 3- and 4-NO₂ regioisomers, followed by reduction of the desired 3-NO₂ regioisomer with Fe in EtOH to give 2,3-dibromoaniline. The compounds obtained by these methods were subjected to condensation with the potassium salt of ethylxanthic acid in DMF at 160° C. for 4 h to give substituted benzothiazole-2-thioles in good yield. The 6-Cl-, 5-Cl-, 4-Cl- and 7-H-benzothiazole isomers were purchased from Acros.

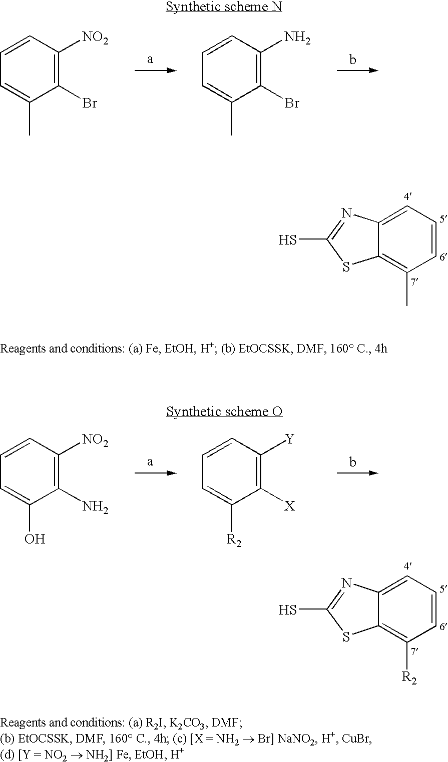

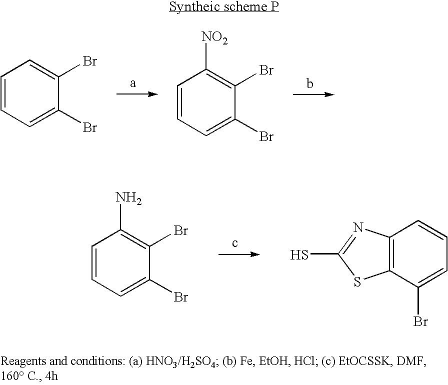

### Synthesis of thiazole[4,5-c]pyridine-2-thiols

Halogenation (Nantka-Namirski et. al. Acta Poloniae Pharmaceutica; 1961; 18, 391-399) of 3-nitro-pyridin-4-ol (Scheme Q) introduced the 5-Cl or 5-Br groups. Subsequently, the 4-OH group was converted to the 4-Cl by treating with POCl₃ (Molecules: EN; 7; 1; 2002; 7-17) and the nitro group was reduced to amino group (Chaudhun et.al. Synth. Commun. 26, 20; 1996; 3783-3790) with SnCl₂. Finally, cyclization with the potassium salt of ethylxanthic acid gave the target thiazole[4,5-c]pyridine-2-thiols.

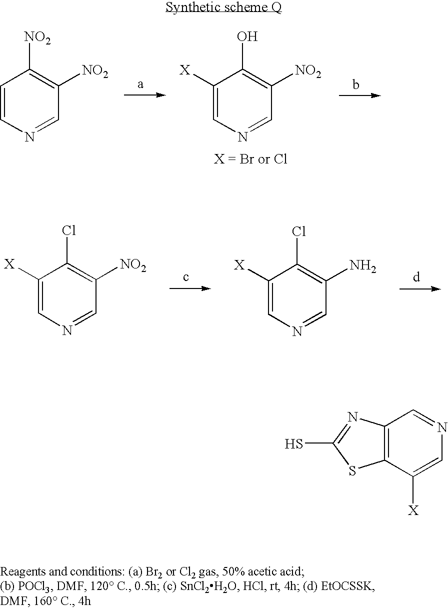

Further modification of the 9-alkyl-side chains is shown in Scheme R. The 9-alkyl esters were hydrolized by treatment with NH₃ in MeOH to form the 9-hydroxy substituted purines, which were treated with MsCl in DMF and the resulting crude mesylates reacted with appropriate amines to give the final compounds.

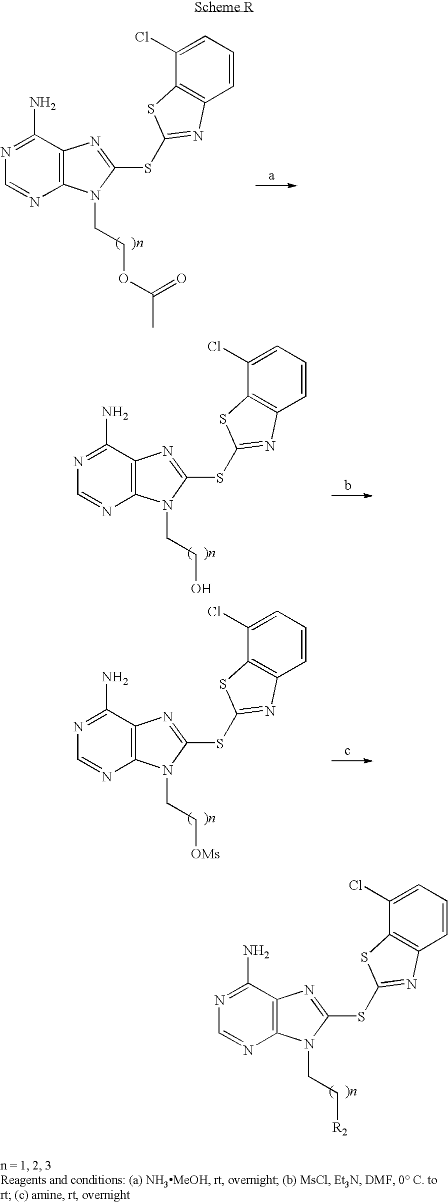

The following examples are offered by way of illustration only and are not intended to be limiting of the full scope and spirit of the invention.

The chemical reagents used below are all available commercially, e.g., from Aldrich Chemical Co., Milwaukee, Wis., USA, and/or their facile preparation known to one of ordinary skill in the art, or otherwise described or referenced herein. Unless otherwise stated, all reactions were carried out under a nitrogen atmosphere. The organic solvents were purchased from Fisher Scientic. Thin layer chromatography (TLC) was performed with Whatman K6F silica Gel 60A plates; 1H-NMR spectra were determined on Bruker 400 MHz instruments. HPLC method used for these compounds: Agilent Zorbax 300 SB C18, 4.6×150 mm, 5 μm; Column Temperature: Ambient; Flow Rate: 1.0 ml/min, Gradient: 5% acetonitrile (0.05% TFA) in water (0.1% TFA) to 100% acetonitrile (0.05% TFA) in 7 minutes, hold at 100% for 2 minutes) (method: 5-100-7), or Gradient: 5% acetonitrile (0.05% TFA) in water (0.1% TFA) to 100% acetonitrile (0.05% TFA) in 15 minutes, hold at 100% for 2 minutes) (method: 5-100-15).

### Synthesis of Compounds

### EXAMPLE 1 Synthesis of 8-(2,5-dimethoxybenzyl)-N-9-butyladenine (1)

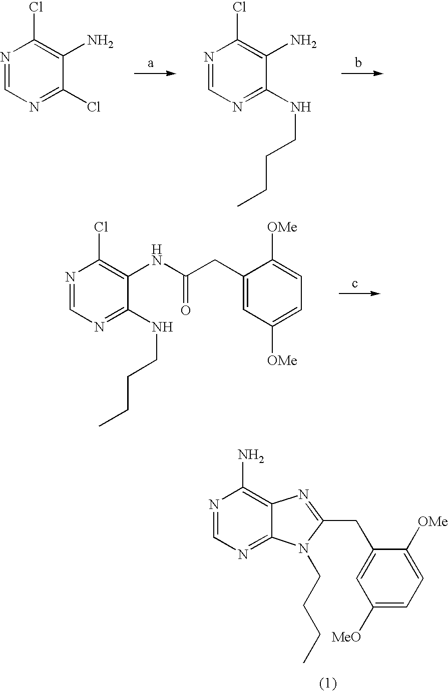 Step 1: A solution of 5-amino-4,6-dichloropyrimidine (1 mmol) in n-BuOH was treated with Et₃N (1.2 mmol) and n-Butylamine (1.0 mmol) at 80 C. After 16 h, solvent was removed under reduced pressure. The residue was dissolved in EtOAc, the organic layer washed with water and then dried (MgSO₄). Filtration and removal of solvent gave 6-chloro-5-amino-4-butyl pyrimidine as a brown solid. R_{f}=0.5 in 1:1 EtOAc:hexane. ¹H NMR (CDCl₃) δ 8.07 (s, 1H), 4.88 (br s, 1H), 3.49 (m, 2H), 3.35 (br s, 2H), 1.6 (m, 2H), 1.44 (m, 2H), 0.95 (t, 3H).
Step 2: To a solution of 2,5-dimethoxyphenylacetic acid (1 mmol) and Et₃N (1 mmol) in CH₂Cl₂ was added p-toluenesulfonyl chloride (1 mmol) at rt. After 1 h, the mixture was treated with a solution of the product of step 1, 6-chloro-5-amino-4-butyl pyrimidine (1 mmol in CH₂Cl₂), followed by addition of Et₃N (2 mmol). The resultant mixture was refluxed for 20 h. Solvent was removed and the residue dissolved into EtOAc, the organic layer washed with water and dried. The crude compound was taken into acetone, and precipitated product filtered out and washed with a small amount of acetone to give N-(4-butylamino-6-chloro-pyrimidin-5-yl)-2-(2,5-dimethoxyphenyl)acetamide. R_{f}=0.45 in 1:1 EtOAc:hexane. ¹H NMR (DMSO-d₆) δ 9.37 (s, 1H), 8.17 (s, 1H), 7.11 (t, 1H), 6.9 (d, 1H), 6.88 (d, 1H), 6.78(dd, 1H), 3.73 (s, 3H), 3.69 (s, 3H), 3.63 (s, 3H), 3.35 (m, 2H), 1.48 (m, 2H), 1.29 (m, 2H), 0.88 (t, 3H).
Step 3: A mixture of N-(4-butylamino-6-chloro-pyrimidin-5-yl)-2-(2,5-dimethoxyphenyl)acetamide (1 mmol) and p-TSA (0.5 mmol) in toluene was refluxed for 72 h. Solvent was removed, diluted with EtOAc and washed with water, bicarbonate and dried. Purification on a silica gel column (200-400 mesh, Fisher Scientific, Tustin, Calif., USA) gave 6-chloro-8-(2,5-dimethoxybenzyl)-N9-butyl purine. R_{f}=0.65 in 1:1 EtOAc:hexane. ¹H NMR (DMSO-d₆) δ 8.7 (s, 1H), 6.96 (d, 1H), 6.84 (m, 1H), 6.8(dd, 1H), 4.28 (s, 2H), 4.23 (t, 2H), 3.69 (s, 3H), 3.67 (s, 3H), 1.62 (m, 2H), 1.25 (m, 2H), 0.88 (t, 3H).
Step 4: To a solution of 6-chloro-8-(2,5-dimethoxybenzyl)-N9-butyl purine (1 mmol) in dioxane was added 28% NH₄OH (50 mmol) and the mixture was then heated at 100 C in a seal tube for 48 h. Solvent was removed by azeotrope distillation with toluene. Purification on a silica gel column (see above) gave pure 8-(2,5-dimethoxybenzyl)-9-butyl adenine, 1.1. R_{f}=0.35 in 5% MeOH in EtOAc. ¹H NMR (DMSO-d₆) δ 8.08 (s, 1H), 7.04 (br s, 2H), 6.94 (d, 1H), 6.80 (dd, 1H), 6.66(d, 1H), 4.14 (s, 2H), 4.04 (t, 2H), 3.72 (s, 3H), 3.63 (s, 3H), 1.52 (m, 2H), 1.22 (m, 2H), 0.82 (t, 3H).

Alternatively, 8-(2,5-dimethoxybenzyl)-9-butyl adenine can also be prepared from N-(4-butylamino-6-chloro-pyrimidin-5-yl)-2-(2,5-dimethoxyphenyl)acetamide according to the following procedure: A solution of N-(4-butylamino-6-chloro-pyrimidin-5-yl)-2-(2,5-dimethoxyphenyl)acetamide (1 mmol) is taken into 7M N₃ in MeOH (70 mmol) and the mixture heated at 120 C in a steel bomb for 72 h. Solvent is removed by azeotrope distillation with toluene. Purification on the silica gel column gave pure 8-(2,5-dimethoxybenzyl)-9-butyl adenine.

### EXAMPLE 2 Synthesis of 8-(2,5-dimethoxybenzyl)-N9-pentynyl-2-fluoro adenine

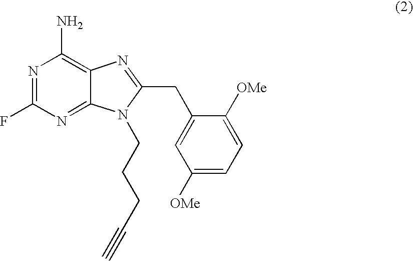

### Step 1: 2-(2,5-Dimethoxy-phenyl)-N-(2,5,6-triamino-pyrimidin-4-yl)-acetamide, HCl

A solution of 2,4,5,6-tetraminopyrimidine (52.8 g, 378 mmol) in NMP (750 ml) was treated at 70° C. with 2,5-dimethoxyphenyl acetyl chloride (90 g, 419 mmol). After cooling to r.t., the precipitate was collected by filtration and washed with EtOAc to give the title compounds as a pale yellow powder (127 g, 95%). 1H NMR (DMSO-d₆) δ 9.12 (s, 1H), 7.80-7.40 (m, 3H), 6.22 (s, 2H), 6.04 (s, 4H), 4.41 (s, 3H), 4.29 (s, 3H), 4.25 (s, 2H); MS 319 (M+1).

### Step 2: 8-(2,5-Dimethoxy-benzyl)-9H-purine-2,6-diamine

Sodium metal (2.3 g, 100 mmol) was dissolved in n-BuOH (50 ml) at 70° C. To this was added the acetamide of step 1, above (5.0 g, 14.1 mmol), and the mixture was heated to reflux for 1.5 h. Neutralization with 6N HCl to pH 8-9, extraction with EtOAc, drying, and evaporation gave the title compound as a pale yellow powder (3.2 g, 76%). R_{f}=0.45 in 1:3 MeOH:EtOAc. 1H NMR (DMSO-d₆) δ 12.3-11.7 (br. s, 1H), 6.92 (d, J=10.0 Hz, 1H), 6.82 (dd, J=10.0 & 3.0 Hz, 1H), 6.73 (s, 1H), 6.70-6.50 (br. s, 2H), 5.85-5.70 (br. s, 2H), 3.95 (s, 2H), 3.74 (s, 3H), 3.67 (s, 3H); MS 301 (M+1).

### Step 3: 8-(2,5-Dimethoxy-benzyl)-9-pent-4-ynyl-9H-purine-2,6-diamine

A mixture of the purine 8-(2,5-Dimethoxy-benzyl)-9H-purine-2,6-diamine (19.0 g, 63 mmol), 5-chloro-pent-1-yne (12.3 ml, 116 mmol), and Cs₂CO₃ (37.8 g, 116 mmol) in DMF (180 g) was heated to 50° C. for 16 h. Filtration and washing (2×200 ml H₂O) afforded some desired product (5.8 g, 25%). The mother liquor was concentrated, diluted with EtOAc, and heated to reflux for 1 h to yield additional product (6.0 g, 26%). After cooling to room temperature, addition of 1 volume hexane to the EtOAc mother liquor gave additional product (2.6 g, 11%). Final work-up (CH₂Cl₂:MeOH 4:1—water) yielded additional product (5.3 g, containing 1 equivalent pentyl-4-yn-1-ol, 18%). R_{f}=0.65 in 1:10 MeOH:EtOAc. 1H NMR (DMSO-d₆) δ 6.92 (d, J=8.9 Hz, 1H), 6.98 (dd, J=8.9 & 3.0 Hz, 1H), 6.59 (s, J=2.9 Hz, 1H), 6.58-6.53 (br. s, 2H), 5.72-5.68 (br. s, 2H), 4.02 (s, 2H), 3.92 (t, J=7.4 Hz, 2H), 3.73 (s, 3H), 3.62 (s, 3H), 2.84 (t, J=2.5 Hz, 1H), 2.13 (td, J=7.0 & 1.7 Hz, 2H), 1.74 (quint., J=7.3 Hz, 2H); MS 367 (M+1).

### Step 4: 8-(2,5-Dimethoxy-benzyl)-2-fluoro-9-pent-4-ynyl-9H-purin-6-ylamine

A solution of the above purine-2,6-diamine (11.8 g, 32.2 mmol) in 48% aq. HBF₄ (250 ml) was treated at −10° C. with iso-amyl nitrite (5.20 ml, 38.8 mmol), and warmed to r.t over 2.5 h. The reaction mixture was diluted with MeOH (400 ml) and CH₂Cl₂ (1500 ml), and carefully neutralized with a solution of K₂CO₃ (125 g) in water (500 ml). Caution: vigorous gas evolution. The aqueous layer was re-extracted with MeOH:CH₂Cl₂ (500 ml, 1:5). Concentration of the organic phase and two flash chromatography purifications (CH₂Cl₂:EtOAc:hexane:MeOH:Et₃N 1500:750:750:50:10→1500:750:750:150:10) yielded 8-(2,5-Dimethoxy-benzyl)-2-fluoro-9-pent-4-ynyl-9H-purin-6-ylamine (4.5 g, 38%), 2.1 as a colorless powder. R_{f}=0.45 in 1:1 EtOAc:hexane. ¹H NMR (DMSO-d₆) δ 6.82 (d, J=8.9 Hz, 1H), 6.75 (dd, J=8.9 & 3.0 Hz, 1H), 6.68 (d, J=2.9 Hz, 1H), 6.25-6.10 (br. s, 2H), 4.20 (s, 2H), 4.13 (t, J=7.4 Hz, 2H), 3.79 (s, 3H), 3.70 (s, 3H), 2.16 (td, J=7.0 & 2.6 Hz, 2H), 1.97 (t, J=2.6 Hz, 1H), 1.95 (quint., J=7.3 Hz, 2H); MS 370 (M+1).

The following compounds 3-5, were prepared using essentially the same procedures described for Example 2, except that in step 3 the electrophiles 1-bromo-4-methyl-pent-3-ene, 1-chloro-pent-4-ene, and 1,5-bromopentane were used in place of 5-chloro-pent-1-yne:

### EXAMPLE 3 8-(2,5-Dimethoxy-benzyl)-2-fluoro-9-(4-methyl-pent-3-enyl)-9H-purin-6-ylamine

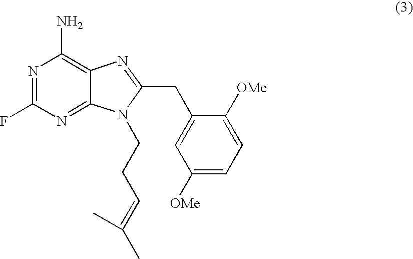

isolated as solid, retention time=7.70.

### EXAMPLE 4 8-(2,5-Dimethoxy-benzyl)-2-fluoro-9-pent-4-enyl-9H-purin-6-ylamine (4)

isolated as solid, retention time=7.61.

### EXAMPLE 5 8-(2,5-Dimethoxy-benzyl)-2-fluoro-9-(5-bromo-pentyl)-9H-purin-6-ylamine

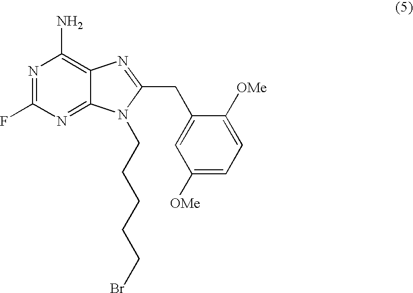

isolated as solid, retention time=7.86.

### EXAMPLE 6 8-(2,5-Dimethoxy-benzyl)-2-chloro-9-pent-4-ynyl-9H-purin-6-ylamine (6)

This compound was prepared analogously to the method described in example 5, step 4 using HCl and CuCl in place of HBF₄. Rt=8.02 ¹H NMR (CDCl3) δ 6.83 (d, J=8.9 Hz, 1H), 6.77 (dd, J=8.9 & 3.0 Hz, 1H), 6.68 (d, J=3.0 Hz, 1H), 6.18-6.00 (s, 2H), 4.20 (s, 2H), 4.18 (t, J=7.4 Hz, 2H), 3.78 (s, 3H), 4.93 (s, 3H), 2.20 (td, J=7.0 & 2.4 Hz, 2H), 2.63 (t, 2.4 Hz, 1H), 1.97 (quint., J=7.3 Hz, 2H).

HPLC method: Agilent Zorbax 300 SB C18, 4.6×150 mm, 5 μm; Column Temperature: Ambient; Flow Rate: 1.0 ml/min, Gradient: 10% acetonitrile (0.05% TFA) in water (0.1% TFA) to 100% acetonitrile (0.05% TFA) in 10 minutes, hold at 100% for 1 minutes); Retention times are measured in minutes.

The above procedures can similarly be applied to produce compounds wherein the 2 position is unsubstituted (i.e. is H) by starting with 4,5,6, triaminopyrimidine sulfate and using the appropriate electrophile.

### EXAMPLE 7 9-(4-Chloro-butyl)-8-(2,5-dimethoxy-benzyl)-9H-purin-6-ylamine (7)

isolated as solid; rt=6.34.

### EXAMPLE 8 8-(2,5-Dimethoxy-benzyl)-9-pent-4-ynyl-9H-purin-6-ylamine

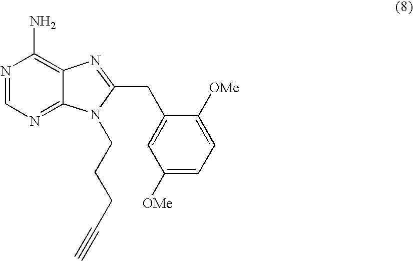

isolated as solid rt=5.88 min.

### EXAMPLE 9 8-(2,5-Dimethoxy-benzyl)-9-(2-[1,3]dioxolan-2-yl-ethyl)-9H-purin-6-ylamine (9)

isolated as solid, rt=5.36.

### EXAMPLE 10 8-(2,5-Dimethoxy-benzyl)-9-(4-methyl-pent-3-enyl)-9H-purin-6-ylamine

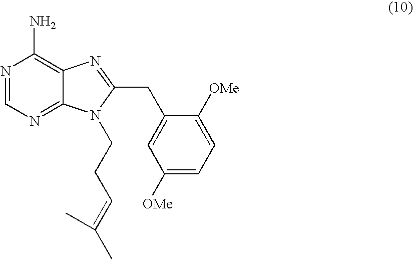

isolated as solid., rt=6.60.

### EXAMPLE 11 9-(5-Bromo-pentyl)-8-(2,5-dimethoxy-benzyl)-9H-purin-6-ylamine (11)

isolated as solid, rt=6.94.

### EXAMPLE 12 9-(5-Bromo-3-methyl-pentyl)-8-(2,5-dimethoxy-benzyl)-9H-purin-6-ylamine (12)

isolated as solid, rt=7.32.

### EXAMPLE 13 9-(5-Chloro-pentyl)-8-(2,5-dimethoxy-benzyl)-9H-purin-6-ylamine (13)

isolated as solid, rt=6.34.

### EXAMPLE 14 8-(2,5-Dimethoxy-benzyl)-9-(4-ethylamino-butyl)-9H-purin-6-ylamine (14)

isolated as solid, rt=3.9.

### EXAMPLE 15 6-[6-Amino-8-(2,5-dimethoxy-benzyl)-purin-9-yl]-hexan-1-ol (15)

The alkylation was done with 1-bromo-4-chlorobutane followed by treatment with ethylamine to give the 4-ethylaminobutyl isolated as solid.

### EXAMPLE 16 8-(2,5-Dimethoxy-benzyl)-9-[2-(dimethyl-bicyclo[3.1.1]hept-2-en-2-yl)-ethyl]-9H-purin-6-ylamine (16)

isolated as solid.

### EXAMPLE 17 Acetic acid 5-[6-amino-8-(2,5-dimethoxy-benzyl)-purin-9-yl]-pentyl ester (17)

isolated as solid; rt=6.06.

### EXAMPLE 18 8-(2,5-Dimethoxy-benzyl)-9-(3,3,3-trifluoro-propyl)-9H-purin-6-ylamine (19)

isolated as solid

### EXAMPLE 19 8-(2,5-Dimethoxy-benzyl)-9-pent-4-ynyl-9H-purin-6-ylamine (20)

isolated as solid; rt=5.88.

### EXAMPLE 20 9-Butyl-8-(2-iodo-5-methoxy-benzyl)-9H-purin-6-ylamine (20)

To a solution of 9-butyl-8-(3-methoxy-benzyl)-9H-purin-6-ylamine (1.24 g, 4 mmol) in AcOH (6 ml) was added N-iodo-succinamide (NIS) (1.8 g, 8 mmol). After 3 h at r.t., additional NIS (1.8 g, 8 mmol) was added, and the mixture was stirred for another 24 h. The reaction mixture was diluted with CH₂Cl₂ (500 ml), and carefully neutralized with a solution of sat. aq. K₂CO₃ (2×100 ml), then washed with 0.1 N Na₂S₂O₃ (3×100 ml), brine (3×100 ml), dried (Na₂SO₄), evaporated, and purified by flash chromatography (CH₂Cl₂:MeOH=100:5) to give the 9-Butyl-8-(2-iodo-5-methoxy-benzyl)-9H-purin-6-ylamine (20), as a colorless powder (0.53 g, 30%); rt=7.7 min.; ¹H NMR (CDCl₃-d) δ 8.36 (s, 1H), 7.77 (d, J=7.9 Hz, 1H), 6.68 (s, 1H), 6.61 (d, J=7.9 Hz, 1H), 5.62 (s, 2H), 4.33 (s, 2H), 4.06 (t, J=7.7 Hz, 2H), 3.72 (s, 3H), 1.67 (quint., J=7.7 Hz, 2H), 1.36 (sext., J=7.5 Hz, 2H), 0.92 (t, J=7.4 Hz, 3H).

Bromo and chloro derivatives were made using the same procedure, substituting NBS and NCS for NIS as appropriate. The following compounds were also synthesized according to essentially the same procedure, using as appropriate NIS, NCS or NBS:

### EXAMPLE 21 9-Butyl-8-(5-iodo-2-methoxy-benzyl)-9H-purin-6-ylamine (21)

was made from 9-Butyl-8-(2-methoxy-benzyl)-9H-purin-6-ylamine as starting material in 48% yield ¹H NMR (CDCl₃) δ 8.32 (s, 1H), 7.55 (dd, J=8.7, 2.2 Hz, 1H), 7.37 (d, J=2.2 Hz, 1H), 6.68 (d, J=8.7 Hz, 1H), 6.05-5.85 (br. s, 2H), 4.17 (s, 2H), 4.07 (t, J=7.6 Hz, 2H), 3.82 (s, 3H), 1.62 (quint., J=7.5 Hz, 2H), 1.30 (sext., J=7.5 Hz, 2H), 0.89 (t, J=7.4 Hz, 3H).

### EXAMPLE 22 9-Butyl-8-(5-ethyl-2-methoxy-benzyl)-9H-purin-6-ylamine (22)

Rt=7.59; ¹HNMR (CDCl3-d) δ 8.35 (s, 1H), 7.34 (d, J=8.8 Hz, 1H), 6.79 (dd, J=8.7, 2.8 Hz, 1H), 6.69 (d, J=2.7 Hz, 1H), 5.64 (s, 2H), 4.36 (s, 2H), 4.07 (t, J=7.7 Hz, 2H), 3.73 (s, 3H), 1.64 (quint., J=7.7 Hz, 2H), 1.32 (sext., J=7.5 Hz, 2H), 0.90 (t, J=7.4 Hz, 3H).

### EXAMPLE 23 8-(2-Bromo-5-methoxy-benzyl)-9-butyl-9H-purin-6-ylamine (23)

Rt=7.66; ¹HNMR (CDCl3-d) δ 8.36 (s, 1H), 7.52 (d, J=8.7 Hz, 1H), 6.74 (dd, J=8.7, 3.0 Hz, 1H), 6.89 (d, J=3.0 Hz, 1H), 5.64 (s, 2H), 4.36 (s, 2H), 4.07 (t, J=7.7 Hz, 2H), 3.72 (s, 3H), 1.64 (quint., J=7.6 Hz, 2H), 1.34 (sext., J=7.5 Hz, 2H), 0.90 (t, J=7.4 Hz, 3H).

9-Butyl-8-(2-methoxy-benzyl)-9H-purin-6-ylamine and 9-butyl-8-(3-methoxy-benzyl)-9H-purin-6-ylamine were prepared from 4,5,6-triaminopyrimidine sulfate and, respectively 2-methoxyphenyl acetyl chloride or 3-methoxyphenyl acetic acid, by procedures analogous to the one described above. 2-Fluoro purine analogs were also prepared from 2,4,5,6-tetraminopyrimidine, by procedures analogous to those described above. See Example 2, step 4.

For compounds 24-29, in which the N9 substituent is sensitive to halogenation, addition of the N9 substituent was done as a final step:

### EXAMPLE 24 8-(2-Bromo-5-methoxy-benzyl)-9-(4-methyl-pent-3-enyl)-9H-purin-6-ylamine (24)

Rt=8.22; ¹HNMR (CDCl3-d) δ 8.37 (s, 1H), 7.51 (d, J=8.7 Hz, 1H), 6.73 (dd, J=8.7 Hz, 3.0 Hz, 1H), 6.65 (d, J=3.0 Hz, 1H), 5.53 (s, 2H), 5.12 (t, J=7.1 Hz, 2H), 4.35 (s, 2H), 4.07 (t, J=7.1 Hz, 2H), 3.72 (s, 3H), 2.43 (quart., J=7.1 Hz, 2H), 1.65 (s, 3H), 1.40 (s, 3H).

### EXAMPLE 25 8-(2-Bromo-5-methoxy-benzyl)-9-pent-4-ynyl-9H-purin-6-ylamine (25)

Rt=8.17; ¹HNMR (CDCl3-d) δ 8.35 (s, 1H), 7.52 (d, J=8.8 Hz, 1H), 6.74 (dd, J=8.8 Hz, 2.9 Hz, 1H), 6.66 (d, J=2.9 Hz, 1H), 5.61 (s, 2H), 4.39 (s, 2H), 4.21 (t, J=7.4 Hz, 2H), 3.73 (s, 3H), 2.24 (td, J=6.8 Hz, 2.5 Hz, 2H), 2.03 (t, J=2.5 Hz, 1H), 1.99 (quint., J=7.2 Hz, 2H).

### EXAMPLE 26 8-(2-Iodo-5-methoxy-benzyl)-9-pent-4-ynyl-9H-purin-6-ylamine

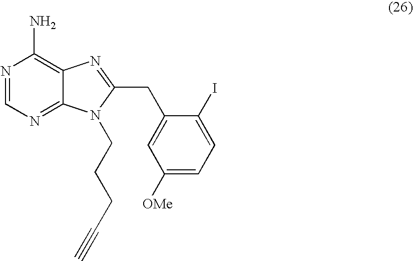

Rt=7.35; ¹HNMR (CDCl3-d) δ 8.36 (s, 1H), 7.77 (d, J=8.5 Hz, 1H), 6.64-6.60 (m, 2H), 5.56 (s, 2H), 4.35 (s, 2H), 4.20 (t, J=7.4 Hz, 2H), 3.73 (s, 3H), 2.26 (td, J=6.9 Hz, 2.7 Hz, 2H), 2.03 (t, J=2.7 Hz, 1H), 2.02 (quint., J=7.0 Hz, 2H).

### EXAMPLE 27 8-(2-Iodo-5-methoxy-benzyl)-9-(4-methyl-pent-3-enyl)-9H-purin-6-yl amine

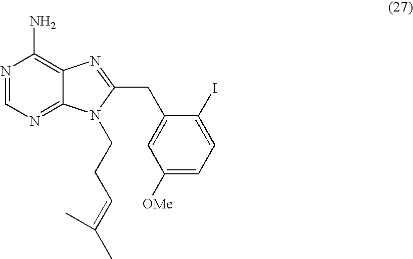

Rt=8.17; ¹HNMR (CDCl3-d) δ 8.58 (s, 1H), 8.33 (d, J=8.6 Hz, 1H), 6.60 (d, J=2.9 Hz, 1H), 6.57 (dd, J=8.6, 2.9 Hz, 1H), 6.15 (s, 2H), 5.12 (t, J=7.4 Hz, 2H), 4.29 (s, 2H), 4.04 (t, J=7.3 Hz, 2H), 3.67 (s, 3H), 2.42 (quart., J=7.2 Hz, 2H), 1.65 (s, 3H), 1.39 (s, 3H).

### EXAMPLE 28 2-Fluoro-8-(2-iodo-5-methoxy-benzyl)-9-(4-methyl-pent-3-enyl)-9H-purin-6-ylamine

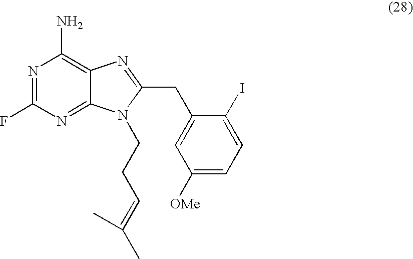

Rt=10.04; 1HNMR (CDCl3-d) δ 7.76 (d, J=8.6 Hz, 1H), 6.65 (d, J=2.5 Hz, 1H), 6.60 (dd, J=8.6, 2.5 Hz, 1H), 6.14 (s, 2H), 5.13 (t, J=6.9 Hz, 1H), 4.26 (s, 2H), 4.01 (t, J=7.0 Hz, 2H), 3.72 (s, 3H), 2.43 (quint., J=7.0 Hz, 2H), 1.68 (s, 3H), 1.42 (s, 3H).

### EXAMPLE 29 2-Fluoro-8-(2-iodo-5-methoxy-benzyl)-9-pent-4-ynyl-9H-purin-6-ylamine

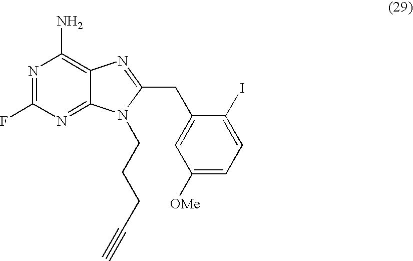

Rt=8.75; 1HNMR (CDCl3-d) δ 7.77 (d, J=8.7 Hz, 1H), 6.67 (d, J=2.7 Hz, 1H), 6.62 (dd, J=8.7, 2.7 Hz, 1H), 5.99 (s, 2H), 4.32 (s, 2H), 4.16 (t, J=7.2 Hz, 2H), 3.74 (s, 3H), 2.26 (td, J=6.7, 2.6 Hz, 2H), 2.02 (t, J=2.4 Hz, 1H), 1.99 (quint., J=6.9 Hz, 2H); MP: 172-177° C.

General Procedure for Palladium-Mediated Couplings

A mixture of 9-Butyl-8-(5-iodo-2-methoxy-benzyl)-9H-purin-6-ylamine (50 mg, 0.1 mmol) and Pd(PPh₃)₄ (12 mg, 0.01 mmol) was treated under N₂ at r.t. with a 1M solution of the organometallic coupling partner (0.5 ml, 0.5 mmol). Reactions were performed typically in THF at r.t. for 10 min with organomagnesium compounds in THF at r.t. for 16 h with organozinc compounds, or in DMF at 80° C. for 3 h with organostannanes. After work-up, the product was purified by chromatography on preparative plates (1000 uM, SiO₂), eluting with CH₂Cl₂:EtOAc:hexane:MeOH:Et₃N 1500:750:750:50:10.

Compounds 30, 31, 32 were prepared using the corresponding commercially available organozinc compound; the skilled artisan will recognize that equivalent organnostannane, and organoboron, and organomagnesium coupling partners may be used in place of organozinc compounds. A general review of appropriate methodologies may be found in “Palladium Reagents in Organic Synthesis” Richard F. Heck, Academic Press, 1990.

### EXAMPLE 30 9-Butyl-8-(5-ethyl-2-methoxy-benzyl)-9H-purin-6-ylamine (30)

Rt=8.23; ¹H NMR (CDCl3) δ 8.30 (s, 1H), 7.07 (dd, J=8.4 & 2.0 Hz, 1H), 6.91 (d, J=2.0 Hz, 1H), 6.83 (d, J=8.4 Hz, 1H), 5.65-5.55 (s, 2H), 4.23 (s, 2H), 4.04 (t, J=7.6 Hz, 2H), 3.83 (s, 3H), 2.51 (q, J=7.6 Hz, 2H) 1.65-1.55 (m, 2H), 1.30-1.25 (m, 2H), 1.41 (t, J=7.6 Hz, 3H), 0.86 (t, J=7.3 Hz, 3H).

### EXAMPLE 31 9-Butyl-8-(5-butyl-2-methoxy-benzyl)-9H-purin-6-ylamine (31)

Rt=9.24; ¹H NMR (CDCl3) δ 8.33 (s, 1H), 7.05 (dd, J=8.4 & 1.9 Hz, 1H), 6.88 (d, J=1.8 Hz, 1H), 6.82 (d, J=8.3 Hz, 1H), 5.58-5.48 (s, 2H), 4.23 (s, 2H), 4.04 (t, J=7.6 Hz, 2H), 3.83 (s, 3H), 2.47 (q, J=7.6 Hz, 2H), 1.57 (quint., J=7.5 Hz, 2H), 1.48 (quint., J=7.6 Hz, 2H), 1.32-1.22 (m, 4H), 0.87 (t, J=7.3 Hz, 3H), 0.86 (t, J=7.3 Hz, 3H).

### EXAMPLE 32 9-Butyl-8-(2-methoxy-5-vinyl-benzyl)-9H-purin-6-ylamine (32)

Rt=7.91; ¹H NMR (CDCl3) δ 8.31 (s, 1H), 7.31 (dd, J=8.5 & 2.3 Hz, 1H), 7.16 (d, J=2.2 Hz, 1H), 6.87 (d, J=8.5 Hz, 1H), 6.59 (dd, J=17.6 & 10.9 Hz, 1H), 5.82-5.72 (s, 2H), 5.53 (dd, J=17.6 & 0.7 Hz, 1H), 5.09 (dd, J=10.9 & 0.7 Hz, 1H), 4.22 (s, 2H), 4.06 (t, J=7.6 Hz, 2H), 3.85 (s, 3H), 1.62 (quint., J=7.7 Hz, 2H), 1.30 (sext., J=7.4 Hz, 2H), 0.87 (t, J=7.4 Hz, 3H).

General Procedure for the Nitration of Benzene Ring and Derivatizations

A solution of the purine analog in H₂SO₄ or in H₂SO₄:AcOH 1:4 was treated at 0° C. with 1 equiv HNO₃. The mixture was diluted with EtOAc, neutralized with NaHCO₃ and purified by chromatography on SiO₂ preparative plates (1000 uM) with CH₂Cl₂:EtOAc:hexane:MeOH:Et₃N 1500:750:750:50:10.

Nitro derivatives (20 mg) can be reduced with 10% Pd/C (Aldrich) (20 mg) under H₂ atmosphere in THF at r.t. over 16 h. The resulting aniline can be further monoalkylated (Acetylchloride, CH₂Cl₂) or reductively alkylated (RCHO, NaBH(OAc)₃, 1,2-dichloroethane, r.t.)

Compounds 33-38 were prepared by this method:

### EXAMPLE 33 8-(2,5-Dimethoxy-4-nitro-benzyl)-2-fluoro-9-pent-4-ynyl-9H-purin-6-ylamine (33)

Rt=8.05; ¹H NMR (CDCl3) δ 7.94 (s, 1H), 6.85 (s, 1H), 6.37-6.27 (s, 2H), 4.06 (s, 2H), 4.01 (t, J=7.3 Hz, 2H), 3.69 (s, 3H), 3.66 (s, 3H), 2.13 (td, J=7.0 & 2.6 Hz, 2H), 1.87 (t, J=2.6 Hz, 1H), 1.82 (quint., J=7.3 Hz, 2H).

### EXAMPLE 34 9-Butyl-8-(3,5-dimethoxy-2-nitro-benzyl)-9H-purin-6-ylamine; sulfuric acid salt (34)

Rt=7.33; ¹H NMR (DMSO-d6) δ 8.27 (s, 1H), 8.15-7.90 (br. s, 2H), 6.78 (d, J=2.4 Hz, 1H), 6.55 (d, J=2.4 Hz, 1H), 4.32 (s, 2H), 4.12 (t, J=7.3 Hz, 2H), 3.88 (s, 3H), 3.81 (s, 3H), 1.58 (quint., J=7.5 Hz, 2H), 1.21 (sext., J=7.5 Hz, 2H), 0.84 (t, J=7.4 Hz, 3H).

### EXAMPLE 35 8-(4-Amino-3,5-dimethoxy-benzyl)-9-butyl-9H-purin-6-ylamine (35)

Rt=805; ¹H NMR (CDCl3) δ 8.31 (s, 1H), 7.31 (dd, J=8.5 & 2.3 Hz, 1H), 7.16 (d, J=2.2 Hz, 1H), 6.87 (d, J=8.5 Hz, 1H), 6.59 (dd, J=17.6 & 10.9 Hz, 1H), 5.82-5.72 (s, 2H), 5.53 (dd, J=17.6 & 0.7 Hz, 1H), 5.09 (dd, J=10.9 & 0.7 Hz, 1H), 4.22 (s, 2H), 4.06 (t, J=7.6 Hz, 2H), 3.85 (s, 3H), 1.62 (quint., J=7.7 Hz, 2H), 1.30 (sext., J=7.4 Hz, 2H), 0.87 (t, J=7.4 Hz, 3H).

### EXAMPLE 36 8-(4-Amino-2,5-dimethoxy-benzyl)-9-butyl-9H-purin-6-ylamine (36)

Rt=6.95; 1H NMR (CDCl3) δ 8.33 (s, 1H), 6.57 (s, 1H), 6.33 (s, 1H), 6.37-6.27 (s, 2H), 4.20 (s, 2H), 4.01 (t, J=7.3 Hz, 2H), 3.74 (s, 3H), 3.68 (s, 3H), 1.59 (quint., J=7.5 Hz, 2H), 1.32 (sext., J=7.5 Hz, 2H), 0.86 (t, J=7.4 Hz, 3H).

### EXAMPLE 37 8-(2-Amino-3,5-dimethoxy-benzyl)-9-butyl-9H-purin-6-ylamine (37)

¹H NMR (CDCl3) δ 8.28 (s, 1H), 6.40 (d, J=2.5 Hz, 1H), 6.30 (d, J=2.5 Hz, 1H), 5.85-5.75 (s, 2H), 4.14 (s, 2H), 4.13 (t, J=7.6 Hz, 2H), 3.80 (s, 3H), 3.73 (s, 3H), 1.62 (quint., J=7.5 Hz, 2H), 1.48 (sext., J=7.5 Hz, 2H), 0.91 (t, J=7.4 Hz, 3H).

### EXAMPLE 38 2-(6-Amino-9-butyl-9H-purin-8-ylmethyl)-4-methoxy-benzaldehyde-O-methyl-oxime

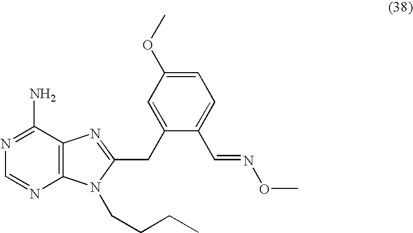

Rt=7.69; ¹H NMR (CDCl3) δ 8.88 (s, 1H), 8.31 (s, 1H), 7.72 (d, J=7.9 Hz, 1H),), 6.80 (d, J=8.0 Hz, 1H), 6.74 (s, 1H), 5.80-5.76 (s, 2H), 4.24 (s, 2H), 4.00 (t, J=7.7 Hz, 2H), 3.94 (s, 3H), 3.76 (s, 3H), 1.58 (quint., J=7.7 Hz, 2H), 1.28 (sext., J=7.5 Hz, 2H), 0.86 (t, J=7.3 Hz, 3H).

Formylation of Benzene Ring and Derivatization

A solution of 9-butyl-8-(3-methoxy-benzyl)-9H-purin-6-ylamine (100 mg, 0.32 mmol), 1,1-dichlorodimethyl ether (40 mg, 0.35 mmol) and TiCl₄ (133 mg, 0.70 mmol) in CH₂Cl₂ (10 ml) was prepared at 0° C. and stirred at r.t. overnight. Dilution with CH₂Cl₂, washing (Na₂SO₄, NH₄Cl), drying, and preparative thin layer chromatography gave the title aldehyde as a yellow glass (47 mg, 43%).

Standard procedures can give the corresponding alcohol (NaBH₄, MeOH, r.t.), tosyl hydrazone (TsNHNH₂, EtOH, reflux), oximes (RONH₂.HCl, DMF, 60° C.), amines (R₁R₂NH, NaBH(OAc)₃, Cl—(CH₂)₂—Cl r.t.), homoallylic alcohol (AllSiMe₃, TiCl₄), CH₂Cl₂, −78° C.), or alkenes.

### EXAMPLE 39 2-(6-Amino-9-butyl-9H-purin-8-ylmethyl)-4-methoxy-benzaldehyde

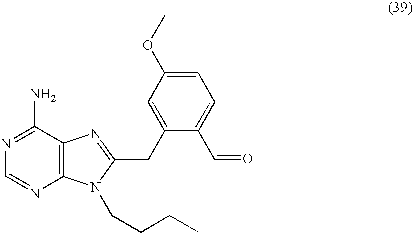

Rt=6.52; ¹HNMR (CDCl3-d) δ 10.39 (s, 1H), 8.32 (s, 1H), 7.76 (d, J=7.8 Hz, 1H), 6.87 (m, 2H), 6.22 (s, 2H), 4.28 (s, 2H), 4.03 (t, J=7.6 Hz, 2H), 3.85 (s, 3H), 1.61 (quint., J=7.3 Hz, 2H), 1.29 (sext., J=7.4 Hz, 2H), 0.86 (t, J=7.2 Hz, 3H).

Negishi Couplings

A mixture of 3,4-dichlorobenzyl bromide (0.47 g, 1.96 mmol) and Rieke Zinc (3.0 ml, 5 g/100 ml THF, 2.35 mmol) was stirred overnight at r.t. in a flame-dried Schlenk tube and decanted to provide a 0.65M stock solution of 3,4-dichlorobenzyl zinc bromide. A solution of 8-bromo-9-butyl-9H-purin-6-ylamine (42.7 mg, 0.158 mol), Pd(dppf)Cl₂ (16.8 mg, 0.020 mmol), and 3,4-dichlorobenzyl zinc bromide (0.61 ml, 0.65M in THF) was stirred in a flame-dried Schlenk tube at 66° C. overnight, quenched with sat. aq. NH₄Cl and sat. aq. EDTA., extracted into EtOAc, dried and concentrated. Preparative TLC purification (EtOAc/CH₂Cl₂/MeOH 14:14:2) provided the title compound as a colorless oil (approx. 15 mg, 20%).

### EXAMPLE 40 9-Butyl-8-(3,4-dichloro-benzyl)-9H-purin-6-ylamine (40)

compound isolated as solid, Rt=7.98.

### EXAMPLE 41 3-(6-Amino-9-butyl-9H-purin-8-ylsulfanyl)-phenol

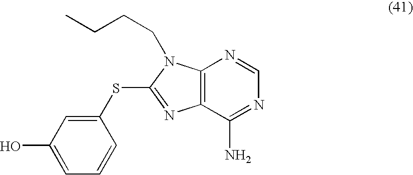 Step 1: Adenine (47 g, 0.35 mole) was suspended in 200 ml of CHCl3 before adding bromine (180 ml, 3.5 mole) in one portion. The suspension was left stirring at room temperature for 72 hours in a closed system that was vented by a 20 G needle. The reaction was worked up by adding shaved ice into the suspension before slowly neutralizing with aqueous ammonia to pH 8-9, followed by precipitation of the desired product with acetic acid. The crude product was dried under reduced pressure for 2 days to give 8-Bromoadenine as a light brown powder (45 g, 60% yield). ¹H NMR (DMSO-d₆) δ 8.12 (s, 1H), 7.22 (s, 2H). Rf (75% EtOAc/Hex)=0.4.
Step 2: 8-Bromopurine (2.2 g, 10 mmole) was dissolved in 50 ml of DMF before adding 1-bromo-butane (2.2 ml, 20 mmol) and cesium carbonate (6.7 g, 20 mmol) into the solution. The reaction mixture was left stirring at room temperature for 16 hours before quenching with water and extracting with EtOAc. The organic layer was washed with water and dried with MgSO₄ before removing solvent under reduced pressure. A white powder (0.9 g, 33%) of 8-Bromo-9-butyl-9H-purin-6-ylamine was isolated using silica gel column chromatography (50% EtOAc/Hexanes). ¹H NMR (CDCl₃) δ 8.32, (s, 1H), 5.81 (s, 2H), 4.20 (t, 2H), 1.82 (m, 2H), 1.40 (m, 2H), 0.96 (t, 3H). Rf (75% EtOAc/Hex)=0.6.
Step 3: To a mixture of sodium hydride (96 mg, 4 mmol) in DMF (4 ml) was added 3-methoxy-benzenethiol (1.12 g, 8 mmol). After 30 min, a solution of 8-bromo-9-butyl-9H-purin-6-ylamine (0.54 g, 2 mmol) in DMF (6 ml) was added and stirred for 12 h at 70° C. The reaction mixture was quenched by addition of MeOH (4 ml), diluted with EtOAc (400 ml), washed with Na₂CO₃ (3×100 ml), brine (3×100 ml), dried (Na₂SO₄), evaporated, purified with flash chromatography (CH₂Cl₂:MeOH=100:5) to give 3-(6-Amino-9-butyl-9H-purin-8-ylsulfanyl)-phenol as a colorless powder (0.59 g, 89%). Rt=6.75 min ¹HNMR (DMSO-d₆): δ 9.69 (s, 1H), 8.17 (s, 1H), 7.45 (s, 2H), 7.17 (t, J=7.9 Hz, 1H), 6.76(d, J=7.4 Hz, 1H), 6.68 (d, J=8.2 Hz, 1H), 6.62 (s, 1H), 4.11 (t, J=7.0 Hz, 2H), 1.57 (quint., J=7.3 Hz, 2H), 1.19 (sext., J=6.8 Hz, 2H), 0.81 (t, J=7.4 Hz, 3H).

HPLC method used for these compounds: Agilent Zorbax 300 SB C18, 4.6×150 mm, 5 μm; Column Temperature: Ambient; Flow Rate: 1.0 ml/min, Gradient: 5% acetonitrile (0.05% TFA) in water (0.1% TFA) to 100% acetonitrile (0.05% TFA) in 15 minutes, hold at 100% for 2 minutes).

The following compounds were prepared as for example 41, using the corresponding thiol in place of the 3-methoxybenzene thiol used in step 3:

### EXAMPLE 42 9-Butyl-8-(3-methoxy-phenylsulfanyl)-9H-purin-6-ylamine (42)

Rt=8.6 min; ¹H NMR (DMSO-d₆) δ 0.80 (t, J=7.4 Hz, 3H, CH₃), 1.20 (m, 2H, CH₂), 1.61 (m, 2H, CH₂), 3.60 (s, 3H, OCH₃), 3.76 (s, 3H, OCH₃), 4.13 (t, J=7.4 Hz, 2H, CH₂), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H).

### EXAMPLE 43 9-Butyl-8-(2,5-dimethoxy-phenylsulfanyl)-9H-purin-6-ylamine) (43)

Rt=7.62 min; ¹HNMR (CDCl₃-d₆): δ 8.30 (s, 1H), 7.18 (t, J=8.2 Hz, 1H), 6.90 (m, 2H), 6.77 (m, 3H), 4.17 (t, J=7.6 Hz, 2H), 3.70 (s, 3H), 1.67 (quint., J=7.5 Hz, 2H), 1.28 (sext., J=7.5 Hz, 2H), 0.86 (t, J=7.4 Hz, 3H).

### EXAMPLE 44 9-Butyl-8-(2-iodo-5-methoxy-phenylsulfanyl)-9H-purin-6-ylamine

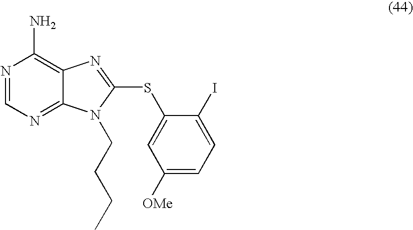 and

### EXAMPLE 45 9-Butyl-8-(4-iodo-3-methoxy-phenylsulfanyl)-9H-purin-6-ylamine

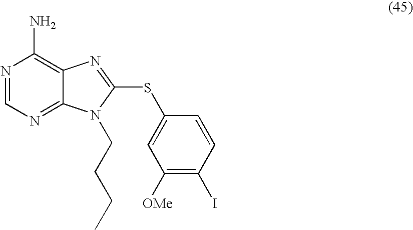

As for example 42, followed by:

Step 4: To a solution of 9-butyl-8-(3-methoxy-phenylsulfanyl)-9H-purin-6-ylamine (0.26 g, 0.73 mmol) in AcOH (4 ml) was added NIS (0.53 g, 2.19 mmol) in portions. The mixture was stirred for 24 h at r.t. The reaction mixture was diluted with EtOAc (200 ml), and carefully neutralized with a solution of K₂CO₃ (2×50 ml), them washed with Na₂S₂O₃ (3×50 ml), brine (3×50 ml), dried (Na₂SO₄), evaporated, purified by preparative TLC chromatography (CH₂Cl₂:MeOH=100:5) to give

the 2-iodo isomer (60 mg), and

Rt=8.45 min; ¹HNMR (CDCl₃-d): δ 8.38 (s, 1H), 7.73 (d, J=8.7 Hz, 1H), 6.71 (d, J=2.7 Hz, 1H), 6.58 (dd, J=8.7, 2.7 Hz, 1H), 5.91 (s, 2H), 4.22 (t, J=7.4 Hz, 2H), 3.68 (s, 3H), 1.75 (quint., J=7.7 Hz, 2H), 1.34 (sext., J=7.5 Hz, 2H), 0.93 (t, J=7.4 Hz, 3H).

the 4-iodo isomer (65 mg).

Rt=8.63 min; ¹HNMR (CDCl₃-d): δ 8.38 (s, 1H), 7.72 (d, J=8.1 Hz, 1H), 6.92 (d, J=1.8 Hz, 1H), 6.58 (dd, J=8.1, 1.8 Hz, 1H), 5.82 (s, 2H), 4.22 (t, J=7.4 Hz, 2H), 3.85 (s, 3H), 1.75 (quint., J=7.7 Hz, 2H), 1.37 (sext., J=7.5 Hz, 2H), 0.93 (t, J=7.4 Hz, 3H).

For compounds in which the N9 substituent is sensitive to halogenation conditions, these may be prepared using iodide already present in the benzenethiol moiety:

To a suspension of sodium hydride (96 mg, 4 mmol) in DMF (3 ml) was added 2-iodo-5-methoxy-benzenethiol (1.06 g, 4 mmol; J Org. Chem, 2001, 66(13), 4525-4542). After 30 min, a solution of 8-bromo-9-(4-methyl-pent-3-enyl)-9H-purin-6-ylamine (296 mg, 1 mmol) in DMF (3 ml) was added, and the mixture was stirred for 12 h at 70° C. The reaction was quenched by addition of MeOH (2 ml), diluted with EtOAc (200 ml), washed with Na₂CO₃ (3×50 ml), brine (3×50 ml), dried (Na₂SO₄), evaporated, and purified by flash chromatography (CH₂Cl₂:MeOH=100:5) to give 8-(2-Iodo-5-methoxy-phenylsulfanyl)-9-(4-methyl-pent-3-enyl)-9H-purin-6-ylamine as a colorless powder (280 mg, 58%).

The following compounds were prepared by this method using, respectively, the electrophiles 1-bromo-4-methyl-pent-3-ene and 1-chloro-pent-4-yn:

### EXAMPLE 46 8-(2-Iodo-5-methoxy-phenylsulfanyl)-9-(4-methyl-pent-3-enyl)-9H-purin-6-ylamine

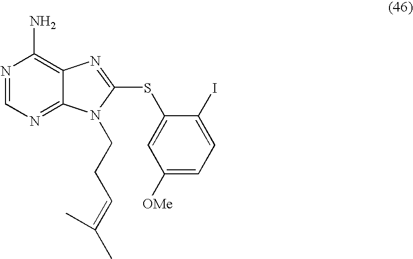

Rt=9.14 min; ¹HNMR (CDCl₃-d): δ 8.39 (s, 1H), 7.72 (d, J=8.7 Hz, 1H), 6.72 (d, J=2.7 Hz, 1H), 6.58 (dd, J=8.7, 2.7 Hz, 1H), 5.81 (s, 2H), 5.15 (t, J=7.3 Hz, 1H), 4.25 (t, J=7.4 Hz, 2H), 3.69 (s, 3H), 2.50 (quint., J=7.3 Hz, 2H), 1.66 (s, 3H), 1.44 (s, 3H); MP: 167-167.5° C.

### EXAMPLE 47 8-(2-Iodo-5-methoxy-phenylsulfanyl)-9-pent-4-ynyl-9H-purin-6-ylamine

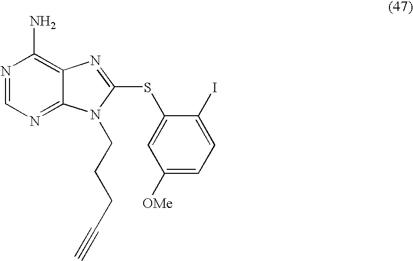

Rt=7.93 min; ¹HNMR (CDCl₃-d): δ 8.38 (s, 1H), 7.75 (d, J=8.7 Hz, 1H), 6.74 (d, J=2.7 Hz, 1H), 6.60 (dd, J=8.7, 2.7 Hz, 1H), 5.72 (s, 2H), 4.32 (t, J=7.3 Hz, 2H), 3.70 (s, 3H), 2.28 (td, J=6.8, 2.6 Hz, 2H), 2.06 (quint., J=7.3 Hz, 2H), 2.00 (t, J=2.4 Hz, 1H); MP: 168-169° C.

The following compounds were prepared using the corresponding thiol in place of the 3-methoxybenzene thiol and base t-BuOK in place of NaH used in step 3:

### EXAMPLE 48 8-(Benzothiazole-2-ylsulfanyl)-9-butyl-9H-purin-6-ylamine (48)

Rt=6.53 min; ¹H NMR (CDCl₃) δ 8.41 (s, 1H), 7.94 (d, 1H), 7.74 (d, 1H), 7.47 (t, 1H), 7.38 (t, 1H), 6.01 (s, 2H), 4.32 (t, 2H), 1.79 (m, 2H), 1.35 (m, 2H), 0.89 (t, 3H).

### EXAMPLE 49 9-Butyl-8-(5-chloro-benzothiazole-2-ylsulfanyl)-9H-purin-6-ylamine (49)

Mass (M+1)=391.8 et (M+3)=393.8; ¹H NMR (CDCl₃) δ 8.43 (s, 1H), 7.92 (s, 1H), 7.65 (d, 1H), 7.35 (d, 1H), 6.01 (s, 2H), 4.32 (t, 2H), 1.79 (m, 2H), 1.35 (m, 2H), 0.89 (t, 3H).

### EXAMPLE 50 9-Butyl-8-(5-methoxy-benzothiazole-2-ylsulfanyl)-9H-purin-6-ylamine (50)

¹H NMR (CDCl₃) δ 8.42 (s, 1H), 7.60 (d, 1H), 7.43 (s, 1H), 7.02 (d, 1H), 5.82 (s, 2H), 4.33 (t, 2H), 3.99 (s, 3H), 1.80 (m, 2H), 1.35 (m, 2H), 0.89 (t, 3H).

### EXAMPLE 51 9-Butyl-8-(2,5-dichloro-phenylylsulfanyl)-9H-purin-6-ylamine (51)

¹H NMR (CDCl₃) δ 8.37 (s, 1H), 7.35 (d, 1H), 7.20 (dd, 1H), 7.14 (d, 1H), 5.72 (s, 2H), 4.24 (t, 2H), 1.79 (m, 2H), 1.35 (m, 2H), 0.89 (t, 3H).

### EXAMPLE 529 Butyl-8-(2,4,5-trichloro-phenylylsulfanyl)-9H-purin-6-ylamine (52)

Rt=7.8 min; ¹H NMR (CDCl₃) δ 8.37 (s, 1H), 7.62 (s, 1H), 7.35 (s, 1H), 5.98 (s, 2H), 4.27 (t, 2H), 1.80 (m, 2H), 1.36 (m, 2H), 0.92 (t, 3H).

General Procedure

8-(2,5-dimethoxy-phenylsulfanyl)-2-fluoro-9(4-methyl-pent-3-enyl)-9H-purin-6-ylamine and 8-(2,5-dimethoxy-phenylsulfanyl)-2-amino-9(4-methyl-pent-3-enyl)-9H-purin-6-ylamine were prepared from 2,6-diaminopurine by procedures analogous to the one described above. The final conversion of amino to fluoro was done by a method similar to that reported in Example 2, step 4.

### EXAMPLE 53 8-(2,5-dimethoxy-phenylsulfanyl)-2-amino-9(4-methyl-pent-3-enyl)-9H-purin-6-ylamine (53)

¹H NMR (DMSO-d6) δ 1.28 (s, 3H, CH₃), 1.58 (s, 3H, CH₃), 2.35 (m, 2H, CH₂), 3.60 (s, 3H, OCH₃), 3.76 (s, 3H, OCH₃), 4.12 (t, J=7.0 Hz, 2H, CH₂), 5.05 (t, J=7 Hz, 1H, CH═), 6.50(s, 1H, Ar—H), 6.91 (d, J=8.9 Hz, 1H, Ar—H), 7.05 (d, J=8.9 Hz, 1H, Ar—H).

### EXAMPLE 54 8-(2,5-dimethoxy-phenylsulfanyl)-2-fluoro-9(4-methyl-pent-3-enyl)-9H-purin-6-ylamine (54)

¹H NMR (DMSO-d6) δ 1.30 (s, 3H, CH₃), 1.55 (s, 3H, CH₃), 2.35 (m, 2H, CH₂), 3.60 (s, 3H, OCH₃), 3.76 (s, 3H, OCH₃), 4.10 (t, J=7.0 Hz, 2H, CH₂), 5.05 (t, J=7 Hz, 1H, CH═), 6.47(s, 1H, Ar—H), 6.86 (d, J=8.9 Hz, 1H, Ar—H), 7.02 (d, J=8.9 Hz, 1H, Ar—H); MS (m/z) 426 (M+Na).

The following 12 compounds were prepared analogously to the method described above in Example 41 using various electrophiles to generate a library of N9 substituted compounds. N9 alkylation was done as a final step after the bromine displacement of 8-bromopurine with 2,5-dimethoxy thiophenol.

### EXAMPLE 55 8-(2,5-dimethoxy-phenylsulfanyl)-9H-purin-6-ylamine (55)

¹H NMR (DMSO-d₆) δ 3.62 (s, 3H, OCH₃), 3.76 (s, 3H, OCH₃), 6.61(s, 1H, Ar—H), 6.85 (d, J=8.9 Hz, 1H, Ar—H), 7.02 (d, J=8.9 Hz, 1H, Ar—H), 7.24 (bs, 2H, NH₂), 8.13 (s, 1H, purine-H) 13.33 (s, 1H, purine-NH); electrophile: No substitution on N9.

### EXAMPLE 56 8-(2,5-dimethoxy-phenylsulfanyl)-9-pentyl-9H-purin-6-ylamine (56)

¹H NMR (DMSO-d₆) δ 0.80 (t, J=7.4 Hz, 3H, CH₃), 1.20 (m, 4H, 2CH₂), 1.61 (m, 2H, CH₂), 3.60 (s, 3H, OCH₃), 3.76 (s, 3H, OCH₃), 4.13 (t, J=7.4 Hz, 2H, CH₂), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H); electrophile: 1-bromopentyl.

### EXAMPLE 57 8-(2,5-dimethoxy-phenylsulfanyl)-9-pent-4-ynyl-9H-purin-6-ylamine

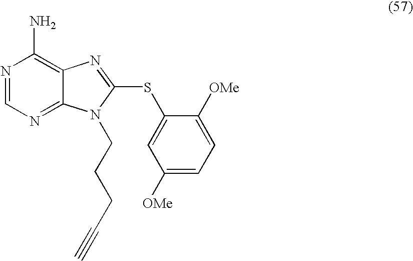

¹H NMR (DMSO-d₆) δ 1.89(m, 2H, CH₂), 2.20(t, J=8.0 Hz, 2H, CH₂), 2.78(s, 1H, CH═), 3.62 (s, 3H, OCH₃), 3.76 (s, 3H, OCH₃), 4.23 (t, J=7.4 Hz, 2H, CH₂), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H); electrophile: 1-chloro-pent-4-yne.

### EXAMPLE 58 8-(2,5-dimethoxy-phenylsulfanyl)-9(3,3,3-trifluoromethylpropyl)-9H-purin-6-ylamine (58)

¹H NMR (DMSO-d₆) δ 2.54(t, J=8.0 Hz, 2H, CH₂), 3.62 (s, 3H, OCH₃), 3.74 (s, 3H, OCH₃), 4.46 (t, J=8.0 Hz, 2H, CH₂), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.30 (s, 1H, purine-H); electrophile: 1-bromo-3,3,3-trifluoro-propane.

### EXAMPLE 59 8-(2,5-dimethoxy-phenylsulfanyl)-9(4-chlorobutyl)-9H-purin-6-ylamine (59)

¹H NMR (DMSO-d₆) δ 1.82(m, 2H, CH₂), 1.98(m, 2H, CH₂), 3.56(t, J=6.4 Hz, 2H, CH₂), 3.75 (s, 3H, OCH₃), 3.78 (s, 3H, OCH₃), 4.23 (t, J=7.4 Hz, 2H, CH₂), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H); electrophile: 1-bromo-4-chlorobutane.

### EXAMPLE 60 8-(2,5-dimethoxy-phenylsulfanyl)-9(4-acetyloxybutyl)-9H-purin-6-ylamine (60)

¹H NMR (DMSO-d₆) δ 1.70(m, 2H, CH₂), 1.90(m, 2H, CH₂), 2.02(s, 3H, CH₃), 3.75 (s, 3H, OCH₃), 3.78 (s, 3H, OCH₃), 4.10 (t, J=6.4 Hz, 2H, CH₂), 4.30 (t, J=7.4 Hz, 2H, CH₂), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H); electrophile: 1-bromo-4-acetyloxybutane.

### EXAMPLE 61 8-(2,5-dimethoxy-phenylsulfanyl)-9(5-bromopentyl)-9H-purin-6-ylamine (61)

¹H NMR (DMSO-d₆) δ 1.46 (m, 2H, CH₂), 1.85(m, 4H, 2CH₂), 3.36(t, J=6.7 Hz, 2H, CH₂), 3.72 (s, 3H, OCH₃), 3.80 (s, 3H, OCH₃), 4.30 (t, J=7.4 Hz, 2H, CH₂), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H); electrophile: 1,5-dibromopentane.

### EXAMPLE 62 8-(2,5-dimethoxy-phenylsulfanyl)-9(2-[1,3]dioxolan-2-yl-ethyl)-9H-purin-6-ylamine (62)

¹H NMR (DMSO-d₆) δ2.26 (m, 2H, CH₂), 3.75 (s, 3H, OCH₃), 3.77 (s, 3H, OCH₃), 3.85(t, J=7.0 Hz, 2H, CH₂), 3.98(t, J=7.0 Hz, 2H, CH₂), 4.46 (t, J=7.4 Hz, 2H, CH₂), 4.96(t, J=4.1 Hz, 1H, CH), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H); electrophile: 2-(2-Chloro-ethyl)-[1,3]dioxolane.

### EXAMPLE 63 8-(2,5-dimethoxy-phenylsulfanyl)-9-(4-methyl-pent-3-enyl)-9H-purin-6-ylamine

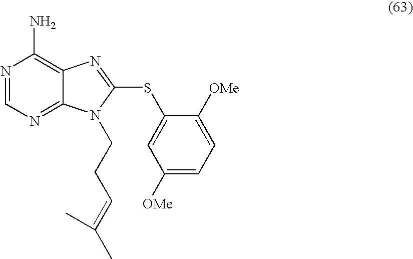

¹H NMR (DMSO-d₆) δ 1.28 (s, 3H, CH₃), 1.54 (s, 3H, CH₃), 2.35 (m, 2H, CH₂), 3.60 (s, 3H, OCH₃), 3.76 (s, 3H, OCH₃), 4.15 (t, J=7.0 Hz, 2H, CH₂), 5.05 (t, J=7 Hz, 1H, CH═), 6.46(s, 1H, Ar—H), 6.86 (d, J=8.9 Hz, 1H, Ar—H), 7.02 (d, J=8.9 Hz, 1H, Ar—H), 7.42(bs, 2H, NH₂), 8.17 (s, 1H, purine-H); electrophile: 1-bromo-4-methyl-pent-3-ene; MP: 148-150° C.

### EXAMPLE 64 8-(2,5-dimethoxy-phenylsulfanyl)-9-(pent-4-enyl)-9H-purin-6-ylamine (64)

¹H NMR (DMSO-d₆) δ 1.89(m, 2H, CH₂), 2.19(t, J=8.0 Hz, 2H, CH₂), 3.62 (s, 3H, OCH₃), 3.76 (s, 3H, OCH₃), 4.23 (t, J=7.4 Hz, 2H, CH₂), 5.05(m, 2H, CH₂═), 5.82(m, 1H, CH═), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H); electrophile: 1-chloro-pent-4-yne.

### EXAMPLE 65 8-(2,5-dimethoxy-phenylsulfanyl)-9-(3-hydroxypropyl)-9H-purin-6-ylamine (65)

¹H NMR (DMSO-d₆) δ 1.82(m, 2H, CH₂), 3.60 (s, 3H, OCH₃), 3.76 (s, 3H, OCH₃), 4.12(m, 2H, CH₂), 4.21 (t, J=7.0 Hz, 2H, CH₂), 6.47(s, 1H, Ar—H), 6.86 (d, J=8.9 Hz, 1H, Ar—H), 7.02 (d, J=8.9 Hz, 1H, Ar—H); 8.15 (s, 1H, purine-H); electrophile: 1-bromo-3-hydroxypropane.

### EXAMPLE 66 4-[6-Amino-8(2,5-dimethoxysulfanyl)-purin-9-yl]-butyronitrile (66)

¹H NMR (DMSO-d₆) δ 1.89(m, 2H, CH₂), 2.20(t, J=8.0 Hz, 2H, CH₂), 3.62 (s, 3H, OCH₃), 3.76 (s, 3H, OCH₃), 4.23 (t, J=7.4 Hz, 2H, CH₂), 6.46(s, 1H, Ar—H), 6.85 (d, J=8.9 Hz, 1H, Ar—H), 7.02 (d, J=8.9 Hz, 1H, Ar—H), 7.41(bs, 2H, NH₂), 8.15 (s, 1H, purine-H); electrophile: 1-bromobutyronitrile.

### EXAMPLE 67 9-Butyl-8-(2-iodo-5-methoxy-phenylsulfanyl)-9H-purin-6-ylamine (67)

This compound was prepared using diazonium salts and thiols as coupling partners.

Step 1: A suspension of 8-bromo-9-butyl-9H-purin-6-ylamine (0.50 g, 1.85 mmol) and thiourea (1.49 g, 19.6 mmol) in n-butanol (10 ml) was heated to reflux for 14 h. Dilution with CH₂Cl₂ (70 ml), washing with water and concentration afforded 6-amino-9-butyl-7,9-dihydro-purine-8-thione as a white powder (0.42 g, 1.87 mmol, 100%). ¹H NMR (DMSO-d₆) δ 12.35-12.25 (br. s, 1H), 8.13 (s, 1H), 6.92-6.72 (br. s., 2H), 4.09 (t, J=7.6 Hz, 2H), 1.71 (quint., J=7.5 Hz, 2H), 1.29 (sext., J=7.5 Hz, 2H), 0.87 (t, J=7.4 Hz, 3H).

Step 2: A solution of the above thione (30.8 mg, 0.138 mmol) and t-BuOK (15.5 mg, 0.138 mmol) in MeOH (0.55 ml) was treated portion-wise with crude 2-iodo-5-methoxy-benzenediazonium tetrafluoroborate (48 mg, 0.138 mmol). The vigorous N₂ evolution ceased after 2 min. Work-up and preparative TLC (MeOH:CH₂Cl₂ 5:95) yielded the title sulfide. Example 68 2-Fluoro-8-(2-iodo-5-methoxy-phenylsulfanyl)-9-pent-4-ynyl-9H-purin-6-ylamine (68)

### Step 1 8-Bromo-9-pent-4-ynyl-9H-purine-2,6-diamine

A mixture of 8-bromo-9H-purine-2,6-diamine (600 mg; Beaman et al, J. Org. Chem., 1962, 27, 986), Cs₂CO₃ (1.94 g), 5-chloro-pent-1-yne (0.56 mL), and DMF (5 mL) was heated to 85° C. overnight. Work-up and evaporation gave the title compound as a crude solid. ¹H NMR (CDCl₃) δ 6.80 (s, 2H), 5.95 (s, 2H), 3.98 (t, 2H), 2.81 (t, 1H), 2.22 (t, 2H), 1.96 (quint., 2H).

### Step 2 8-(2-Iodo-5-methoxy-phenylsulfanyl)-9-pent-4-ynyl-9H-purine-2,6-diamine

A mixture of 8-bromo-9-pent-4-ynyl-9H-purine-2,6-diamine (500 mg), 2-iodo-5-methoxy-benzenethiol (1.34 g), t-BuOK (475 mg) and DMF (7 mL) was heated to 100° C. overnight. Extraction and chromatography gave the title compound. Rt=7.85 min. ¹H NMR (CDCl₃) δ 7.72 (d, 1H), 6.98 (s, 2H), 6.63 (d, 1H), 6.22 (dd, 1H), 6.01 (s, 2H), 4.01 (t, 2H), 3.60 (s, 3H), 2.67 (t, 1H), 2.12 (dt, 2H), 1.78 (quint., 2H), 1.97 (t, 1H).

### Step 3 2-Fluoro-8-(2-iodo-5-methoxy-phenylsulfanyl)-9-pent-4-ynyl-9H-purin-6-ylamine

A mixture of 8-(2-iodo-5-methoxy-phenylsulfanyl)-9-pent-4-ynyl-9H-purine-2,6-diamine (79 mg) and 48% aq. HBF₄ (0.5 mL) in THF (0.5 mL) was treated at −20° C. with iso-amyl nitrite (22 uL). The reaction mixture was allowed to reach rt and was further heated to 40° C. for 10 min. Work-up (DCM/aq. K₂CO₃) and chromatography (EtOAc/Hexane 1:4) gave the title compound as a solid. Rt=9.43 min. ¹H NMR (CDCl₃) δ 7.72 (d, 1H), 6.70 (d, 1H), 6.59 (dd, 1H), 4.25 (t, 2H), 3.69 (s, 3H), 2.25 (dt, 2H), 2.02 (quint., 2H), 1.97 (t, 1H).

### EXAMPLE 69 9-(tert-Butyl-dimethyl-silanyloxymethyl)-8-(2-iodo-5-methoxy-phenylsulfanyl)-9H-purin-6-ylamine (69)

A solution of 9-(tert-Butyl-dimethyl-silanyloxymethyl)-8-iodo-9H-purin-6-ylamine (817 mg; Lang, J. Org. Chem. 2000, 65, 7825) and of potassium 2-iodo-5-methoxy-benzenethiolate (920 mg; Flynn, Org. Lett. 2001, 3, 651) in DMF (10 mL) was heated to 60° C. for 1 h and to 100° C. for another 1 h. Work-up and flash chromatography (CH₂Cl₂:EtOAc 67:33→0:100) gave the title compound as a white solid. Rt=10.47 min. ¹H NMR (CDCl₃) δ 8.37 (s, 1H), 7.70, (d, 1H), 6.83 (d, 1H), 6.56 (dd, 1H), 5.83 (s, 2H), 5.75 (s, 2H), 3.67 (s, 3H), 0.83 (s, 9H), 0.09 (s, 6H).

### EXAMPLE 70 9-(2-Chloro-ethyl)-8-(2-Iodo-5-methoxy-phenylsulfanyl)-9H-purin-6-ylamine (70)

### Step 1 6-Amino-7,9-dihydro-purine-8-thione

3,4,5-Triaminopyrimidine (50 g) and thiourea (60 g, 2 equiv.) were ground together in a mortar, and heated until molten (Tᵢₙₜ=150° C.), whereupon an endothermic reaction took place. The reaction mixture was stirred at that temperature until solidification occurred (2 h), cooled to room temperature, finely ground, and stirred in water overnight to remove the excess thiourea. The desired material was obtained by filtration (88-94% yield, 100% purity). Rt=1.99 min. ¹H-NMR (DMSO-d₆) δ 13.04 (s, 1H), 12.05 (s, 1H), 8.07 (s, 1H), 6.75 (s, 2H). ¹³C-NMR (DMSO-d₆) δ 167.0, 153.1, 150.33, 147.8, 108.5.

### Step 2 8-(2-Iodo-5-methoxy-phenylsulfanyl)-9h-purin-6-ylamine

A suspension of the finely ground 6-amino-7,9-dihydro-purine-8-thione (46 g) in DMF (700 ml) was cooled to −60° C., and treated with 2-iodo-5-methoxybenzenediazonium tetrafluoroborate (100 g, 1.1 equiv; (a) Ma, J. Org. Chem. 2001, 66, 4525 (b) Flynn, Org. Lett, 2001, 3, 651). The mixture was allowed to warm up gradually. When it reached −10° C., a gas evolution was observed, as well as the formation of a deep red color due to a minor but highly colored by-product. The reaction mixture was allowed to reach room temperature, before being neutralized with NaHCO₃ (38 g, 1.7 equiv.) concentrated, suspended in chloroform, filtered until no more red dye could be washed off, and further washed with water to afford the crude title material (64 g, “61%”). This material could be used without further purification. Alternatively, a work-up (extraction into NaOH 1M, EtOAc washing, acidification with HCl, EtOAc washing, neutralization, extraction into EtOAc) is feasible. Rt=6.08 min (5-100-12). ¹HNMR (DMSO) δ (br. s, 1H), 8.13 (s, 1H), 7.79 (d, 1H), 7.38 (br., s, 2H), 6.71 (d, 1H), 6.62 (s, 2H), 3.65 (s, 3H).

### Step 3 9-(2-Chloro-ethyl)-8-(2-iodo-5-methoxy-phenylsulfanyl)-9H-purin-6-ylamine

8-(2-Iodo-5-methoxy-phenylsulfanyl)-9H-purin-6-ylamine (7.3 g) was treated with 1-bromo-2-chloroethane (3.7 ml, 2.5 equiv.) and K₂CO₃ (7.6 g, 3 equiv.) in DMF at 40° C. for 16 h. The reaction mixture was concentrated, dissolved in MeOH: CH2Cl2 10:90 and washed with water. Chromatography (EtOAcCH₃CN:MeOH 800:200:2) gave 1.7 g (21%) of the desired material. Combining the impure fractions and crystallization (70 ml EtOH) gave an additional 0.9 g (11%). Rt=7.61 min (5-100-12). ¹HNMR (CDCl₃) δ (s, 1H), 7.73 (d, J=8.4 Hz, 1H), 6.77 (s, 1H), 6.60 (d, 1H), 5.93(br., s, 2H), 4.61 (t, J=4.4 Hz, 2H), 3.90 (t, J=4.4 Hz, 2H), 3.70 (s, 3H).

### EXAMPLE 71 9-(3-chloro-propyl)-8-(2-iodo-5-methoxy-phenylsulfanyl)-9H-purin-6-ylamine (71)

The title compound was obtained by reacting 8-(2-iodo-5-methoxy-phenylsulfanyl)-9H-purin-6-ylamine with 1-bromo-3-chloro-propane as in Example 15 Step 1. Rt=7.93 min (5-100-12). ¹H NMR (CDCl₃) δ 8.36 (s, 1H), 7.73 (d, 1H), 6.72 (d, 1H), 6.59 (dd, 1H), 6.07 (br.s, 2H), 4.38 (t, 2H), 3.68 (s, 3H), 3.55(t, 2H), 2.27 (quint. 2H).

### EXAMPLE 72 9-(4-Chloro-butyl)-8-(2-iodo-5-methoxy-phenylsulfanyl)-9H-purin-6-ylamine (72)

The title compound was obtained by reacting 8-(2-iodo-5-methoxy-phenylsulfanyl)-9H-purin-6-ylamine with 1-bromo-4-chloro-butane as in Example 15 Step 1. Rt=8.27 min (5-100-12). ¹H NMR (CDCl₃) δ 8.36 (s, 1H), 7.72 (d, 1H), 6.73 (s, 1H), 6.58 (d, 1H), 6.30 (br.s, 2H), 4.40 (m, 2H), 3.68 (s, 3H), 3.53 (m, 2H), 1.93 (m, 2H), 1.79 (m, 2H).

General Procedure A

A mixture of the alkyl chloride and the appropriate amine (5-30 eq in DMF or neat) was heated to 40-120° C. in a sealed tube overnight. Evaporation, work-up (CH₂Cl₂/sat. aq. NaHCO₃) and preparative TLC gave the desired amine.

Compounds 73-82, 84-120 were prepared in this manner.

### EXAMPLE 73 8-(2-Iodo-5-methoxy-phenylsulfanyl)-9-[3-(4-methyl-piperazin-1-yl)-propyl]-9H-purin-6-ylamine (73)

The title compound was obtained by reacting 9-(3-chloro-propyl)-8-(2-Iodo-5-methoxy-phenylsulfanyl)-9H-purin-6-ylamine with N-methylpiperazine according to the general procedure A. Rt=5.24 min. ¹H NMR (CDCl₃) δ 8.37 (s, 1H), 7.70, (d, 1H), 6.62 (d, 1H), 6.52 (dd, 1H), 5.78 (s, 2H), 4.30 (t, 2H), 3.62 (t, 3H), 2.30 (m, 10H), 2.22 (s, 3H), 1.95 (quint., 2H).

### EXAMPLE 74 9-(3-Dimethylamino-propyl)-8-(2-iodo-5-methoxy-phenylsulfanyl)-9H-purin-6-ylamine (74)

The title compound was obtained by reacting 9-(3-chloro-propyl)-8-(2-iodo-5-methoxy-phenylsulfanyl)-9H-purin-6-ylamine with dimethylamine (generated from dimethylamine hydrochloride and t-BuOK in DMF) according to the general procedure A. Rt=5.37 min. ¹H NMR (CDCl₃) δ 8.34 (s, 1H), 7.71, (d, 1H), 6.71 (d, 1H), 6.56 (dd, 1H), 5.83 (s, 2H), 4.31 (t, 2H), 3.68 (s, 3H), 2.29 (t, 3H), 2.37 (s, 6H), 2.11 (quint, 2H).

### EXAMPLE 75 8-(2-Iodo-5-methoxy-phenylsulfanyl)-9-(3-piperidin-1-yl-propyl)-9h-purin-6-ylamine (75)

The title compound was obtained by reacting 9-(3-chloro-propyl)-8-(2-iodo-5-methoxy-phenylsulfanyl)-9H-purin-6-ylamine with piperidine according to the general procedure A. Rt=5.78 min. ¹H NMR (CDCl₃/CD₃OD 10:1) δ 8.17 (s, 1H), 7.71 (d, 1H), 6.80 (d, 1H), 6.57 (dd, 1H), 4.20 (t, 2H), 3.66 (s, 3H), 2.30 (m, 4H), 1.94 (quint., 2H), 1.46 (quint., 4H), 1.21 (m, 2H).

### EXAMPLE 76 9-(3-Cyclopropylamino-propyl)-8-(2-iodo-5-methoxy-phenylsulfanyl)-9H-purin-6-ylamine (76)

The title compound was obtained by reacting 9-(3-chloro-propyl)-8-(2-iodo-5-methoxy-phenylsulfanyl)-9H-purin-6-ylamine with cyclopropylamine according to the general procedure A. Rt=5.58 min. ¹H NMR (CDCl₃) δ 8.34 (s, 1H), 7.72 (d, 1H), 6.79 (d, 1H), 6.57 (dd, 1H), 5.80 (s, 2H), 4.28 (t, 2H), 3.66 (s, 3H), 2.67 (t, 2H), 2.05 (m, 1H), 1.99 (quint., 2H), 0.42 (m, 4H).

### EXAMPLE 77 8-(2-Iodo-5-methoxy-phenylsulfanyl)-9-(3-morpholin-4-yl-propyl)-9H-purin-6-ylamine (77)

The title compound was obtained by reacting 9-(3-chloro-propyl)-8-(2-iodo-5-methoxy-phenylsulfanyl)-9H-purin-6-ylamine with morpholine according to the general procedure A. Rt=5.34 min. ¹H NMR (CDCl₃/CD₃OD 10:1) δ 8.21 (s, 1H), 7.72 (d, 1H), 6.79 (d, 1H), 6.57 (dd, 1H), 4.26 (t, 2H), 3.67 (s, 3H), 3.61 (t, 4H), 2.36 (m, 6H), 1.96 (quint., 2H).

### EXAMPLE 78 8-(2-Iodo-5-methoxy-phenylsulfanyl)-9-(3-methylamino-propyl)-9H-purin-6-ylamine (78)

The title compound was obtained by reacting 9-(3-chloro-propyl)-8-(2-Iodo-5-methoxy-phenylsulfanyl)-9H-purin-6-ylamine with 40% aq. methylamine in DMF according to the general procedure A. Rt=5.34 min. ¹H NMR (CDCl₃) δ 8.35 (s, 1H), 7.71 (d, 1H), 6.68 (d, 1H), 6.56 (dd, 1H), 5.82 (s, 2H), 4.29 (t, 2H), 3.66 (s, 3H), 2.53 (t, 2H), 2.87 (s, 3H), 2.73 (quint., 2H).

### EXAMPLE 79 9-(3-Ethylamino-propyl)-8-(2-iodo-5-methoxy-phenylsulfanyl)-9H-purin-6-ylamine (79)

The title compound was obtained by reacting 9-(3-chloro-propyl)-8-(2-iodo-5-methoxy-phenylsulfanyl)-9H-purin-6-ylamine with 70% aq. ethylamine in DMF according to the general procedure A. Rt=5.53 min. ¹H NMR (CDCl₃) δ 8.35 (s, 1H), 7.72 (d, 1H), 6.69 (d, 1H), 6.57 (dd, 1H), 5.78 (s, 2H), 4.31 (t, 2H), 3.67 (s, 3H), 2.56 (m, 4H), 1.96 (quint., 2H), 1.08 (t, 3H).

### EXAMPLE 80 8-(2-Iodo-5-methoxy-phenylsulfanyl)-9-[2-(4-methyl-piperazin-1-yl)-ethyl]-9H-purin-6-ylamine (80)

The title compound was obtained by reacting 9-(2-chloro-ethyl)-8-(2-iodo-5-methoxy-phenylsulfanyl)-9H-purin-6-ylamine with N-methyl piperazine according to the general procedure A. Rt=5.56 min. ¹H NMR (CDCl₃) δ 8.34 (s, 1H), 7.69 (d, 1H), 6.67 (d, 1H), 6.54 (dd, 1H), 5.73 (s, 2H), 4.34 (t, 2H), 3.66 (s, 3H), 2.69 (t, 2H), 2.50 (m, 4H), 2.30 (m, 4H), 2.24 (s, 3H).

### EXAMPLE 81 8-(2-Iodo-5-methoxy-phenylsulfanyl)-9-(2-piperidin-1-yl-ethyl)-9H-purin-6-ylamine (81)

The title compound was obtained by reacting 9-(2-chloro-ethyl)-8-(2-iodo-5-methoxy-phenylsulfanyl)-9H-purin-6-ylamine with piperidine according to the general procedure A. Rt=5.80 min. ¹H NMR (CDCl₃) δ 8.34 (s, 1H), 7.69 (d, 1H), 6.69 (d, 1H), 6.52 (dd, 1H), 5.68 (s, 2H), 4.33 (t, 2H), 3.66 (s, 3H), 2.63 (t, 2H), 2.41 (m, 4H), 1.51 (m, 4H), 1.30 (m, 2H).

### EXAMPLE 82 8-(2-Iodo-5-methoxy-phenylsulfanyl)-9-(2-propylamino-ethyl)-9H-purin-6-ylamine (82)

The title compound was obtained by reacting 9-(2-chloro-ethyl)-8-(2-iodo-5-methoxy-phenylsulfanyl)-9H-purin-6-ylamine with propylamine according to the general procedure A. Rt=5.69 min. ¹H NMR (CDCl₃) δ 8.34 (s, 1H), 7.69 (d, 1H), 6.69 (d, 1H), 6.60 (dd, 1H), 5.85 (s, 2H), 4.33 (t, 2H), 3.65 (s, 3H), 2.97 (t, 2H), 2.53 (t, 2H), 1.39 (sext., 2H), 0.85 (t, 3H).

### EXAMPLE 83 8-(2,5-Dimethoxy-phenylsulfanyl)-9-(3-dimethylamino-propyl)-9H-purin-6-ylamine (83)

A suspension of 8-(2,5-dimethoxy-phenylsulfanyl)-9H-purin-6-ylamine (127 mg), Me₂N—(CH₂)₃—Cl.HCl (236 mg), Cs₂CO₃ (680 mg) in DMF (2 mL) was heated to 90° C. for 2 h. Work-up and preparative TLC (MeOH:DCM 1:10) gave the title compound. Rt=4.83 min. ¹H NMR (CDCl₃) δ 8.33 (s, 1H), 6.85 (d, 1H), 6.81 (d, 1H), 6.75 (d, 1H), 5.68 (s, 2H), 4.42 (t, 2H), 3.79 (s, 3H), 3.70 (s, 3H), 2.35 (t, 2H), 2.22 (s, 6H), 1.99 (quint., 2H).

### EXAMPLE 84 8-(2-Iodo-5-methoxy-phenylsulfanyl)-9-(2-isopropylamino-ethyl)-9H-purin-6-ylamine (84)

The title compound was obtained by reacting 9-(2-chloro-ethyl)-8-(2-Iodo-5-methoxy-phenylsulfanyl)-9H-purin-6-ylamine with iso-propylamine according to the general procedure A. Rt=5.61 min. ¹H NMR (CDCl₃) δ 8.34 (s, 1H), 7.69 (d, 1H), 6.69 (d, 1H), 6.52 (dd, 1H), 5.68 (s, 2H), 4.33 (t, 2H), 3.66 (s, 3H), 3.02 (t, 2H), 3.85 (sept., 1H), 0.95 (d, 6H).

### EXAMPLE 85 9-(2-Butylamino-ethyl)-8-(2-iodo-5-methoxy-phenylsulfanyl)-9H-purin-6-ylamine (85)

The title compound was obtained by reacting 9-(2-chloro-ethyl)-8-(2-iodo-5-methoxy-phenylsulfanyl)-9H-purin-6-ylamine with butylamine according to the general procedure A. Rt=6.10 min. ¹H NMR (CDCl₃) δ 8.34 (s, 1H), 7.70 (d, 1H), 6.72 (d, 1H), 6.55 (dd, 1H), 5.78 (s, 2H), 4.32 (t, 2H), 3.67 (s, 3H), 3.00 (t, 2H), 2.60 (t, 2H), 1.40 (sext., 2H), 1.28 (quint., 2H), 0.87 (t, 3H).

### EXAMPLE 86 9-(2-sec-Butylamino-ethyl)-8-(2-iodo-5-methoxy-phenylsulfanyl)-9H-purin-6-ylamine (86)

The title compound was obtained by reacting 9-(2-chloro-ethyl)-8-(2-Iodo-5-methoxy-phenylsulfanyl)-9H-purin-6-ylamine with sec-butylamine according to the general procedure A. Rt=5.91 min. ¹H NMR (CDCl₃) δ 8.35 (s, 1H), 7.70 (d, 1H), 6.72 (d, 1H), 6.55 (dd, 1H), 5.67 (s, 2H), 4.35 (t, 2H), 3.68 (s, 3H), 3.03 (m, 1H), 2.95 (m, 1H), 2.54 (sext., 1H), 1.38 (sext., 1H), 1.24 (m, 1H), 0.96 (d, 3H), 0.82 (t, 3H).

### EXAMPLE 87 9-[2-(1-Ethyl-propylamino)-ethyl]-8-(2-iodo-5-methoxy-phenylsulfanyl)-9H-purin-6-ylamine (87)

The title compound was obtained by reacting 9-(2-chloro-ethyl)-8-(2-iodo-5-methoxy-phenylsulfanyl)-9H-purin-6-ylamine with 1-ethyl-propylamine according to the general procedure A. Rt=6.34 min. ¹H NMR (CDCl₃) δ 8.33 (s, 1H), 7.69 (d, 1H), 6.71 (d, 1H), 6.54 (dd, 1H), 6.07 (s, 2H), 4.33 (t, 2H), 3.65 (s, 3H), 2.96 (t, 2H), 2.34 (quint., 1H), 1.31 (m, 4H), 0.77 (t, 6H).

### EXAMPLE 88 9-(2-Cyclopropylamino-ethyl)-8-(2-iodo-5-methoxy-phenylsulfanyl)-9H-purin-6-ylamine (88)

The title compound was obtained by reacting 9-(2-chloro-ethyl)-8-(2-iodo-5-methoxy-phenylsulfanyl)-9H-purin-6-ylamine with cyclopropylamine according to the general procedure A. Rt=5.51 min. ¹H NMR (CDCl₃) δ 8.36 (s, 1H), 7.69 (d, 1H), 6.71 (d, 1H), 6.54 (dd, 1H), 5.71 (s, 2H), 4.33 (t, 2H), 3.66 (s, 3H), 3.07 (t, 2H), 2.11 (sept., 1H), 0.34 (m, 2H), 0.23 (m, 2H).

### EXAMPLE 89 8-(2-Iodo-5-methoxy-phenylsulfanyl)-9-(2-isobutylamino-ethyl)-9H-purin-6-ylamine (89)

The title compound was obtained by reacting 9-(2-chloro-ethyl)-8-(2-iodo-5-methoxy-phenylsulfanyl)-9H-purin-6-ylamine with iso-butylamine according to the general procedure A. Rt=6.10 min (5-100-12). ¹H NMR (DMSO-d₆) δ 8.16 (s, 1H), 7.74 (d, 1H), 6.45 (br. s, 1H), 6.67 (dd, 1H), 6.46 (d, 1H), 4.19 (t, 2H), 3.60 (s, 3H), 2.77 (t, 2H), 2.22 (d, 2H), 1.41 (m, 1H), 0.75 (d, 6H).

### EXAMPLE 90 8-(2-Iodo-5-methoxy-phenylsulfanyl)-9-[2-(3-methyl-butylamino)-ethyl]-9H-purin-6-ylamine (90)

The title compound was obtained by reacting 9-(2-chloro-ethyl)-8-(2-iodo-5-methoxy-phenylsulfanyl)-9H-purin-6-ylamine with iso-amylamine according to the general procedure A. Rt=6.53 min (5-100-12). ¹H NMR (CDCl₃) δ 8.34 (s, 1H), 7.98 (d, 1H), 6.71 (d, 1H), 6.65 (dd, 1H), 5.70 (br. s, 2H), 4.33 (t, 2H), 3.68 (s, 3H), 3.25 (q, 2H), 2.97 (t, 2H), 2.70 (t, 2H), 1.95 (m, 1H), 0.91 (d, 6H).

### EXAMPLE 91 9-[2-(3,3-Dimethyl-butylamino)-ethyl]-8-(2-iodo-5-methoxy-phenylsulfanyl)-9H-purin-6-ylamine (91)

The title compound was obtained by reacting 9-(2-chloro-ethyl)-8-(2-iodo-5-methoxy-phenylsulfanyl)-9H-purin-6-ylamine with 3,3-dimethyl-butylamine according to the general procedure A. Rt=6.87 min (5-100-12).

### EXAMPLE 92 {2-[6-Amino-8-(2-iodo-5-methoxy-phenylsulfanyl)-purin-9-yl]-ethylamino}-acetonitrile (92)

The title compound was obtained by reacting 9-(2-chloro-ethyl)-8-(2-iodo-5-methoxy-phenylsulfanyl)-9H-purin-6-ylamine with amino-acetonitrile according to the general procedure A. Rt=6.31 min (5-100-12). ¹H NMR (CDCl₃) δ 8.37 (s, 1H), 7.74 (d, 1H), 6.73 (d, 1H), 6.60 (dd, 1H), 5.63 (br. s, 2H), 4.38 (t, 2H), 3.67 (s, 3H), 2.58 (m, 2H), 3.10 (m, 2H).

### EXAMPLE 93 2-{2-[6-Amino-8-(2-iodo-5-methoxy-phenylsulfanyl)-purin-9-yl]-ethylamino}-ethanol (93)

The title compound was obtained by reacting 9-(2-chloro-ethyl)-8-(2-iodo-5-methoxy-phenylsulfanyl)-9H-purin-6-ylamine with ethanolamine according to the general procedure A. Rt=5.14 min (5-100-12). ¹H NMR (CDCl₃) δ 8.37 (s, 1H), 7.70 (d, 1H), 6.76 (d, 1H), 6.56 (dd, 1H), 5.71 (br. s, 2H), 4.33 (t, 2H), 3.68 (s, 3H), 3.56 (t, 2H), 3.01 (t, 2H), 2.97 (3, 2H).

### EXAMPLE 94 8-(2-Iodo-5-methoxy-phenylsulfanyl)-9-[2-(2-methoxy-ethylamino)-ethyl]-9H-purin-6-ylamine (94)

The title compound was obtained by reacting 9-(2-chloro-ethyl)-8-(2-iodo-5-methoxy-phenylsulfanyl)-9H-purin-6-ylamine with MeO—(CH₂)₂—NH₂ according to the general procedure A. Rt=5.52 min (5-100-12). ¹H NMR (CDCl₃) δ 8.32 (s, 1H), 7.68 (d, 1H), 6.72 (d, 1H), 6.56 (dd, 1H), 5.68 (br. s, 2H), 4.32 (t, 2H), 3.68 (s, 3H), 3.42 (t, 2H), 3.32 (s, 3H), 3.03 (t, 2H), 2.81 (3, 2H).

### EXAMPLE 95 9-(3-tert-Butylamino-propyl)-8-(2-iodo-5-methoxy-phenylsulfanyl)-9H-purin-6-ylamine (95)

Step 1 Methanesulfonic acid 3-[6-amino-8-(2-iodo-5-methoxy-phenylsulfanyl)-purin-9-yl]-propyl ester was reacted with tert-butylamine according to the general procedure A. The crude reaction product was extracted into aq. HCl, and the aqueous solution was washed ten times with CHCl₃. Neutralization (NaHCO₃) and back-extraction into CHCl₃ gave the title compound as a crude oil. The free base (4.34 g) was dissolved in MeOH (100 mL), treated with conc. HCl (2.7 mL) and the solution was evaporated to dryness. The hydrochloride salt was re-dissolved in refluxing MeOH and precipitated with acetone. Filtration gave pure hydrochloride salt. The salt was dissolved in water, neutralized with sat. aq. NaHCO₃, and extracted with CHCl₃. Drying and concentration gave the pure title compound in its free base form. Rt=5.87 min (5-100-12). ¹H NMR (CDCl₃) δ 8.33 (s, 1H), 7.70 (d, 1H), 6.69 (d, 1H), 6.55 (dd, 1H), 5.90 (br. s, 2H), 4.30 (t, 2H), 3.66 (s, 3H), 2.50 (t, 2H), 1.96 (quint, 2H), 1.05 (s, 9H).

### Step 2 9-(3-tert-Butylamino-propyl)-8-(2-iodo-5-methoxy-phenylsulfanyl)-9H-purin-6-ylamine, phosphoric acid salt

9-(3-tert-Butylamino-propyl)-8-(2-iodo-5-methoxy-phenylsulfanyl)-9H-purin-6-ylamine (7.29 g) was dissolved in refluxing EtOH, and treated with a solution of H₃PO₄ (0.84M in EtOH, 17.0 mL) whereupon a precipitate immediately appeared. This was collected by filtration, washed (EtOH), and dried to give the phosphate salt. Rt=4.77 min (5-100-7). δ 8.04 (br. s, 1H), 7.72 (br. d, 1H), 6.89 (br. s, 1H), 6.65 (br. d, 1H), 4.21 (br. t, 2H), 3.61 (br. s, 3H), 2.86 (br. t, 2H), 2.04 (br. quint., 2H), 1.15 (s, 9H).

### EXAMPLE 96 9-(2-Cyclopentylamino-ethyl)-8-(2-iodo-5-methoxy-phenylsulfanyl)-9H-purin-6-ylamine (96)

The title compound was obtained by reacting 9-(2-chloro-ethyl)-8-(2-Iodo-5-methoxy-phenylsulfanyl)-9H-purin-6-ylamine with cyclopentylamine according to the general procedure A. Rt=6.05 min (5-100-12). ¹H NMR (CDCl₃) δ 8.34 (s, 1H), 7.70 (d, 1H), 6.71 (d, 1H), 6.58 (dd, 1H), 5.85 (br. s, 2H), 4.38 (t, 2H), 3.65 (s, 3H), 3.03 (quint., 2H), 2.98 (t, 2H), 1.80 (m, 4H), 1.60 (m, 4H).

### EXAMPLE 97 9-(2-Cyclohexylamino-ethyl)-8-(2-iodo-5-methoxy-phenylsulfanyl)-9H-purin-6-ylamine (97)

The title compound was obtained by reacting 9-(2-chloro-ethyl)-8-(2-iodo-5-methoxy-phenylsulfanyl)-9H-purin-6-ylamine with cyclohexylamine according to the general procedure A. Rt=6.40 min (5-100-12). ¹H NMR (CDCl₃) δ 8.34 (s, 1H), 7.70 (d, 1H), 6.75 (d, 1H), 6.62 (dd, 1H), 5.80 (br. s, 2H), 4.25 (t, 2H), 3.65 (s, 3H), 3.03 (m, 1H), 2.98 (t, 2H), 1.301.10 (m, 10H).

### EXAMPLE 98 9-(2-Cycloheptylamino-ethyl)-8-(2-Iodo-5-methoxy-phenylsulfanyl)-9H-purin-6-ylamine (98)

The title compound was obtained by reacting 9-(2-chloro-ethyl)-8-(2-iodo-5-methoxy-phenylsulfanyl)-9H-purin-6-ylamine with cycloheptylamine according to the general procedure A. Rt=6.80 min (5-100-12). ¹H NMR (CDCl₃) δ 8.34 (s, 1H), 7.68 (d, 1H), 6.75 (d, 1H), 6.32 (dd, 1H), 5.80 (br. s, 2H), 4.30 (t, 2H), 3.65 (s, 3H), 2.98 (t, 2H), 2.80 (m, 1H), 1.73 (m, 4H), 1.55 (m, 4H), 1.42 (m, 4H).

### EXAMPLE 99 9-(2-Cyclooctylamino-ethyl)-8-(2-iodo-5-methoxy-phenylsulfanyl)-9H-purin-6-ylamine (99)

The title compound was obtained by reacting 9-(2-chloro-ethyl)-8-(2-iodo-5-methoxy-phenylsulfanyl)-9H-purin-6-ylamine with cyclooctylamine according to the general procedure A. Rt=7.10 min (5-100-12). ¹H NMR (CDCl₃) δ 8.34 (s, 1H), 7.68 (d, 1H), 6.82 (d, 1H), 6.66 (dd, 1H), 5.72 (br. s, 2H), 4.40 (t, 2H), 3.65 (s, 3H), 3.00 (t, 2H), 2.72 (m, 1H), 1.60-1.10 (m, 14H).

### EXAMPLE 100 9-[2-(Cyclopropylmethyl-amino)-ethyl]-8-(2-iodo-5-methoxy-phenylsulfanyl)-9H-purin-6-ylamine (100)

The title compound was obtained by reacting 9-(2-chloro-ethyl)-8-(2-iodo-5-methoxy-phenylsulfanyl)-9H-purin-6-ylamine with C-cyclopropyl-methyl-amine according to the general procedure A. Rt=5.82 min (5-100-12). ¹H NMR (CDCl₃/CD₃OD 3:1) δ 8.13 (s, 1H), 7.67 (d, 1H), 6.82 (d, 1H), 6.59 (dd, 1H), 4.24 (t, 2H), 3.63 (s, 3H), 2.91 (t, 2H), 2.37 (d, 2H), 0.78 (m, 1H), 036 (m, 2H), 0.00 (m, 2H).

### EXAMPLE 101 8-(2-Iodo-5-methoxy-phenylsulfanyl)-9-[2-(2-methyl-allylamino)-ethyl]-9H-purin-6-ylamine (101)

The title compound was obtained by reacting 9-(2-chloro-ethyl)-8-(2-iodo-5-methoxy-phenylsulfanyl)-9H-purin-6-ylamine with 2-methyl-allylamine according to the general procedure A. ¹H NMR (CDCl₃/CD₃OD 10:1) δ 8.16 (s, 1H), 7.69 (d, 1H), 6.84 (d, 1H), 6.58 (dd, 1H), 4.76 (s, 2H), 4.32 (t, 2H), 3.66 (s, 3H), 3.12 (br. s, 2H), 2.94 (t, 2H), 1.61 (s, 3H).

### EXAMPLE 102 9-(2-tert-Butylamino-ethyl)-8-(2-iodo-5-methoxy-phenylsulfanyl)-9H-purin-6-ylamine (102)

The title compound was obtained by reacting toluene-4-sulfonic acid 2-[6-amino-8-(2-iodo-5-methoxy-phenylsulfanyl)-purin-9-yl]-ethyl ester with tert-butylamine according to the general procedure A. Solid, Rt=4.73 min (5-100-7).

### EXAMPLE 103 9-(3-Amino-propyl)-g-(2-Iodo-5-methoxy-phenylsulfanyl)-9H-purin-6-ylaminen (103)

The title compound was obtained by reacting 9-(3-chloro-propyl)-8-(2-iodo-5-methoxy-phenylsulfanyl)-9H-purin-6-ylamine with NH₃ (7M in MeOH) according to the general procedure A. Rt=5.45 min (5-100-12). ¹HNMR (CD₃OD) δ 8.21 (s, 1H), 7.86 (d, 1H), 7.00 (s, 1H), 6.79 (d, 1H), 4.31 (t, 2H), 3.76 (s, 3H), 2.74 (t, 2H), 1.88 (quint., 2H), 1.57 (quint., 2H).

### EXAMPLE 104 9-(2-Cyclopropylamino-ethyl)-8-(2-iodo-5-methoxy-phenylsulfanyl)-9H-purin-6-ylamine (104)

The title compound was obtained by reacting 9-(2-chloro-ethyl)-8-(2-iodo-5-methoxy-phenylsulfanyl)-9H-purin-6-ylamine with cyclopropylamine according to the general procedure A. Rt=5.48 min. ¹H NMR (CD₃OD) δ 8.21 (s, 1H), 7.83 (d, 1H), 6.94 (d, 1H), 6.57 (dd, 1H), 4.40 (t, 2H), 3.72 (s, 3H), 3.06 (t, 2H), 2.05 (m, 1H), 0.69 (m, 2H), 0.44 (m, 2H).

### EXAMPLE 105 9-(2-Allylamino-ethyl)-8-(2-iodo-5-methoxy-phenylsulfanyl)-9H-purin-6-ylamine (105)

The title compound was obtained by reacting 9-(2-chloro-ethyl)-8-(2-iodo-5-methoxy-phenylsulfanyl)-9H-purin-6-ylamine with allylamine according to the general procedure A. Rt=5.62 min. ¹H NMR (CD₃OD) δ 8.21 (s, 1H), 7.85 (d, 1H), 6.96 (d, 1H), 6.48 (dd, 1H), 5.90 (m, 1H), 5.20 (m, 2H), 4.41 (m, 2H), 3.74 (s, 3H), 3.24 (m, 2H), 3.06 (t, 2H), 2.05 (m, 1H), 0.69 (m, 2H), 0.44 (m, 2H).

### EXAMPLE 106 8-(2-Iodo-5-methoxy-phenylsulfanyl)-9-(2-morpholin-4-yl-ethyl)-9H-purin-6-ylamine (106)

The title compound was obtained by reacting 9-(2-chloro-ethyl)-8-(2-iodo-5-methoxy-phenylsulfanyl)-9H-purin-6-ylamine with morpholine according to the general procedure A. Rt=5.33 min. ¹H NMR (CDCl₃) δ 8.36 (s, 1H), 7.69 (d, 1H), 6.70 (d, 1H), 6.53 (dd, 1H), 5.76 (s, 2H), 4.36 (t, 2H), 3.68 (s, 3H), 2.70 (t, 2H), 2.49 (m, 4H), 1.82 (m, 4H).

### EXAMPLE 107 8-(2-Iodo-5-methoxy-phenylsulfanyl)-9-(3-propylamino-propyl)-9H-purin-6-ylamine (107)

The title compound was obtained by reacting 9-(3-chloro-propyl)-8-(2-iodo-5-methoxy-phenylsulfanyl)-9H-purin-6-ylamine with n-propylamine according to the general procedure A. Rt=5.78 min (5-100-12M). ¹H NMR (MeOH) δ 8.21 (s, 1H), 7.85 (d, 1H), 6.97 (d, 1H), 6.77(dd, 1H), 4.34 (t, 2H), 3.75 (s, 3H), 2.57 (t, 2H), 2.47 (t, 2H), 2.03 (quint., 2H), 1.51 (q, 2H), 0.94 (t, 3H).

### EXAMPLE 108 9-[3-(1-Ethyl-propylamino)-propyl]-8-(2-iodo-5-methoxy-phenylsulfanyl)-9H-purin-6-ylamine (108)

The title compound was obtained by reacting 9-(3-chloro-propyl)-8-(2-iodo-5-methoxy-phenylsulfanyl)-9H-purin-6-ylamine with 1-ethyl-propylamine according to the general procedure A. Rt=6.28 min (5-100-12). ¹H NMR (MeOH) δ 8.20 (s, 1H), 7.82 (d, 1H), 6.96 (d, 1H), 6.75(dd, 1H), 4.35 (t, 2H), 3.73 (s, 3H), 2.74 (t, 2H), 2.55 (quint., 1H), 2.08 (s, quint., 2H), 1.52 (m, 4H), 0.91 (t, 6H).

### EXAMPLE 109 9-(3-sec-Butylamino-propyl)-8-(2-iodo-5-methoxy-phenylsulfanyl)-9H-purin-6-ylamine (racemate) (109)

The title compound was obtained by reacting 9-(3-chloro-propyl)-8-(2-iodo-5-methoxy-phenylsulfanyl)-9H-purin-6-ylamine with sec-butylamine according to the general procedure A. The same procedure was also used with enantiomerically pure (S- or R)-sec-butylamine to give the corresponding enantiomer. Rt=5.93 min (5-100-12). ¹H NMR (MeOH) δ 8.21 (s, 1H), 7.85 (d, 1H), 6.96 (d, 1H), 6.76(dd, 1H), 4.35 (t, 2H), 3.73 (s, 3H), 2.70-2.64 (m, 3H), 2.07 (quint., 2H), 1.58 (m, 1H), 1.34 (m, 1H), 1.08 (d, 3H), 0.92 (s, 3H).

### EXAMPLE 110 9-(3-Heptylamino-propyl)-8-(2-iodo-5-methoxy-phenylsulfanyl)-9H-purin-6-ylamine (110)

The title compound was obtained by reacting 9-(3-chloro-propyl)-8-(2-iodo-5-methoxy-phenylsulfanyl)-9H-purin-6-ylamine with n-heptylamine according to the general procedure A. Rt=7.50 min (5-100-12). ¹H NMR (MeOH) δ 8.21 (s, 1H), 7.85 (d, 1H), 6.99 (d, 1H), 6.79(dd, 1H), 4.39 (t, 2H), 3.75 (s, 3H), 2.99 (t, 2H), 2.89 (t, 2H), 2.19 (quint., 2H), 1.65 (m, 2H), 1.34 (m, 8H), 0.90(t, 3H).

### EXAMPLE 111 9-(3-Cyclopentylamino-propyl)-8-(2-iodo-5-methoxy-phenylsulfanyl)-9H-purin-6-ylamine (111)

The title compound was obtained by reacting 9-(3-chloro-propyl)-8-(2-iodo-5-methoxy-phenylsulfanyl)-9H-purin-6-ylamine with cyclopentylamine according to the general procedure A. Rt=6.12 min (5-100-12). ¹H NMR (CDCl₃) δ 8.30 (s, 1H), 7.69 (d, 1H), 6.68 (d, 1H), 6.55(dd, 1H), 6.06 (br.s., 2H), 4.29 (t, 2H), 3.65 (s, 3H), 2.98 (quint., 1H), 2.56 (t, 2H), 1.99 (quint., 2H), 1.64-1.40 (m, 8H).

### EXAMPLE 112 9-(3-Cyclooctylamino-propyl)-8-(2-iodo-5-methoxy-phenylsulfanyl)-9H-purin-6-ylamine (112)

The title compound was obtained by reacting 9-(3-chloro-propyl)-8-(2-iodo-5-methoxy-phenylsulfanyl)-9H-purin-6-ylamine with cyclooctylamine according to the general procedure A. Rt=7.07 min (5-100-12). ¹H NMR (CDCl₃) δ 8.31 (s, 1H), 7.70 (d, 1H), 6.68 (d, 1H), 6.55 (dd, 1H), 6.00 (br.s., 2H), 4.30 (t, 2H), 3.65 (s, 3H), 2.70 (quint., 1H), 2.59 (t, 2H), 1.53-1.40 (m, 14H).

### EXAMPLE 113 8-(2-Iodo-5-methoxy-phenylsulfanyl)-9-(3-isobutylamino-propyl)-9H-purin-6-ylamine (113)

The title compound was obtained by reacting 9-(3-chloro-propyl)-8-(2-iodo-5-methoxy-phenylsulfanyl)-9H-purin-6-ylamine with isobutylamine according to the general procedure A. Rt=6.00 min (5-100-12). ¹H NMR (CDCl₃) δ 8.32 (s, 1H), 7.70 (d, 1H), 6.67 (d, 1H), 6.56 (dd, 1H), 5.97 (br.s., 2H), 4.30 (t, 2H), 3.65 (s, 3H), 2.56(t, 2H), 2.33 (d, 2H), 1.79 (quint., 2H), 1.72(m, 1H), 0.90(d, 14H).

### EXAMPLE 114 8-(2-Iodo-5-methoxy-phenylsulfanyl)-9-[3-(1,2,2-trimethyl-propylamino)-propyl]-9H-purin-6-ylamine (114)

The title compound was obtained by reacting 9-(3-chloro-propyl)-8-(2-iodo-5-methoxy-phenylsulfanyl)-9H-purin-6-ylamine with 1,2,2-trimethyl-propylamine according to the general procedure A. Rt=6.56 min (5-100-12). ¹H NMR (CDCl₃) δ 8.35 (s, 1H), 7.72 (d, 1H), 6.70 (d, 1H), 6.59 (dd, 1H), 5.95 (br.s., 2H), 4.33 (t, 2H), 3.67 (s, 3H), 2.79 (m, 1H), 2.50 (m, 1H), 2.21 (m, 1H), 1.99 (m, 2H), 0.90 (s, 9H).

### EXAMPLE 115 4-{3-[6-Amino-8-(2-iodo-5-methoxy-phenylsulfanyl)-purin-9-yl]-propylamino}-piperidine-1-carboxylic acid tert-butyl ester (115)

The title compound was obtained by reacting 9-(3-chloro-propyl)-8-(2-iodo-5-methoxy-phenylsulfanyl)-9H-purin-6-ylamine with 4-amino-piperidine-1-carboxylic acid tert-butyl ester according to the general procedure A. Rt=6.14 min (5-100-12). ¹H NMR (CDCl₃) δ 8.29 (s, 1H), 7.69 (d, 1H), 6.66 (d, 1H), 6.55 (dd, 1H), 6.29 (br.s., 2H), 4.29 (t, 2H), 3.64 (s, 3H), 3.15 (quint., 1H), 2.79 (t, 2H), 2.57 (m, 4H), 1.96 (m, 2H), 1.80 (m, 2H), 1.23 (s, 9H).

### EXAMPLE 116 9-(2-Benzylamino-ethyl)-8-(2-iodo-5-methoxy-phenylsulfanyl)-9H-purin-6-ylamine (116)

The title compound was obtained by reacting 9-(2-chloro-ethyl)-8-(2-iodo-5-methoxy-phenylsulfanyl)-9H-purin-6-ylamine with benzylamine according to the general procedure A. Rt=6.31 min. ¹H NMR (CDCl₃) δ 8.35 (s, 1H), 7.71 (d, 1H), 7.36-7.22 (m, 5H), 6.75 (d, 1H), 6.57 (dd, 1H), 5.84 (br.s, 2H), 4.39 (t, 2H), 3.79 (s, 2H), 3.67 (s, 3H), 3.04 (t, 2H).

### EXAMPLE 117 (R)-9-(3-sec-Butylamino-propyl)-8-(2-iodo-5-methoxy-phenylsulfanyl)-9H-purin-6-ylamine (117)

The title compound was obtained by reacting 9-(2-chloro-ethyl)-8-(2-iodo-5-methoxy-phenylsulfanyl)-9H-purin-6-ylamine with R-(−)-2-aminobutane according to the general procedure A. Rt=5.915 min. ¹H NMR (CDCl₃) δ 8.27 (s, 1H), 7.63 (d, 1H), 6.74 (br.s., 2H), 6.61(d, 1H), 6.48 (dd, 1H), 4.26 (t, 2H), 3.58 (s, 3H), 2.57-2.14 (m,3H), 1.92 (quint., 2H), 1.43 (7, 1H), 1.28 (7, 1H), 1.02 (t, 2H), 0.94 (d, 3H), 0.81 (t, 3H).

### EXAMPLE 118 (S)-9-(3-sec-Butylamino-propyl)-8-(2-iodo-5-methoxy-phenylsulfanyl)-9H-purin-6-ylamine (118)

The title compound was obtained by reacting 9-(2-chloro-ethyl)-8-(2-iodo-5-methoxy-phenylsulfanyl)-9H-purin-6-ylamine with S-(−)-2-aminobutane according to the general procedure A. Rt=5.941 min. ¹H NMR (CDCl₃) δ 8.32 (s, 1H), 7.68 (d, 1H), 6.65(d, 1H), 6.54 (dd, 1H), 6.39 (br. s., 2H), 4.29 (t, 2H), 3.63 (s, 3H), 2.59-2.44 (m,3H), 1.95 (quint., 2H), 1.45 (7, 1H), 1.28 (7, 1H), 1.05 (t, 2H), 0.97 (d, 3H), 0.84 (t, 3H).

### EXAMPLE 119 9-[3-(1,1-Dimethyl-propylamino)-propyl]-8-(2-iodo-5-methoxy-phenylsulfanyl)-9H-purin-6-ylamine (119)

The title compound was obtained by reacting 9-(2-chloro-ethyl)-8-(2-iodo-5-methoxy-phenylsulfanyl)-9H-purin-6-ylamine with 1,1-dimethyl-propylamine according to the general procedure A. Rt=6.218 min. ¹H NMR (CDCl₃) δ 8.35 (s, 1H), 7.71 (d, 1H), 6.68(d, 1H), 6.56 (dd, 1H), 6.12 (br. s., 2H), 4.31 (t, 2H), 3.66 (s, 3H), 2.50 (t,3H), 1.95 (quint., 2H), 1.07 (q, 2H), 0.99 (s, 6H), 0.80 (t, 3H).

### EXAMPLE 120 9-(3-Cyclobutylamino-propyl)-8-(2-iodo-5-methoxy-phenylsulfanyl)-9H-purin-6-ylamine (120)

The title compound was obtained by reacting 9-(2-chloro-ethyl)-8-(2-iodo-5-methoxy-phenylsulfanyl)-9H-purin-6-ylamine with cyclobutylamine according to the general procedure A. Rt=5.785 min. ¹H NMR (CDCl₃) δ 8.37 (s, 1H), 7.72 (d, 1H), 6.67(d, 1H), 6.56 (dd, 1H), 6.02 (br. s., 2H), 4.30 (t, 2H), 3.67 (s, 3H), 3.17 (quint., 1H), 2.50 (t, 3H), 2.16 (d, 2H), 1.94 (quint., 2H), 1.64 (m, 4H).

### EXAMPLE 121 9-(3-Amino-propyl)-8-(2-iodo-5-methoxy-phenylsulfanyl)-9H-purin-6-ylamine (121)

### Step 1 {3-[6-Amino-8-(2-iodo-5-methoxy-phenylsulfanyl)-purin-9-yl]-propyl}-carbamic acid tert-butyl ester

A mixture of 8-(2-iodo-5-methoxy-phenylsulfanyl)-9H-purin-6-ylamine (260 mg), BocNH—(CH₂)₃—Cl, and Cs₂CO₃ (1.29 g) in DMF (3 mL) was heated to 50° C. for 16 h. Work-up and flash chromatography (1% MeOH in DCM, then EtOAc, then 5% MeOH in EtOAc) gave the desired product. Rt=8.23 min. ¹H NMR (CDCl₃) δ 8.34 (s, 1H), 7.70 (d, 1H), 6.70 (d, 1H), 6.55 (dd, 1H), 6.15 (s, 2H), 5.59 (t, 1H), 4.28 (t, 2H), 3.65 (t, 3H), 3.01 (q, 2H), 1.90 (quint., 2H), 1.44 (s, 9H).

### Step 2 9-(3-Amino-propyl)-8-(2-iodo-5-methoxy-phenylsulfanyl)-9H-purin-6-ylamine

A solution of {3-[6-amino-8-(2-iodo-5-methoxy-phenylsulfanyl)-purin-9-yl]-propyl}-carbamic acid tert-butyl ester (54 mg) in DCM (3 mL) was treated with TFA (0.5 mL) for 30 min and evaporated. Reverse-phase MPLC (C₁₈, gradient H₂O/CH₃CN, 1% TFA) gave the title compound as a TFA salt which was diluted in DCM, washed with NaHCO₃ and concentrated to give the title compound as a free base. Rt=5.21 min. ¹H NMR (CDCl₃/CD₃OD) δ 8.18 (s, 1H), 7.77 (d, 1H), 7.12 (d, 1H), 6.68 (dd, 1H), 4.30 (t, 2H), 3.74 (s, 3H), 2.89 (t, 2H), 2.16 (quint., 2H).

### EXAMPLE 122 {2-[6-Amino-8-(2-iodo-5-methoxy-phenylsulfanyl)-purin-9-yl]-ethyl}-carbamic acid tert-butyl ester (122)

### Step 1 (2-Chloro-ethyl)-carbamic acid tert-butyl ester

A suspension of Cl—(CH₂)₂—NH₂.HCl in DCM (10 mL) was treated at 0° C. with Et3N (1.39 mL) and (tBoc)₂O (2.18 g). The reaction was then stirred at rt overnight, concentrated, and worked-up (EtOAc/½ sat. brine) to give the title compound.

### Step 2 {2-[6-Amino-8-(2-iodo-5-methoxy-phenylsulfanyl)-purin-9-yl]-ethyl}-carbamic acid tert-butyl ester

A mixture of 8-(2-iodo-5-methoxy-phenylsulfanyl)-9H-purin-6-ylamine (0.15 g), (2-Chloro-ethyl)-carbamic acid tert-butyl ester (0.10 g) and Cs₂CO₃ (0.45 g) in DMF (1 mL) was heated to 80° C. for 2 h, and to 100° C. for another 1.5 h. Flash chromatography (EtOAc/Hexane 1:1→1:0) gave the title compound. Rt=7.85 min. ¹H NMR (CDCl₃) δ 8.30 (s, 1H), 7.80 (d, 1H), 6.80 (d, 1H), 6.65 (dd, 1H), 6.02 (s, 2H), 4.40 (t, 2H), 3.66 (s, 3H), 3.53 (q, 2H), 1.23 (s, 9H).

### EXAMPLE 123 9-(2-Amino-ethyl)-8-(2-iodo-5-methoxy-phenylsulfanyl)-9H-purin-6-ylamine (123)

{2-[6-amino-8-(2-iodo-5-methoxy-phenylsulfanyl)-purin-9-yl]-ethyl}-carbamic acid tert-butyl ester (6.4 mg) was treated with TFA/DCM 1:10 for 1 h at rt, washed with aq NaHCO₃, and evaporated. Rt=5.16 min. ¹H NMR (CDCl₃) δ 8.24 (s, 1H), 7.82 (d, 1H), 7.20 (d, 1H), 6.72 (dd, 1H), 4.61 (t, 2H), 3.77 (s, 3H), 3.42 (t, 2H).

### EXAMPLE 124 2-[6-Amino-8-(2-iodo-5-methoxy-phenylsulfanyl)-purin-9-yl]-acetamide (124)

A mixture of 8-(2-iodo-5-methoxy-phenylsulfanyl)-9H-purin-6-ylamine (80 mg), 2-bromoacetamide (55 mg) and Cs₂CO₃ (316 mg) in DMF (1 mL) was stirred at rt overnight. Preparative TLC gave the title compound. Rt=5.70 min. ¹H NMR (DMSO-d₆) δ 8.26 (s, 1H), 8.08 (s, 1H), 7.67 (d, 1H), 7.50 (s, 1H), 6.96 (d, 1H), 6.51 (dd, 1H), 4.93 (s, 2H), 3.59 (s, 2H), 3.31 (s, 3H).

### EXAMPLE 125 1-[6-Amino-8-(2-iodo-5-methoxy-phenylsulfanyl)-purin-9-yl]-propan-2-one (125)

A mixture of 8-(2-iodo-5-methoxy-phenylsulfanyl)-9H-purin-6-ylamine (80 mg), chloroacetone (32 uL) and Cs₂CO₃ (350 mg) in DMF (1 mL) was stirred at 75° C. overnight. Work-up and preparative TLC (5% MeOH in DCM) gave the title compound. Rt=6.74 min. ¹H NMR (CDCl₃) δ 8.32 (s, 1H), 7.68 (d, 1H), 6.74 (d, 1H), 6.55 (dd, 1H), 5.67 (s, 2H), 5.05 (s, 2H), 3.67 (s, 3H), 2.23 (s, 3H).

### EXAMPLE 126 8-(2-Iodo-5-methoxy-phenylsulfanyl)-9-(3-isopropylamino-propyl)-9H-purin-6-ylamine H₃PO₄ salt (126)

### Step 1 3-[6-Amino-8-(2-iodo-5-methoxy-phenylsulfanyl)-purin-9-yl]-propan-1-ol

A solution of acetic acid 3-[6-amino-8-(2-iodo-5-methoxy-phenylsulfanyl)-purin-9-yl]-propyl ester (831 mg; Biamonte, J. Org. Chem., 2005, 70, 717) in MeOH (10 mL) was treated with K₂CO₃ (88 mg) at rt for 2 h and concentrated. The solid was stirred in a mixture of water (4 mL) and Et₂O (15 mL) at rt for 1 h. Filtration afforded the desired product. Rt=5.04 min (5-100-7). ¹H NMR (DMSO-d₆) δ 8.18 (s, 1H), 7.78 (d, 1H), 7.48 (s, 2H), 6.71 (dd, 1H), 6.47 (d, 1H), 4.66 (t, 1H), 4.21 (t, 2H), 3.61 (s, 3H), 3.40 (q, 2H), 1.82 (quint, 2H).

### Step 2 Methanesulfonic acid 3-[6-amino-8-(2-iodo-5-methoxy-phenylsulfanyl)-purin-9-yl]-propyl ester

3-[6-Amino-8-(2-iodo-5-methoxy-phenylsulfanyl)-purin-9-yl]-propan-1-ol was dissolved in anhydrous 1,4-dioxane at 80 C. Triethylamine (3 equiv.) were added, and the mixture was cooled to 40 C before adding MsCl (1.5 equiv.). After 15 min, the solvent and triethylamine were evaporated in vacuo, to give the title compound as a crude oil which was used immediately in the next step, without additional purification.

### Step 3 8-(2-Iodo-5-methoxy-phenylsulfanyl)-9-(3-isopropylamino-propyl)-9H-purin-6-ylamine

Methanesulfonic acid 3-[6-amino-8-(2-iodo-5-methoxy-phenylsulfanyl)-purin-9-yl]-propyl ester was reacted with isopropylamine according to the general procedure A. The crude reaction product was extracted into aq. HCl, and the aqueous solution was washed ten times with CHCl₃. Neutralization (NaHCO₃) and back-extraction into CHCl₃ gave the title compound as a crude oil. The free base was dissolved isopropanol. Addition of HBr 48% induced crystallization, and the crystals were washed with acetone. The crystals were dissolved in a mixture of CH₂Cl₂ and sat. aq. NaHCO₃. The organic layer was dried (Na₂SO₄) and concentrated to afford the pure title compound as the free base. Rt=5.61 min (5-100-12). ¹H NMR (CD₃OD) δ 8.24 (s, 1H), 7.86 (d, 1H), 6.87 (d, 1H), 6.64 (dd, 1H), 4.42 (t, 2H), 3.77 (s, 3H), 3.37-3.33 (m, 3H), 3.08 (t, 2H), 2.24 (quint., 2H), 1.34 (d, 6H).

### Step 4 8-(2-Iodo-5-methoxy-phenylsulfanyl)-9-(3-isopropylamino-propyl)-9H-purin-6-ylamine, H₃PO₄ salt

A solution of 8-(2-iodo-5-methoxy-phenylsulfanyl)-9-(3-isopropylamino-propyl)-9H-purin-6-ylamine (1.03 g) in refluxing EtOH (30 mL) was treated with a 0.84 M solution of H₃PO₄ in EtOH (2.1 mL). The phosphate salt precipitated immediately, and was collected by filtration, and washed with EtOH. Rt=4.46 min (5_{—}100_{—}7). ¹H NMR (D₂O) δ 8.04 (br. s, 1H), 7.71 (br. d, 1H), 6.87 (br. s, 1H), 6.64 (br. d, 1H), 4.18 (br. t, 2H), 3.62 (br. s, 3H), 3.13 (sept., 1H), 2.88 (br. t., 2H), 2.03 (br. quint., 2H), 1.13 (d, 6H).

### EXAMPLE 127 N-{2-[6-Amino-8-(2-iodo-5-methoxy-phenylsulfanyl)-purin-9-yl]-ethyl}-acetamide

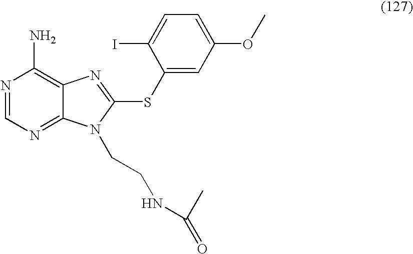

A solution of 9-(2-amino-ethyl)-8-(2-iodo-5-methoxy-phenylsulfanyl)-9H-purin-6-ylamine (123; 12 mg) in 1,2-dichloroethane (200 uL) was treated with Ac₂O (40 uL) at rt overnight. Concentration and preparative TLC (EtOAC/DCM/MeOH 7:7:1) gave the title compound. Rt=5.97 min (5-100-12). ¹H NMR (CDCl₃/CD₃OD 5:1) δ 8.32 (s, 1H), 7.68 (d, 1H), 7.30 (t, 1H), 6.88 (d, 1H), 6.60 (dd, 1H), 4.28 (t, 2H), 3.65 (s, 3H), 3.48 (q, 2H), 1.80 (s, 3H).

### EXAMPLE 128 N-{2-[6-Amino-8-(2-iodo-5-methoxy-phenylsulfanyl)-purin-9-yl]-ethyl}-methanesulfonamide

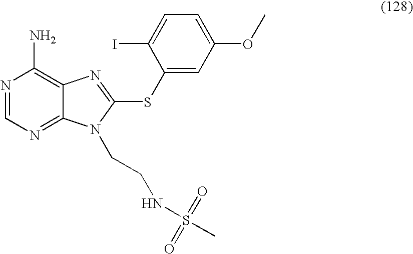

A solution of 9-(2-amino-ethyl)-8-(2-iodo-5-methoxy-phenylsulfanyl)-9H-purin-6-ylamine (123) and triethylamine (3 equiv.) in 1,2-dichloroethane was treated with MsCl (1.5 equiv) at rt overnight. Concentration ad preparative TLC (EtOAC/DCM/MeOH 7:7:1) gave the title compound. Rt=6.20 min (5-100-12). ¹H NMR (CDCl₃/CD₃OD 5:1) δ 8.33 (s, 1H), 7.70 (d, 1H), 6.90 (t, 1H), 6.50 (d, 1H), 4.27 (dd, 1H), 3.65 (s, 3H), 3.38 (y, 2H), 2.74 (s, 3H).

### EXAMPLE 129 N-{2-[6-Amino-8-(2-iodo-5-methoxy-phenylsulfanyl)-purin-9-yl]-ethyl}-N-isobutyl-acetamide

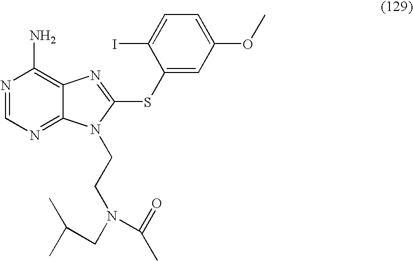

A solution of 8-(2-iodo-5-methoxy-phenylsulfanyl)-9-(2-isobutylamino-ethyl)-9H-purin-6-ylamine (15 mg) in 1,2-dichloroethane (500 uL) was treated with Ac₂O (60 uL) at rt for 45 min. Concentration and preparative TLC (EtOAC/DCM/MeOH 70:70:4) gave the title compound. Rt=7.70 min (5-100-12). ¹H NMR (CDCl₃) 3:1 mixture of s-trans and s-cis rotamers. Major rotamer: δ 8.32 (s, 1H), 7.72 (d, 1H), 6.80 (d, 1H), 6.58 (dd, 1H), 5.78 (br. s, 2H), 4.42 (t, 2H), 3.68 (t, 2H), 3.65 (s, 3H), 2.75 (d, 2H), 1.72 (m, 1H), 0.80 (d, 6H).

### EXAMPLE 130 N-{2-[6-Amino-8-(2-iodo-5-methoxy-phenylsulfanyl)-purin-9-yl]-ethyl}-N-isobutyl-

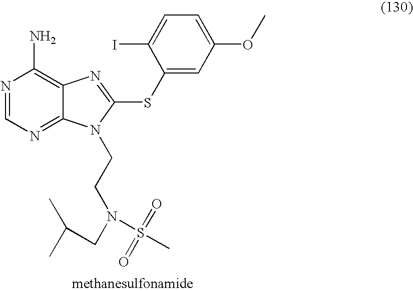

A solution of 9-(2-amino-ethyl)-8-(2-iodo-5-methoxy-phenylsulfanyl)-9H-purin-6-ylamine (123; 34 mg) and triethylamine (35 uL) in 1,2-dichloroethane (0.5 mL) was treated with MsCl (10 uL) at rt for 10 min. Concentration and preparative TLC (EtOAC/DCM/MeOH 70:70:4) gave the title compound. Rt=8.03 min (5-100-12). ¹H NMR (CDCl₃/CD₃OD 5:1) δ 8.20 (s, 1H), 7.70 (d, 1H), 6.83 (d, 1H), 6.59 (dd, 1H), 4.39 (t, 2H), 3.66 (s, 3H), 3.49 (t, 2H), 2.95 (d, 2H), 1.78 (m, 1H), 0.84 (d, 6H).

### EXAMPLE 131 2-Chloro-8-(2-iodo-5-methoxy-phenylsulfanyl)-9-pent-4-ynyl-9H-purin-6-ylamine

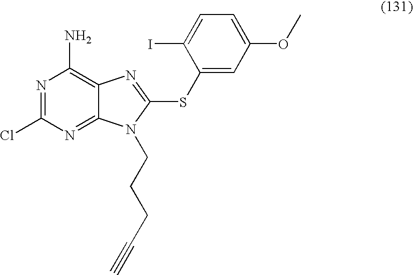

A suspension of 8-(2-iodo-5-methoxy-phenylsulfanyl)-9-pent-4-ynyl-9H-purine-2,6-diamine (55 mg; see example 15) in DCM (2 mL) was treated with TMSCl (205 uL) and Et₃N (30 uL) at rt for 10 min. A solution of BnEt₃N⁺NO₂⁻ (83 mg; Francom, J. Org. Chem. 2003, 68, 666) in DCM (1.5 mL) was added, and the reaction mixture was stirred for 30 min at rt. Work-up and flash chromatography (EtOAc/Hexane 1:1→1:0) gave the title compound. Rt=9.73 min (5-100-12). ¹H NMR (CDCl₃) δ 7.74 (d, 1H), 6.75 (d, 1H), 6.60 (dd, 1H), 4.27 (t, 2H), 3.70 (s, 3H), 2.26 (dt, 2H), 2.04 (quint., 2H), 1.96 (t, 1H).

### EXAMPLE 132 9-[2-(2,2-Dimethyl-propylamino)-ethyl]-8-(2-iodo-5-methoxy-phenylsulfanyl)-9H-purin-6-ylamine (132)

### Step 1 Acetic acid 2-(6-amino-purin-9-yl)-ethyl ester

A mixture of adenine (60.0 g), Cs₂CO₃ (223 g), AcO—CH₂—CH₂—Br (75.8 ml) and DMF (187 g) was stirred at 45° C. for 5 h. The DMF was evaporated and the residue was added to a mixture of AcOH (50 ml, 2 equiv.) water (100 ml) and ice (100 g). The solid was filtered, washed with 100 ml ice-cold water, and dried under high vacuum on a rotary evaporator to give the title compound as a white powder (62.4 g, 67%). Rt=3.05 min (5-100-12). ¹H NMR (DMSO) δ 8.13 (s, 1H), 8.12 (s, 1H), 4.37 (s, 4H), 191 (s, 3H).

### Step 2 Acetic acid 2-(6-amino-8-bromo-purin-9-yl)-ethyl ester

Acetic acid 2-(6-amino-purin-9-yl)-ethyl ester (16.6 g) was dissolved in a mixture of AcOH buffer (100 ml, note 1), MeOH (30 ml), and THF (30 ml) using magnetic stirring at rt. Bromine (7.0 ml) was added over 1 min, and the stirring was stopped, whereupon the desired product gently crystallized out of solution. After 1 h the crystals were collected by filtration, washed (H₂O) and air-dried to give the title compound (11.6 g, 51%) as purple prisms. Rt=4.01 min (5-100-12). ¹H NMR (DMSO) δ 8.18 8.32 (s, 1H), 5.69 (s, 2H), 4.47 (m, 4H), 2.00 (s, 3H).

### Step 3 2-[6-Amino-8-(2-iodo-5-methoxy-phenylsulfanyl)-purin-9-yl]-ethanol

A solution of acetic acid 2-(6-amino-8-bromo-purin-9-yl)-ethyl ester (16.5 g) and potassium 2-iodo-5-methoxy-benzenethiolate (33 g; (a) Ma, J. Org. Chem. 2001, 66, 4525 (b) Flynn, Org. Lett, 2001, 3, 651) in DMF (600 ml) was heated to 50° C. overnight. The reaction mixture was concentrated, dissolved in MeOH, and treated with a catalytic amount of K₂CO₃ for 3 h at 50° C. to cleave the acetyl group in situ. The mixture was concentrated again, add stirred in a mixture of water and Et₂O overnight. The desired alcohol, which was soluble neither in Et₂O nor in water, was recovered by filtration. Washing with ether and drying gave the title compound as an orange-brown powder (9 g, 37%). Rt=6.13 min (5-100-12). ¹H NMR (DMSO) δ 8.18 (s, 1H), 7.76 (d, 1H), 7.35 (s, 2H), 6.68 (dd, 1H), 6.55 (d, 1H), 5.03 (s, 1H), 4.26 (t, 2H), 3.70 (t, 2H), 3.69 (s, 3H).

### Step 4 9-[2-(2,2-Dimethyl-propylamino)-ethyl]-8-(2-iodo-5-methoxy-phenylsulfanyl)-9H-purin-6-ylamine

A suspension of 2-[6-amino-8-(2-iodo-5-methoxy-phenylsulfanyl)-purin-9-yl]-ethanol (6.0 g) in dioxane (400 ml) was heated to 80° C. until fully dissolved. The solution was cooled to 40° C., treated with Et₃N (3 equiv.) and MsCl (1.5 equiv.). The reaction was cooled to r.t., filtered to remove the Et₃N.HCl, and evaporated to give the crude mesylate which was immediately taken in dioxane (75 mL) and neopentylamine (25 ml) and heated to 70° C. in a pressure vessel for 4 h. Concentration gave the desired crude amine which was diluted with water, acidified with aq HCl to pH 1, and washed with 1% MeOH in CHCl₃ 10 times. The aqueous layer was neutralized with solid NaHCO₃ and the amine was extracted in 1% MeOH in CHCl₃ to give the title compound as a pale brown oil (approx. yield: 50%). Recrystallization from MeOH gave the title compound as fine off-white needles. Rt=5.13 min (5-100-12). ¹H NMR (CDCl₃/CD₃OD 3:1) δ 8.03 (s, 1H), 7.65 (d, 1H), 6.80 (d, 1H), 6.59 (dd, 1H), 4.48 (br. t, 2H), 3.62 (s, 3H), 3.20 (br. t, 2H), 2.76 (s, 2H), 0.88 (s, 9H).

### Step 5 9-[2-(2,2-Dimethyl-propylamino)-ethyl]-8-(2-iodo-5-methoxy-phenylsulfanyl)-9H-purin-6-ylamine, H₃PO₄ salt

The purified amine (1.0 g) was dissolved in refluxing EtOH (30 ml) and with very vigorous agitation a H₃PO₄ solution (0.84M in EtOH, 2.3 mL, 1 equiv) was added in one shot. The crystallization was immediate. After cooling, filtration gave the desired phosphate as off-white fine needles (1.0 g, 83%). Rt=5.08 min (5-100-12). ¹H NMR (D₂O) δ 8.09 (s, 1H), 7.75 (d, 1H), 6.89 (d, 1H), 6.68 (dd, 1H), 4.51 (br. t, 2H), 3.63 (s, 3H), 3.36 (br. t, 2H), 2.83 (s, 2H), 0.95 (s, 9H).

General Procedure C

A suspension of 8-(2-iodo-5-methoxy-phenylsulfanyl)-9H-purin-6-ylamine (1 equiv.), alkyl halide (RCl, RBr, or RI; 1.1-3 equiv.) and Cs₂CO₃, (3-5 equiv.) in DMF was heated to 40-80° C. for 2-16 h to give, typically, a 2:1 mixture of the N(9)- and N(3)-alkylated isomer. The reaction mixture was diluted with EtOAc, and washed water and brine. Drying (Na₂SO₄), evaporation, and preparative TLC or flash chromatography (e.g. AcOEt/Hexane/Et₃N 80:20:3) gave the desired compound.

### EXAMPLE 133 9-(2-Dimethylamino-ethyl)-8-(2-iodo-5-methoxy-phenylsulfanyl)-9H-purin-6-ylamine (133)

The title compound was obtained by reacting 8-(2-iodo-5-methoxy-phenylsulfanyl)-9H-purin-6-ylamine with (2-chloro-ethyl)-dimethyl-amine according to the general procedure C. Rt=5.18 min. ¹H NMR (CDCl₃) δ 8.33 (s, 1H), 7.67 (d, 1H), 6.69 (d, 1H), 6.52 (dd, 1H), 6.11 (s, 2H), 4.31 (t, 2H), 3.67 (s, 3H), 2.67(t, 2H), 2.63 (s, 6H).

### EXAMPLE 134 9-(2-Diethylamino-ethyl)-8-(2-iodo-5-methoxy-phenylsulfanyl)-9H-purin-6-ylamine (134)

The title compound was obtained by reacting 8-(2-iodo-5-methoxy-phenylsulfanyl)-9H-purin-6-ylamine with (2-chloro-ethyl)-diethyl-amine according to the general procedure C. Rt=5.60 min. ¹H NMR (CDCl₃) δ 8.38 (s, 1H), 7.68 (d, 1H), 6.72 (d, 1H), 6.54 (dd, 1H), 5.86 (s, 2H), 4.30 (t, 2H), 3.67 (s, 3H), 2.74 (t, 2H), 2.53(q, 4H), 1.07 (t, 6H).

### EXAMPLE 135 8-(2-Iodo-5-methoxy-phenylsulfanyl)-9-(2-pyrrolidin-1-yl-ethyl)-9H-purin-6-ylamine (135)

The title compound was obtained by reacting 8-(2-iodo-5-methoxy-phenylsulfanyl)-9H-purin-6-ylamine with the hydrochloride salt of 1-(2-chloro-ethyl)-pyrrolidine according to the general procedure C. Rt=5.55 min. ¹H NMR (CDCl₃) δ 8.35 (s, 1H), 7.68 (d, 1H), 6.68 (d, 1H), 6.53 (dd, 1H), 6.14 (s, 2H), 4.38 (t, 2H), 3.65 (s, 3H), 2.83 (t, 2H), 2.55(m, 4H), 1.74 (m, 4H).

### EXAMPLE 136 8-(2-Iodo-5-methoxy-phenylsulfanyl)-9-(2-vinyloxy-ethyl)-9H-purin-6-ylamine (136)

The title compound was obtained by reacting 8-(2-iodo-5-methoxy-phenylsulfanyl)-9H-purin-6-ylamine with (2-chloro-ethoxy)-ethene according to the general procedure C.

Rt=9.91 min. ¹H NMR (CDCl₃) δ 7.97 (s, 1H), 7.69 (d, 1H), 7.20 (s, 1H), 6.54-6.35 (m, 4H), 4.78 (t, 2H), 4.05(t, 2H), 3.74 (s, 3H).

### EXAMPLE 137 8-(2-Iodo-5-methoxy-phenylsulfanyl)-9-(2-isopropoxy-ethyl)-9H-purin-6-ylamine (137)

The title compound was obtained by reacting 8-(2-iodo-5-methoxy-phenylsulfanyl)-9H-purin-6-ylamine with 2-(2-chloro-ethoxy)-propane according to the general procedure C.

Rt=8.20 min. ¹H NMR (CDCl3) δ 8.37 (s, 1H), 8.08 (d, 1H), 7.12 (d, J=2.6 Hz 1H), 6.84 (dd, 1H), 5.80 (s, 2H), 4.48 (m, 2H), 3.85-3.80(m, 3H), 3.74 (s, 3H), 1.18(d, 6H).

### EXAMPLE 138 {3-[6-Amino-8-(2-iodo-5-methoxy-phenylsulfanyl)-purin-9-yl]-propyl}-methyl-carbamic acid tert-butyl ester (138)

The title compound was obtained by reacting 8-(2-iodo-5-methoxy-phenylsulfanyl)-9H-purin-6-ylamine reacting with (3-chloro-propyl)-carbamic acid tert-butyl ester (see previous example, step 1) according to the general procedure C. Rt=8.18 min (5-100-12). ¹H NMR (CDCl₃) δ 8.28 (s, 1H), 7.73 (d, 1H), 6.74 (d, 1H), 6.59 (dd, 1H), 6.01 (br.s., 2H), 4.29 (t, 2H), 3.68 (s, 3H), 2.96(t, 2H), 1.99 (quint., 2H), 1.45 (s, 9H).

### EXAMPLE 139 8-(2-Iodo-5-methoxy-phenylsulfanyl)-9-(3-pyrrol-1-yl-propyl)-9H-purin-6-ylamine (139)

The title compound was obtained by reacting 8-(2-iodo-5-methoxy-phenylsulfanyl)-9H-purin-6-ylamine with 1-(3-bromo-propyl)-1H-pyrrole according to the general procedure C. Rt=8.27 min (5-100-12). ¹H NMR (CDCl₃) δ 8.38 (s, 1H), 7.74 (d, 1H), 6.70 (d, 1H), 6.65 (s, 2H), 6.59 (dd, 1H), 6.15 (s, 2H), 6.00 (br.s., 2H), 4.25 (t,3H), 3.95 (t, 3H), 3.69 (s, 3H), 2.26 (quint., 2H).

### EXAMPLE 140 {3-[6-Amino-8-(2-iodo-5-methoxy-phenylsulfanyl)-purin-9-yl]-propyl}-carbamic acid tert-butyl ester (140)

### Step 1 (3-Chloro-propyl)-carbamic acid tert-butyl ester

Di-tert-butyldicarbonate 21.8 g (0.1 mol) was added to a mixture of triethylamine (12.6 g, 0.12 mol) and 3-chloropropylamine hydrochloride (14.0 g, 0.11 mol) in THF. The mixture was stirred at 0° C. for 20 min, then warmed to rt for 18 h, diluted with aq. NaHCO₃, and extracted with ether (2×80 mL). The extract was washed with brine, dried, and evaporated to give the title compound. ¹H NMR (DMSO) δ 6.84 (br.s., 1H), 3.59 (t, 2H), 3.02 (t, 3H), 1.81 (quint., 2H), 1.36 (s, (H).

### Step 2 (3-Chloro-propyl)-methyl-carbamic acid tert-butyl ester

NaH (0.24 g, 10 mmol) was added to a solution of (3-chloro-propyl)-carbamic acid tert-butyl ester (1.0 3 g, 5 mmol) and CH₃I (1.07 g, 7.5 mmol) in THF (5 mL) under N₂. The reaction mixture was stirred at rt overnight, and quenched with water (3 mL). Extraction (3×100 mL EtOAc), evaporation, and chromatography gave the title compound. ¹HNMR (DMSO) δ 3.60 (t, 2H), 3.28 (t, 3H), 2.78 (s, 2H), 1.91 (quint., 2H), 1.39 (s, (H).

### Step 3 {3-[6-Amino-8-(3-methoxy-1-methyl-buta-1,3-dienylsulfanyl)-purin-9-yl]-propyl}-methyl-carbamic acid tert-butyl ester

The title compound was obtained by reacting 8-(2-iodo-5-methoxy-phenylsulfanyl)-9H-purin-6-ylamine with (3-chloro-propyl)-methyl-carbamic acid tert-butyl ester according to the general procedure C. Rt=8.51 min (5-100-12). ¹H NMR (CDCl₃) δ 8.24 (s, 1H), 7.58 (d, 1H), 6.96 (br.s., 2H), 6.45 (d, 1H), 6.36 (dd, 1H), 4.14 (s, 2H), 3.53 (t, 3H), 2.72 (s, 3H), 2.51(t, 2H), 2.22 (s, 6H), 1.99 (quint., 2H), 1.32 (s, 9H).

### EXAMPLE 141 8-(2-Iodo-5-trifluoromethoxy-phenylsulfanyl)-9-pent-4-ynyl-9H-purin-6-ylamine (141)

### Step 1 1-Iodo-2-nitro-4-trifluoromethoxy-benzene

A vigorously stirred solution of 2-nitro-4-trifluoromethoxy-phenylamine (4.4 g) in conc. HCl (19 mL) and water (19 mL) was cooled with ice (33 g), and sodium nitrite (1.5 g) was added in one portion. The reaction mixture was poured into a cold solution of KI (4.9 g) and to give a solid which was collected by filtration. The solid was washed with 6N HCl and water to afford the crude product, which was crystallized from hexane to give the pure title compound (5.6 g). Rt=7.26 (5-100-7). ¹H NMR (CDCl₃) δ 8.11(d, 1H), 7.77 (d, 1H), 7.20 (dd, 1H).

### Step 2 2-Iodo-5-trifluoromethoxy-phenylamine

To a solution of 1-iodo-2-nitro-4-trifluoromethoxy-benzene (5.58 g) in methanol (100 μL) were added FeCl₃.6H₂O (70 mg) and active carbon (35 mg). The mixture was heated to reflux, hydrazine monohydrate (1.6 g) was added slowly and the reaction progress was monitored by TLC. The catalyst was removed by filtration, and the methanol was evaporated in vacuo. The residue was dissolved in DCM and washed with water and brine, and the organic layer was concentrated to afford the title product (4.5 g). Rt=7.10 min (5-100-7). ¹H NMR (CDCl₃) δ 7.61 (d, 1H), 6.59 (s, 1H), 6.37 (d, 1H), 4.32 (br.s., 2H).

### Step 3 2-Iodo-5-trifluoromethoxy-benzenediazonium tetrafluoroborate

A slurry of 2-iodo-5-trifluoromethoxy-phenylamine (4.5 g) in water (2 mL) and 48% aq. HBF₄ (10.3 mL) was cooled to −10° C. and treated dropwise with a solution of NaNO₂ (1.1 g) in water (1 mL). The solid diazonium salt was collected by filtration, washed with diethyl ether, and air-dried to give the title compound 2.9 g, which was used without further purification.

### Step 4 8-(2-Iodo-5-trifluoromethoxy-phenylsulfanyl)-9H-purin-6-ylamine

A suspension of 6-amino-7,9-dihydro-purine-8-thione (0.99 g) in DMF (10 mL) was cooled to −35° C. and treated with 2-iodo-5-trifluoromethoxy-benzenediazonium tetrafluoroborate (2.86 g). The mixture was allowed to reach rt, and was neutralized with solid NaHCO₃ (465 mg). The mixture was evaporated, triturated in CHCl₃, filtered, and the solid was washed sequentially with CHCl₃, H₂O, Et₂O, and DCM to give the title compound (0.72 g).

### Step 5 8-(2-Iodo-5-trifluoromethoxy-phenylsulfanyl)-9-pent-4-ynyl-9H-purin-6-ylamine

A mixture of 8-(2-iodo-5-trifluoromethoxy-phenylsulfanyl)-9H-purin-6-ylamine (0.45 g), Cs₂CO₃ (0.65 g), 4-chloro-but-1-yne (0.12 g) in DMF (2 mL) was stirred at 80° C. The reaction progress was monitored by TLC. The DMF was evaporated and the residue was purified by chromatography to give the title compound. ¹H NMR (CDCl₃) δ 8.40 (s, 1H), 7.89 (d, 1H), 7.03 (d, 1H), 6.90(dd, 1H), 5.80 (br.s., 2H), 4.37 (t, 2H), 2.28 (td, 2H), 2.06 (quint., 2H), 2.00 (t, 1H).

### EXAMPLE 142 8-(2-Iodo-5-trifluoromethyl-phenylsulfanyl)-9-pent-4-ynyl-9H-purin-6-ylamine (142)

### Step 1 1-Iodo-2-nitro-4-trifluoromethyl-benzene

A vigorously stirred solution of 2-nitro-4-trifluoromethyl-aniline (4.7 g) in conc. HCl (19 mL) and water (19 mL) was cooled by addition of ice (33 g), and sodium nitrite (1.7 g) was added in one portion. The reaction mixture was poured into a cold solution of KI (5.6 g) and to give a solid which was collected by filtration. The solid was washed with 6N HCl and water to afford the crude product, which was crystallized from hexane to give pure the iodide (4.9 g). Rt=7.10 (5-100-7). ¹H NMR (CDCl₃) δ 8.24 (d, 1H), 8.13(d, 1H), 7.54 (dd, 1H).

### Step 2 2-Iodo-5-trifluoromethyl-aniline

To a solution of 1-iodo-2-nitro-4-trifluoromethyl-benzene (4.9 g) in methanol (100 mL) were added FeCl₃.6H₂O (65 mg) and active carbon (33 mg). The mixture was heated to reflux, hydrazine monohydrate (1.5 g) was added slowly and the reaction progress was monitored by TLC. The catalysts were removed by filtration, and the methanol was removed in vacuo. The solid residue was dissolved in DCM and washed with water and brine, and the organic layer was concentrated to afford the title compound (3.8 g). Rt=6.95 min (5-100-7). ¹H NMR (CDCl₃) δ 7.74 (d, 1H), 6.95 (s, 1H), 6.70 (d, 1H), 4.35 (br.s., 2H).

### Step 3 2-Iodo-5-trifluoromethyl-benzenediazonium tetrafluoroborate

A slurry of 2-iodo-5-trifluoromethyl-aniline (3.8 g) in water (2 mL) and 48% aq. HBF₄ (9.2 mL) was cooled to −10° C. and treated dropwise with a solution of NaNO₂ (1.0 g) in water (1 mL). The solid diazonium salt was collected by filtration, washed with diethyl ether, and air-dried to give the title compound (2.8 g), which was used without further purification.

### Step 4 8-(2-Iodo-5-trifluoromethyl-phenylsulfanyl)-9H-purin-6-ylamine

A suspension of 6-amino-7,9-dihydro-purine-8-thione (1.02 g) in DMF (10 mL) was cooled to −35° C. and treated with 2-iodo-5-trifluoromethyl-benzenediazonium tetrafluoroborate (2.8 g). The mixture was allowed to reach rt, and was neutralized with solid NaHCO₃ (460 mg). The mixture was evaporated, triturated in CHCl₃, filtered, and the solid was washed sequentially with CHCl₃, H₂O, Et₂O, and DCM to give the title compound (1.02 g).

### Step 5 8-(2-Iodo-5-trifluoromethyl-phenylsulfanyl)-9-pent-4-ynyl-9H-purin-6-ylamine

A mixture of 8-(2-iodo-5-trifluoromethyl-phenylsulfanyl)-9H-purin-6-ylamine (0.44 g), CS₂CO₃ (0.65 g), 4-chloro-but-1-yne (0.12 g), and DMF (2 mL) was stirred at 80° C. The reaction progress was monitored by TLC. The DMF was evaporated and the residue was purified by chromatography to give the desired product. ¹H NMR (CDCl₃) δ 8.39 (s, 1H), 8.04 (d, 1H), 7.52 (d, 1H), 7.25 (dd, 1H), 5.76 (br.s., 2H), 4.38 (t, 2H), 2.29 (dt, 2H), 2.08 (quint., 2H), 1.99 (t, 1H).

### EXAMPLE 143 8-(2,4-Diiodo-5-methoxy-phenylsulfanyl)-9-pent-4-ynyl-9H-purin-6-ylamine (143)

### Step 1 2-Iodo-5-methoxy-aniline and 2,4-Diiodo-5-methoxy-aniline

To a solution of 1-iodo-4-methoxy-2-nitro-benzene (100 g) in methanol (1000 mL) were added FeCl₃.6H₂O (1.5 g) and active carbon (0.76 g). The mixture was heated to reflux, hydrazine monohydrate (35 mL) was added slowly and the reaction progress was monitored by TLC. The catalyst was removed by filtration and methanol was removed under vacuo. The residue was dissolved in DCM, washed with water and brine, and the organic layer was concentrated to afford the crude product (83 g), which was purified by flash chromatography to give 2-iodo-5-methoxy-aniline (80 g) and 2,4-diiodo-5-methoxy-aniline (1 g).

### Step 2 2,4-diiodo-5-methoxy-benzenediazonium tetrafluoroborate

A slurry of 2,4-diiodo-5-methoxy-aniline (0.84 g) in water (3 mL) and 48% aq. HBF₄ (1.5 mL) was cooled to −10° C. and treated dropwise with a solution of NaNO₂ (0.17 g) in water (1 mL). The solid diazonium salt was collected by filtration, washed with diethyl ether, and air-dried to give the title compound (0.77 g), which was used without further purification.

### Step 3 8-(2,4-diiodo-5-methoxy-phenylsulfanyl)-9H-purin-6-ylamine

A suspension of 6-amino-7,9-dihydro-purine-8-thione (0.27 g) in DMF (5 mL) was cooled to −35° C. and treated with 2,4-diiodo-5-methoxy-benzenediazonium tetrafluoroborate (0.77 g). The mixture was allowed to reach rt, and was neutralized with solid NaHCO₃ (465 mg). The mixture was evaporated, triturated in CHCl₃, filtered, and the solid was washed sequentially with CHCl₃, H₂O, Et₂O, and DCM to give the title compound (0.14 g).

### Step 4 8-(2,4-diiodo-5-methoxy-phenylsulfanyl)-9-pent-4-ynyl-9H-purin-6-ylamine

A mixture of 8-(2,4-diiodo-5-methoxy-phenylsulfanyl)-9H-purin-6-ylamine (0.14 g), Cs₂CO₃ (0.173 g), 4-chloro-but-1-yne (0.3 mL), and DMF (3 mL) was stirred at 80° C. for 3 h. The DMF was evaporated and the residue was purified by chromatography to give the desired product. Rt=8.84 min. ¹H NMR (DMSO) δ 8.21 (s, 1H), 8.18 (s, 1H), 7.44 (s, 3H), 4.25 (t, 2H), 3.62 (s, 3H), 2.79 (d, 1H), 2.22 (td, 2H), 1.90 (quint., 2H).

### EXAMPLE 144 8-(2,5-Dimethoxy-biphenyl-3-ylsulfanyl)-9-pent-4-ynyl-9H-purin-6-ylamine (144)

### Step 1 2,5-Dimethoxy-3-nitro-biphenyl

A mixture of 1-bromo-2,5-dimethoxy-3-nitro-benzene (1.35 g), PhB(OH)₂ (1.00 g), K₃PO₄ (2.3 g), Pd(PPh₃)₄ (0.33 g) and toluene (20 mL) was heated to 108° C. for 24 h. The organic layer was diluted with toluene, washed with NaOH 1M, and water, and concentrated to afford the title product. ¹H NMR (CDCl₃) δ7.67 (dd, 1H), 7.55 (dd, 2H), 7.44 (dd, 2H), 7.24 (d, 1H), 7.10 (d, 1H), 3.85 (s, 3H), 3.46 (s, 3H).

### Step 2 2,5-Dimethoxy-biphenyl-3-ylamine

A mixture of 2,5-dimethoxy-3-nitro-biphenyl (1.88 g), 10% Pd/C (0.55 g) in EtOAc (40 mL) was shaken in a Parr hydrogenator under H₂ (4.8 atm) for 2 h. Filtration and chromatography gave the title compound. ¹H NMR (CDCl₃) δ7.59 (d, 2H), 7.57 (t, 2H), 7.40 (d, 1H), 6.32 (d, 1H), 6.27 (d, 1H), 3.76 (s, 3H), 3.34 (s, 3H).

### Step 3 2,5-Dimethoxy-3-phenyl-benzenediazonium tetrafluoroborate

A slurry of 3,5-dimethoxy-biphenyl-3-ylamine (970 mg) in water (2 mL) and 48% aq. HBF₄ (2 mL) was cooled to −10° C. and treated dropwise with a solution of NaNO₂ (380 mg) in water (1 mL) for 30 min. The solid diazonium salt was collected by filtration, washed with diethyl ether, and air-dried to give the title compound. ¹H NMR (DMSO-d₆) δ7.98 (d, 1H), 7.76 (d, 1H), 7.56 (m, 5H), 3.96 (s, 3H), 3.70 (s, 3H).

### Step 4 8-(2,5-Dimethoxy-biphenyl-3-ylsulfanyl)-9H-purin-6-ylamine

A suspension of 6-amino-7,9-dihydro-purine-8-thione (346 mg; Biamonte, J. Org. Chem. 2005, 70, 717) in DMF (2 mL) was cooled to −40° C. and treated with 2,5-dimethoxy-3-phenyl-benzenediazonium tetrafluoroborate (1.0 g). The mixture was allowed to reach rt, and was neutralized with solid NaHCO₃ (529 mg). The mixture was evaporated, triturated in CHCl₃, filtered, and the solid was washed sequentially with CHCl₃, H₂O, Et₂O, and DCM. The solid was redissolved in DMF, diluted with EtOAc, washed with NaOH 1M, and concentrated to give the desired product, free of unreacted starting material. ¹H NMR (CDCl₃) δ8.01 (s, 1H), 7.42 (d, 2H), 7.26 (m, 5H), 6.59 (s, 2H), 3.55 (s, 3H), 3.29 (s, 3H).

### Step 5 8-(2,5-Dimethoxy-biphenyl-3-ylsulfanyl)-9-pent-4-ynyl-9H-purin-6-ylamine

A suspension of 8-(2,5-Dimethoxy-biphenyl-3-ylsulfanyl)-9H-purin-6-ylamine (39 mg), 5-chloro-pent-1-yne (130 uL) and K₂CO₃ (167 mg) in DMF (2 mL) was heated to 70° C. overnight. Work-up and reverse-phase chromatography (C18; gradient water/CH₃CN) afforded the title compound. Rt=8.80 min (5-100-12). ¹H NMR (CDCl₃/CD₃OD 10:1) δ8.23 (s, 1H), 7.52 (d, 2H), 7.53 (m, 3H), 6.94 (d, 2H), 6.92 (d, 1H), 4.36 (t, 2H), 3.79 (s, 3H), 3.34 (s, 3H), 2.32 (dt, 2H), 2.11 (quint., 2H), 1.96 (t, 1H).

### EXAMPLE 145 8-(3-Bromo-2,5-dimethoxy-phenylsulfanyl)-9-pent-4-ynyl-9H-purin-6-ylamine

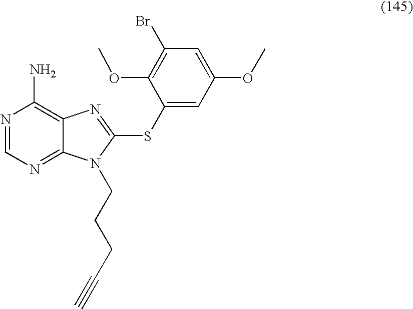

A suspension of crude 8-(3-bromo-2,5-dimethoxy-phenylsulfanyl)-9H-purin-6-ylamine (640 mg), 5-chloro-pent-1-yne (420 uL) and K₂CO₃ (550 mg) in DMF (2 mL) was heated to 75° C. overnight. Work-up and reverse-phase chromatography (C18; gradient water/CH₃CN) afforded the title compound. Rt=7.84 min (5-100-12). ¹H NMR (CDCl₃/CD₃OD 10:1) δ8.25 (s, 1H), 7.06 (d, 1H), 6.74 (d, 1H), 4.36 (t, 2H), 3.85 (s, 3H), 3.71 (s, 3H), 2.28 (dt, 2H), 2.05 (quint., 2H), 1.97 (t, 1H).

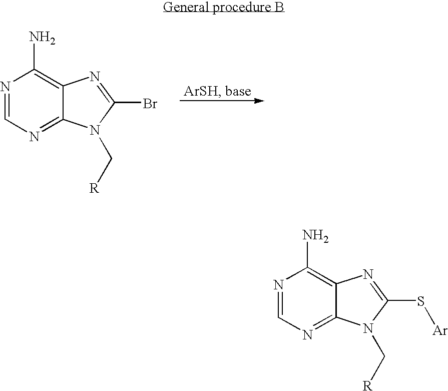

A solution of Ar—SH (14 equiv.) in DMF was treated with one equivalent of base (NaH or t-BuOK, 1-4 equiv.) for 10 min at rt. The 8-bromoadenine (1 equiv.) was added, and the mixture was stirred at 50-140° C. for 2-16 h. The reaction mixture was diluted with EtOAc, washed with NaOH 1M, water, and brine. Drying Na₂SO₄, evaporation, and preparative TLC or flash chromatography (e.g. AcOEt/Hexane/Et₃N 80:20:3) gave the desired compound. The following compounds were prepared in this manner.

### EXAMPLE 146 8-(Benzothiazol-2-ylsulfanyl)-9-pent-4-ynyl-9H-purin-6-ylamine

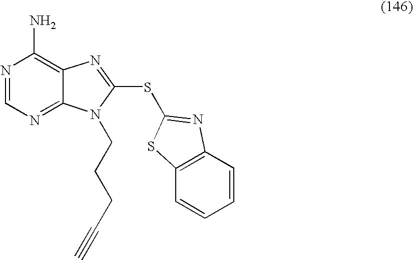

The title compound was obtained by reacting 8-bromo-9-(4-methyl-pent-3-enyl)-9H-purin-6-ylamine with benzothiazole-2-thiol according to the general procedure B. Rt=7.098 min (5-100-7). ¹H NMR (CDCl₃) δ 8.41 (s, 1H), 7.92 (d, 1H), 7.73 (d, 1H), 7.46 (t, 1H0, 7.35 (t, 1H), 6.42 (br. s. 2H), 4.45 (t, 2H), 2.25 (d, 2H), 2.13 (quint, 2H), 1.94 (s, 1H).

### EXAMPLE 147 9-Pent-4-ynyl-8-(quinolin-2-ylsulfanyl)-9H-purin-6-ylamine

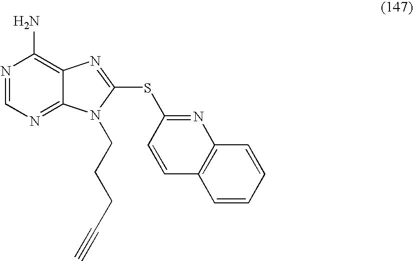

The title compound was obtained by reacting 8-bromo-9-(4-methyl-pent-3-enyl)-9H-purin-6-ylamine with quinoline-2-thiol according to the general procedure B. Rt=7.064 min (5-100-7). ¹H NMR (CDCl₃) δ 8.40 (s, 1H), 8.00 (d, 1H), 7.82 (d, 1H), 7.73 (d, 1H), 7.64 (t, 1H), 7.48 (t, 1H), 7.21 (d, 1H), 6.50 (br. s. 2H), 4.39 (t, 2H), 2.20 (d, 2H), 2.10 (quint, 2H), 1.84 (s, 1H).

### EXAMPLE 148 8-(3-Bromo-2,5-dimethoxy-phenylsulfanyl)-9-(4-methyl-pent-3-enyl)-9H-purin-6-ylamine (148)

### Step 1 1-Bromo-2,5-dimethoxy-3-nitro-benzene

A solution of 2-bromo-4-methoxy-6-nitro-phenol (17.3 g; Guay, J. Heterocycl. Chem. 1987, 24, 1649) in acetone (173 mL) was treated with Cs₂CO₃ (55 g) and Me₂SO₄ (16 mL) at reflux for 1 h. The mixture was filtered through a silica gel pad, concentrated, and chromatographed (toluene/hexane 1:1) to give the title compound as a pale yellow solid. ¹H NMR (CDCl₃) δ7.55 (s, 2H), 4.01 (s, 3H), 3.90 (s, 3H).

### Step 2 3-Bromo-2,5-dimethoxy-phenylamine

A suspension of 1-bromo-2,5-dimethoxy-3-nitro-benzene (1.82 g), AcOH (250 uL) and Fe powder (325 mesh; 2.7 g) in water (8 mL) was heated to reflux for 2 h. The organic materials were extracted with DCM, washed (NaHCO₃) and evaporated to give the title compound. ¹H NMR (CDCl₃) δ6.46 (d, 1H), 6.24 (d, 1H), 3.91 9s, 2H), 3.78 (s, 3H), 3.71 (s, 3H).

### Step 3 3-Bromo-2,5-dimethoxy-benzenediazonium tetrafluoroborate

A slurry of 3-bromo-2,5-dimethoxy-phenylamine (1.2 g) in water (2 mL) and 48% aq. HBF₄ (2 mL) was cooled to −10° C. and treated dropwise with a solution of NaNO₂ (411 mg) in water (1 mL). The solid diazonium salt was collected by filtration, washed with diethyl ether, and air-dried to give the title compound, which was used without further purification.

### Step 4 8-(3-Bromo-2,5-dimethoxy-phenylsulfanyl)-9H-purin-6-ylamine

A suspension of 6-amino-7,9-dihydro-purine-8-thione (458 mg; Biamonte, J. Org. Chem. 2005, 70, 717) in DMF (3 mL) was cooled to −35° C. and treated with 3-bromo-2,5-dimethoxy-benzenediazonium tetrafluoroborate (1.0 g). The mixture was allowed to reach rt, and was neutralized with solid NaHCO₃ (465 mg). The mixture was evaporated, triturated in CHCl₃, filtered, and the solid was washed sequentially with CHCl₃, H₂O, Et₂O, and DCM to give a 1:1 mixture of unreacted starting material and desired product.

### Step 5 8-(3-Bromo-2,5-dimethoxy-phenylsulfanyl)-9-(4-methyl-pent-3-enyl)-9H-purin-6-ylamine

A suspension of crude 8-(3-bromo-2,5-dimethoxy-phenylsulfanyl)-9H-purin-6-ylamine (183 mg), 5-bromo-2-methyl-pent-2-ene (172 uL) and Cs₂CO₃ (422 mg) in DMF (2 mL) was heated to 50° C. for 1 h. Work-up and chromatography (EtOAc/MeOH/Et₃N 100:3:3) afforded the title compound. Rt=9.01 min (5-100-12). ¹H NMR (CDCl₃/CD₃OD 10:1) δ 8.38 (s, 1H), 7.00 (d, 1H), 6.51 (d, 1H), 5.10 (t, 1H), 4.25 (t, 2H), 3.88 (s, 3H), 3.67 (s, 3H), 2.48 (q, 2H), 1.39 (s, 3H), 1.25 (s, 3H).

### EXAMPLE 149 9-(4-Methyl-pent-3-enyl)-8-(thiazol-2-ylsulfanyl)-9H-purin-6-ylamine (149)

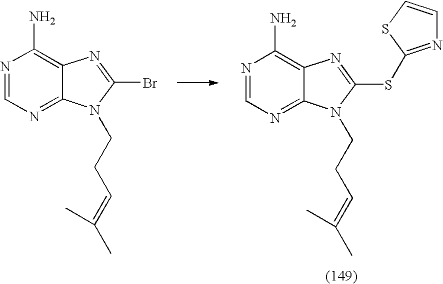

The title compound was obtained by reacting 8-bromo-9-(4-methyl-pent-3-enyl)-9H-purin-6-ylamine with 2-mercaptothiazole according to the general procedure B.

Rt=6.80 min (5-100-12). ¹H NMR (CDCl₃) δ 8.35 (s, 1H), 7.73 (d, 1H), 7.34 (d, 1H), 6.24 (s, 2H), 5.10 (t, 1H), 4.30 (t, 2H), 2.48 (q, 2H), 1.63 (s, 3H), 1.34 (s, 3H).

### EXAMPLE 150 8-(Benzothiazol-2-ylsulfanyl)-9-(4-methyl-pent-3-enyl)-9H-purin-6-ylamine

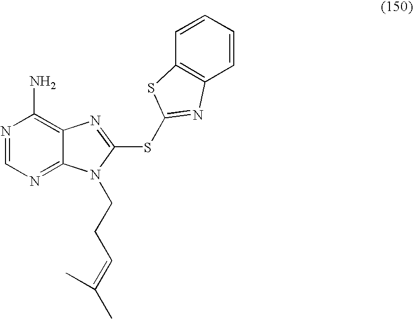

The title compound was obtained by reacting 8-bromo-9-(4-methyl-pent-3-enyl)-9H-purin-6-ylamine with 2-mercaptobenzothiazole according to the general procedure B.

Rt=8.25 min (5-100-12). ¹H NMR (CDCl₃) δ 8.39 (s, 1H), 7.91 (d, 1H), 7.70 (d, 1H), 7.42 (t, 2H), 7.33 (t, 2H), 6.19 (s, 2H), 5.09 (t, 1H), 4.32 (t, 2H), 2.51 (q, 2H), 1.58 (s, 3H), 1.28 (s, 3H).

### EXAMPLE 151 8-(1H-Benzoimidazol-2-ylsulfanyl)-9-(4-methyl-pent-3-enyl)-9H-purin-6-ylamine

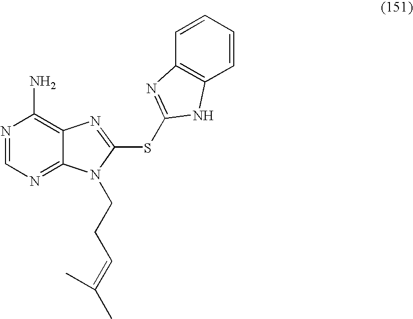

The title compound was obtained by reacting 8-bromo-9-(4-methyl-pent-3-enyl)-9H-purin-6-ylamine with 2-mercaptobenzimidazole according to the general procedure B.

Rt=5.18 min (5-100-7). ¹H NMR (CDCl₃) δ 12.80 (s, 1H), 8.12 (s, 1H), 7.70 (br. s, 1H), 7.40 (br. s, 1H), 7.12 m (2H), (d, 1H), 6.72 (s, 2H), 5.10 (t, 1H), 4.12 (t, 2H), 2.39 (q, 2H), 1.52 (s, 3H), 1.25 (s, 3H).

### EXAMPLE 152 8-(1-Allyl-1H-benzoimidazol-2-ylsulfanyl)-9-(4-methyl-pent-3-enyl)-9H-purin-6-ylamine

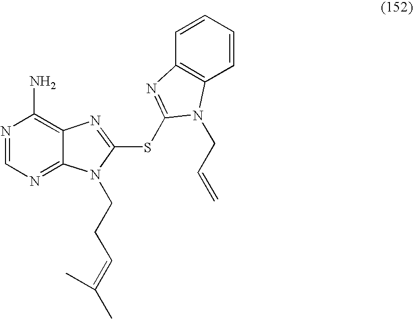

8-(1H-Benzoimidazol-2-ylsulfanyl)-9-(4-methyl-pent-3-enyl)-9H-purin-6-ylamine (151; 72 mg) was treated with allyl bromide (35 uL) and Cs₂CO₃ (237 mg) in DMF (4 mL) at 65° C. for 15 min. Work-up and flash chromatography (0-8% MeOH in DCM) gave the title compound. Rt=5.90 min (5-100-7). ¹H NMR (CDCl₃) δ 8.28 (s, 1H), 7.70 (d, 1H), 7.26 (m, 3H), 6.20 (s, 2H), 5.88 (m, 1H), 5.15 (m, 2H), 4.96 (m, 3H), 4.33 (t, 2H), 2.51 (q, 2H), 1.62 (s, 3H), 1.36 (s, 3H).

### EXAMPLE 153 8-(1-Methyl-1H-benzoimidazol-2-ylsulfanyl)-9-(4-methyl-pent-3-enyl)-9H-purin-6-ylamine

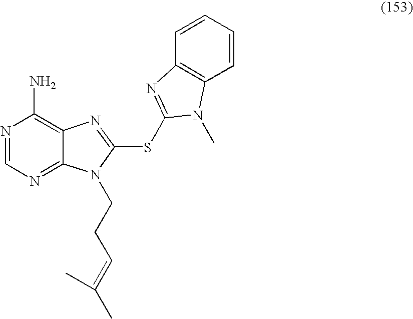

8-(1H-Benzoimidazol-2-ylsulfanyl)-9-(4-methyl-pent-3-enyl)-9H-purin-6-ylamine (151; 62 mg) was treated with Me₂SO₄ (35 uL) and Cs₂CO₃ (238 mg) in DMF (4 mL) at 65° C. for 15 min. Work-up and flash chromatography (0-8% MeOH in DCM) gave the title compound. Rt=5.43 min (5-100-7). ¹H NMR (CDCl₃) δ 8.29 (s, 1H), 7.71 (d, 1H), 7.31 (m, 3H), 5.84 (s, 2H), 5.11 (t, 1H), 4.35 (t, 2H), 3.88 (s, 3H), 2.54 (q, 2H), 1.64 (s, 3H), 1.39 (s, 3H).

### EXAMPLE 154 2-[6-Amino-8-(naphthalen-2-ylsulfanyl)-purin-9-yl]-ethanol

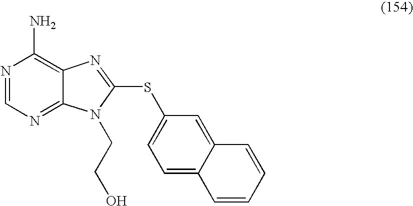

A solution of acetic acid 2-[6-amino-8-(naphthalen-2-ylsulfanyl)-purin-9-yl]-ethyl ester (157; 20 mg) in refluxing EtOH (1 mL) was treated with one drop NaOH 1M and let cool to rt, whereupon the desired compound crystallized. Filtration and washing (EtOH) gave the title compound. Rt=4.94 min (5-100-7). ¹H NMR (CDCl₃/CD₃OD 10:1) δ 8.15 (s, 1H), 7.97 (s, 1H), 7.80 (m, 3H), 7.49 (m, 2H), 7.42 (m, 3H), 4.32 (t, 2H), 3.84 (t, 2H).

### EXAMPLE 155 2-[6-Amino-8-(naphthalen-1-ylsulfanyl)-purin-9-yl]-ethanol

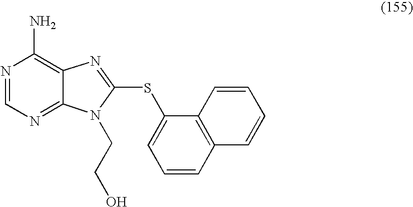

A solution of acetic acid 2-[6-amino-8-(naphthalen-1-ylsulfanyl)-purin-9-yl]-ethyl ester (158; 20 mg) in refluxing EtOH (1 mL) was treated with one drop NaOH 1M and let cool to rt, whereupon the desired compound crystallized. Filtration and washing (EtOH) gave the title compound. Rt=4.81 min (5-100-7). ¹H NMR (CDCl₃/CD₃OD 10:1) δ 8.25 (s, 1H), 8.13 (s, 1H), 7.95 (d, 1H), 7.89 (m, 1H), 7.78 (dd, 1H0, 7.53 (m, 2H), 7.47 (dd, 1H), 4.31 (t, 2H), 3.90 (t, 2H).

### EXAMPLE 156 2-[6-Amino-8-(quinolin-8-ylsulfanyl)-purin-9-yl]-ethanol

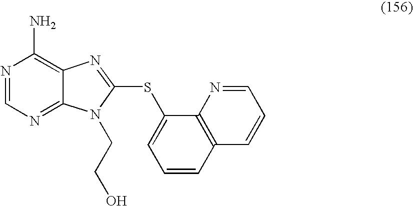

A solution of acetic acid 2-[6-amino-8-(quinolin-8-ylsulfanyl)-purin-9-yl]-ethyl ester (159; 32 mg) in refluxing EtOH (4.5 mL) was treated with one drop NaOH 1M and let cool to rt, whereupon the desired compound crystallized. Filtration and washing (EtOH) gave the title compound. Rt=3.93 min (5-100-7). ¹H NMR (CDCl₃/CD₃OD 10:1) δ 8.85 (dd, 1H), 8.20 (d, 1H), 8.18 (s, 1H), 7.78 (d, 1H), 7.43 (m, 3H), 2.14 (t, 2H0, 3.87 (t, 2H).

### EXAMPLE 157 Acetic acid 2-[6-amino-8-(naphthalen-2-ylsulfanyl)-purin-9-yl]-ethyl ester

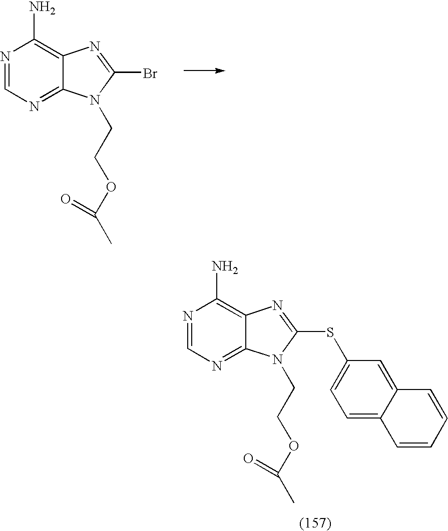

The title compound was obtained by reacting acetic acid 2-(6-amino-8-bromo-purin-9-yl)-ethyl ester with 2-naphtalenethiol according to the general procedure B. Rt=5.65 min (5-100-7). ¹H NMR (DMSO-d₆) δ 8.17 (s, 1H), 8.02 (s, 1H), 7.91 (m, 3H), 7.55 (m, 2H), 7.42 (m, 3H), 4.46 (t, 2H), 4.35 (t, 2H), 1.83 (s, 3H).

### EXAMPLE 158 Acetic acid 2-[6-amino-8-(naphthalen-1-ylsulfanyl)-purin-9-yl]-ethyl ester

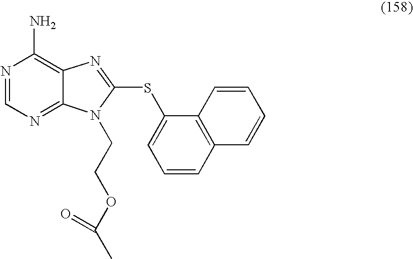

The title compound was obtained by reacting acetic acid 2-(6-amino-8-bromo-purin-9-yl)-ethyl ester with 1-naphtalenethiol according to the general procedure B. Rt=5.51 min (5-100-7). ¹H NMR (CDCl₃) δ 8.28 (s, 1H), 8.15 (s, 1H), 8.02 (m, 2H), 7.66 (dt, 1H), 7.63 (dt, 1H), 7.60 (dd, 1H), 7.51 (t, 1H), 7.31 (s, 2H), 4.47 (t, 2H), 4.33 (t, 2H), 1.87 (s, 3H).

### EXAMPLE 159 Acetic acid 2-[6-amino-8-(quinolin-8-ylsulfanyl)-purin-9-yl]-ethyl ester

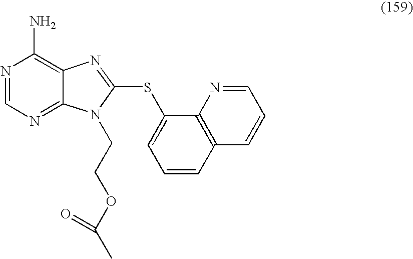

The title compound was obtained by acetic acid 2-(6-amino-8-bromo-purin-9-yl)-ethyl ester with 8-quinolinethiol hydrochloride according to the general procedure B. Rt=4.60 min (5-100-7). ¹H NMR (DMSO-d₆) δ 8.98 (dd, 1H), 8.45 (dd, 1H), 7.83 (dd, 1H), 7.67 (dd, 1H), 7.52 (s, 2H), 7.48 (t, 1H), 7.08 (dd, 1H), 4.47 (t, 2H), 4.35 (t, 2H), 1.77 (s, 3H).

### EXAMPLE 160 Acetic acid 2-[6-amino-8-(1H-indol-2-ylsulfanyl)-purin-9-yl]-ethyl ester

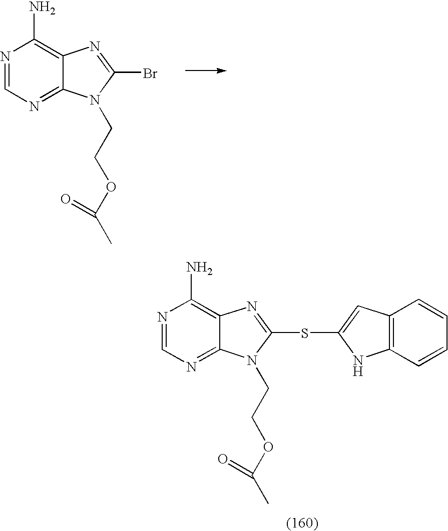

The title compound was obtained by reacting acetic acid 2-(6-amino-8-bromo-purin-9-yl)-ethyl ester with 1,3-dihydro-indole-2-thione (Takada, Chem. Phar. Bull. 1984, 32, 877) according to the general procedure B. Rt=5.30 min (5-100-7). ¹H NMR (DMSO-d₆) δ 8.13 (s, 1H), 7.53 (d, 1H), 7.34 (dd, 1H), 7.28 (s, 2H), 7.15 (ddd, 1H), 6.81 (dd, 1H), 4.48 (t, 2H), 4.35 (t, 2H), 1.92 (s, 3H).

### EXAMPLE 161 Acetic acid 2-[6-amino-8-(2,5-dimethoxy-phenylsulfanyl)-purin-9-yl]-ethyl ester

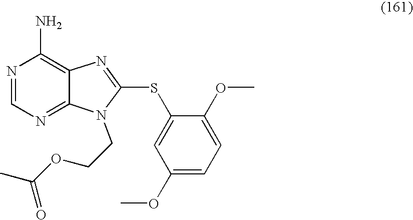

The title compound was obtained by reacting acetic acid 2-(6-amino-8-bromo-purin-9-yl)-ethyl ester with 2,5-dimethoxybenzenethiol according to the general procedure B. Rt=5.94 min (5-100-7). ¹H NMR (CDCl₃) δ 8.32 (s, 1H), 8.21 (d, 1H), 8.04 (d, 1H), 7.58 (m, 1H), 7.51 (m, 1H), 5.82 (s, 2H), 4.55 (t, 2H), 4.43 (t, 2H), 3.98 (s, 3H), 3.85 (s, 3H), 1.97 (s, 3H).

### EXAMPLE 162 Acetic acid 2-[6-amino-8-(benzo[b]thiophen-2-ylsulfanyl)-purin-9-yl]-ethyl ester

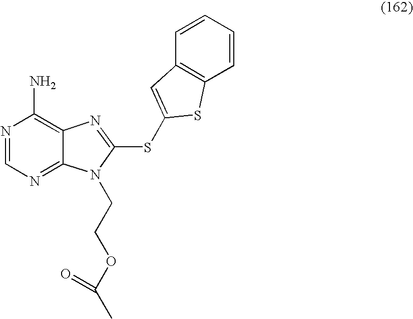

The title compound was obtained by reacting acetic acid 2-(6-amino-8-bromo-purin-9-yl)-ethyl ester with benzo[b]thiophene-2-thiol (Mitra, J. Sci. Indust. Res., 1957, 16B, 348) according to the general procedure B. Rt=5.58 min (5-100-7). ¹H NMR (CDCl₃) δ 8.21 (s, 1H), 7.71 (m, 2H), 7.58 (s, 1H), 7.32 (m, 2H), 6.04 (s, 2H), 4.55 (t, 2H), 4.44 (t, 2H), 2.00 (s, 3H).

### EXAMPLE 163 Acetic acid 2-[6-amino-8-(3-chloro-1H-indol-2-ylsulfanyl)-purin-9-yl]-ethyl ester

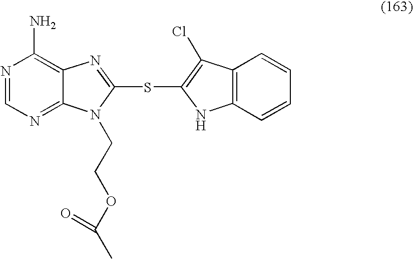

A solution of acetic acid 2-[6-amino-8-(1H-indol-2-ylsulfanyl)-purin-9-yl]-ethyl ester (160; 49 mg) in THF (2 mL) was treated with NCS (21 mg) at 60° C. for 1 h. Work-up (EtOAc/NaHCO₃) and preparative TLC (EtOAc/Et₃N 100/3) gave the title compound. Rt=5.68 min (5-100-7). ¹H NMR (CDCl₃) δ 8.23 (s, 1H), 7.53 (d, 1H), 7.23 (d, 1H), 7.15 (t, 1H), 7.10 (t, 1H), 6.16 (s, 2H), 4.45 (t, 2H), 4.38 (t, 2H), 1.98 (s, 3H).

### EXAMPLE 164 Acetic acid 2-[6-amino-8-(3-bromo-1H-indol-2-ylsulfanyl)-purin-9-yl]-ethyl ester

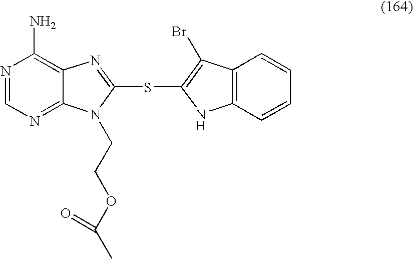

A solution of acetic acid 2-[6-amino-8-(1H-indol-2-ylsulfanyl)-purin-9-yl]-ethyl ester (160; 49 mg) in DCM (2 mL) and MeOH (0.5 mL) was treated with NBS (40 mg) at rt for 15 min. Work-up (EtOAc/NaHCO₃) and preparative TLC (EtOAc/Et₃N 100/3) gave the title compound. Rt=5.77 min (5-100-7). ¹H NMR (CDCl₃) δ 9.90 (s, 1H), 8.39 (s, 1H), 7.64 (m, 1H), 7.28 (m, 3H), 6.30 (s, 2H), 4.53 (t, 2H), 4.48 (s, 3H), 1.97 (s, 3H).

### EXAMPLE 165 Acetic acid 2-[6-amino-8-(3-iodo-1H-indol-2-ylsulfanyl)-purin-9-yl]-ethyl ester

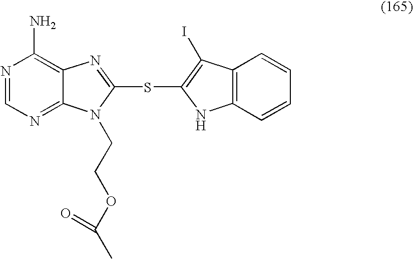

A solution of acetic acid 2-[6-amino-8-(1H-indol-2-ylsulfanyl)-purin-9-yl]-ethyl ester (160; 46 mg) in THF (2 mL) was treated with NIS (36 mg) at 60° C. for 1 h. Work-up (EtOAc/NaHCO₃) and preparative TLC (EtOAc/Et₃N 100/3) gave the title compound.

Rt=5.81 min (5-100-7). ¹H NMR (CDCl₃) δ 8.30 (s, 1H), 7.38 (d, 1H), 7.26 (m, 2H), 7.18 (t, 1H), 5.73 (s, 2H), 4.45 (t, 2H), 4.57 (t, 2H), 4.45 (s, 3H), 2.00 (s, 3H).

### EXAMPLE 166 Acetic acid 2-[6-amino-8-(1-propyl-1H-indol-2-ylsulfanyl)-purin-9-yl]-ethyl ester

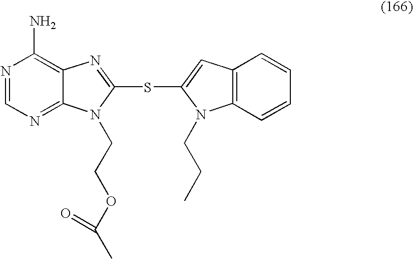

A solution of acetic acid 2-[6-amino-8-(1H-indol-2-ylsulfanyl)-purin-9-yl]-ethyl ester (160; 49 mg) in DMF (2 mL) was treated with 95% NaH (36 mg) and 1-iodopropane (370 uL) at rt for 1 h. Work-up (EtOAc/NaHCO₃) and preparative TLC (EtOAc) gave the title compound. Rt=6.04 min (5-100-7). ¹H NMR (CDCl₃) δ 8.26 (s, 1H), 7.60 (d, 1H), 7.34 (d, 1H), 7.26 (t, 1H), 7.11 (t, 1H), 6.95 (s, 1H), 5.80 (s, 2H), 4.55 (t, 2H), 4.44 (t, 2H), 4.25 (t, 2H), 2.03 (s, 3H), 1.71 (sext., 2H), 0.89 (t, 3H).

### EXAMPLE 167 Acetic acid 2-[6-amino-8-(3-iodo-1-propyl-1H-indol-2-ylsulfanyl)-purin-9-yl]-ethyl ester

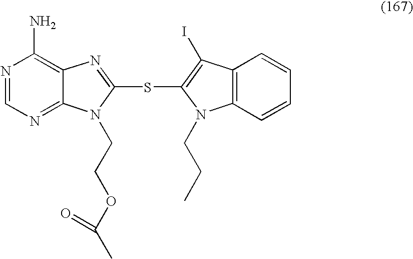

A solution of acetic acid 2-[6-amino-8-(1-propyl-1H-indol-2-ylsulfanyl)-purin-9-yl]-ethyl ester (166; 163 mg) in THF (2 mL) was treated with NIS (131 mg) at rt for 1 h. Preparative TLC (EtOAc) gave the title compound. Rt=6.48 min (5-100-7). ¹H NMR (CDCl₃) δ 8.24 (s, 1H), 7.44 (m, 2H), 7.15 (m, 1H), 2.04 (s, 2H), 4.63 (t, 2H), 4.44 (t, 2H), 4.36 (t, 2H), 2.03 (s, 3H), 1.75 (sext., 2H), 0.89 (t, 3H).

### EXAMPLE 168 Acetic acid 2-[6-amino-8-(1,4-dimethoxy-naphthalen-2-ylsulfanyl)-purin-9-yl]-ethyl ester

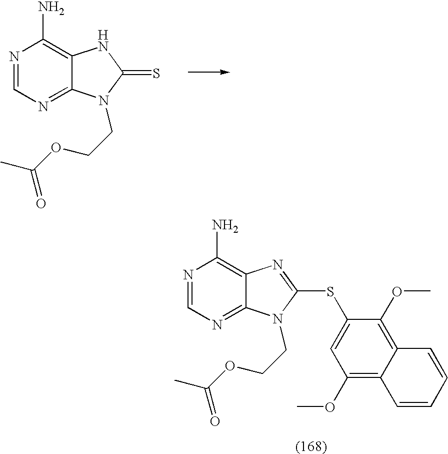

A solution of acetic acid 2-(6-amino-8-thioxo-7,8-dihydro-purin-9-yl)-ethyl ester (40 mg) and 1,4-dimethoxynaphthalene (30 mg) in 1,1,1,3,3,3-hexafluoro-2-propanol (400 uL) was degassed by bubbling N2 through the solution. Bis(trifluoroacetoxy)iodo]benzene (PIFA, 70 mg) was added at rt, whereupon the reaction turned from purple to deep green (radical cation) and then brown. After 1 h, the reaction mixture was evaporated, diluted with EtOAc, washed (diluted K₂CO₃, Na₂S₂O₃, brine). Preparative TLC (EtOAc) afforded the title compound. Rt=5.08 min (5-100-7). ¹H NMR (CDCl₃) δ 8.34 (s, 1H), 6.81 (m, 3H), 5.53 (s, 2H), 4.55 (t, 2H), 4.41 (t, 2H), 3.78 (s, 3H), 3.71 (s, 3H), 1.98 (s, 3H).

### EXAMPLE 169 3-[6-Amino-8-(benzo[1,3]dioxol-5-ylsulfanyl)-purin-9-yl]-propan-1-ol

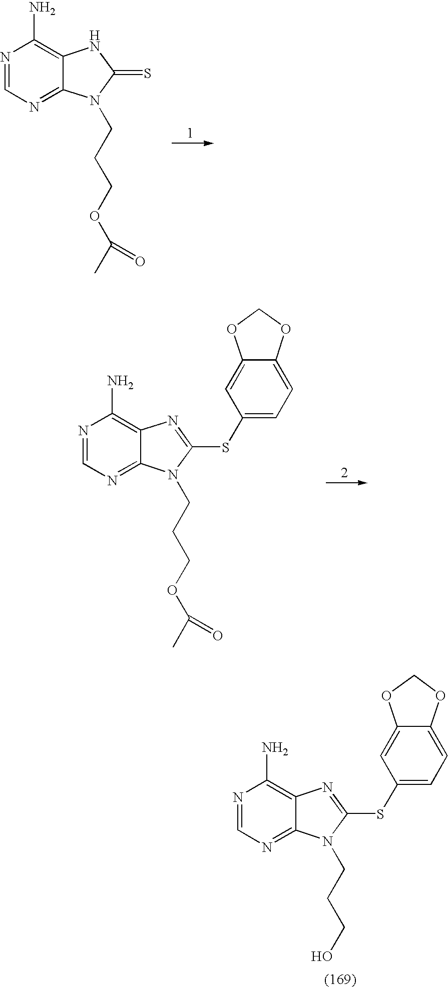

### Step 1 Acetic acid 3-[6-amino-8-(benzo[1,3]dioxol-5-ylsulfanyl)-purin-9-yl]-propyl ester

A solution of acetic acid 3-(6-amino-8-thioxo-7,8-dihydro-purin-9-yl)-propyl ester (174 mg) and 1,3-benzodioxole (75 uL) in 1,1,1,3,3,3-hexafluoro-2-propanol (0.8 mL) was treated with bis(trifluoroacetoxy)iodo]benzene (PIFA, 280 mg) for 30 min at rt. Work-up and preparative TLC (EtOAc/DCM/MeOH 50:50:3) gave the title compound. ¹H NMR (CDCl₃) δ 8.27 (s, 1H), 7.00 (dd, 1H), 6.95 (d, 1H), 6.75 (d, 1H), 6.18 (s 2H), 5.94 (s, 2H), 4.29 (t, 2H), 4.07 (t, 2H), 2.10 (quint., 2H), 2.02 (s, 3H).

### Step 2 3-[6-Amino-8-(benzo[1,3]dioxol-5-ylsulfanyl)-purin-9-yl]-propan-1-ol

A solution of acetic acid 3-[6-amino-8-(benzo[1,3]dioxol-5-ylsulfanyl)-purin-9-yl]-propyl ester (33 mg) in THF (0.5 mL) and MeOH (3 mL) was treated with K₂CO₃ (100 mg) at rt for 30 min to give, after work-up, the title compound. Rt=4.47 min (5-100-7). ¹H NMR (CDCl₃/CD₃OD 5:1) δ 8.12 (s, 1H), 7.05 (dd, 1H), 6.95 (d, 1H), 6.80 (d, 1H), 5.98 (s 2H), 4.25 (t, 2H), 3.45 (t, 2H), 1.85 (quint., 2H).

### EXAMPLE 170 3-[6-Amino-8-(2,3-dihydro-benzo[1,4]dioxin-6-ylsulfanyl)-purin-9-yl]-propan-1-ol (170)

### Step 1 Acetic acid 3-[6-amino-8-(2,3-dihydro-benzo[1,4]dioxin-6-ylsulfanyl)-purin-9-yl]-propyl ester

A solution of acetic acid 3-(6-amino-8-thioxo-7,8-dihydro-purin-9-yl)-propyl ester (337 mg) and 1,4-benzodioxane (225 uL) in 1,1,1,3,3,3-hexafluoro-2-propanol (2 mL) was degassed by bubbling N2 through the solution. Bis(trifluoroacetoxy)iodo]benzene (PIFA, 814 mg) was added at rt, whereupon the reaction turned deep blue (radical cation). After 30 min, the reaction mixture was evaporated, diluted with EtOAc, and washed (diluted K₂CO₃, Na₂S₂O₃, brine). Flash chromatography (0-15% MeOH in EtOAc) afforded the title compound. Rt=5.22 min (5-100-7). ¹H NMR (CDCl₃) δ 8.24 (s, 1H), 6.99 (dd, 1H), 6.92 (d, 1H), 6.80 (d, 1H), 6.28 (s 2H), 4.30 (t, 2H), 4.20 (s, 4H), 4.05 (t, 2H), 2.12 (quint., 2H), 2.01 (s, 3H).

### Step 2 3-[6-Amino-8-(2,3-dihydro-benzo[1,4]dioxin-6-ylsulfanyl)-purin-9-yl]-propan-1-ol

A solution of acetic acid 3-[6-amino-8-(2,3-dihydro-benzo[1,4]dioxin-6-ylsulfanyl)-purin-9-yl]-propyl ester (54 mg) in MeOH (5 mL) was treated with K₂CO₃ (113 mg) at rt for 30 min. Work-up and reverse-phase chromatography (C18; gradient water/CH₃CN) afforded the title compound. Rt=4.56 min (5-100-7). ¹H NMR (CDCl₃) δ 8.27 (s, 1H), 7.04 (d, 1H), 6.97 (dd, 1H), 6.86 (d, 1H), 5.87 (s, 2H), 4.38 (t, 2H), 4.25 (m, 4H), 3.40 (t, 2H), 1.82 (quint., 2H).

### EXAMPLE 171 8-(7-Bromo-benzothiazol-2-ylsulfanyl)-9-butyl-9H-purine-6-ylamine

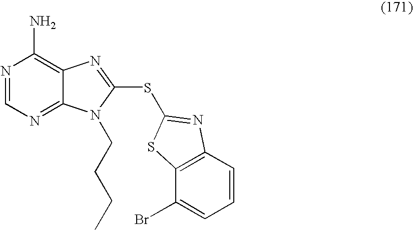

### Step 1 7-Bromo-benzothiazole-2-thiol

To a mixture of HNO₃ (90 ml) and H₂SO₄ (45 ml) cooled to 0° C. was added 1,2-dibromo-benzene (148.3 mmol, 35 g). The mixture was stirred at 0° C. for 30 min and then poured into 1.4 L of ice water. A solid precipitated and filtered. The solid was washed with water and dried over vacuum pump to give a mixture of 2,3-dibromonitrobenzene and 3,4-dibromonitrobenzene in 1:4 ratio (95% yield). The mixture of both regioisomers (142 mmol, 40 g) were treated with Fe (427 mmol, 23.9 g) in a solution comprised of 50% EtOH/H₂O (270 ml) and HCl (15 ml). The mixture was heated to 85° C. for 2 hours. Then the mixture was cooled to room temperature and solvent was removed. The crude material was extracted with EtOAC. The combined extracts were washed with water and brine, dried over MgSO₄, and concentrated to give a mixture of 2,3-dibromoaniline and 3,4-dibromoaniline in 1:4 ratio (95% yield). The mixture (34.86 mmol, 8.75 g) was added to a solution of O-ethylxanthic acid, potassium salt (52.3 mmol, 33.47 g) in DMF (150 ml) and heated to 160° C. for 4 hours. The work-up was the same as that described in 7-chloro-benzothiazole-2-thiol. 7-Bromo-benzothiazole-2-thiol was isolated from the unreacted 3,4-dibromoaniline as white solid with 94% yield. ¹H NMR (CD₃OD) δ 7.21 (t, J=6.34 Hz, 1H), 7.29 (d, J=6.30 Hz, 1H), 7.34 (d, J=7.34 Hz, 1H).

### Step 2 8-(7-Bromo-benzothiazol-2-ylsulfanyl)-9-butyl-9H-purine-6-ylamine

8-(7-Bromo-benzothiazol-2-ylsulfanyl)-9-butyl-9H-purine-6-ylamine was prepared by the same method described in example 232 except that 7-bromo-benzothiazole-2-thiol was used instead of 7-chloro-benzothiazole-2-thiol. ¹H NMR (CDCl₃) δ 0.92 (t, J=7.45 Hz, 3H), 1.38(m, 2H), 1.83(m, 2H), 4.34 (t, J=7.45 Hz, 2H), 5.83 (s, 2H, NH₂), 7.36 (d, 8.12 Hz, 1H), 7.50(t, J=8.12 Hz, 1H), 7.87 (d, J=8.12 Hz, 1H), 8.44 (s, 1H). HPLC: RT=6.51 min (method: 5-100-7).

### EXAMPLE 172 9-Butyl-8-(7-methyl-benzothiazol-2-ylsulfanyl)-9H-purine-6-ylamine

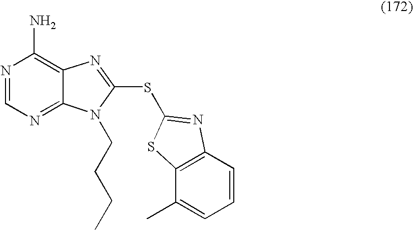

### Step 1 7-Methyl-benzothiazole-2-thiol

To a solution of 2-bromo-3-nitrotoluene (2.5 g, 11.57 mmol) in EtOH (18 ml) was added Fe (1.94 g, 34.7 mmol) and con. HCl (1 ml) at room temperature. The reaction mixture was heated to reflux for 1.5 hr and then cooled to room temperature. The solvent was removed. The residue was diluted with NH₄Cl (sat.) and extracted with EtOAC. The combined extracts were washed with water and brine, dried over MgSO₄, concentrated to give crude material 2-bromo-3-methylaniline with 90% yield. The compound 2-bromo-3-methylaniline was reacted with O-ethylxanthic acid, potassium salt to form 7-methyl-benzothiazole-2-thiol in 89% yield. ¹H NMR (CD₃OD) δ 7.10 (m, 2H), 7.29(t, J=8.08 Hz, 1H).

### Step 2 9-Butyl-g-(7-methyl-benzothiazol-2-ylsulfanyl)-9H-purine-6-ylamine

9-Butyl-8-(7-methyl-benzothiazol-2-ylsulfanyl)-9H-purine-6-ylamine was prepared by the same method described in example 49.1 except that 7-methyl-benzothiazole-2-thiol was used instead of 7-chloro-benzothiazole-2-thiol. ¹H NMR (CDCl₃) δ 0.88 (t, J=7.45 Hz, 3H), 1.34(m, 2H), 1.80(m, 2H), 2.47 (s, 3H), 4.33 (t, J=7.45 Hz, 2H), 6.10(s, 2H, NH₂), 7.16 (d, 7.34 Hz, 1H), 7.39(t, J=7.34 Hz, 1H), 7.78 (d, J=7.34 Hz, 1H), 8.42 (s, 1H). HPLC: RT=6.27 min (method: 5-100-7).

### EXAMPLE 173 9-Butyl-8-(7-methoxy-benzothiazol-2-ylsulfanyl)-9H-purine-6-ylamine

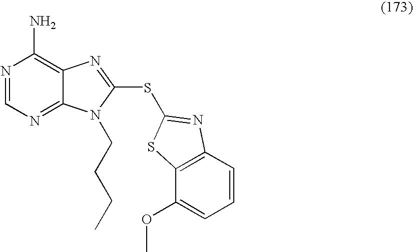

### Step 1 7-Methoxy-benzothiazole-2-thiol

To a solution of 2-amino-3-nitrophenol (10 g, 64.9 mmol) in DMF at room temperature was added K₂CO₃ (9.86 g, 71.4 mmol) and iodomethane (10.13 g, 71.4 mmol). The reaction mixture was stirred overnight, and the solvent removed under reduced pressure. The residue was diluted with NH₄Cl (sat.) and extracted with EtOAC. The combined extracts were washed with water and brine, dried over MgSO₄, concentrated and recrystallized from EtOAC and hexane to give 2-amino-3-nitroanisole. The 2-amino-3-nitroanisole was converted to 2-bromo-3-nitroanisole using the usual NaNO₂, aq. H₂SO₄ and CuBr, aq. HBr method. Reduction of 2-bromo-3-nitroanisole was achieved by treatment with iron in EtOH/HCl to give 2-bromo-3-aminoanisole which upon reaction with O-ethylxanthic acid, potassium salt gave 7-methoxy-benzothiazole-2-thiol in 89% yield. ¹H NMR (CD₃OD) δ 3.95 (s, 3H), 6.74 (d, J=8.20 Hz, 1H), 6.95 (d, J=8.08 Hz, 1H), 7.34 (t, J=8.15 Hz, 1H).

### Step 2 9-Butyl-8-(7-methoxy-benzothiazol-2-ylsulfanyl)-9H-purine-6-ylamine

9-Butyl-8-(7-methoxy-benzothiazol-2-ylsulfanyl)-9H-purine-6-ylamine was prepared by the same method described in example 232 except that 7-methoxy-benzothiazole-2-thiol was used instead of 7-chloro-benzothiazole-2-thiol. ¹H NMR (CDCl₃) δ 0.91 (t, J=7.45 Hz, 3H), 1.35(m, 2H), 1.82(m, 2H), 3.96(s, 3H), 4.33 (t, J=7.45 Hz, 2H), 5.68(s, 2H, NH₂), 6.83 (d, J=8.00 Hz, 1H), 7.43(t, J=8.00 Hz, 1H), 7.58 (d, J=8.00 Hz, 1H), 8.44(s, 1H). HPLC: RT=6.14 min (method: 5-100-7).

### EXAMPLE 174 9-Butyl-8-(7-ethoxy-benzothiazol-2-ylsulfanyl)-9H-purine-6-ylamine

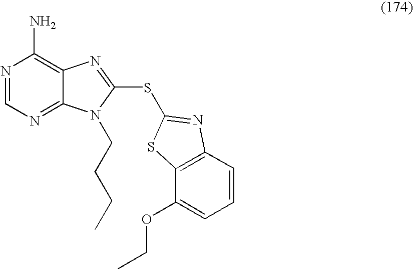

### Step 1 7-Ethoxy-benzothiazole-2-thiol

7-Ethoxy-benzothiazole-2-thiol was prepared by the method described for 7-methoxy-benzothiazole-2-thiol (example 173, step 1) except that iodoethane was used instead of iodomethane. 7-Ethoxy-benzothiazole-2-thiol was obtained as a white powder. ¹H NMR (CD₃OD) δ 1.44 (t, J=7 Hz, 3H), 4.21 (m, 2H), 6.84 (d, J=8.09 Hz, 1H), 6.91 (d, J=8.08 Hz, 1H), 7.34 (t, J=8.15 Hz, 1H).

### Step 2 9-Butyl-8-(7-ethoxy-benzothiazol-2-ylsulfanyl)-9H-purine-6-ylamine

9-Butyl-8-(7-ethoxy-benzothiazol-2-ylsulfanyl)-9H-purine-6-ylamine was prepared by the same method described example 232 except that 7-ethoxy-benzothiazole-2-thiol was used instead of 7-chloro-benzothiazole-2-thiol. ¹H NMR (CDCl₃) δ 0.90 (t, J=7.45 Hz, 3H), 1.35(m, 2H), 1.45 (t, J=7.45 Hz, 3H), 1.82(m, 2H), 4.21(t, J=7.45 Hz, 2H), 4.33 (t, J=7.45 Hz, 2H), 5.76(s, 2H, NH₂), 6.80 (d, J=8.00 Hz, 1H), 7.41(t, J=8.00 Hz, 1H), 7.56 (d, J=8.00 Hz, 1H), 8.44(s, 1H). HPLC: RT=6.10 min (method 5-100-7)

### EXAMPLE 175 9-Butyl-8-(7-fluoro-benzothiazol-2-ylsulfanyl)-9H-purine-6-ylamine

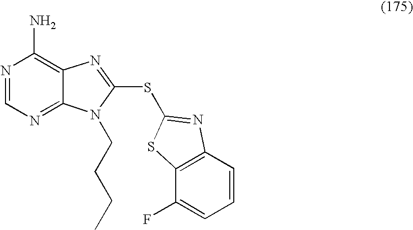

### Step 1 7-Fluoro-benzothiazole-2-thiol

7-Fluoro-benzothiazole-2-thiol was prepared by the same method described in 7-chloro-benzothiazole-2-thiol (example 232, step 3) except that 2,3-difluoro-phenylamine was used instead of 2,3-dichloro-phenylamine. 7-fluoro-benzothiazole-2-thiol was obtained as a white powder (92% yield). ¹H NMR (CDCl₃) δ 7.0 (t, J=8.3 Hz, 1H), 7.10 (d, J=8.13 Hz, 1H), 7.38(m, 1H).

### Step 2 9-Butyl-8-(7-fluoro-benzothiazol-2-ylsulfanyl)-9H-purine-6-ylamine

9-Butyl-8-(7-fluoro-benzothiazol-2-ylsulfanyl)-9H-purine-6-ylamine was prepared by the same method described example 232 except that 7-fluoro-benzothiazole-2-thiol was used instead of 7-chloro-benzothiazole-2-thiol. ¹H NMR (CDCl₃) δ 0.90 (t, J=7.45 Hz, 3H), 1.35(m, 2H), 1.82(m, 2H), 4.33 (t, J=7.45 Hz, 2H), 5.71(s, 2H, NH₂), 7.1 (t, J=8.30 Hz, 1H), 7.44(m, 1H), 7.75 (d, J=8.30 Hz, 1H), 8.44(s, 1H). HPLC: RT=6.14 min. (method: 5-100-7).

### EXAMPLE 176 9-Butyl-8-(7-trifluoromethyl-benzothiazol-2-ylsulfanyl)-9H-purine-6-ylamine

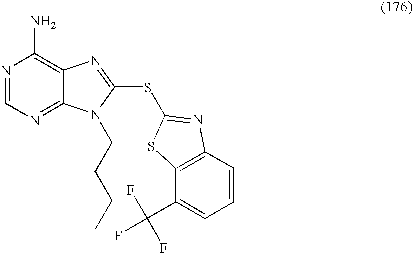

### Step 1 7-Trifluoromethyl-benzothiazole-2-thiol

7-Trifluoromethyl-benzothiazole-2-thiol was prepared by the same method described in 7-chloro-benzothiazole-2-thiole (example 232, step 3) except that 2-fluoro-3-trifluoromethyl-phenylamine was used instead of 2,3-dichloro-phenylamine. 7-trifluoromethyl-benzothiazole-2-thiol was obtained as a white powder (85% yield). ¹H NMR (CDCl₃) δ 7.50 (m, 2H), 7.57 (d, J=6.6 Hz, 1H).

### Step 2 9-Butyl-8-(7-trifluoromethyl-benzothiazol-2-ylsulfanyl)-9H-purine-6-ylamine

9-Butyl-8-(7-trifluoromethyl-benzothiazol-2-ylsulfanyl)-9H-purine-6-ylamine was prepared by the same method described example 232 except that 7-trifluoromethyl-benzothiazole-2-thiol was used instead of 7-chloro-benzothiazole-2-thiol. ¹H NMR (CDCl₃) δ 0.90 (t, J=7.45 Hz, 3H), 1.30 (m, 2H), 1.84 (m, 2H), 4.36 (t, J=7.45 Hz, 2H), 5.78 (s, 2H, NH₂), 7.60 (t, J=8.12 Hz, 1H), 7.68 (d, J=8.10 Hz, 1H), 8.11 (d, J=8.10 Hz, 1H), 8.45 (s, 1H). HPLC: RT=6.57 min (method: 5-100-7).

### EXAMPLE 177 9-Butyl-8-(7-chloro-thiazole[4,5-c]pyridin-2-ylsulfanyl)-9H-purin-6-ylamine (177)

9-Butyl-8-(7-chloro-thiazole[4,5-c]pyridin-2-ylsulfanyl)-9H-purin-6-ylamine was prepared by the same method described in example 232 except that 7-chloro-thiazole[4,5-c]pyridine-2-thiol (see 206, step 1) was used instead of 7-chloro-benzothiazole-2-thiol. ¹H NMR (CDCl₃) δ 0.94 (t, J=7.45 Hz, 3H), 1.38 (m, 2H), 1.86 (m, 2H), 4.35 (t, J=7.45 Hz, 2H), 5.74 (s, 2H, NH₂), 8.46 (s 1H), 8.49 (s, 1H), 9.10 (s, 1H). HPLC: RT=5.74 min (5-100-7).

### EXAMPLE 178 8-(Benzothiazol-2-ylsulfanyl)-9-butyl-9H-purine-6-ylamine

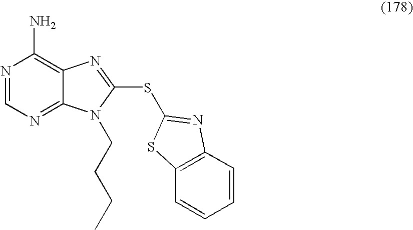

8-(Benzothiazol-2-ylsulfanyl)-9-butyl-9H-purine-6-ylamine was prepared by the same method described in example 232 except that benzothiazole-2-thiol (purchased from Acros) was used instead of 7-chloro-benzothiazole-2-thiol. ¹H NMR (CDCl₃) δ 0.87 (t, 3H), 1.32 (m, 2H), 1.79 (m, 2H), 4.33 (t, 2H), 6.62 (s, 2H, NH₂), 7.33 (m, 1H), 7.44(m, 1H), 7.70 (d, 1H), 7.90 (d, 1H), 8.40 (s, 1H). HPLC: RT=8.63 min (method: 5-100-15 min).

### EXAMPLE 179 9-Butyl-8-(6-chloro-benzothiazol-2-ylsulfanyl)-9H-purine-6-ylamine (179)

9-Butyl-8-(6-chloro-benzothiazol-2-ylsulfanyl)-9H-purine-6-ylamine was prepared by the same method described in example 232 except that 6-chloro-benzothiazole-2-thiol (purchased from Acros) was used instead of 7-chloro-benzothiazole-2-thiol. ¹H NMR (CDCl₃) δ 0.92 (t, 3H), 1.27 (m, 2H), 1.84 (m, 2H), 4.33 (t, 2H), 5.82 (s, 2H, NH₂), 7.42 (d, 1H), 7.74(s, 1H), 7.85 (d, 1H), 8.43 (s, 1H). MS: 391 (M+1), HPLC: RT=9.743 min (method: 5-100-15 min).

### EXAMPLE 180 9-Butyl-8-(5-chloro-benzothiazol-2-ylsulfanyl)-9H-purine-6-ylamine (180)

9-Butyl-8-(5-chloro-benzothiazol-2-ylsulfanyl)-9H-purine-6-ylamine was prepared by the same method described in example 232 except that 5-chloro-benzothiazole-2-thiol (purchased from Acros) was used instead of 7-chloro-benzothiazole-2-thiol. ¹H NMR (CDCl₃) δ 0.91 (t, 3H), 1.33 (m, 2H), 1.83 (m, 2H), 4.33 (t, 2H), 6.01 (s, 2H, NH₂), 7.34 (dd, 1H), 7.65(d, 1H), 7.92 (d, 1H), 8.43 (s, 1H). MS: 391.8 (M+1), 383.83 (M+3).

### EXAMPLE 181 9-Butyl-8-(4-chloro-benzothiazol-2-ylsulfanyl)-9H-purine-6-ylamine (181)

### Step 1 4-Chloro-benzothiazole-2-thiol

4-Chloro-benzothiazole-2-thiol was prepared by the same method described for 7-chloro-benzothiazole-2-thiole (example 232, step 3) except that 2,6-dichloro-phenylamine was used instead of 2,3-dichloro-phenylamine. 6-Chloro-benzothiazole-2-thiol was obtained as a white powder (94% yield). ¹H NMR (CDCl₃) δ 7.21 (t, J=8.0 Hz, 1H), 7.36 (m, 2H). HPLC

### Step 2 9-Butyl-8-(4-chloro-benzothiazol-2-ylsulfanyl)-9H-purine-6-ylamine

9-Butyl-8-(4-chloro-benzothiazol-2-ylsulfanyl)-9H-purine-6-ylamine was prepared by the same method described in example 232 except that 4-chloro-benzothiazole-2-thiol was used instead of 7-chloro-benzothiazole-2-thiol. ¹H NMR (CDCl₃) δ 0.90 (t, 3H), 1.35 (m, 2H), 1.83 (m, 2H), 4.35 (t, 2H), 5.98 (s, 2H, NH₂), 7.29 (t, 1H), 7.51 (d, 1H), 7.65 (d, 1H), 8.43 (s, 1H). HPLC: RT=9.43 min (method: 5-100-15).

### EXAMPLE 182 9-Butyl-8-(4-chloro-benzothiazol-2-ylsulfanyl)-9H-purine-6-ylamine (182)

9-Butyl-8-(4-chloro-benzothiazol-2-ylsulfanyl)-9H-purine-6-ylamine was prepared by the same method described in example 232. ¹H NMR (CDCl₃) δ 1.33 (s, 3H), 1.73 (s, 3H), 2.54 (m, 2H), 4.35 (t, 2H), 5.13 (m, 1H), 5.86 (s, 2H, NH₂), 7.34 (d, 1H), 7.42(m, 1H), 7.84 (d, 1H), 8.45 (s, 1H). HPLC: RT=6.71 min (method: 5-100-7).

### EXAMPLE 183 9-Butyl-8-(thiazolo[5,4-b]pyridin-2-ylsulfanyl)-9H-purine-6-ylamine (183)

9-Butyl-8-(thiazolo[5,4-b]pyridin-2-ylsulfanyl)-9H-purine-6-ylamine was prepared by the same method described in example 232. ¹H NMR (CDCl₃) δ 0.92 (t, 3H), 1.34 (m, 2H), 1.85 (m, 2H), 4.36 (t, 2H), 5.74 (s, 2H), 7.44 (dd, 1H), 8.15(d, 1H), 8.45 (s, 1H), 8.54(d, 1H). HPLC: RT=:5.33 min (method: 5-100-7).

### EXAMPLE 184 8-(4-Bromo-6,7-difluoro-benzothiazol-2-ylsulfanyl)-9-butyl-9H-purine-6-ylamine (184)

8-(4-Bromo-6,7-difluoro-benzothiazol-2-ylsulfanyl)-9-butyl-9H-purine-6-ylamine was prepared by the same method described in example 232. ¹H NMR (CDCl₃) δ 0.94 (t, 3H), 1.38 (m, 2H), 1.83 (m, 2H), 4.35 (t, 2H), 5.90 (s, 2H), 7.56 (dd, 1H), 8.70 (s, 1H). HPLC: RT=6.83 min (method: 5-100-7).

### EXAMPLE 185 9-Butyl-8-(6,7-dichloro-benzothiazol-2-ylsulfanyl)-9H-purine-6-ylamine (185)

9-Butyl-8-(6,7-dichloro-benzothiazol-2-ylsulfanyl)-9H-purine-6-ylamine was prepared by the same method described in example 232. ¹H NMR (CDCl₃) δ 0.89 (t, 3H), 1.35 (m, 2H), 1.83 (m, 2H), 4.35 (t, 2H), 6.14(s, 2H), 7.58(d, 1H), 7.75 (d, 1H), 8.40 (s, 1H). HPLC: RT=6.92 min (method: 5-100-7).

### EXAMPLE 186 9-Butyl-8-(6,7-difluoro-benzothiazol-2-ylsulfanyl)-9H-purine-6-ylamine (186)

9-Butyl-8-(6,7-difluoro-benzothiazol-2-ylsulfanyl)-9H-purine-6-ylamine was prepared by the same method described in example 232. ¹H NMR (CDCl₃) δ 0.92 (t, 3H), 1.35 (m, 2H), 1.83 (m, 2H), 4.35 (t, 2H), 6.26(s, 2H), 7.34(dd, 1H), 7.67 (dd, 1H), 8.43 (s, 1H). HPLC: RT=6.32 min (method: 5-100-7).

### EXAMPLE 187 9-Butyl-8-(7-methoxymethoxymethyl-benzothiazol-2-ylsulfanyl)-9H-purine-6-ylamine

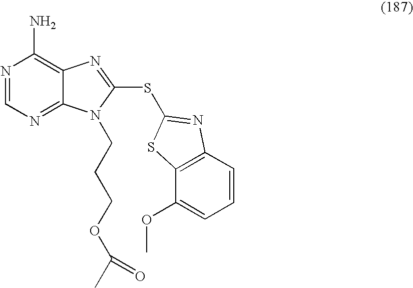

9-Butyl-8-(7-methoxymethoxymethyl-benzothiazol-2-ylsulfanyl)-9H-purine-6-ylamine was prepared by the same method described in example 232. ¹H NMR (CDCl₃) δ 0.89 (t, 3H), 1.35 (m, 2H), 1.83 (m, 2H), 3.40 (s, 3H), 4.35 (t, 2H), 4.68 (s,2H), 4.77(s,2H), 5.65 (s, 2H), 7.31(d, 1H), 7.55 (t, 1H), 7.90(d,1H), 8.45 (s, 1H). HPLC: RT=6.06 min

### EXAMPLE 188 Acetic acid 4-[6-amino-8-(7-fluoro-benzothiazol-2-ylsulfanyl)-purin-9-yl]-butyl ester

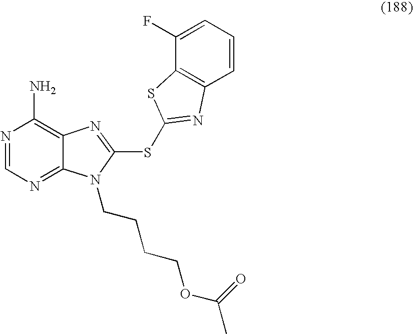

Acetic acid 4-[6-amino-8-(7-fluoro-benzothiazol-2-ylsulfanyl)-purin-9-yl]-butyl ester was prepared by the same method described in example 232. ¹H NMR (CDCl₃) δ 1.69(m, 2H), 1.95(m, 2H), 1.98 (s, 3H), 4.06(t, 2H), 4.39 (t,2H), 5.69(s, 2H), 7.29 (t, 1H), 7.44(dd,1H), 7.74(d, 1H), 8.44 (s,1H); HPLC: RT=5.66 min(5-100-7).

### EXAMPLE 189 Acetic acid 3-[6-amino-8-(6,7-dichloro-benzothiazol-2-ylsulfanyl)-purin-9-yl]-propyl ester (189)

Acetic acid 3-[6-amino-8-(6,7-dichloro-benzothiazol-2-ylsulfanyl)-purin-9-yl]-propyl ester was prepared by the same method described in example 232. ¹H NMR (CDCl₃) δ 1.99 (s, 3H), 2.22 (m, 2H), 4.07 (t, 2H), 4.46 (t, 2H), 5.70 (s, 2H), 7.54 (d, 1H), 7.73 (d, 1H), 7.82 (d, 1H), 8.41(s, 1H). HPLC: RT=6.19 min. (method: 5-100-7).

### EXAMPLE 190 8-(6,7-Dichloro-benzothiazol-2-ylsulfanyl)-9-pent-4-ynyl-9H-purin-6-ylamine

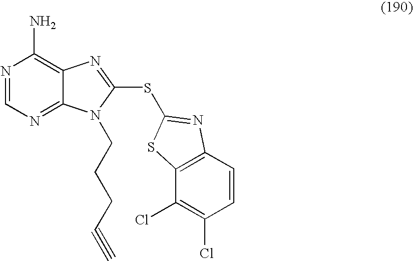

8-(6,7-Dichloro-benzothiazol-2-ylsulfanyl)-9-pent-4-ynyl-9H-purin-6-ylamine was prepared by the same method described in example 232. ¹H NMR (CDCl₃) δ 1.90(s, 1H), 2.14 (m, 2H), 2.28(m, 2H), 4.43 (t, 2H), 5.74 (s, 2H), 7.53 (d, 1H), 7.73(d, 1H), 8.43 (s, 1H). HPLC: RT=6.48 min (method: 5-100-7).

### EXAMPLE 19 1-(7-Methoxy-benzothiazol-2-ylsulfanyl)-9-pent-4-ynyl-9H-purin-6-ylamine (191)

8-(7-Methoxy-benzothiazol-2-ylsulfanyl)-9-pent-4-ynyl-9H-purin-6-ylamine was prepared by the same method described in example 232. ¹H NMR (CDCl₃) δ 1.95(s, 1H), 2.15 (m, 2H), 2.28(m, 2H), 4.47 (t, 2H), 5.66 (s, 2H), 6.82 (d, 1H), 7.40(t, 1H), 7.58 (d, 1H), 8.43 (s, 1H). HPLC: RT=6.78 min (method: 5-100-7).

### EXAMPLE 192 8-(7-Methyl-benzothiazol-2-ylsulfanyl)-9-pent-4-ynyl-9H-purin-6-ylamine (192)

8-(7-Methyl-benzothiazol-2-ylsulfanyl)-9-pent-4-ynyl-9H-purin-6-ylamine was prepared by the same method described in example 232 except that 7-methyl-benzothiazole-2-thiol was used instead of 7-chloro-benzothiazole-2-thiol. ¹H NMR (CDCl₃) δ 1.94(s, 1H), 2.13 (m, 2H), 2.28(m, 2H), 2.48 (s, 3H), 4.47 (t, 2H), 5.98 (s, 2H), 7.16 (d, 1H), 7.40(t, 1H), 7.78 (d, 1H), 8.43 (s, 1H). HPLC: RT=5.90 min (method: 5-100-7).

### EXAMPLE 193 8-(4-Amino-7-fluorol-benzothiazol-2-ylsulfanyl)-9-pent-4-ynyl-9H-purin-6-ylamine (193)

8-(4-Amino-7-fluorol-benzothiazol-2-ylsulfanyl)-9-pent-4-ynyl-9H-purin-6-ylamine was prepared by the same method described in example 232. ¹H NMR (CDCl₃) δ 2.04 (t, 1H), 2.16 (m, 2H), 2.36(m, 2H), 4.43 (t, 2H), 5.58 (s, 2H), 7.03 (m, 2H), 8.41 (s, 1H). HPLC: RT=5.19 min (method: 5-100-7).

### EXAMPLE 194 8-(7-Ethoxy-benzothiazol-2-ylsulfanyl)-9-pent-4-ynyl-9H-purin-6-ylamine (194)

8-(7-Ethoxy-benzothiazol-2-ylsulfanyl)-9-pent-4-ynyl-9H-purin-6-ylamine was prepared by the same method described in example 232 except that 7-ethoxy-benzothiazole-2-thiol was used instead of 7-chloro-benzothiazole-2-thiol. ¹H NMR (CDCl₃) δ 1.43 (t, 3H), 1.95(s, 1H), 2.15 (m, 2H), 2.28(m, 2H), 4.19(m, 2H), 4.47 (t, 2H), 6.14 (s, 2H), 6.78 (d, 1H), 7.40(t, 1H), 7.55 (d, 1H), 8.42 (s, 1H). HPLC: RT=6.08 min (method: 5-100-7).

### EXAMPLE 195 Acetic acid 2-[6-amino-8-(7-methoxy-benzothiazol-2-ylsulfanyl)-purin-9-yl]-ethyl ester (195)

Acetic acid 2-[6-amino-8-(7-methoxy-benzothiazol-2-ylsulfanyl)-purin-9-yl]-ethyl ester was prepared by the same method described in example 232. ¹H NMR (CDCl₃) δ 1.93 (s, 3H), 3.94(s, 3H), 4.45 (t, 2H), 4.62(t, 2H), 5.72 (bs, 2H), 6.81 (d, 1H), 7.41 (t, 1H), 7.55 (d, 1H), 8.41 (s, 1H). HPLC: RT=5.36 min (5-100-7).

### EXAMPLE 196 Acetic acid 3-[6-amino-8-(7-methoxy-benzothiazol-2-ylsulfanyl)-purin-9-yl]-propyl ester (196)

Acetic acid 3-[6-amino-8-(7-methoxy-benzothiazol-2-ylsulfanyl)-purin-9-yl]-propyl ester was prepared by the same method described in example 232. ¹H NMR (CDCl₃) δ 2.06(s, 3H), 2.25 (m, 2H), 3.95(s, 3H), 4.13 (t, 2H), 4.47 (t, 2H), 5.90 (s, 2H), 6.81 (d, 1H), 7.42(t, 1H), 7.58 (d, 1H), 8.50 (s, 1H). HPLC: RT=5.47 min (method: 5-100-7).

### EXAMPLE 197 Acetic acid 3-[6-amino-8-(7-methyl-benzothiazol-2-ylsulfanyl)-purin-9-yl]-propyl ester (197)

Acetic acid 3-[6-amino-8-(7-methyl-benzothiazol-2-ylsulfanyl)-purin-9-yl]-propyl ester was prepared by the same method described in example 232. ¹H NMR (CDCl₃) δ 1.99 (s, 3H), 2.22 (m, 2H), 2.48 (s, 3H), 4.00 (t, 2H), 4.48 (t, 2H), 6.07 (s, 2H), 7.16 (d, 1H), 7.42(t, 1H), 7.78 (d, 1H), 8.42 (s, 1H). HPLC: RT=5.59 min (method: 5-100-7).

### EXAMPLE 198 8-(7-Bromo-thiazole[4,5-c]pyridin-2-ylsulfanyl)-2-chloro-9-methyl-9H-purin-6-ylamine (198)

8-(7-Bromo-thiazole[4,5-c]pyridin-2-ylsulfanyl)-2-chloro-9-methyl-9H-purin-6-ylamine was prepared by the same method described as in 232. ¹H NMR (MeOD) δ 3.96 (s, 3H), 8.31 (s, 2H), 8.56 (s 1H), 9.15 (s, 1H). HPLC: RT=5.86 min (5-100-7).

### EXAMPLE 199 Acetic acid 2-[6-amino-8-(7-bromo-thiazole[4,5-c]pyridin-2-ylsulfanyl)-purin-9-yl]-ethyl ester

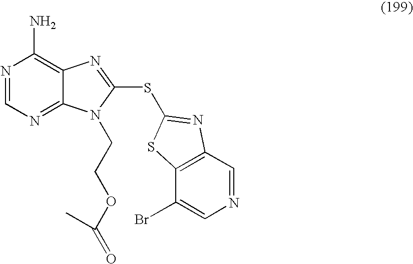

Acetic acid 2-[6-amino-8-(7-bromo-thiazole[4,5-c]pyridin-2-ylsulfanyl)-purin-9-yl]-ethyl ester was prepared by the same method described as in 232. ¹H NMR (CDCl₃) δ 1.98(s, 3H), 4.53 (t, 2H), 4.64 (t, 2H), 5.73 (s, 2H), 8.43 (s, 1H), 8.72(s, 1H), 9.2 (s, 1H). HPLC: RT=4.98 min (method: 5-100-7).

### EXAMPLE 200 Acetic acid 3-[6-amino-8-(7-bromo-thiazole[4,5-c]pyridin-2-ylsulfanyl)-purin-9-yl]-propyl ester

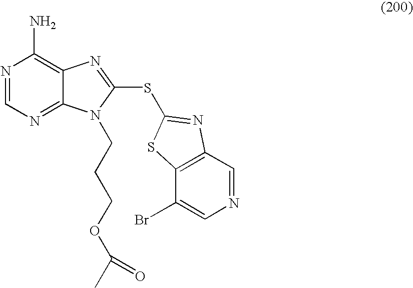

Acetic acid 3-[6-amino-8-(7-bromo-thiazole[4,5-c]pyridin-2-ylsulfanyl)-purin-9-yl]-propyl ester was prepared by the same method described in 232. ¹H NMR (CDCl₃) δ 1.98 (s, 3H), 2.22 (m, 2H), 4.53 (t, 2H), 4.64 (t, 2H), 6.14 (s, 2H), 8.40 (s, 1H), 8.57(s, 1H), 9.1 (s, 1H). HPLC: RT=5.10 min (method: 5-100-7).

### EXAMPLE 201 8-(7-Bromo-thiazole[4,5-c]pyridin-2-ylsulfanyl)-9-(4-methyl-pent-3-enyl)-9H-purin-6-ylamine

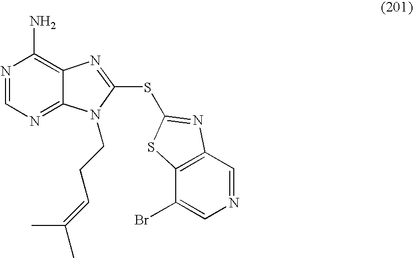

8-(7-Bromo-thiazole[4,5-c]pyridin-2-ylsulfanyl)-9-(4-methyl-pent-3-enyl)-9H-purin-6-ylamine was prepared by the same method described in 232. ¹H NMR (CDCl₃) δ 9.12(s, 1H), 8.58 (s, 1H), 8.47 (s, 1H), 5.80 (bs, 2H, NH₂), 5.14(t, J=1.37 Hz, 1H, CH), 4.37(t, J=6.87 Hz, 2H, CH₂), 2.56(m, 2H, CH₂), 1.38(s, 6H, 2CH₃). HPLC: RT=6.116 (5-100-7).

### EXAMPLE 202 {2-[6-amino-8-(7-bromo-thiazole[4,5-c]pyridin-2-ylsulfanyl)-purin-9-yl]ethyl}-phosphonic acid diethyl ester

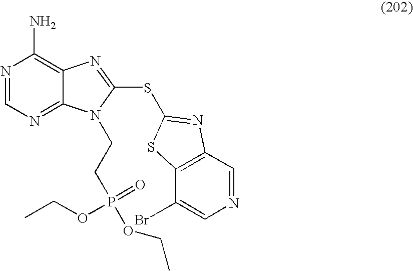

{2-[6-Amino-8-(7-bromo-thiazole[4,5-c]pyridin-2-ylsulfanyl)-purin-9-yl]ethyl}-phosphonic acid diethyl ester was prepared by the same method described in 232. ¹H NMR (CDCl₃) δ 9.25(s, 1H), 8.58 (s, 1H), 8.46 (s, 1H), 5.71(bs, 2H, NH₂), 4.65(m, 2H, CH₂), 4.07(m, 4H, 2CH₂), 2.52(m, 2H, CH₂), 1.28(t, J=7.1 Hz, 6H, 2CH₃). HPLC: RT=5.013 (5-100-7).

### EXAMPLE 203 {3-[6-Amino-8-(7-bromo-thiazole[4,5-c]pyridin-2-ylsulfanyl)-purin-9-yl]propyl}-phosphonic acid diethyl ester (203)

{3-[6-Amino-8-(7-bromo-thiazole[4,5-c]pyridin-2-ylsulfanyl)-purin-9-yl]propyl}-phosphonic acid diethyl ester was prepared by the same method described as in 232. ¹H NMR (CDCl₃) δ 9.12(s, 1H), 8.58 (s, 1H), 8.44 (s, 1H), 5.83(bs, 2H, NH₂), 4.45(t, J=7.23 Hz, 2H, CH₂), 4.04(m, 4H, 2CH₂), 2.21(m, 2H, CH₂), 1.35(m, 2H, CH₂), 1.26(t, J=7.06 Hz, 6H, 2CH₃). HPLC: RT=4.925 (Method: 5-100-7).

### EXAMPLE 204 Acetic acid 2-[6-amino-8-(7-bromo-thiazole[4,5-c]pyridin-2-ylsulfanyl)-purin-9-yl]-ethyl ester (204)

Acetic acid 2-[6-amino-8-(7-bromo-thiazole[4,5-c]pyridin-2-ylsulfanyl)-purin-9-yl]-ethyl ester was prepared by the same method described as in 232. ¹H NMR (CDCl₃) δ 2.02 (s, 3H), 4.47 (t, 2H), 4.64 (t, 2H), 5.71 (s, 2H), 8.45 (s, 1H), 8.50(s, 1H), 9.1 (s, 1H). HPLC: RT=4.90 min (method: 5-100-7).

### EXAMPLE 205 Acetic acid 3-[6-amino-8-(7-chloro-thiazolo[4,5-c]pyridin-2-ylsulfanyl)-purin-9-yl]-propyl ester

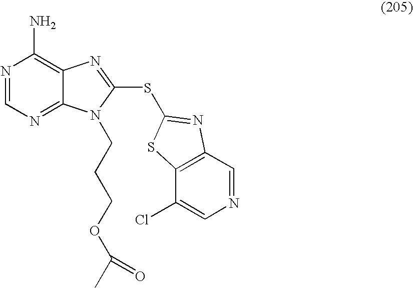

Acetic acid 3-[6-amino-8-(7-chloro-thiazolo[4,5-c]pyridin-2-ylsulfanyl)-purin-9-yl]-propyl ester was prepared by the same method described in 177. NMR (CDCl₃) δ 2.02 (s, 3H), 2.26 (m, 2H), 4.09 (t, J=5.9 Hz, 2H), 4.47 (t, J=7.0 Hz, 2H), 5.75 (s, 2H, NH₂), 8.45 (s, 1H), 8.50 (s, 1H), 9.09 (s, 1H). HPLC: RT=5.06 min (5-100-7).

### EXAMPLE 206 {2-[6-Amino-8-(7-chloro-thiazolo[4,5-c]pyridinl-2-ylsulfanyl)-purin-9-yl]-ethyl}-phosphonic acid diethyl ester

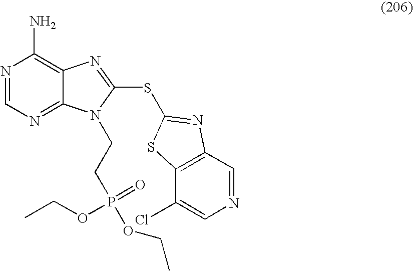

### Step 1 7-Chloro-thiazole[4,5-c]pyridine-2-thiol

To a solution of 3-nitro-pyridin-4-ol (15 g, 107 mmol) in 50% of Acetic acid (200 ml) was bubbled with Cl₂ gas for 20 min at room temperature. The precipitate was filtered off and washed with water. Pure 3-nitro-4-chloro-pyridine was obtained after recrystallized from EtOH with 95% yield. To a solution of 3-nitro-4-chloro-pyridine (14 g, 79.8 mmol) in DMF (30 ml) at room temperature was added POCl₃ (7.42 ml, 79.8 mmol). The mixture was heated to 120° C. for 30 min and then cooled to room temperature. The reaction mixture was neutralized with NaHCO₃ (sat.) and then extracted with EtOAC. The combined extracts were washed with water and brine, dried over MgSO₄, concentrated to give 3-nitro-4,5-dichloro-pyridine with 94% yield. To a solution of 3-nitro-4,5-dichloro-pyridine (14 g, 72.16 mmol) in HCl (160 ml) and ether (80 ml) at room temperature was added SnCl₂ (162.8 g, 721.6 mmol) to give 3-amino-4,5-dichloro-pyridine with 85% yield, which was further reacted with O-ethylxanthic acid, potassium salt to form chloro-thiazole[4,5-c]pyridine-2-thiol with 89% yield, (see scheme Q). ¹H NMR (DMSO) δ 8.09(s, 1H), 8.35(s, 1H).

### Step 2 {2-[6-Amino-8-(7-chloro-thiazolo[4,5-c]pyridinl-2-ylsulfanyl)-purin-9-yl]-ethyl}-phosphonic acid diethyl ester

{2-[6-Amino-8-(7-chloro-thiazolo[4,5-c]pyridinl-2-ylsulfanyl)-purin-9-yl]-ethyl}-phosphonic acid diethyl ester was prepared by the same method described in example 232 except that chloro-thiazole[4,5-c]pyridine-2-thiol was used instead of 7-chloro-benzothiazole-2-thiol. ¹H NMR (CDCl₃) δ 9.08(s, 1H), 8.25 (s, 1H), 8.46(s, 1H), 5.76 (bs, 2H, NH₂), 4.65(m, 2H, CH₂), 4.07(m, 2H, CH₂), 2.52(m, 2H, CH₂), 1.28(t, J=7.1 Hz, 3H, CH₃). HPLC: RT=4.969 min (5-100-7).

### EXAMPLE 207 8-(7-Bromo-thiazolo[5,4-b]pyridin-2-ylsulfanyl)-9-butyl-9H-purine-6-ylamine

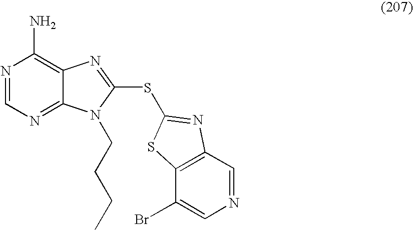

8-(7-Bromo-thiazolo[5,4-b]pyridin-2-ylsulfanyl)-9-butyl-9H-purine-6-ylamine was prepared by the same method described as in example 232. ¹H NMR (CDCl₃) δ 0.92 (t, 3H), 1.34 (m, 2H), 1.85 (m, 2H), 4.36 (t, 2H), 5.74 (s, 2H), 8.50 (s, 1H), 8.70(s, 1H), 9.20 (s, 1H). HPLC: RT=5.79 min (method: 5-100-7).

### EXAMPLE 208 8-(7-Bromo-thiazolo[5,4-b]pyridin-2-ylsulfanyl)-9-butyl-9H-purine-6-ylamine

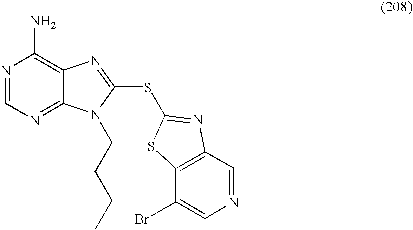

8-(7-Bromo-thiazolo[5,4-b]pyridin-2-ylsulfanyl)-9-butyl-9H-purine-6-ylamine was prepared by the same method described in example 232. ¹H NMR (CDCl₃) δ 0.92 (t, 3H), 1.34 (m, 2H), 1.85 (m, 2H), 4.36 (t, 2H), 5.74 (s, 2H), 8.50 (s, 1H), 8.70(s, 1H), 9.20 (s, 1H). HPLC: RT=5.79 min (method: 5-100-7).

### EXAMPLE 209 8-(7-Bromo-thiazolo[5,4-b]pyridin-2-ylsulfanyl)-2-chloro-9-methyl-9H-purine-6-ylamine

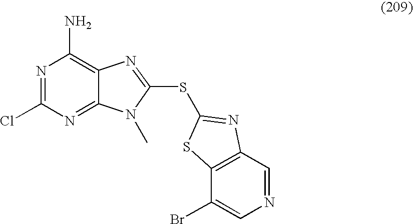

8-(7-Bromo-thiazolo[5,4-b]pyridin-2-ylsulfanyl)-2-chloro-9-methyl-9H-purine-6-ylamine was prepared by the same method described in example 232. ¹H NMR (MeOD) δ 3.97 (s, 3H), 8.31 (s, 2H), 8.59 (s, 1H), 9.15 (s, 1H). HPLC: RT=:5.86 min (method: 5-100-7).

### EXAMPLE 210 9-Butyl-8-(7-chloro-benzooxazol-2-ylsulfanyl)-9H-purine-6-ylamine

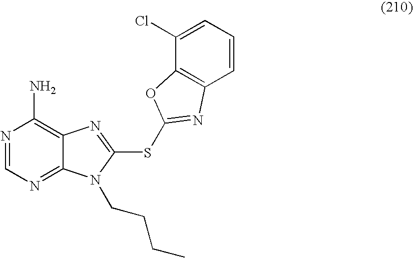

9-Butyl-8-(7-chloro-benzooxazol-2-ylsulfanyl)-9H-purine-6-ylamine was prepared by the same method described in example 232 except that 7-chloro-benzooxazole-2-thiol was used instead of 7-chloro-benzothiazole-2-thiol. ¹H NMR (CDCl₃) δ 0.92 (t, 3H), 1.37 (m, 2H), 1.88 (m, 2H), 4.37 (t, 2H), 5.78 (s, 2H), 7.42 (d, 1H), 7.27(t, 1H), 7.32 (d, 1H), 7.53 (d, 1H), 8.45(s, 1H). HPLC: RT=6.155 min (method: 5-100-15 min).

### EXAMPLE 211 Acetic acid 2-[6-amino-8-(7-chloro-benzooxazol-2-ylsulfanyl)-purin-9-yl]-ethyl ester

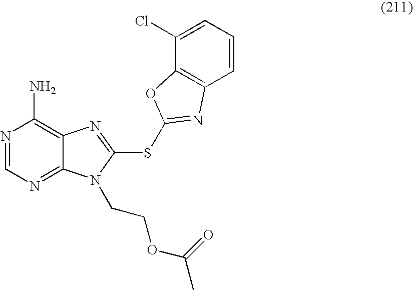

Acetic acid 2-[6-amino-8-(7-chloro-benzooxazol-2-ylsulfanyl)-purin-9-yl]-ethyl ester was prepared by the same method described in example 232 except that 7-chloro-benzooxazole-2-thiol was used instead of 7-chloro-benzothiazole-2-thiol. ¹H NMR (CDCl₃) δ 2.00 (s, 3H), 4.52 (t, 2H), 4.67 (t, 2H), 5.78 (s, 2H), 7.29 (t, 1H), 7.35 (d, 1H), 7.52 (d, 1H), 8.44(s, 1H). HPLC: RT=5.376 min (method: 5-100-7).

### EXAMPLE 212 Acetic acid 3-[6-amino-8-(7-chloro-benzooxazol-2-ylsulfanyl)-purin-9-yl]-propyl ester

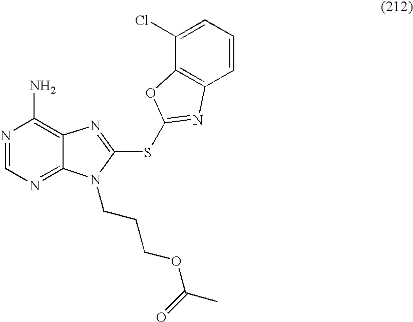

Acetic acid 3-[6-amino-8-(7-chloro-benzooxazol-2-ylsulfanyl)-purin-9-yl]-propyl ester was prepared by the same method described in example 232 except that 7-chloro-benzooxazole-2-thiol was used instead of 7-chloro-benzothiazole-2-thiol. ¹H NMR (CDCl₃) δ=1.97 (s, 3H), 2.31 (m, 2H), 4.11 (t, 2H), 4.49 (t, 2H), 5.78 (s, 2H), 7.29 (t, 1H), 7.32 (d, 1H), 7.52 (d, 1H), 8.44(s, 1H). HPLC: RT=5.478 min (method: 5-100-7).

### EXAMPLE 213 Acetic acid 3-[6-amino-8-(7-bromo-benzothiazol-2-ylsulfanyl)-purin-9-yl]-propyl ester

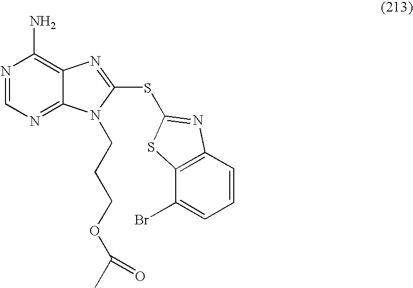

Acetic acid 3-[6-amino-8-(7-bromo-benzothiazol-2-ylsulfanyl)-purin-9-yl]-propyl ester was prepared by the same method described in 241. ¹H NMR (CDCl₃) δ=2.00 (s, 3H), 2.22 (m, 2H), 4.07 (t, 2H), 4.46 (t, 2H), 5.70 (s, 2H), 7.54 (t, 1H), 7.73 (d, 1H), 7.92 (d, 1H), 8.41(s, 1H). HPLC: RT=5.81 min (method: 5-100-7).

### EXAMPLE 214 3-[6-Amino-8-(6,7-dichloro-benzothiazol-2-ylsulfanyl)-purin-9-yl]-propan-1-ol

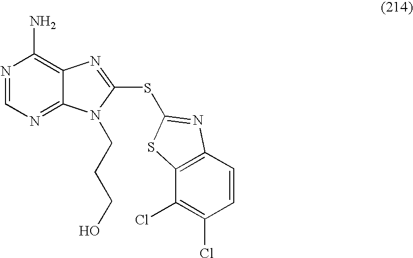

3-[6-Amino-8-(6,7-dichloro-benzothiazol-2-ylsulfanyl)-purin-9-yl]-propan-1-ol was prepared by the same method described in example 244. ¹H NMR (CDCl₃) δ=2.06(m, 2H), 3.48(m, 2H), 4.53 (t, 2H), 4.47 (t, 2H), 5.80 (s, 2H), 7.55 (d, 1H), 7.76 (d, 1H), 8.43 (s, 1H). HPLC: RT=5.56 min (method: 5-100-7).

### EXAMPLE 215 3-[6-Amino-8-(7-bromo-benzothiazol-2-ylsulfanyl)-purin-9-yl]-propan-ol

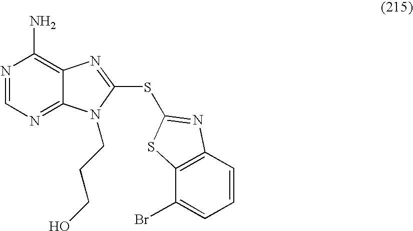

3-[6-Amino-8-(7-bromo-benzothiazol-2-ylsulfanyl)-purin-9-yl]-propan-ol was prepared by the same method described in example 244. ¹H NMR (CDCl₃) δ 1.95(m, 2H), 3.47(t, 2H), 4.54 (t, 2H), 5.92 (bs, 2H), 7.13 (t, 1H), 7.44 (dd, 1H), 7.74 (d, 1H), 8.44 (s, 1H); HPLC: RT=5.19 min (method 5-100-7).

### EXAMPLE 216 3-[6-Amino-8-(7-methoxy-benzothiazol-2-ylsulfanyl)-purin-9-yl]-propan-ol (216)

3-[6-Amino-8-(7-methoxy-benzothiazol-2-ylsulfanyl)-purin-9-yl]-propan-ol was prepared by the same method described in example 244. ¹H NMR (CDCl₃) δ 1.91(m, 2H), 3.45(t, 2H), 3.90 (s, 3H), 4.54 (t, 2H), 5.88 (bs, 2H), 6.82 (d, 1H), 7.45 (t,1H), 7.57 (d, 1H), 8.41 (s,1H); HPLC: RT=4.86 min (method 5-100-7).

### EXAMPLE 217 2-[6-Amino-8-(7-methoxy-benzothiazol-2-ylsulfanyl)-purin-9-yl]-ethanol (217)

2-[6-Amino-8-(7-methoxy-benzothiazol-2-ylsulfanyl)-purin-9-yl]-ethanol was prepared by the same method described in example 244. ¹H NMR (CDCl₃) δ 8.35 (s, 1H), 7.52(d, J=1.08 Hz, 1H), 7.41(t, J=8.4 Hz, 1H), 6.81(d, J=1.08 Hz, 1H), 4.48(t, J=5.09, 2H, CH2), 3.94(m, 5H, CH2+CH3), 3.40(s, 1H, OH). HPLC: RT=4.803 (Method: 5-100-7).

### EXAMPLE 218 2-[6-amino-8-(7-bromo-thiazolo[4,5-c]pyridin-2-ylsulfanyl)-purin-9-yl]-ethanol (218)

2-[6-amino-8-(7-bromo-thiazolo[4,5-c]pyridin-2-ylsulfanyl)-purin-9-yl]-ethanol was prepared by the same method described in 244. ¹H NMR (DMSO) δ 3.79 (m, 2H), 4.29 (t, 2H), 7.63 (s 2H), 8.25 (s, 1H), 8.66 (s, 1H), 9.17 (s, 1H), 8.24 (s, 1H). HPLC: RT=4.42 min (5-100-7).

### EXAMPLE 219 2-[6-Amino-8-(7-chloro-thiazolo[5,4-b]pyridin-2-ylsulfanyl)-purin-9-yl]-ethanol (219)

2-[6-Amino-8-(7-chloro-thiazolo[5,4-b]pyridin-2-ylsulfanyl)-purin-9-yl]-ethanol was prepared by the same method described in 244. ¹H NMR (MeOD) δ 3.91 (t, 2H), 4.52 (t, 2H), 8.14 (s, 1H), 8.49 (s, 1H), 9.04 (s, 1H). HPLC: RT=4.36 min (method: 5-100-7).

### EXAMPLE 220 8-(7-Fluoro-benzothiazol-2-ylsulfanyl)-9-(2-vinyloxy-ethyl)-9H-purin-6-ylamine (220)

8-(7-Fluoro-benzothiazol-2-ylsulfanyl)-9-(2-vinyloxy-ethyl)-9H-purin-6-ylamine was prepared by the same method described in example 271. ¹H NMR (CDCl₃) δ 3.98 (dd, 1H), 4.09 (t, 2H), 4.13 (dd, 1H), 4.68 (t, 2H), 5.79 (s, 2H), 6.23(dd, 1H), 7.10 (t, 1H), 7.44 (dd, 1H), 7.76 (d, 1H), 8.44 (s, 1H). HPLC: RT=5.748 min (method: 5-100-7).

### EXAMPLE 221 8-(7-Fluoro-benzothiazol-2-ylsulfanyl)-9-pent-4-ynyl-9H-purin-6-ylamine (221)

8-(7-Fluoro-benzothiazol-2-ylsulfanyl)-9-pent-4-ynyl-9H-purin-6-ylamine was prepared by the same method described in example 271. ¹H NMR (CDCl₃) δ 1.96(s, 1H), 2.18 (m, 2H), 2.26 (m, 2H), 4.47 (t, 2H), 5.79 (s, 2H), 6.23(dd, 1H), 7.10 (t, 1H), 7.44 (dd, 1H), 7.76 (d, 1H), 8.45(s, 1H). HPLC: RT=5.779 min (method: 5-100-7).

### EXAMPLE 222 2-[6-Amino-8-(7-fluoro-benzothiazol-2-ylsulfanyl)-purin-9-yl]-ethanol

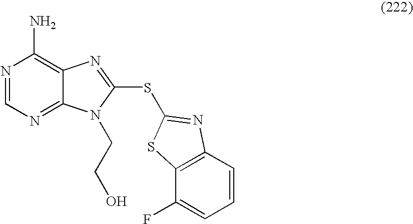

2-[6-Amino-8-(7-fluoro-benzothiazol-2-ylsulfanyl)-purin-9-yl]-ethanol was prepared by the same method described in example 244. ¹H NMR (DMSO) δ 3.67 (m, 2H), 4.32 (t, 2H), 5.07 (t, 1H), 7.31 (m, 1H), 7.55 (m, 1H), 7.73 (bs, 2H), 7.78 (m, 1H), 8.24 (s, 1H). HPLC: RT=4.75 min (5-100-7).

### EXAMPLE 223 4-[6-amino-8-(7-fluoro-benzothiazol-2-ylsulfanyl)-purin-9-yl]-butan-1-ol

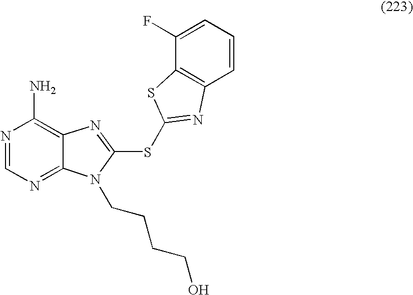

4-[6-amino-8-(7-fluoro-benzothiazol-2-ylsulfanyl)-purin-9-yl]-butan-1-ol was prepared by the same method described in example 244. ¹H NMR (CDCl₃) δ 1.69(m, 2H), 1.95 (m, 2H), 3.79(t, 2H), 4.39 (t, 2H), 5.32 (bs, 2H), 7.29 (t,1H), 7.44 (dd,1H), 7.74 (d, 1H), 8.43 (s,1H); HPLC: RT=4.969 min (5-100-7).

### EXAMPLE 224 3-[6-amino-8-(7-fluoro-benzothiazol-2-ylsulfanyl)-purin-9-yl]-propan-ol

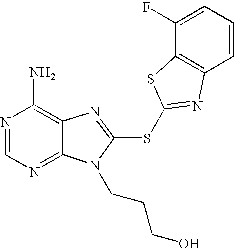

3-[6-amino-8-(7-fluoro-benzothiazol-2-ylsulfanyl)-purin-9-yl]-propan-ol was prepared by the same method described in example 244. ¹H NMR (CDCl₃) δ 1.95(m, 2H), 3.47(t, 2H), 4.54 (t, 2H), 5.92 (bs, 2H), 7.13 (t,1H), 7.44 (dd,1H), 7.74 (d, 1H), 8.44 (s,1H); HPLC: RT=4.863 min (5-100-7).

### EXAMPLE 225 9-Butyl-8-(7-fluoro-benzooxazol-2-ylsulfanyl)-9H-purine-6-ylamine

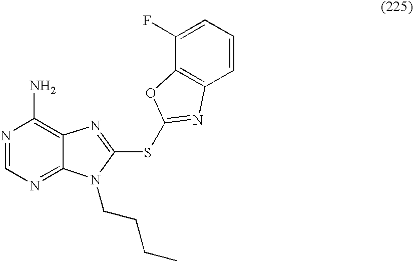

9-Butyl-8-(7-fluoro-benzooxazol-2-ylsulfanyl)-9H-purine-6-ylamine was prepared by the same method described in example 232 except that 7-fluoro-benzooxazole-2-thiol was used instead of 7-chloro-benzothiazole-2-thiol. ¹H NMR (CDCl₃) δ 0.92 (t, 3H), 1.37 (m, 2H), 1.88 (m, 2H), 4.37 (t, 2H), 5.80 (s, 2H), 7.12 (t, 1H), 7.29(d, 1H), 7.43 (d, 1H), 8.45(s, 1H). HPLC: RT=5.900 min (method: 5-100-15 min).

### EXAMPLE 226 Acetic acid 2-[6-amino-8-(7-fluoro-benzothiazol-2-ylsulfanyl)-purin-9-yl]-ethyl ester

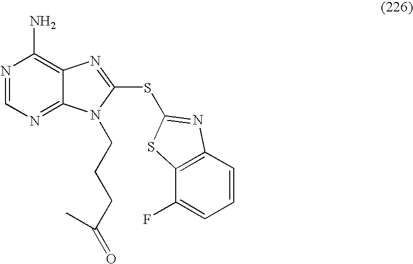

Acetic acid 2-[6-amino-8-(7-fluoro-benzothiazol-2-ylsulfanyl)-purin-9-yl]-ethyl ester was prepared by the same method described in example 241. ¹H NMR (CDCl₃) δ 4.13 (t, 2H), 4.64 (t, 2H), 5.73 (s, 2H), 7.09 (t, 1H), 7.42(m, 1H), 7.74 (d, 1H), 8.43 (s, 1H). HPLC: RT=5.31 min (method: 5-100-7).

### EXAMPLE 227 Acetic acid 3-[6-amino-8-(7-fluoro-benzothiazol-2-ylsulfanyl)-purin-9-yl]-propyl ester

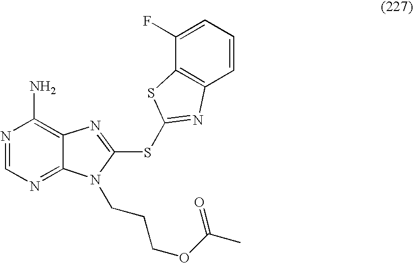

Acetic acid 3-[6-amino-8-(7-fluoro-benzothiazol-2-ylsulfanyl)-purin-9-yl]-propyl ester was prepared by the same method described in example 241. (CDCl₃) δ 2.00 (s, 3H), 2.25(m, 2H), 4.09(t, 2H), 4.47 (t, 2H), 5.92(s, 2H), 7.13 (t, 1H), 7.44(dd,1H), 7.74(d, 1H), 8.43 (s, 1H); HPLC: RT=5.448 min (5-100-7).

### EXAMPLE 228 2-Chloro-8-(7-chloro-benzothiazol-2-ylsulfanyl)-9-methyl-9H-purine-6-ylamine

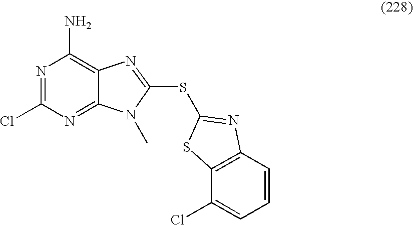

2-Chloro-8-(7-chloro-benzothiazol-2-ylsulfanyl)-9-methyl-9H-purine-6-ylamine was prepared by the same method described in example 232. ¹H NMR (CDCl₃) δ 3.81 (s, 3H), 5.83 (s, 2H, NH₂), 7.37 (d, 1H), 7.44(t, 1H), 7.83 (d, 1H). HPLC: RT=6.99 min (method: 5-100-7).

### EXAMPLE 229 9-ethyl-8-(7-chloro-benzothiazol-2-ylsulfanyl)-9H-purine-6-ylamine (229)

9-Ethyl-8-(7-chloro-benzothiazol-2-ylsulfanyl)-9H-purine-6-ylamine was prepared by the same method described in example 232. ¹H NMR (CDCl₃) δ 1.46 (t, 3H), 4.43 (q, 2H), 5.88 (bs, 2H), 7.38 (d, 1H), 7.43 (t, 1H), 7.83 (d, 1H), 8.43 (s, 1H). HPLC: RT=5.84 min (5-100-7)

### EXAMPLE 230 8-(7-Chloro-benzothiazol-2-ylsulfanyl)-9-ethyl-9H-purine-6-ylamine (230)

8-(7-Chloro-benzothiazol-2-ylsulfanyl)-9-ethyl-9H-purine-6-ylamine was prepared by the same method described in example 232. ¹H NMR (CDCl₃) δ 0.95 (t, J=7.45 Hz, 3H), 4.32 (t, J=7.45 Hz, 2H), 5.83 (s, 2H, NH₂), 7.35 (d, 8.06 Hz, 1H), 7.43(t, J=8.06 Hz, 1H), 7.83 (d, J=7.84 Hz, 1H), 8.44 (s, 1H). HPLC: RT=5.844 min (method: 5-100-7).

### EXAMPLE 231 9-Propyl-8-(7-chloro-benzothiazol-2-ylsulfanyl)-9H-purine-6-ylamine

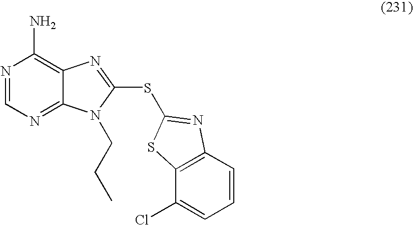

9-Propyl-8-(7-chloro-benzothiazol-2-ylsulfanyl)-9H-purine-6-ylamine (CF1905) was prepared by the same method described in example 232. ¹H NMR (CDCl₃) δ 0.95 (t, J=7.45 Hz, 3H), 1.91 (m, 2H), 4.32 (t, J=7.45 Hz, 2H), 5.83 (s, 2H, NH₂), 7.35 (d, 8.06 Hz, 1H), 7.43(t, J=8.06 Hz, 1H), 7.83 (d, J=7.84 Hz, 1H), 8.44 (s, 1H). HPLC: RT=6.09 min (method: 5-100-7).

### EXAMPLE 232 9-Butyl-8-(7-chloro-benzothiazol-2-ylsulfanyl)-9H-purine-6-ylamine

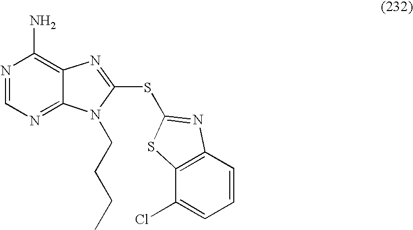

### Step 1 9-Butyl-9H-purin-6-ylamine

To a mixture of Adenine (10 g, 74 mmol) and cesium carbonate (28.93 g, 88.8 mmol) in DMF (100 ml) was added 1-iodobutane (10.15 ml, 88.8 mmol) at room temperature. The reaction mixture was left stirring at room temperature for 16 hours before quenching with water (200 ml). The precipitate was filtered off and dried under vacuum pump to give title compound with 95% yield (13.5 g, 70 mmol). ¹H NMR (DMSO) δ 0.89 (t, J=7.36 Hz, 3H), 1.20 (m, 2H), 1.77 (m, 2H), 4.13 (t, J=7.34 Hz, 2H), 7.17 (s, 1H), 8.14 (s, 1H).

### Step 2 8-Bromo-9-butyl-9H-purin-6-ylamine

9-Butyl-9H-purin-6-ylamine (10 g, 52.35 mmol) was suspended in HOAC/NaOAC buffer (6 ml), THF (6 ml) and MeOH (6 ml) before adding Br₂ (16.75 g, 104.7 mmol) slowly at room temperature. After added Br₂, the reaction mixture became clear and continued to stir at rt for 0.5 h. Then the reaction mixture was concentrated to ⅓ of original volume followed by extracted with EtOAc, wash with water, brine, dried over MgSO₄ and concentrated to give crude material. Pure material (11 g, 40.6 mmol) was obtained by recrystallization from MeOH with 77.6% yield. ¹H NMR (MeOH) δ 0.98 (t, J=7.36 Hz, 3H), 1.40 (m, 2H), 1.80 (m, 2H), 4.24 (t, J=7.34 Hz, 2H), 8.28 (s, 1H).

### Step 3 7-Chloro-benzothiazole-2-thiol

To a solution of 2,3-dichloro-phenylamine (2 g, 12.34 mmol) in DMF (10 ml) at room temperature was added O-ethylxanthic acid, potassium salt (1.98 g, 12.34 mmol). The reaction mixture was then heated to 150° C. for 4 hours. The reaction mixture was cooled to room temperature and the solvent was removed in vacue. The crude material was diluted with NH₄Cl (sat.) and a solid precipitate. The solid was filtered off, washed with water (50 ml ×2) and dried under vacuum to give 7-chloro-benzothiazole-2-thiol with 89% yield (2.2 g, 10.94 mmol). ¹H NMR (CDCl₃) δ 7.16 (d, J=7.8 Hz, 1H), 7.38 (m, 2H), 10.0(s, 1H).

### Step 4 9-Butyl-8-(7-chloro-benzothiazol-2-ylsulfanyl)-9H-purine-6-ylamine

To 7-Chloro-benzothiazole-2-thiol (222 mg, 1.1 mmol) in DMF (5 ml) was added potassium t-butoxide (124 mg, 1.1 mmol) at room temperature. After 15 min., a solution of 8-bromo-9-Butyl-9H-purin-6-ylamine (100 mg, 0.37 mmol) in DMF (1 ml) was added and stirred for 6 h at 130° C. The reaction mixture was cooled to room temperature, diluted with water. Extract with EtOAC (200 ml), washed with brine (50 ml), dried over MgSO₄, concentrated, and purified from fresh chromatography (silica gel) (5% MeOH/CH₂Cl₂) to give the final product (110 mg, 0.28 mmol) as a white powder with 75.7% yield. ¹H NMR (CDCl₃) δ0.91 (t, J=7.36 Hz, 3H), 1.37 (m, 2H), 1.83 (m, 2H), 4.33 (t, J=7.34 Hz, 2H), 6.03 (s, 2H, NH₂), 7.36 (dd, J=1.0 Hz, 7.84 Hz, 1H), 7.42(t, J=7.89 Hz, 1H), 7.83 (dd, J=1.0 Hz, 7.84 Hz, 1H), 8.43 (s, 1H). HPLC: RT=9.73 min (method: 5-100-15)

### EXAMPLE 233 9-Pentyl-8-(7-chloro-benzothiazol-2-ylsulfanyl)-9H-purine-6-ylamine

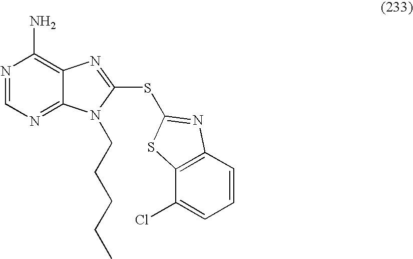

9-Pentyl-8-(7-chloro-benzothiazol-2-ylsulfanyl)-9H-purine-6-ylamine was prepared by the same method described in example 232. ¹H NMR (CDCl₃) δ 0.81 (t, J=7.45 Hz, 3H), 0.94 (m, 2H), 1.31(m, 2H), 1.86(m, 2H), 4.35 (t, J=7.45 Hz, 2H), 5.97 (s, 2H, NH₂), 7.35 (d, 8.06 Hz, 1H), 7.43(t, J=8.06 Hz, 1H), 7.83 (d, J=7.84 Hz, 1H), 8.44 (s, 1H). HPLC: RT=6.80 min, (method: 5-100-7)

### EXAMPLE 234 8-(7-Chloro-benzothiazol-2-ylsulfanyl)-9-pent-4-ynyl-9H-purin-6-ylamine (234)

8-(7-Chloro-benzothiazol-2-ylsulfanyl)-9-pent-4-ynyl-9H-purin-6-ylamine was prepared by the same method described in example 232. ¹H NMR (CDCl₃) δ 1.96 (t, 1H), 2.14 (m, 2H), 2.31(m, 2H), 4.47 (t, 2H), 5.75 (s, 2H), 7.36 (d, 1H), 7.43(t, 1H), 7.82 (d, 1H), 8.44 (s, 1H). HPLC: RT=6.01 min (method: 5-100-7).

### EXAMPLE 235 8-(7-Chloro-benzothiazol-2-ylsulfanyl)-9-pent-4-ynyl-9H-purin-6-ylamine (235)

8-(7-Chloro-benzothiazol-2-ylsulfanyl)-9-pent-4-ynyl-9H-purin-6-ylamine was prepared by the same method described in example 232, except that 7-bromo-benzothiazole-2-thiol was used instead of 7-chloro-benzothiazole-2-thiol. ¹H NMR (CDCl₃) δ 1.96(s, 1H), 2.29 (m, 2H), 2.58(m, 2H), 4.49 (t, 2H), 5.88 (s, 2H), 7.35 (t, 1H), 7.60(d, 1H), 7.90 (d, 1H), 8.45 (s, 1H). HPLC: RT=6.10 min (method: 5-100-7).

### EXAMPLE 236 8-(7-Chloro-benzothiazol-2-ylsulfanyl)-9-(2-methoxy-ethyl)-9H-purin-6-ylamine (236)

8-(7-Chloro-benzothiazol-2-ylsulfanyl)-9-(2-methoxy-ethyl)-9H-purin-6-ylamine was prepared by the same method described in example 232. ¹H NMR (CDCl₃) δ 3.23(s, 3H), 3.75 (t, 2H), 4.56(t, 2H), 5.88 (s, 2H), 7.35 (d, 1H), 7.43(t, 1H), 7.84 (d, 1H), 8.43 (s, 1H). HPLC: RT=5.61 min (method: 5-100-7).

### EXAMPLE 237 8-(7-Chloro-benzothiazol-2-ylsulfanyl)-9-(2-vinyloxy-ethyl)-9H-purin-6-ylamine (237)

8-(7-Chloro-benzothiazol-2-ylsulfanyl)-9-(2-vinyloxy-ethyl)-9H-purin-6-ylamine was prepared by the same method described in example 232. ¹H NMR (CDCl₃) δ 3.96 (m, 1H), 4.09 (m, 3H), 4.69 (t, 2H), 5.77 (s, 2H), 6.25(m, 1H), 7.35 (d, 1H), 7.43(t, 1H), 7.84 (d, 1H), 8.44 (s, 1H). HPLC: RT=6.01 min (method: 5-100-7).

### EXAMPLE 238 {2-[6-amino-8-(7-chloro-benzothiazol-2-ylsulfanyl)-purin-9-yl]-ethyl}-phosphonic acid diethyl ester (238)

{2-[6-amino-8-(7-chloro-benzothiazol-2-ylsulfanyl)-purin-9-yl]-ethyl}-phosphonic acid diethyl ester was prepared by the same method described in example 232. ¹H NMR (CDCl₃) δ 1.28 (t, 6H), 2.52 (m 2H), 4.07(m, 4H), 4.65 (m, 2H), 5.67 (bs, 2H), 7.38 (d, 1H), 7.43 (t, 1H), 7.83 (d, 1H), 8.53 (s, 1H). HPLC: RT=5.66 min (5-100-7).

### EXAMPLE 239 {2-[6-Amino-8-(7-chloro-benzothiazol-2-ylsulfanyl)-purin-9-yl]-ethyl}-phosphonic acid diethyl ester

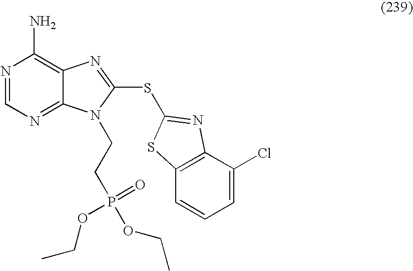

{2-[6-Amino-8-(7-chloro-benzothiazol-2-ylsulfanyl)-purin-9-yl]-ethyl}-phosphonic acid diethyl ester was prepared by the same method described in example 232. ¹H NMR (CDCl₃) δ 8.53 (s, 1H), 7.83(d, J=7.0 Hz, 1H), 7.43(t, J=7.9 Hz, 1H), 7.38(d, J=7.0 Hz, 1H), 5.67(bs, 2H, NH₂), 4.65(m, 2H, CH₂), 4.07(m, 4H, 2CH₂), 2.52(m, 2H, CH₂), 1.28(t, J=7.1 Hz, 6H, 2CH₃). HPLC: RT=5.667 min(5-100-7).

### EXAMPLE 240 {2-[6-Amino-8-(7-chloro-benzothiazol-2-ylsulfanyl)-purine-9-yl]-ethyl}-phosphoramidic acid diethyl ester (240)

{2-[6-Amino-8-(7-chloro-benzothiazol-2-ylsulfanyl)-purine-9-yl]-ethyl}-phosphoramidic acid diethyl ester was prepared by the same method described in example 271. ¹H NMR (CDCl₃) δ 1.19(t, 6H), 3.50 (m 2H), 3.80 (m, 1H), 3.90 (m 4H), 4.47 (t, 2H), 6.00 (bs, 2H), 7.36 (d, 1H), 7.41 (t, 1H), 7.83 (d, 1H), 8.38 (s, 1H). HPLC: RT=5.49 min (5-100-7).

### EXAMPLE 241 Acetic acid 2-[6-amino-8-(7-chloro-benzothiazol-2-ylsulfanyl)-purin-9-yl]-ethyl ester

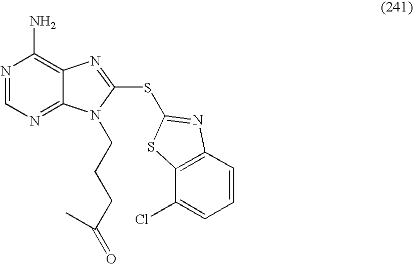

### Step 1 Acetic acid 2-(6-amino-purin-9-yl)-ethyl ester

Acetic acid 2-(6-amino-purin-9-yl)-ethyl ester was prepared by the same method described in example 232, step 1 except that 2-bromo-ethanol acetate was used instead of 1-iodobutane. Acetic acid 2-(6-amino-purin-9-yl)-ethyl ester was obtained in 95% yield as white powder. ¹H NMR (CDCl₃) δ 2.06 (s, 3H), 4.47 (m, 4H), 5.62 (s, 2H, NH₂), 7.85 (s 1H), 8.39 (s, 1H).

### Step 2 Acetic acid 3-(6-amino-8-bromo-purin-9-yl)-ethyl ester

Acetic acid 3-(6-amino-8-bromo-purin-9-yl)-ethyl ester was prepared by the same method described in example 49, step 2 and was obtained in 90% yield. ¹H NMR (CDCl₃) δ 2.06 (s, 3H), 4.51 (m, 4H), 582(s, 2H, NH₂), 8.34 (s, 1H).

### Step 3 Acetic acid 2-[6-amino-8-(7-chloro-benzothiazol-2-ylsulfanyl)-purin-9-yl]-ethyl ester

Acetic acid 2-[6-amino-8-(7-chloro-benzothiazol-2-ylsulfanyl)-purin-9-yl]-ethyl ester was prepared by the same method described in example 232, step 3 using acetic acid 3-(6-amino-8-bromo-purin-9-yl)-ethyl ester. Title compound was obtained as a white powder (85% yield). ¹H NMR (CDCl₃) δ 1.96 (s, 3H), 4.48 (t, J=5.0 Hz, 2H), 4.64 (t, J=5.0 Hz, 2H), 5.87 (s, 2H, NH₂), 7.37 (d, J=7.79 Hz, 1H), 7.44 (t, J=7.80 Hz, 1H), 7.83 (d, J=7.80 Hz, 1H), 8.43 (s, 1H). HPLC: RT=5.57 min (method: 5-100-7).

### EXAMPLE 242 Acetic acid 2-[6-amino-8-(7-chloro-benzothiazol-2-ylsulfanyl)-purin-9-yl]-propyl ester

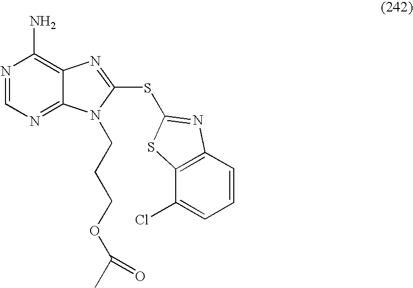

Acetic acid 2-[6-amino-8-(7-chloro-benzothiazol-2-ylsulfanyl)-purin-9-yl]-propyl ester was prepared by the same method described in example 241. ¹H NMR (CDCl₃) δ 2.00 (s, 3H), 2.26 (m, 2H), 4.08 (t, J=5.9 Hz, 2H), 4.47 (t, J=7.0 Hz, 2H), 6.06 (s, 2H, NH₂), 7.36 (d, J=7.79 Hz, 1H), 7.42 (t, J=7.80 Hz, 1H), 7.82 (d, J=7.80 Hz, 1H), 8.42 (s, 1H). HPLC: RT=5.76 min (method: 5-100-7).

### EXAMPLE 243 Acetic acid 4-[6-amino-8-(7-chloro-benzothiazol-2-ylsulfanyl)-purin-9-yl]-butyl ester

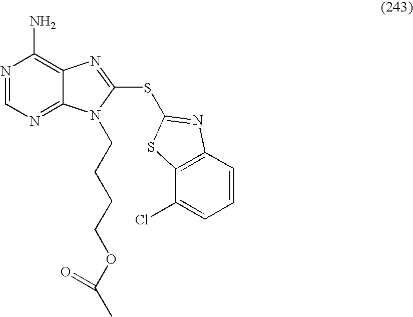

Acetic acid 4-[6-amino-8-(7-chloro-benzothiazol-2-ylsulfanyl)-purin-9-yl]-butyl ester was prepared by the same method described in example 241. ¹H NMR (CDCl₃) δ 1.66 (m, 2H), 1.95 (m, 2H), 1.96 (s, 3H), 4.11 (t, 2H), 4.39(t, 2H), 6.31 (s, 2H), 7.33 (d, 1H), 7.42 (t, 1H), 7.82 (d, 1H), 8.40(s, 1H). HPLC: RT=5.89 min (method: 5-100-7).

### EXAMPLE 244 2-[6-Amino-8-(7-chloro-benzothiazol-2-ylsulfanyl)-purin-9-yl]-ethanol

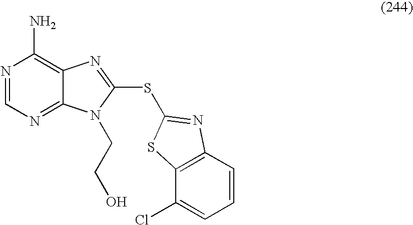

A solution of acetic acid 2-[6-amino-8-(7-chloro-benzothiazol-2-ylsulfanyl)-purin-9-yl]-ethyl ester, (241; 200 mg, 0.476 mmol) in 10 ml of 7N N₃ in methanol was stirred at room temperature for 4 h. The solvent was evaporated and the residue was purified with flash chromatography (5% MeOH/CH₂Cl₂) to yield 2-[6-amino-8-(7-chloro-benzothiazol-2-ylsulfanyl)-purin-9-yl]-ethanol, as a white powder (172 mg, 0.455 mmol) with 94% yield. ¹H NMR (DMSO) δ 3.58 (m, 2H), 4.33 (t, J=5.9 Hz, 2H), 5.04 (t, J=5.0 Hz, 1H, OH), 7.55(m, 2H), 7.64 (s, 2H, NH₂), 7.93 d, J=5.0 Hz, 1H), 8.26 (s, 1H). HPLC: RT=5.01 min (method: 5-100-7).

### EXAMPLE 245 2-[6-Amino-8-(7-chloro-benzothiazol-2-ylsulfanyl)-purin-9-yl]-propan-1-ol

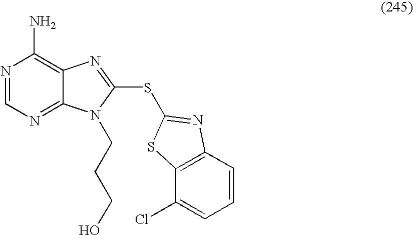

2-[6-Amino-8-(7-chloro-benzothiazol-2-ylsulfanyl)-purin-9-yl]-propan-1-ol was prepared by the same method described in example 244, and was obtained as a white powder (94% yield). ¹H NMR (CDCl₃) δ 1.93 (m, 2H), 3.47 (m, 2H), 4.52 (t, J=7.0 Hz, 2H), 5.78 (s, 2H, NH₂), 7.39 (d, J=7.79 Hz, 1H), 7.42 (t, J=7.80 Hz, 1H), 7.82 (d, J=7.80 Hz, 1H), 8.43 (s, 1H). HPLC: RT=5.134 min (5-100-7).

### EXAMPLE 246 4-[6-Amino-8-(7-chloro-benzothiazol-2-ylsulfanyl)-purin-9-yl]-butan-1-ol (246)

4-[6-Amino-8-(7-chloro-benzothiazol-2-ylsulfanyl)-purin-9-yl]-butan-1-ol was prepared by the same method described in example 244. ¹H NMR (CDCl₃) δ 1.59(m,2H), 2.02(m,2H), 3.72 (t, 2H), 4.47 (t, 2H), 5.83 (s, 2H), 7.36 (d, 1H), 7.43 (t, 1H), 7.84 (d, 1H), 8.43 (s, 1H). HPLC: RT=5.21 min (method: 5-100-7).

### EXAMPLE 247 8-(7-Chloro-benzothiazol-2-ylsulfanyl)-9-[2-(2,2-dimethyl-propylamino)-ethyl]-9H-purin-6-yl amine (247)

### Step 1 Methanesulfonic acid 2-[6-amino-8-(7-chloro-benzothiazol-2-ylsulfanyl)-purin-9-yl]-ethyl ester

To a solution of 2-[6-Amino-8-(7-chloro-benzothiazol-2-ylsulfanyl)-purin-9-yl]-ethanol (244; 100 mg, 0.29 mmol) in 5 ml of DMF at 0° C. was slowly added methanesulfonyl chloride (33.7 ul, 0.45 mmol) and triethyl amine (48.6 ul, 0.35 mmol). The reaction mixture was stirred for 10 min at 0° C. Most of solvent was then removed. The product was obtained after quenching the crude with water followed by filtration, to yield the title compound as a white powder (94% yield). ¹H NMR (CDCl₃) δ 2.9 (s, 3H), 4.68 (t, J=4.87 Hz, 2H), 4.75 (t, J=4.90 Hz, 2H), 5.88 (s, 2H, NH₂), 7.37 (d, J=7.79 Hz, 1H), 7.45 (t, J=7.80 Hz, 1H), 7.82 (d, J=7.80 Hz, 1H), 8.44 (s, 1H).

### Step 2 8-(7-Chloro-benzothiazol-2-ylsulfanyl)-9-[2-(2,2-dimethyl-propylamino)-ethyl]-9H-purin-6-yl amine

To Methanesulfonic acid 2-[6-amino-8-(7-chloro-benzothiazol-2-ylsulfanyl)-purin-9-yl]-ethyl ester (100 mg, 0.22 mmol) was added 1 ml of 2,2-Dimethyl-propylamine. The reaction mixture was stirred overnight at room temperature. The excess amine was removed. The residue was purified by flash chromatography (silica gel) (5% MeOH/CH₂Cl₂) to give 8-(7-chloro-benzothiazol-2-ylsulfanyl)-9-[2-(2,2-dimethyl-propylamino)-ethyl]-9H-purin-6-yl amine (30 mg, 0.067 mmol) as a white powder with 30.5% yield. ¹H NMR (CDCl₃) δ 0.82 (s, 3H), 2.31 (s, 2H), 3.05 (t, J=4.87 Hz, 2H), 4.47 (t, J=4.90 Hz, 2H), 5.74 (s, 2H, NH₂), 7.35 (d, J=7.79 Hz, 1H), 7.43 (t, J=7.80 Hz, 1H), 7.84 (d, J=7.80 Hz, 1H), 8.44 (s, 1H). HPLC: RT=5.38 min (method: 5-100-7).

### EXAMPLE 248 8-(7-Chloro-benzothiazol-2-ylsulfanyl)-9-(2-isopropylamino)-ethyl)-9H-purin-6-yl amine (248)

8-(7-Chloro-benzothiazol-2-ylsulfanyl)-9-(2-isopropylamino)-ethyl)-9H-purin-6-yl amine was prepared by the same method described in example 247. ¹H NMR (CDCl₃) δ 0.92 (d, 6H), 2.85 (m, 1H), 3.05 (t, 2H), 4.47 (t, 2H), 5.76 (s, 2H), 7.35 (d, 1H), 7.41 (t, 1H), 7.81 (d, 1H), 8.43(s, 1H). HPLC: RT=4.81 min (method: 5-100-7).

### EXAMPLE 249 8-(7-Chloro-benzothiazol-2-ylsulfanyl)-9-(2-isobutylamino)-ethyl)-9H-purin-6-yl amine (249)

8-(7-Chloro-benzothiazol-2-ylsulfanyl)-9-(2-isobutylamino)-ethyl)-9H-purin-6-yl amine was prepared by the same method described in example 247. ¹H NMR (CDCl₃) δ 0.82 (d, 6H), 1.65 (m, 1H), 2.39 (d, 2H), 3.05 (t, 2H), 4.46(t, 2H), 5.72 (s, 2H), 7.33 (d, 1H), 7.41 (t, 1H), 7.81 (d, 1H), 8.43(s, 1H). HPLC: RT=5.10 min (method: 5-100-7).

### EXAMPLE 250 8-(7-Chloro-benzothiazol-2-ylsulfanyl)-9-[2-(1-ethyl-propylamino)-ethyl)-9H-purin-6-yl amine

8-(7-Chloro-benzothiazol-2-ylsulfanyl)-9-[2-(1-ethyl-propylamino)-ethyl)-9H-purin-6-yl amine was prepared by the same method described in example 247. ¹H NMR (CDCl₃) δ 0.73 (d, 6H), 1.24 (m, 4H), 2.27 (t, 1H), 3.01 (t, 2H), 4.44(t, 2H), 5.75 (s, 2H), 7.34 (d, 1H), 7.44 (t, 1H), 7.82 (d, 1H), 8.43(s, 1H). HPLC: RT=5.19 min (method: 5-100-7).

### EXAMPLE 251 8-(7-Chloro-benzothiazol-2-ylsulfanyl)-9-(2-propylamino)-ethyl)-9H-purin-6-yl amine (251)

8-(7-Chloro-benzothiazol-2-ylsulfanyl)-9-(2-propylamino)-ethyl)-9H-purin-6-yl amine was prepared by the same method described in example 247. ¹H NMR (CDCl₃) δ 0.83 (t, 3H), 1.40 (m, 2H), 2.57 (t, 2H), 3.05 (t, 2H), 4.48(t, 2H), 5.78(s, 2H), 7.33 (d, 1H), 7.42 (t, 1H), 7.82 (d, 1H), 8.43(s, 1H). HPLC: RT=4.88 min (method: 5-100-7).

### EXAMPLE 252 8-(7-Chloro-benzothiazol-2-ylsulfanyl)-9-[3-(1-ethyl-propylamino)-propyl]-9H-purin-6-yl amine (252)

8-(7-Chloro-benzothiazol-2-ylsulfanyl)-9-[3-(1-ethyl-propylamino)-propyl]-9H-purin-6-yl amine was prepared by the same method described in example 247. ¹H NMR (CDCl₃) δ 0.95 (t, 6H), 1.58 (m, 4H), 2.20 (m, 2H), 2.79 (m, 1H), 2.95 (t, 2H), 4.50(t, 2H), 5.67 (s, 2H), 7.47 (d, 1H), 7.54 (t, 1H), 7.86 (d, 1H), 8.32(s, 1H). HPLC: RT=5.158 min (method: 5-100-7).

### EXAMPLE 253 9-(3-tert-Butylamino-propyl)-8-(7-chloro-benzothiazol-2-ylsulfanyl)-9H-purin-6-yl amine (253)

9-(3-tert-Butylamino-propyl)-8-(7-chloro-benzothiazol-2-ylsulfanyl)-9H-purin-6-yl amine was prepared by the same method described in example 247. ¹H NMR (CDCl₃) δ 1.32 (s, 9H), 2.22 (m, 2H), 3.02 (t, 2H), 4.53 (t, 2H), 5.67 (s, 2H), 7.47 (d, 1H), 7.54 (t, 1H), 7.86 (d, 1H), 8.34(s, 1H). HPLC: RT=5.005 min (method: 5-100-7).

### EXAMPLE 254 8-(7-Chloro-benzothiazol-2-ylsulfanyl)-9-(3-isobutylamino-propyl)-9H-purin-6-yl amine (254)

8-(7-Chloro-benzothiazol-2-ylsulfanyl)-9-(3-isobutylamino-propyl)-9H-purin-6-yl amine was prepared by the same method described in example 247. ¹H NMR (DMSO) δ 8.28 (s, 1H), 7.93(t, J=8.4 Hz, 1H), 7.76(bs, 2H, NH₂), 7.57(d, J=1.08 Hz, 2H), 4.36(t, J=7.35 Hz, 2H), 2.90(t, J=7.35 Hz, 2H), 2.38(d, J=6.89, 2H, CH₂), 2.25(m, 2H, CH₂), 1.64(m, 1H, CH₃), 0.83(s, 6H, 2CH₃). HPLC: RT=5.035 (5-100-7).

### EXAMPLE 255 9-(3-sec-Butylamino-propyl)-8-(7-Chloro-benzothiazol-2-ylsulfanyl)-9H-purin-6-yl amine (255)

9-(3-sec-Butylamino-propyl)-8-(7-Chloro-benzothiazol-2-ylsulfanyl)-9H-purin-6-yl amine was prepared by the same method described in example 247. ¹H NMR (DMSO) δ 8.28(s, 1H), 7.93(t, J=8.4 Hz, 1H), 7.76(bs, 2H, NH₂), 7.57(d, J=1.08 Hz, 2H), 4.36(t, J=7.35 Hz, 2H), 3.17(m, 1H, CH), 2.90(t, J=7.35 Hz, 2H), 2.70(m, 1H, CH), 2.25(m, 2H, CH2), 1.51(m, 2H, CH2), 1.10(m, 6H, 2CH3). HPLC: RT=5.034 (5-100-7).

### EXAMPLE 256 9-[2-(2,2-Dimethyl-propylamino)-ethyl]-8-(7-Chloro-benzothiazol-2-ylsulfanyl)-9H-purin-6-yl amine (256)

9-[2-(2,2-Dimethyl-propylamino)-ethyl]-8-(7-Chloro-benzothiazol-2-ylsulfanyl)-9H-purin-6-yl amine was prepared by the same method described in example 247. ¹H NMR (CD₃OD) δ 8.29 (s, 1H), 7.50(d, J=1.08 Hz, 1H), 7.48(t, J=8.4 Hz, 1H), 6.99(d, J=1.08 Hz, 1H), 4.48(t, J=5.09, 2H, CH2), 3.97(s, 3H, CH3), 2.97(t, J=5.09, 2H, CH2), 2.25(s, 2H, CH2), 0.80(s, 9H, 3CH3). HPLC: RT=5.110 (Method: 5-100-7).

### EXAMPLE 257 8-(7-Chloro-benzothiazol-2-ylsulfanyl)-9-[3-(2,2-dimethyl-propylamino)-propyl]-9H-purin-6-yl amine (257)

8-(7-Chloro-benzothiazol-2-ylsulfanyl)-9-[3-(2,2-dimethyl-propylamino)-propyl]-9H-purin-6-yl amine was prepared by the same method described in example 247. ¹H NMR (CDCl₃) δ 0.88(s, 9H), 2.02 (m, 2H), 2.24 (s, 2H), 2.62 (t, 2H), 4.47 (t, 2H), 5.73 (s, 2H), 7.35 (d, 1H), 7.43 (t, 1H), 7.84 (d, 1H), 8.43(s, 1H). HPLC: RT=5.21 min (method: 5-100-7).

### EXAMPLE 258 8-(7-Chloro-benzothiazol-2-ylsulfanyl)-9-[2-(cyclopropylmethyl-amino)-ethyl]-9H-purin-6-yl amine (258)

8-(7-Chloro-benzothiazol-2-ylsulfanyl)-9-[2-(cyclopropylmethyl-amino)-ethyl]-9H-purin-6-yl amine was prepared by the same method described in example 247. ¹H NMR (CDCl₃) δ 0.09(m, 2H), 0.41(m 2H), 0.85(m, 1H), 2.46 (d, 2H), 3.07 (t, 2H), 4.47 (t, 2H), 5.82 (s, 2H), 7.35 (d, 1H), 7.43 (t, 1H), 7.84 (d, 1H), 8.43(s, 1H). HPLC: RT=4.97 min (method: 5-100-7).

### EXAMPLE 259 8-(7-Chloro-benzothiazol-2-ylsulfanyl)-9-(2-prop-2-ynylamino-ethyl)-9H-purin-6-yl amine (259)

8-(7-Chloro-benzothiazol-2-ylsulfanyl)-9-(2-prop-2-ynylamino-ethyl)-9H-purin-6-yl amine was prepared by the same method described in example 247. ¹H NMR (CDCl₃) δ 2.10 (t, 1H), 3.16 (m, 2H), 3.51 (s, 2H), 4.49 (t, 2H), 5.83 (s, 2H), 7.35 (d, 1H), 7.43 (t, 1H), 7.84 (d, 1H), 8.43(s, 1H). HPLC: RT=4.74 min (method: 5-100-7).

### EXAMPLE 260 8-(7-Chloro-benzothiazol-2-ylsulfanyl)-9-(2-cyclopentylamino-ethyl)-9H-purin-6-yl amine (260)

8-(7-Chloro-benzothiazol-2-ylsulfanyl)-9-(2-cyclopentylamino-ethyl)-9H-purin-6-yl amine was prepared by the same method described in example 247. ¹H NMR (CDCl₃) δ 8.42(s, 1H), 7.83(d, J=7.0 Hz, 1H), 7.43(t, J=7.9 Hz, 1H), 7.38(d, J=7.0 Hz, 1H), 5.70(bs, 2H, NH₂), 4.48(t, J=5.95 Hz, 2H, CH₂), 3.08(t, J=5.95 Hz, 2H, CH₂), 1.75(m, 1H, CH), 1.61(m, 4H, 2CH₂), 1.48(m, 4H, 2CH₂). HPLC: RT=5.090 (Method: 5-100-7

### EXAMPLE 261 8-(7-Chloro-benzothiazol-2-ylsulfanyl)-9-[2-(3-methyl-butylamino)-ethyl]-9H-purin-6-yl amine (261)

8-(7-Chloro-benzothiazol-2-ylsulfanyl)-9-[2-(3-methyl-butylamino)-ethyl]-9H-purin-6-yl amine was prepared by the same method described in example 247. ¹H NMR (CDCl₃) δ 8.44(s, 1H), 7.83(d, J=7.0 Hz, 1H), 7.43(t, J=7.9 Hz, 1H), 7.38(d, J=7.0 Hz, 1H), 5.79(bs, 2H, NH₂), 4.49(t, J=5.95 Hz, 2H, CH₂), 3.06(t, J=5.95 Hz, 2H, CH₂), 2.61(t, J=8.19 Hz, 2H, CH₂), 1.55(m, 3H, CH+CH₂), 0.85(d, J=6.62, 6H, 2CH₃). HPLC: RT=5.323 (Method: 5-100-7).

### EXAMPLE 262 8-(7-Chloro-benzothiazol-2-ylsulfanyl)-9-[2-(1,1-dimethyl-propylamino)-ethyl]-9H-purin-6-yl amine (262)

8-(7-Chloro-benzothiazol-2-ylsulfanyl)-9-[2-(1,1-dimethyl-propylamino)-ethyl]-9H-purin-6-yl amine was prepared by the same method described in example 247. ¹H NMR (CDCl₃) δ 8.44(s, 1H), 7.81(d, J=7.0 Hz, 1H), 7.43(t, J=7.9 Hz, 1H), 7.35(d, J=7.0 Hz, 1H), 5.74(bs, 2H, NH₂), 4.43(t, J=5.95 Hz, 2H, CH₂), 2.95(t, J=5.95 Hz, 2H, CH₂), 1.30(m, 2H, CH₂), 0.92(s, 6H, 2CH3), 0.74(t, J=6.12 Hz, 3H, CH₃). HPLC: RT=5.145 (Method: 5-100-7).

### EXAMPLE 263 9-(2-Allylamino-ethyl)-8-(7-chloro-benzothiazol-2-ylsulfanyl)-9H-purin-6-ylamine (263)

9-(2-Allylamino-ethyl)-8-(7-chloro-benzothiazol-2-ylsulfanyl)-9H-purin-6-ylamine was prepared by the same method described in example 247. ¹H NMR (CDCl₃) δ 3.03(t, 2H), 3.223(d 2H), 4.47 (t, 2H), 5.00(m, 2H), 5.71(m, 1H), 5.80 (s, 2H), 7.35 (d, 1H), 7.43 (t, 1H), 7.84 (d, 1H), 8.43(s, 1H). HPLC: RT=:4.82 min (method: 5-100-7).

### EXAMPLE 264 8-(7-Chloro-benzothiazol-2-ylsulfanyl)-9-(2-cyclopropylamino)-ethyl)-9H-purin-6-yl amine (264)

8-(7-Chloro-benzothiazol-2-ylsulfanyl)-9-(2-cyclopropylamino)-ethyl)-9H-purin-6-yl amine was prepared by the same method described in example 247 except that cyclopropylamine was used instead of 2,2-dimethyl-propylamine. ¹H NMR (CDCl₃) δ 0.16 (m, 2H), 0.36 (m, 2H), 2.15 (m, 1H), 3.15 (t, J=4.87 Hz, 2H), 4.46 (t, J=4.90 Hz, 2H), 5.70 (s, 2H, NH₂), 7.35 (d, J=7.79 Hz, 1H), 7.43 (t, J=7.80 Hz, 1H), 7.84 (d, J=7.80 Hz, 1H), 8.44 (s, 1H). HPLC: RT=4.95 min (method 5-100-7).

### EXAMPLE 265 9-(2-tert-Butylamino-ethyl)-8-(7-chloro-benzothiazole-2-ylsulfanyl)-9H-purin-6-yl amine (265)

9-(2-tert-Butylamino-ethyl)-8-(7-chloro-benzothiazole-2-ylsulfanyl)-9H-purin-6-yl amine was prepared by the same method described in example 247 except that tert-butylamine was used instead of 2,2-dimethyl-propylamine. ¹H NMR (CDCl₃) δ 0.96 (s, 9H), 3.00 (t, J=4.87 Hz, 2H), 4.46 (t, J=4.90 Hz, 2H), 5.77 (s, 2H, NH₂), 7.35 (d, J=7.79 Hz, 1H), 7.43 (t, J=7.80 Hz, 1H), 7.84 (d, J=7.80 Hz, 1H), 8.43 (s, 1H). HPLC: RT=5.04 min (method 5-100-7).

### EXAMPLE 266 8-(7-Chloro-benzothiazol-2-ylsulfanyl)-9-(3-isopropylamino-propyl)-9H-purin-6-yl amine (266)

8-(7-Chloro-benzothiazol-2-ylsulfanyl)-9-(3-isopropylamino-propyl)-9H-purin-6-yl amine was prepared by the same method described in example 247 except that isopropyl amine was used instead of 2,2-dimethyl-propylamine. ¹H NMR (DMSO) δ 1.09 (s, 3H), 1.10 (s, 3H), 2.10 (m, 2H), 2.90 (m, 2H), 3.17 (m, 1H), 4.36(t, 2H), 7.57 (d, 2H), 7.76 (bs, 2H), 7.93 (t, 1H), 8.28(s, 1H). HPLC: RT=4.811 min (method: 5-100-7).

### EXAMPLE 267 8-(7-Chlorol-benzothiazol-2-ylsulfanyl)-9-(3-pyrrol-1-yl-propyl)-9H-purine-6-ylamine (267)

8-(7-Chlorol-benzothiazol-2-ylsulfanyl)-9-(3-pyrrol-1-yl-propyl)-9H-purine-6-ylamine was prepared by the same method described in example 247. ¹H NMR (CDCl₃) δ 2.40 (m, 2H), 3.97 (t, 2H), 4.35 (t, 2H), 5.82 (bs, 2H), 6.11 (d, 2H), 6.66 (d, 2H), 7.37(d, 1H), 7.44 (t, 1H), 7.83(d,1H), 8.45 (s, 1H). HPLC: RT=6.372 min (method: 5-100-7).

### EXAMPLE 268 8-(7-Chloro-benzothiazol-2-ylsulfanyl)-9-[2-(3,3-dimethyl-butylamino)-ethyl]-9H-purin-6-yl amine (268)

8-(7-Chloro-benzothiazol-2-ylsulfanyl)-9-[2-(3,3-dimethyl-butylamino)-ethyl]-9H-purin-6-yl amine was prepared by the same method described in example 247. ¹H NMR (CDCl₃) δ 8.44 (s, 1H), 7.83(d, J=7.0 Hz, 1H), 7.43(t, J=7.9 Hz, 1H), 7.38(d, J=7.0 Hz, 1H), 5.70(bs, 2H, NH₂), 4.47(t, J=5.95 Hz, 2H, CH₂), 3.06(t, J=5.95 Hz, 2H, CH₂), 2.55(t, J=8.19 Hz, 2H, CH₂), 1.22(t, J=8.19 Hz, 2H, CH₂), 0.83(s, 9H, 3CH₃). HPLC: RT=5.558(Method: 5-100-7).

### EXAMPLE 269 8-(7-Chloro-benzothiazol-2-ylsulfanyl)-9-(3-morpholin-4-yl-propyl)-9H-purin-6-ylamine (269)

8-(7-Chloro-benzothiazol-2-ylsulfanyl)-9-(3-morpholin-4-yl-propyl)-9H-purin-6-ylamine was prepared by the same method described in example 247. ¹H NMR (CDCl₃) δ 1.30 (m, 2H), 2.22 (m, 4H), 2.40 (m, 2H), 3.62(m, 4H), 4.48 (t,2H), 5.70 (s, 2H), 7.37 (d, 1H), 7.44(t, 1H), 7.83 (d, 1H), 8.44 (s, 1H). HPLC: RT=4.77 min (method: 5-100-7).

### EXAMPLE 270 8-(7-Chloro-benzothiazol-2-ylsulfanyl)-9-(2-morpholin-4-yl-ethyl)-9H-purin-6-ylamine (270)

8-(7-Chloro-benzothiazol-2-ylsulfanyl)-9-(2-morpholin-4-yl-ethyl)-9H-purin-6-ylamine was prepared by the same method described in example 247. ¹H NMR (CDCl₃) δ 2.50 (t, 4H), 2.76 (t, 2H), 3.64(t, 4H), 4.46 (t, 2H), 5.85 (s, 2H), 7.35 (d, 1H), 7.44(t, 1H), 7.83 (d, 1H), 8.42 (s, 1H). HPLC: RT=4.71 min (method: 5-100-7).

### EXAMPLE 271 9-(2-Bromo-ethyl)-8-(7-chloro-benzothiazol-2-ylsulfanyl)-9H-purine-6-ylamine (271)

### Step 1 8-Bromoadenine was prepared as reported, see Collect. Czech. Chem. Commun. 2000, 65, 1126-1144

### Step 2 8-(7-Chloro-benzothiazol-2-ylsulfanyl)-9H-purine-6-ylamine

Followed the same procedure given in example 232, step 4. ¹H NMR (DMSO) δ 8.23(s, 1H), 8.10(t, 1H), 7.90(d, 1H), 7.82(bs, 2H), 7.59 (d, 1H).

### Step 3 9-(2-Bromo-ethyl)-8-(7-chloro-benzothiazol-2-ylsulfanyl)-9H-purine-6-ylamine

To a mixture of 8-(7-Chloro-benzothiazol-2-ylsulfanyl)-9H-purine-6-ylamine (200 mg, 0.599 mmol) and cesium carbonate (292.6 mg, 0.898 mmol) in DMF (10 ml) was added 1,2-dibromo-ethane (168.7 mg, 0.898 mmol) at room temperature. The reaction mixture was stirred at room temperature for 16 hours before quenched with water (20 ml). The precipitate was filtered off and dried under vacuum. The crude material was purified by flash chromatography with 35% yield. ¹H NMR (CDCl₃) δ 3.82 (t, 2H), 4.78 (t, 2H), 5.71 (s, 2H), 7.36 (d, 1H), 7.42(t, 1H), 7.84 (d, 1H), 8.45 (s, 1H). HPLC: RT=6.05 min (method: 5-100-7).

### EXAMPLE 272 8-(7-Chloro-benzothiazol-2-ylsulfanyl)-9-(2-chloro-ethyl)-9H-purine-6-ylamine (272)

8-(7-Chloro-benzothiazol-2-ylsulfanyl)-9-(2-chloro-ethyl)-9H-purine-6-ylamine was prepared by the same method described in example 271. ¹H NMR (CDCl₃) δ 3.92 (t, 2H), 4.72 (t, 2H), 5.86 (s, 2H), 7.36 (d, 1H), 7.43(t, 1H), 7.83(d, 1H), 8.45 (s, 1H). HPLC: RT=5.93 min (method: 5-100-7).

### EXAMPLE 273 9-(3-Bromo-propyl)-8-(7-chloro-benzothiazol-2-ylsulfanyl)-9H-purine-6-ylamine (273)

9-(3-Bromo-propyl)-8-(7-chloro-benzothiazol-2-ylsulfanyl)-9H-purine-6-ylamine was prepared by the same method described in example 271. ¹H NMR (CDCl₃) δ 2.49 (m, 2H), 3.89 (t, 2H), 4.51 (t, 2H), 5.67 (s, 2H), 7.37 (d, 1H), 7.44(t, 1H), 7.83(d, 1H), 8.43(s, 1H). HPLC: RT=6.261 min (method: 5-100-7).

### EXAMPLE 274 8-(7-Chloro-benzothiazol-2-ylsulfanyl)-9-[2,(2,5-dimethoxy-phenyl)-ethyl]-9H-purine-6-ylamine (274)

8-(7-Chloro-benzothiazol-2-ylsulfanyl)-9-[2,(2,5-dimethoxy-phenyl)-ethyl]-9H-purine-6-ylamine was prepared by the same method described in example 271. ¹H NMR (CDCl₃) δ 3.15 (t, 2H), 3.61(s, 3H), 3.62 (s, 3H), 4.60(t, 2H), 5.7 (bs, 2H), 6.37 (s, 1H), 6.56(d, 1H), 6.61(d, 1H), 7.31(d, 1H), 7.39 (t,1H), 7.77(d,1H), 8.46(s,1H); HPLC: RT=6.515 min (method 5-100-7).

### EXAMPLE 275 9-But-2-ynyl-8-(7-chloro-benzothiazol-2-ylsulfanyl)-9H-purine-6-ylamine (275)

9-But-2-ynyl-8-(7-chloro-benzothiazol-2-ylsulfanyl)-9H-purine-6-ylamine was prepared by the same method described in example 271. ¹H NMR (CDCl₃) δ 1.54(s, 3H), 5.09(t, 2H), 6.00(bs, 2H), 7.36(d,1H), 7.44(t,1H), 7.86(d,1H), 8.47(s,1H); HPLC: RT=5.964 min (method 5-100-7).

### EXAMPLE 276 8-(7-Chloro-benzothiazol-2-ylsulfanyl)-9-(3,4,4-trifluoro-but-3-enyl)-9H-purin-6-ylamine (276)

8-(7-Chloro-benzothiazol-2-ylsulfanyl)-9-(3,4,4-trifluoro-but-3-enyl)-9H-purin-6-ylamine was prepared by the same method described in example 271. ¹H NMR (CDCl₃) δ 2.92 (m, 2H), 4.59 (t, 2H), 5.90 (bs, 2H), 7.35 (d, 1H), 7.44(t, 1H), 7.84 (d, 1H), 8.45 (s, 1H). HPLC: RT=6.29 min (method: 5-100-7).

### EXAMPLE 277 6-[6-Amino-8-(7-chloro-benzothiazol-2-ylsulfanyl)-purin-9-yl]-hexanenitrile (277)

6-[6-Amino-8-(7-chloro-benzothiazol-2-ylsulfanyl)-purin-9-yl]-hexanenitrile was prepared by the same method described in example 271. ¹H NMR (CDCl₃) δ 1.52 (m, 2H), 1.72 (m, 2H), 1.94 (m, 2H), 2.33 (t, 2H), 4.41 (t, 2H), 7.42 (d, 1H), 7.47(t, 1H), 7.86(d, 1H), 8.40 (s, 1H). HPLC: RT=5.879 min (method: 5-100-7).

### EXAMPLE 278 8-(7-Chloro-benzothiazol-2-ylsulfanyl)-9-(3-methyl-but-3-enyl)-9H-purin-6-ylamine (278)

8-(7-Chloro-benzothiazol-2-ylsulfanyl)-9-(3-methyl-but-3-enyl)-9H-purin-6-ylamine was prepared by the same method described in example 271. ¹H NMR (CDCl₃) δ 1.55 (s, 3H), 1.81 (s, 3H), 4.94 (d, 2H), 5.25 (t, 1H), 5.81 (bs, 2H), 7.35 (d, 1H), 7.44(t, 1H), 7.84 (d, 1H), 8.45 (s, 1H). HPLC: RT=6.524 min (method: 5-100-7).

### EXAMPLE 279 4-[6-Amino-8-(7-chloro-benzothiazol-2-ylsulfanyl)-purin-9-yl]-butyronitrile (279)

4-[6-Amino-8-(7-chloro-benzothiazol-2-ylsulfanyl)-purin-9-yl]-butyronitrile was prepared by the same method described in example 271. ¹H NMR (CDCl₃) δ 2.31 (m, 2H), 2.46(t, 2H), 4.50 (t, 2H), 5.74 (bs, 2H), 7.36 (d, 1H), 7.43(t, 1H), 7.83(d, 1H), 8.44 (s, 1H). HPLC: RT=5.516 min (method: 5-100-7).

### EXAMPLE 280 8-(7-Chloro-benzothiazol-2-ylsulfanyl)-9-hex-5-ynyl-9H-purin-6-ylamine (280)

8-(7-Chloro-benzothiazol-2-ylsulfanyl)-9-hex-5-ynyl-9H-purin-6-ylamine was prepared by the same method described in example 271. ¹H NMR (CDCl₃) δ 1.57 (m, 2H), 1.84 (t, 2H), 2.18(m, 2H), 2.22 (m, 2H), 4.39 (t, 2H), 5.71 (s, 2H), 7.36 (d, 1H), 7.44(t, 1H), 7.84 (d, 1H), 8.45 (s, 1H). HPLC: RT=6.276 min (method: 5-100-7).

### EXAMPLE 281 8-(7-Chloro-benzothiazol-2-ylsulfanyl)-9-[3-(tetrahydro-furan-2-yl)-propyl]-9H-purin-6-ylamine (281)

8-(7-Chloro-benzothiazol-2-ylsulfanyl)-9-[3-(tetrahydro-furan-2-yl)-propyl]-9H-purin-6-ylamine was prepared by the same method described in example 271. ¹H NMR (CDCl₃) δ 1.48 (m, 4H), 2.21 (m, 2H), 3.42(m, 2H), 3.81 (m, 2H), 4.52 (m, 3H), 5.92 (s, 2H), 7.37 (d, 1H), 7.44(t, 1H), 7.83 (d, 1H), 8.44 (s, 1H). HPLC: RT=6.395 min (method: 5-100-7).

### EXAMPLE 282 8-(7-Chloro-benzothiazol-2-ylsulfanyl)-9-(tetrahydro-furan-2-ylmethyl)-9H-purin-6-ylamine (282)

8-(7-Chloro-benzothiazol-2-ylsulfanyl)-9-(tetrahydro-furan-2-ylmethyl)-9H-purin-6-ylamine was prepared by the same method described in example 271. ¹H NMR (CDCl₃) δ 1.68 (m, 4H), 3.21 (t, 1H), 3.68(m, 1H), 3.78 (d, 1H), 4.37(d, 2H), 5.78 (s, 2H), 7.37 (d, 1H), 7.44(t, 1H), 7.83 (d, 1H), 8.44 (s, 1H). HPLC: RT=6.404 min (method: 5-100-7).

### EXAMPLE 283 8-(7-Chloro-benzothiazol-2-ylsulfanyl)-9-[2-(2-ethoxy-ethoxy)-ethyl]-9H-purin-6-ylamine (283)

8-(7-Chloro-benzothiazol-2-ylsulfanyl)-9-[2-(2-ethoxy-ethoxy)-ethyl]-9H-purin-6-ylamine was prepared by the same method described in example 271. ¹H NMR (CDCl₃) δ 1.15 (t, 3H), 3.46 (m, 6H), 3.88(t, 2H), 4.58 (t, 2H), 5.84(s, 2H), 7.37 (d, 1H), 7.44(t, 1H), 7.83 (d, 1H), 8.44 (s, 1H). HPLC: RT=5.925 min (method: 5-100-7).

### EXAMPLE 284 5-[6-Amino-8-(7-chloro-benzothiazol-2-ylsulfanyl)-purin-9-yl]-pentanenitrile (284)

5-[6-Amino-8-(7-chloro-benzothiazol-2-ylsulfanyl)-purin-9-yl]-pentanenitrile was prepared by the same method described in example 271. ¹H NMR (CDCl₃) δ 1.71 (m, 2H) 2.05(m, 2H), 2.42(t, 2H), 4.41 (t, 2H), 5.88 (bs, 2H), 7.36 (d, 1H), 7.43(t, 1H), 7.83(d, 1H), 8.44 (s, 1H). HPLC: RT=5.694 min (method: 5-100-7).

### EXAMPLE 285 8-(7-Chlorol-benzothiazol-2-ylsulfanyl)-9-(4-methoxy-3,5-dimethyl-pyridin-2-ylmethyl)-9H-purine-6-ylamine (285)

8-(7-Chlorol-benzothiazol-2-ylsulfanyl)-9-(4-methoxy-3,5-dimethyl-pyridin-2-ylmethyl)-9H-purine-6-ylamine was prepared by the same method described in example 271. ¹H NMR (CDCl₃) δ 2.08(s, 3H), 2.35 (s, 3H), 3.71 (s, 3H), 5.60 (s, 2H), 5.82 (bs, 2H), 7.31(d, 1H), 7.39 (t, 1H), 7.77(d,1H), 8.45 (s, 1H). HPLC: RT=5.570 min (method: 5-100-7).

### EXAMPLE 286 8-(7-Chloro-benzothiazol-2-ylsulfanyl)-9-prop-2-ynyl-9H-purine-6-ylamine (286)

8-(7-Chloro-benzothiazol-2-ylsulfanyl)-9-prop-2-ynyl-9H-purine-6-ylamine was prepared by the same method described in example 271. ¹H NMR (CDCl₃) δ 8.49(s, 1H), 7.84(d, J=1.08 Hz, 1H), 7.46(t, J=8.4 Hz, 1H), 7.36(d, J=1.08 Hz, 1H), 5.83(bs, 2H, NH₂), 5.15(s, 2H, CH₂), 2.25(s, 1H, CH). HPLC: RT=5.700 (Method: 5-100-7).

### EXAMPLE 287 8-(7-Chloro-benzothiazol-2-ylsulfanyl)-9-(2-piperidin-1-yl-ethyl]-9H-purin-6-yl amine (287)

8-(7-Chloro-benzothiazol-2-ylsulfanyl)-9-(2-piperidin-1-yl-ethyl]-9H-purin-6-yl amine was prepared by the same method described in example 271. ¹H NMR (CDCl₃) δ 8.44(s, 1H), 7.83(d, J=7.0 Hz, 1H), 7.43(t, J=7.9 Hz, 1H), 7.38(d, J=7.0 Hz, 1H), 5.70(bs, 2H, NH₂), 4.44(t, J=5.95 Hz, 2H, CH₂), 2.68(t, J=5.95 Hz, 2H, CH₂), 2.42(t, J=8.19 Hz, 4H, 2CH₂), 1.57(t, J=8.19 Hz, 4H, 2CH₂), 1.40(m, 2H, CH2). HPLC: RT=4.923(Method: 5-100-7).

### EXAMPLE 288 8-(7-Chloro-benzothiazol-2-ylsulfanyl)-9-(2-methylsulfanyl-ethyl)-9H-purin-6-ylamine (288)

8-(7-Chloro-benzothiazol-2-ylsulfanyl)-9-(2-methylsulfanyl-ethyl)-9H-purin-6-ylamine was prepared by the same method described in example 271. ¹H NMR (CDCl₃) δ 2.15(s, 3H), 2.99 (t, 2H), 4.57 (t, 2H), 5.72 (s, 2H), 7.35 (d, 1H), 7.43 (t, 1H), 7.84 (d, 1H), 8.43(s, 1H). HPLC: RT=6.04 min (method: 5-100-7).

### EXAMPLE 289 {3-[6-Amino-8-(7-chloro-benzothiazol-2-ylsulfanyl)-9-yl]-propyl}-phosphonic acid diethyl ester (289)

{3-[6-Amino-8-(7-chloro-benzothiazol-2-ylsulfanyl)-9-yl]-propyl}-phosphonic acid diethyl ester was prepared by the same method described in example 271. ¹H NMR (CDCl₃) δ 8.43(s, 1H), 7.83(d, J=7.0 Hz, 1H), 7.43(t, J=7.9 Hz, 1H), 7.38(d, J=7.0 Hz, 1H), 5.80(bs, 2H, NH₂), 4.45(t, J=7.23 Hz, 2H, CH₂), 4.04(m, 4H, 2CH₂), 2.21(m, 2H, CH₂), 1.35(m, 2H, CH₂), 1.26(t, J=7.06 Hz, 6H, 2CH₃). HPLC: RT=5.696 (Method: 5-100-7).

### EXAMPLE 290 8-(7-Chloro-benzothiazol-2-ylsulfanyl)-9-(2-ethylsulfanyl-ethyl)-9H-purin-6-ylamine (290)

8-(7-Chloro-benzothiazol-2-ylsulfanyl)-9-(2-ethylsulfanyl-ethyl)-9H-purin-6-ylamine was prepared by the same method described in example 271. ¹H NMR (CDCl₃) δ 1.21 (t, 3H), 2.58 (m, 2H), 3.00 (t, 2H), 4.57 (t, 2H), 5.72 (s, 2H), 7.35 (d, 1H), 7.43 (t, 1H), 7.84 (d, 1H), 8.43(s, 1H). HPLC: RT=6.34 min (method: 5-100-7).

### EXAMPLE 291 Phosphoric acid 3-[6-amino-8-(7-chloro-benzothiazol-2-ylsulfanyl)-9-yl]-propyl ester diethyl ester (291)

Phosphoric acid 3-[6-amino-8-(7-chloro-benzothiazol-2-ylsulfanyl)-9-yl]-propyl ester diethyl ester was prepared by the same method described in example 271. ¹H NMR (CDCl₃) δ 8.43(s, 1H), 7.83(d, J=7.0 Hz, 1H), 7.43(t, J=7.9 Hz, 1H), 7.38(d, J=7.0 Hz, 1H), 5.80(bs, 2H, NH₂), 4.50(t, J=7.23 Hz, 2H, CH₂), 4.12(m, 6H, 3CH₂), 2.30(m, 2H, CH₂), 1.32(t, J=7.06 Hz, 6H, 2CH₃). HPLC: RT=5.834 (Method: 5-100-7).

### EXAMPLE 292 Phosphoric acid 2-[6-amino-8-(7-chloro-benzothiazol-2-ylsulfanyl)-purin-9-yl]-ester bis-(2-chloro-ethyl ester (292)

Phosphoric acid 2-[6-amino-8-(7-chloro-benzothiazol-2-ylsulfanyl)-purin-9-yl]-ester bis-(2-chloro-ethyl ester was prepared by the same method described in example 271. ¹H NMR (CDCl₃) δ 8.43(s, 1H), 7.83(d, J=7.0 Hz, 1H), 7.43(t, J=7.9 Hz, 1H), 7.38(d, J=7.0 Hz, 1H), 5.80(bs, 2H, NH₂), 4.70(t, J=7.23 Hz, 2H, CH₂), 4.54(t, J=7.23 Hz, 2H, CH₂), 4.19(m, 4H, 2CH₂), 3.62(t, J=7.06 Hz, 4H 2CH₂). HPLC: RT=5.909 (Method: 5-100-7).

### EXAMPLE 293 {3-[6-Amino-8-(7-chloro-benzothiazol-2-ylsulfanyl)-purin-9-yl]-propenyl}-phosphonic acid diethyl ester (293)

{3-[6-Amino-8-(7-chloro-benzothiazol-2-ylsulfanyl)-purin-9-yl]-propenyl}-phosphonic acid diethyl ester was prepared by the same method described in example 271. ¹H NMR (CDCl₃) δ 8.43(s, 1H), 7.83(d, J=7.0 Hz, 1H), 7.43(t, J=7.9 Hz, 1H), 7.38(d, J=7.0 Hz, 1H), 6.85(t, J=8.0 Hz, 1H, CH), 5.86(bs, 2H, NH₂), 5.56(t, J=8.0 Hz, 1H, CH), 5.13(t, J=7.23 Hz, 2H, CH₂), 3.99(m, 4H, 2CH₂), 1.26(t, J=7.06 Hz, 6H, 2CH₃). HPLC: RT=5.622 (Method: 5-100-7).

### EXAMPLE 294 Phosphoric acid 2-[6-amino-8-(7-chloro-benzothiazol-2-ylsulfanyl)-9-yl]-ethyl ester diethyl ester (294)

Phosphoric acid 2-[6-amino-8-(7-chloro-benzothiazol-2-ylsulfanyl)-9-yl]-ethyl ester diethyl ester was prepared by the same method described in example 271. ¹H NMR (CDCl₃) δ 8.43(s, 1H), 7.83(d, J=7.0 Hz, 1H), 7.43(t, J=7.9 Hz, 1H), 7.38(d, J=7.0 Hz, 1H), 5.80(bs, 2H, NH₂), 4.66(t, J=7.23 Hz, 2H, CH₂), 4.48(m, 2H, CH₂), 3.99(m, 4H, 2CH₂), 1.22(t, J=7.06 Hz, 6H, 2CH₃). HPLC: RT=5.713 (Method: 5-100-7).

### EXAMPLE 295 8-(7-Chloro-benzothiazol-2-ylsulfanyl)-9-methyl-9H-purine-6-ylamine (295)

8-(7-Chloro-benzothiazol-2-ylsulfanyl)-9-methyl-9H-purine-6-ylamine was prepared by the same method described in example 271. ¹H NMR (CDCl₃) δ 3.91 (s, 3H), 5.76 (s, 2H, NH₂), 7.36 (d, 1H), 7.45(t, 1H), 7.83 (d, 1H), 8.46(s, 1H). HPLC: RT=5.51 min (method: 5-100-7).

### EXAMPLE 296 4-[6-Amino-8-(7-chloro-benzothiazol-2-ylsulfanyl)-purin-9-yl]-butan-2-one (296)

4-[6-Amino-8-(7-chloro-benzothiazol-2-ylsulfanyl)-purin-9-yl]-butan-2-one was prepared by the same method described in example 271. ¹H NMR (CDCl₃) δ 2.1 (s, 3H), 3.10 (m, 2H), 4.61 (m, 2H), 5.84 (s, 2H), 7.35 (d, 1H), 7.43 (t, 1H), 7.84 (d, 1H), 8.43(s, 1H). HPLC: RT=5.60 min (method: 5-100-7).

### EXAMPLE 297 8-(7-Chloro-benzothiazol-2-ylsulfanyl)-9-(2-ethylsulfinyl-ethyl)-9H-purin-6-ylamine (297)

8-(7-Chloro-benzothiazol-2-ylsulfanyl)-9-(2-ethylsulfinyl-ethyl)-9H-purin-6-ylamine was prepared from 8-(7-Chloro-benzothiazol-2-ylsulfanyl)-9-(2-ethylsulfanyl-ethyl)-9H-purin-6-ylamine (see example 290) by treating it with H₂O₂ in HOAC at rt. ¹H NMR (CDCl₃) δ 1.29 (t, 3H), 2.75 (m, 2H), 3.20(m, 1H), 3.33 (m, 1H), 4.80 (m, 1H), 4.88(m, 1H), 6.11 (s, 2H), 7.35 (d, 1H), 7.43 (t, 1H), 7.84 (d, 1H), 8.43(s, 1H). HPLC: RT=5.05 min (method: 5-100-7).

### EXAMPLE 298 4-[6-Amino-8-(7-chloro-benzothiazol-2-ylsulfanyl)-purin-9-yl]-butan-2-thione (298)

4-[6-Amino-8-(7-chloro-benzothiazol-2-ylsulfanyl)-purin-9-yl]-butan-2-thione was prepared by the same method described in example 297. ¹H NMR (CDCl₃) δ 2.6 (s, 3H), 3.22 (m, 1H), 3.43 (m, 1H), 4.81 (m, 1H), 4.92 (m, 1H), 5.77 (s, 2H), 7.35 (d, 1H), 7.43 (t, 1H), 7.84 (d, 1H), 8.63(s, 1H). HPLC: RT=4.87 min (method: 5-100-7).

### EXAMPLE 299 8-(7-Chloro-benzothiazol-2-ylsulfanyl)-9-(2-ethanesulfonyl-ethyl)-9H-purin-6-ylamine (299)

8-(7-Chloro-benzothiazol-2-ylsulfanyl)-9-(2-ethanesulfonyl-ethyl)-9H-purin-6-ylamine was prepared from 8-(7-Chloro-benzothiazol-2-ylsulfanyl)-9-(2-ethylsulfanyl-ethyl)-9H-purin-6-ylamine (see example 290) by treating it with mCPBA in CH₂Cl₂ at rt. ¹H NMR (CDCl₃) δ 1.37 (t, 3H), 3.00 (m, 2H), 3.28(t, 2H), 4.86(t, 2H), 5.76 (s, 2H), 7.35 (d, 1H), 7.43 (t, 1H), 7.84 (d, 1H), 8.43(s, 1H). HPLC: RT=5.39 min (method: 5-100-7).

### EXAMPLE 300 8-(7-Chloro-benzothiazol-2-ylsulfanyl)-9-(2-methanesulfonyl-ethyl)-9H-purin-6-ylamine (300)

8-(7-Chloro-benzothiazol-2-ylsulfanyl)-9-(2-methanesulfonyl-ethyl)-9H-purin-6-ylamine was prepared by the same method described in example 299. ¹H NMR (CDCl₃) δ 2.96 (s, 3H), 3.76(t, 2H), 4.87(t, 2H), 5.76 (s, 2H), 7.37 (d, 1H), 7.43 (t, 1H), 7.84 (d, 1H), 8.43(s, 1H). HPLC: RT=5.30 min (method: 5-100-7)

### EXAMPLE 301 [2-(6-Amino-9-butyl-9H-purin-8-ylsulfanyl)-benzothiazol-7-yl]-methanol

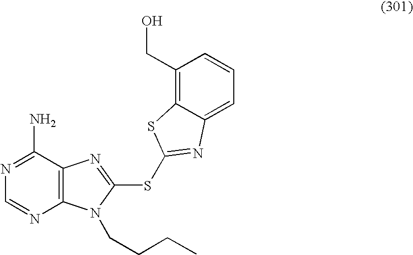

[2-(6-Amino-9-butyl-9H-purin-8-ylsulfanyl)-benzothiazol-7-yl]-methanol was prepared from 9-Butyl-8-(7-methoxymethoxymethyl-benzothiazol-2-ylsulfanyl)-9H-purine-6-ylamine (187) by treating it with HCl in MeOH at 65° C. for 15 min. ¹H NMR (CDCl₃) δ 0.90 (t, 3H), 1.33 (m, 2H), 1.84 (m, 2H), 4.33 (t, 2H), 4.89 (s, 2H), 6.19 (bs, 2H), 7.29(d, 1H), 7.43 (t, 1H), 7.87 (d, 1H), 8.42 (s, 1H). HPLC: RT=5.36 min (method: 5-100-7).

### EXAMPLE 302 9-[2-Isopropylamino-ethyl]-8-(7-methoxy-benzothiazol-2-ylsulfanyl)-9H-purin-6-yl amine (302)

9-[2-Isopropylamino-ethyl]-8-(7-methoxy-benzothiazol-2-ylsulfanyl)-9H-purin-6-yl amine was prepared by the same method described in Example 247. ¹H NMR (CD₃OD) δ 8.29(s, 1H), 7.50(d, J=1.08 Hz, 1H), 7.48(t, J=8.4 Hz, 1H), 6.99(d, J=1.08 Hz, 1H), 4.50(t, J=5.09, 2H, CH₂), 3.97(s, 3H, CH₃), 3.07(t, J=5.09, 2H, CH₂), 2.87(m, 1H, CH), 1.00(d, J=6.32, 6H, 2CH₃). HPLC: RT=4.575 (Method: 5-100-7).

### EXAMPLE 303 9-[2-tert-Butylamino-ethyl]-8-(7-methoxy-benzothiazol-2-ylsulfanyl)-9H-purin-6-yl amine (303)

9-[2-tert-Butylamino-ethyl]-8-(7-methoxy-benzothiazol-2-ylsulfanyl)-9H-purin-6-yl amine was prepared by the same method described in Example 247. ¹H NMR (CD₃OD) δ 8.29(s, 1H), 7.50(d, J=1.08 Hz, 1H), 7.48(t, J=8.4 Hz, 1H), 6.99(d, J=1.08 Hz, 1H), 4.47(t, J=5.09, 2H, CH₂), 3.97(s, 3H, CH₃), 3.07(t, J=5.09, 2H, CH₂), 2.87(m, 1H, CH), 1.02(s, 9H, 3CH₃). HPLC: RT=4.727 (Method: 5-100-7).

### EXAMPLE 304 9-(2-Isobutylamino-ethyl)-8-(7-methoxy-benzothiazol-2-ylsulfanyl)-9H-purin-6-yl amine (304)

9-(2-Isobutylamino-ethyl)-8-(7-methoxy-benzothiazol-2-ylsulfanyl)-9H-purin-6-yl amine was prepared by the same method described in Example 247. ¹H NMR (CD₃OD) δ 8.29(s, 1H), 7.50(d, J=1.08 Hz, 1H), 7.48(t, J=8.4 Hz, 1H), 6.99(d, J=1.08 Hz, 1H), 4.507(t, J=5.09, 2H, CH₂), 3.97(s, 3H, CH₃), 3.02(t, J=5.09, 2H, CH₂), 2.38(d, J=6.89, 2H, CH₂), 1.64(m, 1H, CH), 0.83(s, 6H, 2CH₃). HPLC: RT=4.869 (Method: 5-100-7).

### EXAMPLE 305 6-Amino-8-(7-methyl-benzothiazol-2-ylsulfanyl)-purin-9-yl]-propan-ol (305)

3-[6-Amino-8-(7-methyl-benzothiazol-2-ylsulfanyl)-purin-9-yl]-propan-ol was prepared by the same method described in 244. ¹H NMR (CDCl₃) δ 1.91(m, 2H), 2.50(s, 3H), 3.45(t, 2H), 4.54 (t, 2H), 5.78 (bs, 2H), 7.02 (d, 1H), 7.40 (t,1H), 7.79 (d, 1H), 8.42(s,1H); HPLC: RT=4.96 min (method 5-100-7).

### EXAMPLE 306 9-But-3-enyl-8-(7-chloro-benzothoazol-2-ylsulfanyl)-9H-purine-6-ylamine (306)

9-But-3-enyl-8-(7-chloro-benzothoazol-2-ylsulfanyl)-9H-purine-6-ylamine was prepared by the same method described in example 232. ¹H NMR (CDCl₃) δ2.14 (m, 2H), 4.36 (t, 2H), 5.04 (m, 2H), 5.74(m, 1H), 5.90(bs, 2H), 7.35(d, 1H), 7.44 (t, 1H), 7.84(d,1H), 8.45(s, 1H); HPLC: RT=6.185 min (method: 5-100-7).

### EXAMPLE 307 8-(7-Chloro-benzothoazol-2-ylsulfanyl)-9-pent-4-enyl-9H-purine-6-ylamine (307)

8-(7-Chloro-benzothoazol-2-ylsulfanyl)-9-pent-4-enyl-9H-purine-6-ylamine was prepared by the same method described in example 271. ¹H NMR (CDCl₃) δ1.97 (m, 2H), 2.14(m, 2H), 4.36 (t,2H), 5.04(m,2H), 5.74 (m,1H), 5.90(bs,2H), 7.35(d,1H), 7.44 (t,1H), 7.84(d,1H), 8.45(s,1H); HPLC: RT=6.488 min (method (5-100-7).

### EXAMPLE 308 8-(7-Chloro-benzothoazol-2-ylsulfanyl)-9-hex-5-enyl-9H-purine-6-ylamine (308)

8-(7-Chloro-benzothoazol-2-ylsulfanyl)-9-hex-5-enyl-9H-purine-6-ylamine was prepared by the same method described in example 271. ¹H NMR (CDCl₃) δ1.41 (m, 2H), 1.83(m, 2H), 2.02 (m, 2H), 4.33 (t, 2H), 4.92 (m 2H), 5.64(m, 1H), 5.75(bs,2H), 7.33(d,1H), 7.42 (t,1H), 7.82(d,1H), 8.42(s,1H); HPLC: RT=6.761 min (method 5-100-7).

### EXAMPLE 309 8-(2-iodo-5-methoxy-phenylsulfanyl)-9-pent-4-ynyl-9H-purin-6-ylamine (309)

The preparation of 8-(2-iodo-5-methoxy-phenylsulfanyl)-9-pent-4-ynyl-9H-purin-6-ylamine has been described elsewhere (see Kasibhatla et. al. WO 3037860, 2003 and Llauger et. al. J. Med. Chem. 2005, 48, 2892-2905).

### EXAMPLE 310 9-Butyl-8-(3-methoxy-benzyl)-9H-purin-6-ylamine (310)

### Step 1 N-(5,6-diamino-pyrimidin-4-yl)-2-(3-methoxy-phenyl)-acetamide hydrochloride

4,5,6-Triaminopyrimidine (6.25 g, 50 mmol) was dissolved in N-methyl-2-pyrrolidone (NMP, 70 mL) at 70° C. The solution was cooled to rt, and treated with 3-methoxyphenylacetyl chloride (9.2 g, 50 mmol, 1.0 equiv.) for 3 h at 50° C., whereupon the desired compound precipitated as its HCl salt. The precipitate was collected, washed with EtOAc and acetone, and dried to give the product as a white solid (15.2 g, 98%). HPLC Purity: 97.4%. t_{R}=4.13 min (Conditions I). mp=286-288° C. ¹H NMR (DMSO-d₆) δ 9.22 (s, 1H), 8.20 (s, 1H), 6.75-7.58 (br. s, 4H), 7.21 (t, J=7.9 Hz, 1H), 6.95 (d, J=1.3 Hz, 1H), 6.90 (d, J=7.6 Hz, 1H), 6.80 (dd, J=7.6 & 1.3 Hz, 1H), 3.74 (s, 2H), 3.73 (s, 3H). ¹³C NMR (DMSO-d₆) δ 170.9, 159.4, 156.4 (2C), 147.9, 137.7, 129.3, 122.8, 116.2, 112.2, 93.9, 55.4, 41.8

### Step 2 8-(3-Methoxy-benzyl)-9H-purin-6-ylamine

A solution of crude N-(5,6-diamino-pyrimidin-4-yl)-2-(3-methoxy-phenyl)-acetamide hydrochloride (17.6 g, 57 mmol) and MeONa (12.3 g, 227 mmol, 4.0 equiv.) in n-BuOH (150 mL) was heated to reflux for 2 h, cooled to rt, and neutralized with HCl 2M. Brine was added, which gave a bi-phasic mixture. Concentration of the organic layer afforded the title compound as a solid (10.7 g, 75%). t_{R}=4.70 min (Conditions II). mp=252-254° C. ¹H NMR (DMSO-d₆) δ 8.06 (s, 1H), 7.43 (br. s, 1H), 7.21 (t, J=7.9 Hz, 1H), 7.05 (s, 2H), 6.93 (s, 1H), 6.87 (d, J=7.6 Hz, 1H), 6.79 (dd, J=8.1 & 2.3 Hz, 1H), 4.09 (s, 2H), 3.73 (s, 3H). ¹³C NMR (DMSO-d₆) δ 159.8, 155.5, 152.3, 151.6, 150.8, 139.3, 130.0, 121.4, 119.0. 115.0, 112.4, 55.5, 35.5.

Step 3 A mixture of 8-(3-methoxy-benzyl)-9H-purin-6-ylamine (0.50 g, 2.2 mmol), BuI (0.30 mL, 2.65 mmol, 1.2 equiv.), Cs₂CO₃ (1.43 g, 4.4 mmol, 2.0 equiv.), and DMF (2.5 mL) was stirred at rt for 16 h. Flash chromatography (MeOH:CH₂Cl₂ 5:95) gave the title compound as a white solid (370 mg, 54%). HPLC Purity: 91.0%. t_{R}=6.92 min (Conditions II). mp=163-165° C. ¹H NMR (CDCl₃:CD₃OD 5:1) δ 8.13 (s, 1H), 7.16 (t, J=7.9 Hz, 1H), 6.73-6.67 (m, 3H), 4.13 (s, 2H), 3.95 (t, J=7.7 Hz, 2H), 3.68 (s, 3H), 1.48 (quint., J=7.7 Hz, 2H), 1.20 (sext., J=7.5 Hz, 2H), 0.78 (t, J=7.4 Hz, 3H). ¹³C NMR (CDCl₃:CD₃OD 5:1) δ 159.9, 154.7, 152.0, 150.9, 150.7, 136.6, 129.9, 120.8, 117.8, 114.5, 112.3, 55.07, 42.9, 34.2, 31.5, 19.8, 13.4. HRMS: calcd for C₁₇H₂₂N₅O (MH)⁺ m/z 312.1819, found 312.1817.

### EXAMPLE 311 9-Butyl-8-(2-chloro-5-methoxy-benzyl)-9H-purin-6-ylamine (311)

A solution of 9-butyl-8-(3-methoxy-benzyl)-9H-purin-6-ylamine (310) (100 mg, 0.32 mmol) in THF (4 mL) was treated with SO₂Cl₂ (78 μL, 0.96 mmol, 3.0 equiv.) at rt for 2 h. Work-up and preparative TLC (MeOH:CH₂Cl₂ 10:90) gave the title compound (60.2 mg, 54%). mp=138-139° C. HPLC Purity: 92.4%. t_{R}=7.77 min (Conditions II). ¹H NMR (CDCl₃) δ 8.31 (s, 1H), 7.30 (d, J=8.8 Hz, 1H), 6.75 (dd, J=8.8 & 3.0 Hz, 1H), 6.67 (d, J=3.0 Hz, 1H), 6.26 (s, 2H), 4.32 (s, 2H), 4.04 (t, J=7.7 Hz, 2H), 3.67 (s, 3H), 1.62 (quint., J=7.7 Hz, 2H), 1.30 (sext., J=7.5 Hz, 2H), 0.87 (t, J=7.4 Hz, 3H). ¹³C NMR (CDCl₃) δ 157.1, 153.6, 150.9, 149.8, 148.4, 133.1, 128.8, 123.4, 117.3, 114.7, 112.5, 54.0, 41.5, 30.4, 30.2, 18.6, 12.2. HRMS: calcd for C₁₇H₂₁N₅ClO (MH)⁺ m/z 346.1429, found 346.1426

Biological Testing of Compounds

### EXAMPLE 312 FLUORESCENCE-BASED COMPETITIVE BINDING ASSAY FOR BIOTINYLATED-GELDANAMYCIN TO PURIFIED HSP90

This assay directly measures the binding of biotinylated-geldanamycin (biotin-GM) to purified Hsp90 and thus tests the ability of compounds to compete for binding to Hsp90.

Purified native Hsp90 protein (mixture of alpha and beta) from HeLa cells (Stressgen Biotechnologies Corp., San Diego, Calif., USA) was coated onto 96-well plates by incubating for 1 hr at 37° C. Uncoated Hsp90 was removed and the wells washed twice in 1×PBS (phosphate-buffered saline) buffer. Biotin-GM was then added to the wells, and the reaction was further incubated for 1 hr 37° C. The wells were washed twice with 1×PBS, before the addition of 20 ug/ml streptavidin-phycoerythrin, and incubated for 1 hr at 37° C. The wells were again washed twice with 1×PBS. The fluorescence was then measured in a Gemini spectrofluorometer (Molecular Devices) using an excitation of 485 nm and emission of 580 nm.

### EXAMPLE 313 SCREENING OF COMPOUNDS OF THE INVENTION FOR HSP90 BINDING ABILITY

The compounds in the table below were prepared as described above and evaluated for HSP90 binding ability based on the above assay (example 312).

| | | IC50 |
|---|---|---|
| Example # | Compound # | μM |
| 1.1 | 1 | 10 |
| 2.1 | 2 | 2 |
| 2.2 | 3 | 1.1 |
| 2.4 | 5 | 2.0 |
| 3.2 | 8 | 6 |
| 3.4 | 10 | 2.8 |
| 4.7 | 26 | 1.1 |
| 4.8 | 27 | 0.9 |
| 4.9 | 28 | 2.3 |
| 4.10 | 29 | 0.9 |
| 9.4 | 44 | 1.5 |
| 9.5 | 45 | 1.8 |
| 9.6 | 46 | 0.9 |
| 9.7 | 47 | 0.8 |
| 11.3 | 57 | 4.0 |
| 11.10 | 63 | 1.3 |

### EXAMPLE 314 HER2 INHIBITION ASSAY

MCF-7 cells are seeded in 24 well plates at a density of approximately 30,000 cells/well and allowed to grow for 16 hours in DMEM supplemented with 10% FBS. Drug is then added at a concentration range of 100 uM to 0.01 uM. Cells are incubated for an additional 24. Drug treated cells and untreated control cells are trypsinized, and incubated at room temperature for 15 minutes with anti Her-2 neu Ab conjugated with phycoerythrin (Becton Dickinson, San Jose Calif.; Cat no. 340552) at a concentration of 0.25 ug/ml, or non-specific control IgG1 conjugated with phycoerythrin (Becton Dickinson, San Jose Calif.; Cat no. 340761). Samples were analyzed using a FACS Calibur flow cytometer (Becton Dickinson) equipped with Argon-ion laser that which emits 15 mW of 488 nm light for excitation of the phycoerythrin fluorochrome. 10,000 events were collected per sample. A fluorescence histogram was generated and the mean fluorescence intensity (mfi) of each sample was determined using Cellquest software. The background was defined as the mfi generated from cells incubated with control IgG, and was subtracted from each sample stained with the HER-2/neu Ab Percent degradation of Her-2 was calculated as follows:
% Her-2 degradation=(mfi HER-2 sample)/(mfi HER-2 untreated cells)×100

### EXAMPLE 315 SCREENING OF COMPOUNDS OF THE INVENTION FOR HER-2 DEGRADATION ABILITY

The compounds in the table below were prepared as described above and evaluated for Her-2 degradation ability based on the above assay (example 314).

Inhibitory Concentration 50 (IC₅₀) for this assay is the concentration necessary to degrade 50% of Her 2 expression (protein).

| | | IC50 |
|---|---|---|
| Example # | Cmpound # | μM |
| 1.1 | 1 | 6.0 |
| 2.1 | 2 | 0.6 |
| 2.2 | 3 | 0.5 |
| 2.4 | 5 | 1.0 |
| 3.2 | 8 | 1.5 |
| 3.4 | 10 | 1.5 |
| 4.7 | 26 | 1.5 |
| 4.8 | 27 | 0.8 |
| 4.9 | 28 | 1.0 |
| 4.10 | 29 | 0.8 |
| 9.4 | 44 | 1.5 |
| 9.5 | 45 | 2.0 |
| 9.6 | 46 | 0.3 |
| 9.7 | 47 | 0.3 |
| 11.3 | 57 | 1.4 |
| 11.10 | 63 | 0.7 |

Optimization of Compound Activity

One of the strategies for improving the activity of purine-based Hsp90 inhibitors was to independently optimize the substituents on the benzene ring of 8-benzyladenines and the nature of the linker spanning between the benzene and the purine rings. The preferred structural elements emerging from both optimizations could then be combined and compounds selected for acceptable pharmaceutical properties. This plan allowed us to take full advantage of the known methods for the preparation of 8-benzylpurines, although in some cases refinements proved to be necessary.

### EXAMPLE 316 HER-2 DEGRADATION ASSAY

The potency of the compounds was assessed using a HER-2 degradation assay, which has been described elsewhere, (Le Brazidec et. al. J. Med. Chem. 2004, 47, 3865-3873). Briefly, compounds were incubated for 16 h with MCF-7 cells, a breast cancer cell line expressing on its surface medium levels of the HER-2 receptor, which is a Hsp90 client. Inhibition of Hsp90 induces the degradation of HER-2, which was monitored with a combination of phycoerythrin-labeled antibody and flow cytometry. This assay is highly reproducible, with 17-AAG consistently giving an HER-2 IC₅₀ of 12.9±0.3 nM, wherein the error refers to the standard error of the mean (SEM).

### EXAMPLE 317 EXAMINATION OF THE EFFECT OF SUBSTITUENTS ON THE BENZENE RING

The 2,5-dimethoxy substitution pattern emerged as more potent than the prototypic 3,4,5-trimethoxy pattern. Replacing the 2-MeO group by Cl marginally decreased the activity, but replacing it with Br or I led to an increase in activity. The effect of the linker was investigated. The compounds with a NH or O as linker are inactive, and it was assumed that only the CH₂ linker could be tolerated. However, upon introduction of an S linker it was observed that the sulfur atom was superior to the original CH₂ linker.

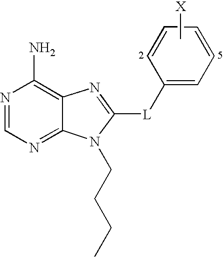

| Cmpd # | ID | L | X | HER-2 IC₅₀ [μM] |
|---|---|---|---|---|
| PU3 | 3a | CH₂ | 3,4,5-triMeO | 40 |
| 1 | 8 | CH₂ | 2,5-diMeO | 12 |
| 311 | 12b | CH₂ | 2-Cl, 5-MeO | 20 |
| 23 | 12c | CH₂ | 2-Br, 5-MeO | 8.0 |
| 20 | 12d | CH₂ | 2-I, 5-MeO | 5.0 |
| 43 | 18 | S | 2,5-diMeO | 3.5 |

All values represent the average of at least three independent observations.

The standard errors of the mean (SEM) are 6-11% of the mean value.

### EXAMPLE 318 EXAMINATION OF THE EFFECT OF N(9) SIDE-CHAIN

The N(9) side-chain was next optimized, and over 100 analogues of the 2,5-dimethoxybenzyl adduct were screened. The homoprenyl side-chain emerged as an equipotent alternative to the already disclosed pent-4-ynyl side-chain, both analogs having an HER-2 IC₅₀=1.5 μM. Thus, having separately optimized the benzene ring substituents (2-iodo-5-methoxy), the linker (—S—), and the side-chain (homoprenyl or pent-4-ynyl), we examined the combination of these preferred structural features. The homoprenyl analog and the pent-4-ynyl analog had similar potencies (HER-2 IC₅₀≈0.3 μM). However, the addition of a 2-F substituent on the adenine ring, an operation known to be favorable in the 8-benzyladenine series, did not bring additional activity to the 8-sulfanyl series.

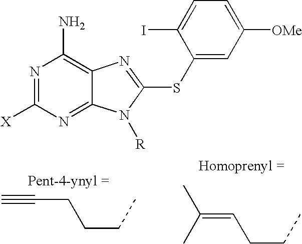

| Entry | ID | X | R | HER-2 IC₅₀ [μM] |
|---|---|---|---|---|
| 309 | 4 | H | Pent-4-ynyl | 0.28 |
| 46 | 23 | H | Homoprenyl | 0.37 |
| 68 | 28 | F | Pent-4-ynyl | 0.36 |

All values represent the average of at least three independent observations. The standard errors of the mean (SEM) are 6-11% of the mean value.

Although these compounds exhibited improvements in potency over previously reported Hsp90 inhibitors, they proved to be poorly water-soluble, especially with the 2-iodo substituent. This hampered their formulation, and rendered them insufficiently orally bioavailable. We therefore sought to incorporate ionizable amino groups in the N(9) side-chain of the inhibitor. The introduction of the amino group not only improved the water solubility, but also increased the potency. The highest potencies were obtained when the amino N atom was separated by 2 or 3 methylene units from the purine ring, and was further substituted with a bulky alkyl group. The most active compound in the 3-atom linker series proved to be the tert-butylamine (HER-2 IC₅₀=140±15 nM), while in the 2-atom linker series the neopentylamine (HER-2 IC₅₀=90±10 nM) showed optimal activity.

### EXAMPLE 319 EXAMINATION OF ABILITY OF AMINE DERIVATIVES TO INHIBIT CELL GROWTH

Amines were tested for their ability to inhibit cell-growth, using a previously described assay to quantify cell proliferation. In brief, MCF-7 breast cancer cells were incubated for 5 days with the test compound, and then treated with MTS (3-(4,5-dimethylthiazol-2-yl)-5-(3-carboxymethoxyphenyl)-2-(4-sulfophenyl)-2H-tetrazolium). The MTS reagent is reduced only by metabolically active cells to the formazan dye, and the number of live cells was deduced by spectrophotometry (490 nM). The MTS IC₅₀ was defined as the concentration of Hsp90 inhibitor that gave rise to 50% less new live cells compared to an untreated culture. In this assay, the control 17-AAG had an MTS IC₅₀ of 32±4 nM, and the standard error of the mean (SEM) associated with this assay ranged from 9-21% of the mean value. The 8-(sulfanyl)adenines proved to be inhibit cell growth with MTS IC₅₀ values typically in the 200-500 nM range, which is roughly within 1 logarithmic unit of the gold standard 17-AAG.

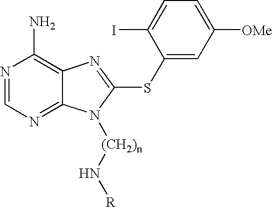

| | | | | HER-2 | MTS |
|---|---|---|---|---|---|
| Cmpd # | ID | n | R | IC₅₀ [μM]^{a} | IC₅₀ [μM]^{b} |
| PU24FCl | 3b | | | 1.7 | 1.2 |
| 309 | 4 | | | 0.29 | 0.7 |
| 108 | 37 | 3 | Et₂CH— | 0.21 | 0.2 |
| 109 | 38 | 3 | EtMeCH— | 0.21 | 0.2 |
| 126 | 39 | 3 | i-Pr- | 0.18 | 0.6 |
| 95 | 40 | 3 | t-Bu- | 0.14 | 0.2 |
| 89 | 41 | 2 | i-Bu- | 0.10 | 0.2 |
| 132 | 42 | 2 | t-BuCH₂— | 0.09 | 0.5 |

(a) For the HER-2 degradation assay, the values represent the average of at least three independent observations, and the standard errors of the mean (SEM) are 6-11% of the mean value. (b) For the growth inhibition assay, the values represent the average of at least three independent observations, and the standard errors of the mean (SEM) are 9-21% of the mean value.

### EXAMPLE 320 SELECTIVITY ASSESSMENT

The selectivity of 132 for Hsp90 over other ATP-binding proteins was assessed with a panel of human kinases (Aurora-A, CHK2, IKKα, MAPK1, MAPK2, MEK1, PDK1, Plk3, PI-3K, c-Raf, c-Src), none of which were significantly inhibited at 10 μM.

### EXAMPLE 321 IN VIVO PHARMACOKINETICS

Perhaps the most important feature of the amine compounds, besides their potency, was their dramatically increased water-solubility. Once converted to their H₃PO₄ salt, these amines provided excellent water solubility (>10 mg/mL), and were readily administered in standard aqueous solutions. For animal studies, they were formulated in a phosphatidylcholine/water dispersion. The pharmacokinetics of these compounds was determined in Balb/C mice. When the compounds were administered orally at 100 mg/kg, peak plasma concentrations (Cₘₐₓ) between 4.8 and 9.7 μg/mL (10-19 μM) were achieved. The plasma concentrations peaked at Tₘₐₓ=30 min, indicating rapid absorption, and dropped below the detection limit (0.5 μg/mL, 1 μM) after 1-4 h to give, when integrated over a 4 h period, AUC values of 240 to 680 min·μg/mL (equivalent to 8-22 μM·hr). The effect of the solubility on the oral bioavailability was striking, and the % F increased from <10% for the pentyne derivative to 14-97% for the amines 95, 89, 108, 109, 126, 132 and 132. When administered intravenously at 10 mg/kg, these amines were cleared at the rate of 33-131 mL/min/kg, which is quite high compared to the total liver blood flow (90 mL/min/kg for mice). We did not determine, however, if the clearance was due to metabolism, distribution, or inadequate protein binding. By analogy with the structurally related adenines, it is also possible that the inhibitors 95, 89, 108, 109, 126, 132 accumulate in the tumor to concentrations exceeding those in the plasma. In spite of their high clearance at 10 mg/kg, the oral bioavailability of compounds 126, 95, and 132 at 100 mg/kg was equal or greater than 50%, suggesting that the clearance was saturated at 100 mg/kg.

Pharmacokinetic Parameters of Selected Amines.

| | | IV Parameters | | | | | PO Parameters | | |
|---|---|---|---|---|---|---|---|---|---|
| | | Cl | T_{1/2} | V_{SS} | Cₘₐₓ | Tₘₐₓ | AUC | T_{1/2} | % F |
| Cmpd # | Compound | [mL/min/kg] | [h] | [L/kg] | [μg/mL] | [h] | [min · μg/mL] | [h] | [%] |
| 108 | 37 | 69 | 0.5 | 2.4 | 5.8 | 0.5 | 590 | 0.6 | 42 |
| 109 | 38 | 33 | 1.3 | 3.4 | 4.8 | 0.5 | 380 | 0.7 | 28 |
| 126 | 39 | 62 | 4.2 | 22 | 4.7 | 0.5 | 450 | 1.5 | 55 |
| 95 | 40 | 131 | 1.6 | 24 | 6.7 | 0.5 | 680 | 0.9 | 97 |
| 89 | 41 | 47 | 0.3 | 0.4 | 6.7 | 0.5 | 290 | 0.3 | 14 |
| 132 | 42 | 64 | 0.4 | 1.6 | 9.5 | 0.5 | 760 | 0.5 | 50 |

The compounds were formulated as H₃PO₄ salts in a phosphatidylcholine/water dispersion and delivered intravenously (IV) at 10 mg/kg or orally (PO) at 100 mg/kg. Plasma concentrations were measured at six time points over 4 hours, and the pharmacokinetic parameters were determined using non-compartmental methods (WinNonlin Professional, Version 4.1). The terminal half-life was calculated using 3-4 data points.

This is to our knowledge the first time that pharmacologically relevant concentrations of Hsp90 inhibitors have been achieved via the oral route, and these results suggested that these inhibitors may be orally active. For instance, a Cₘₐₓ=5.8 μg/mL (108) corresponds to a concentration of 12 μM, which is approximately 50-fold higher than the concentrations required to either induce HER-2 degradation in MCF-7 cells (HER-2 IC₅₀=0.21 μM) or to inhibit the proliferation of MCF-7 cells (MTS IC₅₀=0.2 μM). The plasma concentration of the amines 95, 89, 108, 109, 126, 132 remained above 1 μM, the detection limit, for 1-4 h.

### EXAMPLE 322 IN VIVO INDUCTION OF THE DEGRADATION OF HSP90 CLIENTS

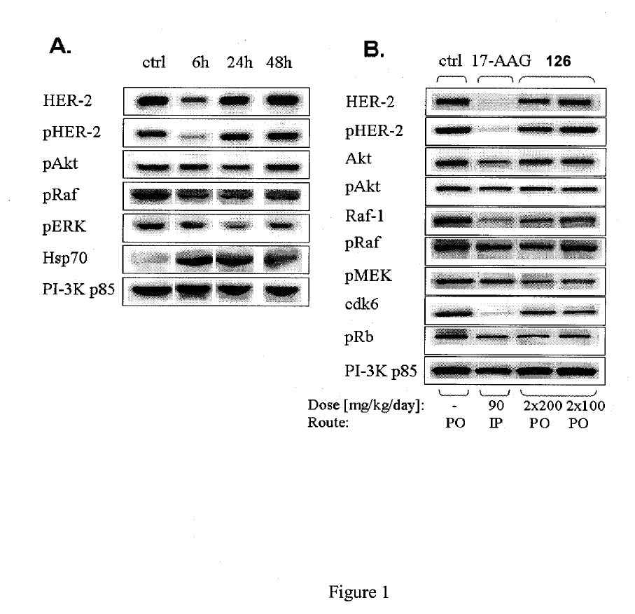

These amines provided a good combination of potency, ease of formulation, and bioavailability, but displayed relatively high clearance values in mice. We next verified the ability of the arbitrarily chosen amine 89 to induce the degradation of Hsp90 clients in vivo. Nude mice were implanted with A549 lung cancer cells, a cell line dependent on the Hsp90 clients Raf-1 and Akt for cell proliferation, and were administered a single oral dose of 89.H₃PO₄ (200 mg/kg). The mice were sacrificed at 6, 24, or 48 hrs, the tumors were harvested, and Hsp90 client proteins were visualized by Western blot. The levels of the Hsp90 clients HER-2 and pHER-2 significantly decreased at 6 h, then gradually reached their normal value after 24-48 h (FIG. 1a). The levels of the Hsp90 clients pAKT and pRaf, and the downstream kinase pERK decreased less dramatically, and were lowest at 24 h. Upregulation of the chaperone Hsp70, a response characteristic of Hsp90 inhibition, was evident and lasted 24-48 h. As expected, the kinase PI-3K, which is not an Hsp90 client, was not affected. These pharmacodynamic data underscore an added benefit of targeting Hsp90, since exposing tumor cells to an Hsp90 inhibitor for a few hours is sufficient to induce the degradation of the client proteins. Once degraded, those client proteins require 6-48 hours to accumulate back to their normal levels, and even if the Hsp90 inhibitor is rapidly cleared as 89, its pharmacological effect can be long lasting. This behavior differs significantly from that of most ATP-competitive kinase inhibitors which, once cleared, allow their target to immediately resume its function.

### EXAMPLE 323 XENOGRAFT MODEL

The pharmacodynamic effect of amine 126 was examined in a N87 xenograft model (FIG. 1b), N87 being a stomach cancer cell line expressing high HER-2 levels. Mice were administered 126.H₃PO₄ orally at two different regimens (2×100 or 2×200 mg/kg/day) for three days, and were sacrificed 24 h after the last dosing. Oral administration of 126.H₃PO₄ at 2×200 mg/kg/day induced the degradation of the Hsp90 clients Akt, pAkt, Raf-1, pRaf, cdk6, and pRb to levels comparable to those obtained with 17-AAG injected intraperitoneally once daily at 90 mg/kg/day. The levels of HER-2 and pHER-2 decreased only partially, probably reflecting the fact that HER-2 and pHER-2 are degraded and re-expressed faster (<24 h) than other Hsp90 clients. A 126.H₃PO₄ dose of 2×100 mg/kg/day was still effective at degrading Akt, pAkt, Raf-1, pMEK, cdk6, and pRb, but promoted little or no degradation of HER-2, pHER-2, and pRaf.

### EXAMPLE 324 REPRESSION OF TUMOR GROWTH

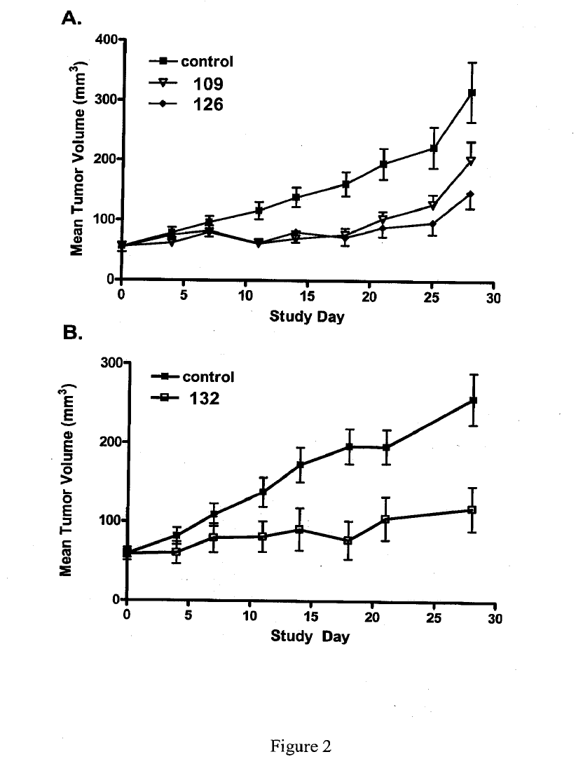

Next, the ability of a subset of amines (109, 126, and 132) to repress tumor growth was examined in murine xenograft models using the N87 stomach cancer cell line, which grew in mice more reproducibly than the A549 cell line. Compounds 109.H₃PO₄ and 126.H₃PO₄ were delivered orally at 200 mg/kg/day (once daily, 5 days/week), in the same experiment (FIG. 2a). Tumor growth inhibition was observed for both compounds, but with a lower statistical significance for 109.H₃PO₄ (p=0.07) compared to 126.H₃PO₄ (p=0.03). At these doses, neither mortality nor weight loss was observed. Similarly, the compound most active in the HER-2 degradation assay, 132.H₃PO₄, was tested in a separate experiment (FIG. 2b), at 200 mg/kg/day but with a different schedule (2×100 mg/kg/day, 5 days/week), and also showed statistically significant (p=0.02) tumor growth inhibition, and no overt toxicity.

The chaperone Hsp90 is a target of interest for the treatment of cancer because of its central regulatory role. Inhibition of Hsp90 induces the degradation of several client proteins, and shuts down multiple oncogenic pathways, which in turn affects a number of critical steps implicated in the genesis of a tumor (proliferation, angiogenesis, acquired immortality, evasion of apoptosis, and metastasis). The simultaneous modulation of various oncogenic effects should reduce the likelihood of the tumor acquiring resistance to Hsp90 inhibitors. In addition, the existence of an activated form of Hsp90 in cancer cells offers the possibility to develop inhibitors selective for malignant cells. Compounds of the present invention as purine-based inhibitors of Hsp90 have been optimized, reaching 90 nM potency as in the HER-2 degradation assay and 200 nM in in vitro growth inhibition assays. The introduction of an amino group in the side-chain dramatically improved their aqueous solubility (>10 mg/kg for their H₃PO₄ salts), which greatly facilitated their formulation for oral delivery. In mice, the oral bioavailability of the compounds of the present invention ranged from 14-97%. These amines reached high plasma concentrations (Cₘₐₓ=10-19 μM; oral dose of 100 mg/kg) but were cleared rapidly (Cl=33-131 mL/min/kg; intravenous dose of 10 mg/kg). When administered orally to mice bearing A549 tumor xenografts (200 mg/kg), compounds of the present invention induced the pharmacodynamic response expected from Hsp90 inhibitors: degradation of the client proteins HER-2, pHER-2, pAKT and pRaf and up-regulation of Hsp70. Similarly, in a murine N87 xenograft model, oral administration of compounds of the present invention (2×200 mg/kg/day) induced the degradation of Hsp90 clients but not of PI-3K. Furthermore, in the N87 model, compounds of the present invention inhibited tumor growth orally at 200 mg/kg/day. These are the first Hsp90 inhibitors reported to inhibit tumor growth upon oral administration, but high doses are currently necessary. Further work is necessary to improve the potency and clearance of these compounds, and to examine alternate xenograft models.

### EXAMPLE 325 OPTIMIZATION OF BENZOLOTHIOPURINE ANALOGS

The structure activity relationship data of the substituted benzolothiopurine analogs is summarized below. The 7′-substitutent is essential for inhibitory activity. When 7′-Cl (5) was moved to alternate sites on the aryl ring (6′-Cl (179) or 5′-Cl (2) or 4′-Cl (3)), the activity dropped from 200 uM to as much as 20 uM. Moreover, replacing the Cl-substituent at the 7′-position with various moieties dramatically affected the potency of Her-2 degradation. For example, HER-2 degradation activity of the 7′-halide, 7′-OCH₃ and 7′-CH₃ substituted benzolothio purine analogs range from 180 to 330 nM, with the 7′-chloro exhibiting the best activity. Replacement of the 7′-OCH₃ group with longer alkyl ethers such as 7′-OCH₂CH₃, reduced the activity by 500 fold. Similarly, replacing the 7′-Cl with 7′-H reduced the activity 25 fold. Disubstitution (6′,7′-dichloro) resulted in a 140 fold loss of activity compared with mono substituted analog. The entirety of these results suggests that the ATP-binding site of Hsp90 is very sensitive to subtle changes at the 7′-position.

Structure activity relationships of the benzothiozole moiety.

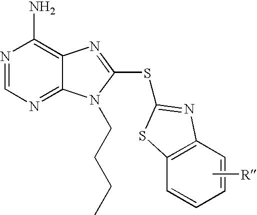

| Compound # | Compound # | R″ | HER-2 IC₅₀(nM) |
|---|---|---|---|
| 178 | 1 | H | 5000 |
| 181 | 3 | 4′-Cl | 15000 |
| 180 | 2 | 5′-Cl | 20000 |
| 179 | 4 | 6′-Cl | 7000 |
| 232 | 5 | 7′-Cl | 180 |
| 171 | 6 | 7′-Br | 330 |
| 175 | 7 | 7′-F | 200 |
| 172 | 8 | 7′-Me | 300 |
| 185 | 9 | 7′Cl, 6′-Cl | 25000 |
| 173 | 10 | 7′-OCH₃ | 190 |
| 174 | 11 | 7′-OCH₂CH₃ | 100000 |

### EXAMPLE 326 Optimization of the 9-N-alkyl substituent

Analysis of the N-alkyl substituent shows that both the chemical nature of the linker at the 9-position as well as its length affect the biological activity. Compounds with 2- to 4-carbon linkers at the 9-position give similar activities, even when substituted with various functional groups, including alcohols, esters and some amines. Increasing the length of the alkyl linker beyond 4 carbons decreased the activity. Surprisingly, among the amine substituents, addition of a tertiary-butyl methyl amine to the 2-carbon linker resulted in significant improvement in HER-2 degradation activity (35 nM) over other amine substituents. Moreover, addition of a diethyl phosphate group to the 2-carbon alcohol also showed similar improvement in HER-2 degradation activity (30 nM). It would seem that two carbon linkers provide the optimal scaffold.

Structure activity relationships of the 9-N-alkyl position.

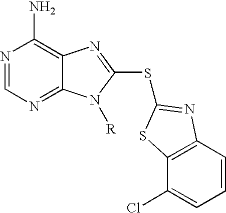

| Compound # | Compound # | R | HER-2 |
|---|---|---|---|
| 229 | 12 | —CH₂CH₃ | 200 |
| 231 | 13 | —CH₂CH₂CH₃ | 250 |
| 232 | 5 | —CH₂CH₂CH₂CH₃ | 180 |
| 233 | 14 | —CH₂CH₂CH₂CH₂CH₃ | 700 |
| 244 | 15 | —CH₂CH₂OH | 300 |
| 245 | 16 | —CH₂CH₂CH₂OH | 150 |
| 246 | 17 | —CH₂CH₂CH₂CH₂OH | 150 |
| 241 | 18 | —CH₂CH₂O₂CCH₃ | 150 |
| 242 | 19 | —CH₂CH₂CH₂O₂CCH₃ | 90 |
| 243 | 20 | —CH₂CH₂CH₂CH₂O₂CCH₃ | 130 |
| 204 | 21 | —CH₂ CH₂PO(OCH₂CH₃)₂ | 30 |
| 284 | 22 | —CH₂CH₂CH₂CH₂CN | 110 |
| 270 | 23 | —CH₂CH₂-morpholine | 250 |
| 265 | 24 | —CH₂CH₂NHC(CH₃)₃ | 140 |
| 247 | 25 | —CH₂CH₂NHCH₂C(CH₃)₃ | 35 |
| 264 | 26 | —CH₂CH₂NHCHCH₂CH₂ | 110 |
| 266 | 27 | —CH₂CH₂CH₂NHCH(CH₃)₂ | 170 |
| 253 | 28 | —CH₂CH₂CH₂NHC(CH₃)₃ | 150 |

### EXAMPLE 327 SUBSTITUTING THE BENZOLOTHIOZOLE WITH A PYRIDOTHIAZOLE RING

Although, the benzothiozole compounds exhibited acceptable potencies in the HER-2-degradation assay, as a class, they were poorly soluble in aqueous media and were subsequently shown to have low oral bioavailability. In an attempt to increase overall oral bioavailability for this class of compounds, we introduced an additional ionizable moiety by substituting the benzolothiozole with a pyridothiazole ring. The HER-2 degradation activity of the most active members of this series was determined. In accordance with the SAR data shown for the benzothiol series, the best analogs contained a 2-carbon linker substituted with the diethyl phosphate moiety.

Structure activity relationships of the pyridothiazole moiety.

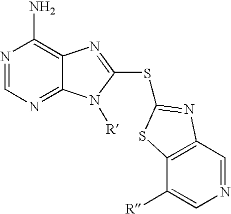

| | Compound # | R′ | R″ | HER-2 |
|---|---|---|---|---|
| 207 | 29 | —CH₂CH₂CH₂CH₃ | Br | 400 |
| 202 | 30 | —CH₂CH₂PO (OCH₂CH₃)₂ | Br | 28 |
| 177 | 31 | —CH₂CH₂CH₂CH₃ | Cl | 280 |
| 206 | 32 | —CH₂CH₂PO (OCH₂CH₃)₂ | Cl | 30 |
| 205 | 33 | —CH₂CH₂CH₂O₂C CH₃ | Cl | 170 |

### EXAMPLE 328 SOLUBILITY ANALYSIS

Comparison of the ethyl-diethyl phosphate analog 206 from the pyridinothiazol series with its benzothioazol analog 204 in a panel of solubility parameters including solubility in simulated gastric fluid, simulated intestinal fluid, and serum, the pyridinothiazol derivative was significantly more soluble in all three solutions.

| | | Gastric | Intestinal | Serum |
|---|---|---|---|---|
| | | (PH 2.0) | (PH 6.5) | (PH 7.4) |
| | Compound # | μg/ml | μg/ml | μg/ml |
| 204 | 21 | 98.5 | * | * |
| 206 | 32 | 124 | 38.3 | 14 |
| 242 | 19 | 220 | 16 | 9 |
| 243 | 20 | 46 | 3 | * |
| 253 | 28 | * | 137.7 | 198.7 |
| 173 | 10 | 35 | 2 | 18 |
| 284 | 22 | 58.7 | 4.4 | 31.3 |
| 247 | 25 | * | 6 | 4.9 |
| 270 | 23 | * | * | * |
| 264 | 26 | 245 | 21 | 59 |

*unable to determine due to degradation

### EXAMPLE 329 IN VIVO PHARMACOKINETICS MEASUREMENTS OF KEY COMPOUNDS

Compounds 242 and 264 also showed biologically relevant concentrations in simulated gastric, intestinal and serum solutions and as a result, 242, 264 and 206 were selected for further examination in vivo. Pharmacokinetic measurements obtained in mice after oral administration at 100 mg/kg are shown below.

| | | Mouse PK PO | Mouse PK PO |
|---|---|---|---|
| | | Cmax at 100 mg/kg | AUC at 100 mg/kg PO |
| | Compound # | ng/mL | (ng/mL*min) |
| 206 | 32 | BLD | BLD |
| 242 | 19 | 603 | 62592 |
| 264 | 26 | 4479 | 430511 |

BLD—Below level of detection

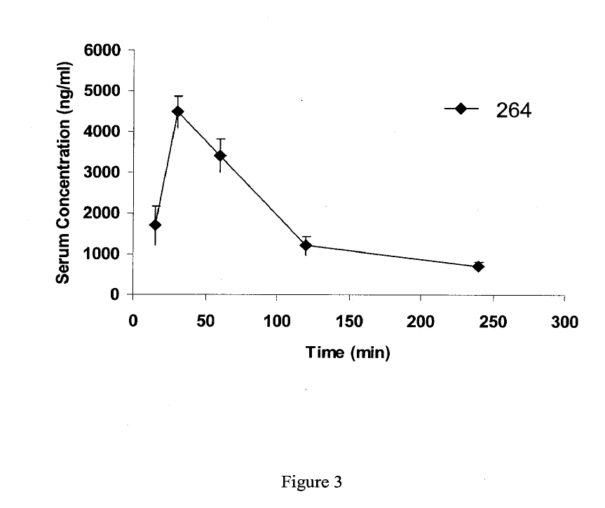

Compound 264 was rapidly absorbed reaching a C-max of 4513 ng/ml 30 min after dosing (see FIG. 3) with half life estimated at 90 minutes. Concentrations for compound 242 were significantly lower than 264. Surprisingly, despite its increased solubility over its benzothiazole analog, concentrations for 206 were not increased and remained below the level of detection perhaps as a result of poor permeability properties.

### EXAMPLE 330 IN VIVO EFFICACY IN THE N87 XENOGRAFT MODEL OF HUMAN STOMACH CANCER

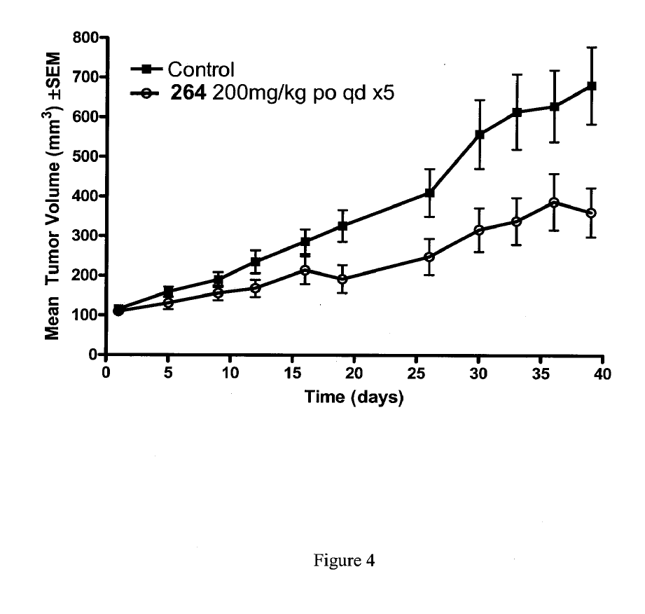

From the sum total of the structure activity data and the pharmaceutics properties in the above studies, compound 264 was notable because it was potent in the HER-2 degradation assay, orally bioavailable and had a reasonable half-life in the mouse. As a result, it was selected for further evaluation in vivo for efficacy in the N87 xenograft model of human stomach cancer. Briefly, N87 tumor fragments were implanted subcutaneously into the flank of athymic nude mice. When the tumors reached an average of 100 mm in size, mice were randomized into groups of 10. Compound 264 was administered orally at 200 mg/kg 5 days/week and on day 39 of this study 56% tumor growth inhibition was observed for 264 as compared with the control group (see FIG. 4). The observed tumor growth inhibition was statistically significant (p<0.05) when analyzed using the t-test.

Hsp90 inhibitors have rapidly become targets of interest for treating cancer as evidenced by numerous recent reports. The ATP-binding site of Hsp90 is amenable to compound optimization and drug development. The compounds of the present invention indicate that the phosphate-binding pocket of the ATP-binding site is large enough to accommodate bicyclic ring systems. However, the ring substitution requirements are very specific, with the 7′-halogens out-performing all other substitution patterns. The bicyclic ring moieties provide the increase in potency necessary for effective inhibition of tumor cell growth while also providing the improvements in pharmaceutics properties required for in vivo activity via the oral route of administration. Since Hsp90 performs a key role not only in regulating proteins associated with oncology pathways, but also in neuropathy and inflammation, it is likely that Hsp90 inhibitors of the class presented here will have additional utility.

The foregoing examples are not limiting and merely illustrative of various aspects and embodiments of the present invention. All documents cited herein are indicative of the levels of skill in the art to which the invention pertains and are incorporated by reference herein in their entireties. None, however, is admitted to be prior art.

One skilled in the art will readily appreciate that the present invention is well adapted to carry out the objects and obtain the ends and advantages mentioned, as well as those inherent therein. The methods and compositions described illustrate preferred embodiments, are exemplary, and are not intended as limitations on the scope of the invention. Certain modifications and other uses will occur to those skilled in the art, and are encompassed within the spirit of the invention, as defined by the scope of the claims.

The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group, and exclusions of individual members as appropriate, or by proviso.

Other embodiments are within the following claims.

What is claimed is:

1. A compound of formula I: 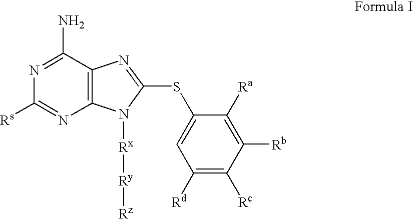
or tautomer or pharmaceutically acceptable salt thereof, wherein
R^{s} is independently selected from H and F;
each R^{a}, R^{b}, R^{c}, and R^{d} is independently selected from H, halo, lower alkyl, OR³, SR³, C(O)N(R⁴)₂, NR⁴R⁴, C(O)R², and —C(O)OR⁴;
R^{x} is independently selected from optionally substituted C₁-C₆ alkyl, optionally substituted C₂-C₆ alkenyl and optionally substituted C₂-C₆ alkynyl;
R^{y} is independently selected from O, NR¹ and a bond;
R^{z} is independently selected from H, C₁-C₆ alkyl, C₂-C₆ alkenyl, C₂-C₆ alkynyl, —P(O)(OR⁴)₂ and C(O)R²;
R¹ is independently selected from H, optionally substituted alkyl, optionally substituted alkenyl, optionally substituted alkynyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;
R² is independently selected from optionally substituted alkyl, optionally substituted cycloalkyl, optionally substituted heteroalkyl, optionally substituted aryl, optionally substituted heterocyclyl and optionally substituted heteroaryl;
R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²; and
R⁴ is independently selected from H, optionally substituted alkyl, optionally substituted cycloalkyl, optionally substituted heteroalkyl, optionally substituted aryl, and optionally substituted heterocyclyl.

2. A compound according to claim 1, wherein at least two of R^{a}, R^{b}, R^{c}, and R^{d} are independently selected from halo and OR³.

3. A compound according to claim 1, wherein at least two of R^{a}, R^{b}, R^{c}, and R^{d} are independently selected from halo and methoxy.

4. A compound according to claim 1, wherein at least three of R^{a}, R^{b}, R^{c}, and R^{d} are independently selected from halo and OR³.

5. A compound according to claim 1, wherein at least three of R^{a}, R^{b}, R^{c}, and R^{d} are independently selected from halo and methoxy.

6. A compound according to claim 1, wherein
R^{a} is halo, and
R^{d} is OR³.

7. A compound according to claims 1 or 5, wherein
R^{x} is optionally substituted C₂-C₃ alkyl, optionally substituted C₂-C₃ alkenyl or optionally substituted C₂-C₃ alkynyl;
R^{y} is NR¹; and
R^{z} is C₁-C₆ alkyl.

8. A compound according to claims 1 or 5, wherein
R^{x} is optionally substituted C₂-C₃ alkyl, optionally substituted C₂-C₃ alkenyl or optionally substituted C₂-C₃ alkynyl;
R^{y} is a bond; and
R^{z} is H.

9. A compound according to claims 1 or 5, wherein
R^{x} is optionally substituted C₂-C₃ alkyl, optionally substituted C₂-C₃ alkenyl or optionally substituted C₂-C₃ alkynyl;
R^{y} is NR¹; and
R^{z} is C(O)R².

10. A compound according to claims 1 or 5, wherein
R^{x} is optionally substituted C₂-C₃ alkyl, optionally substituted C₂-C₃ alkenyl or optionally substituted C₂-C₃ alkynyl;
R^{y} is NH; and
R^{z} is H.

11. A compound according to claim 1 or 5, wherein
R^{x} is optionally substituted C₂-C₃ alkyl, optionally substituted C₂-C₃ alkenyl or optionally substituted C₂-C₃ alkynyl;
R^{y} is NH; and
R^{z} is C₁-C₆ alkyl.

12. A compound according to claim 1 or 5, wherein
R^{x} is optionally substituted C₂-C₃ alkyl;
R^{y} is NH; and
R^{z} is C₁-C₆ alkyl.

13. A compound of formula II: 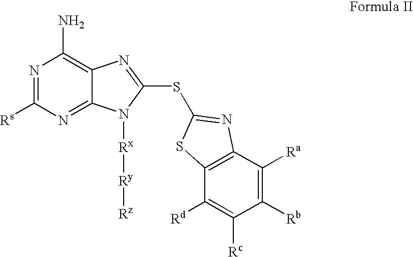
or tautomer or pharmaceutically acceptable salt thereof, wherein
R^{s} is independently selected from H and F;
each R^{a}, R^{b}, R^{c}, and R^{d} is independently selected from H, halo, lower alkyl, OR³, SR³, C(O)N(R⁴)₂, NR⁴R⁴, C(O)R², and —C(O)OR⁴;
R^{x} is independently selected from optionally substituted C₂-C₆ alkyl, optionally substituted C₂-C₆ alkenyl and optionally substituted C₂-C₆ alkynyl;
R^{y} is independently selected from O, NR¹ or a bond;
R^{z} is independently selected from H, C₁-C₆ alkyl, C₂-C₆ alkenyl, C₂-C₆ alkynyl, —P(O)(OR⁴)₂ and C(O)R²;
R¹ is independently selected from H, optionally substituted alkyl, optionally substituted alkenyl, optionally substituted alkynyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;
R² is independently selected from optionally substituted alkyl, optionally substituted cycloalkyl, optionally substituted heteroalkyl, optionally substituted aryl, optionally substituted heterocyclyl and optionally substituted heteroaryl;
R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²; and
R⁴ is independently selected from H, optionally substituted alkyl, optionally substituted cycloalkyl, optionally substituted heteroalkyl, optionally substituted aryl, and optionally substituted heterocyclyl.

14. A compound according to claim 13 wherein
at least one of R^{a}, R^{b}, R^{c}, and R^{d} is halo;
R^{x} is optionally substituted C₂-C₃ alkyl;
R^{y} is a bond; and
R^{z} is H.

15. A compound according to claim 13, wherein
at least one of R^{a}, R^{b}, R^{c}, and R^{d} is halo;
R^{x} is optionally substituted C₂-C₃ alkyl;
R^{y} is NR¹; and
R^{z} is H.

16. A compound according to claim 13, wherein
at least one of R^{a}, R^{b}, R^{c}, and R^{d} is halo;
R^{x} is optionally substituted C₂-C₃ alkyl;
R^{y} is NR¹; and
R^{z} is C₁-C₆ alkyl.

17. A compound according to claim 13, wherein
at least one of R^{a}, R^{b}, R^{c}, and R^{d} is halo;
R^{x} is optionally substituted C₂-C₃ alkyl;
R^{y} is a bond; and
R^{z} is —P(O)(OR⁴)₂.

18. A compound according to claim 13, wherein
at least one of R^{a}, R^{b}, R^{c}, and R^{d} is methoxy;
R^{x} is optionally substituted C₂-C₃ alkyl;
R^{y} is a bond; and
R^{z} is H.

19. A compound of formula III: 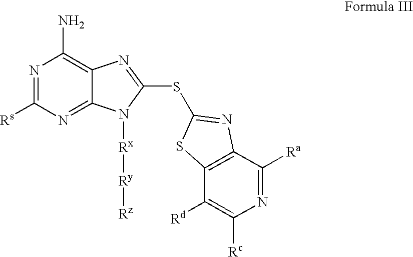
or tautomer or pharmaceutically acceptable salt thereof, wherein
R^{s} is independently selected from H and F;
each R^{a}, R^{c} and R^{d} is independently selected from H, halo, lower alkyl, OR³, SR³, C(O)N(R⁴)₂, NR⁴R⁴, C(O)R², and —C(O)OR⁴;
R^{x} is independently selected from optionally substituted C₂-C₄ alkyl, optionally substituted C₂-C₄ alkenyl and optionally substituted C₂-C₄ alkynyl;
R^{y} is independently selected from O, NR¹ and a bond; and
R^{z} is independently selected from H, C₁-C₆ alkyl, C₂-C₆ alkenyl, C₂-C₆ alkynyl, —P(O)(OR⁴)₂ and C(O)R²;
R¹ is independently selected from H, optionally substituted alkyl, optionally substituted alkenyl, optionally substituted alkynyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;
R² is independently selected from optionally substituted alkyl, optionally substituted cycloalkyl, optionally substituted heteroalkyl, optionally substituted aryl, optionally substituted heterocyclyl and optionally substituted heteroaryl;
R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²; and
R⁴ is independently selected from H, optionally substituted alkyl, optionally substituted cycloalkyl, optionally substituted heteroalkyl, optionally substituted aryl, and optionally substituted heterocyclyl.

20. A compound according to claim 19, wherein
at least one of R^{a}, R^{c}, and R^{d} is halo;
R^{x} is optionally substituted C₂-C₃ alkyl;
R^{y} is a bond; and
R^{z} is H.

21. A compound according to claim 19, wherein
at least one of R^{a}, R^{c}, and R^{d} is halo;
R^{x} is optionally substituted C₂-C₃ alkyl;
R^{y} is NR¹; and
R^{z} is H.

22. A compound according to claim 19, wherein
at least one of R^{a}, R^{c}, and R^{d} is halo;
R^{x} is optionally substituted C₂-C₃ alkyl;
R^{y} is a bond; and
R^{z} is C₁-C₆ alkyl.

23. A compound according to claim 19, wherein
at least one of R^{a}, R^{c}, and R^{d} is halo;
R^{x} is optionally substituted C₂-C₃ alkyl;
R^{y} is a bond; and
R^{z} is —P(O)(OR⁴)₂.

24. A compound of formula IV: 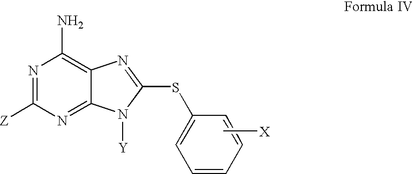
or tautomer or pharmaceutically acceptable salt thereof, wherein
X is independently selected from H, halo, CN, N₃, N(R¹)₂, NR¹S(O)₂R², OR³, SR³, lower alkyl, C(O)N(R⁴)₂, perhaloalkyl, C(O)R², and —C(O)OR⁴;
Y is independently selected from optionally substituted alkyl, optionally substituted alkenyl, optionally substituted alkynyl, optionally substituted aryl, optionally substituted alicyclic, optionally substituted araalkyl, optionally substituted aryloxyalkyl, optionally substituted alkoxyalkyl, optionally substituted heterocyclyl, optionally substituted alkylaminoalkyl (—(CH₂)ₙ—NHR²), optionally substituted alkylaminodialkyl (—(CH₂)ₙ—NR²R²), optionally substituted alkylcarbonylaminoalkyl, (—(CH₂)ₙ—C(O)—NR⁴R⁴), optionally substituted alkylcarbonyloxylalkyl (—(CH₂)ₙ—C(O)—O—R⁴), hydroxyalkyl (—(CH₂)ₙ—OH), haloalkyl (—(CH₂)ₙ-halo), perhaloalkyl, aminoalkyl (—(CH₂)ₙ—NH₂), C(O)R², S(O)₂R², C(O)NR⁴₂, and C(O)OR²;
Z is independently selected from H and halogen;
R¹ is independently selected from H, optionally substituted alkyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;
R² is independently selected from optionally substituted alkyl, optionally substituted cycloalkyl, optionally substituted heteroalkyl, optionally substituted aryl, optionally substituted heterocyclyl and optionally substituted heteroaryl;
R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²;
R⁴ is independently selected from H, optionally substituted alkyl, optionally substituted cycloalkyl, optionally substituted heteroalkyl, optionally substituted aryl, and optionally substituted heterocyclyl; and
n is from 1 to 3.

25. A compound of formula V: 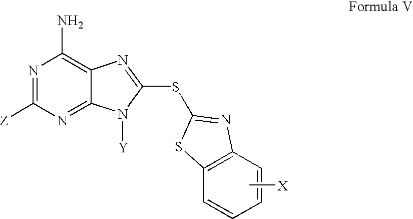
or tautomer or pharmaceutically acceptable salt thereof, wherein
X is independently selected from H, halo, CN, N₃, N(R¹)₂, NR¹S(O)₂R², OR³, SR³, lower alkyl, C(O)N(R⁴)₂, perhaloalkyl, C(O)R², and —C(O)OR⁴;
Y is independently selected from optionally substituted alkyl, optionally substituted alkenyl, optionally substituted alkynyl, optionally substituted aryl, optionally substituted alicyclic, optionally substituted araalkyl, optionally substituted aryloxyalkyl, optionally substituted alkoxyalkyl, optionally substituted heterocyclyl, optionally substituted alkylaminoalkyl (—(CH₂)ₙ—NHR²), optionally substituted alkylaminodialkyl (—(CH₂)ₙ—NR²R²), optionally substituted alkylcarbonylaminoalkyl, (—(CH₂)ₙ—C(O)—NR⁴R⁴), optionally substituted alkylcarbonyloxylalkyl (—(CH₂)ₙ—C(O)—O—R⁴), hydroxyalkyl (—(CH₂)ₙ—OH), haloalkyl (—(CH₂)ₙ-halo), perhaloalkyl, aminoalkyl (—(CH₂)ₙ—NH₂), C(O)R², S(O)₂R², C(O)NR⁴₂, and C(O)OR²;
Z is independently selected from H and halogen;
R¹ is independently selected from H, optionally substituted alkyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;
R² is independently selected from optionally substituted alkyl, optionally substituted cycloalkyl, optionally substituted heteroalkyl, optionally substituted aryl, optionally substituted heterocyclyl and optionally substituted heteroaryl;
R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²;
R⁴ is independently selected from H, optionally substituted alkyl, optionally substituted cycloalkyl, optionally substituted heteroalkyl, optionally substituted aryl, and optionally substituted heterocyclyl; and
n is from 1 to 3.

26. A compound of formula VI: 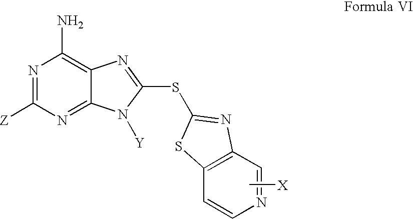
or tautomer or pharmaceutically acceptable salt thereof, wherein
X is independently selected from H, halo, CN, N₃, N(R¹)₂, NR¹S(O)₂R², OR³, SR³, lower alkyl, C(O)N(R⁴)₂, perhaloalkyl, C(O)R², and —C(O)OR⁴;
Y is independently selected from optionally substituted alkyl, optionally substituted alkenyl, optionally substituted alkynyl, optionally substituted aryl, optionally substituted alicyclic, optionally substituted araalkyl, optionally substituted aryloxyalkyl, optionally substituted alkoxyalkyl, optionally substituted heterocyclyl, optionally substituted alkylaminoalkyl (—(CH₂)ₙ—NHR²), optionally substituted alkylaminodialkyl (—(CH₂)ₙ—NR²R²), optionally substituted alkylcarbonylaminoalkyl, (—(CH₂)ₙ—C(O)—NR⁴R⁴), optionally substituted alkylcarbonyloxylalkyl (—(CH₂)ₙ—C(O)—O—R⁴), hydroxyalkyl (—(CH₂)ₙ—OH), haloalkyl (—(CH₂)ₙ-halo), perhaloalkyl, aminoalkyl (—(CH₂)ₙ—NH₂), C(O)R², S(O)₂R², C(O)NR⁴₂, and C(O)OR²;
Z is independently selected from H and halogen;
R¹ is independently selected from H, optionally substituted alkyl, optionally substituted cycloalkyl, optionally substituted heteroalkyl, optionally substituted aryl, optionally substituted heterocyclyl, C(O)R², —C(O)OR², C(O)NR⁴₂, C(S)OR², C(S)NR⁴₂, P(O)(OR⁴)₂, and SO₂R²;
R² is independently selected from optionally substituted alkyl, optionally substituted cycloalkyl, optionally substituted heteroalkyl, optionally substituted aryl, optionally substituted heterocyclyl and optionally substituted heteroaryl;
R³ is independently selected from H, optionally substituted alkyl, optionally substituted cycloalkyl, optionally substituted heteroalkyl, optionally substituted aryl, optionally substituted heterocyclyl, C(O)NR⁴₂, C(O)R², and —C(O)OR²;
R⁴ is independently selected from H, optionally substituted alkyl, optionally substituted cycloalkyl, optionally substituted heteroalkyl, optionally substituted aryl, and optionally substituted heterocyclyl; and
n is from 1 to 3.

27. A pharmaceutical composition comprising the compound, tautomer, or pharmaceutically acceptable salt of any one of claims 1, 13, 19, 24, 25 or 26 and one or more pharmaceutical carriers or excipients.

28. A prodrug of a compound according to any one of claims 1, 13, 19, 24, 25 and 26.

29. A prodrug which is transformed in vivo to a compound according to any one of claims 1, 13, 19, 24, 25 and 26.

30. The prodrug of claim 29 wherein said prodrug is transformed by hydrolysis in blood or in the alimentary tract.

31. A method of inhibiting an HSP90, comprising:
contacting a cell having an HSP90 with a compound, tautomer or pharmaceutically acceptable salt or pharmaceutical composition according to any one of claims 1, 13, 19, 24, 25 and 26.

32. Use of the compound, tautomer thereof, or pharmaceutical salt thereof of any one of claims 1, 13, 19, 24, 25 and 26 for treating one or more of inflammation, infectious disease, autoimmune disease, neurological disorders, cancer and ischemia.

33. The method of claim 31 wherein said contacting is accomplished by oral administration to a subject.

34. The method of claim 31 wherein said contacting is accomplished by topical administration to a subject.

35. The method of claim 31 wherein said cell is a mammalian cell.

36. The method of claim 35 wherein said mammalian cell is human.

37. The method of claim 31 wherein said contacting occurs in vitro.

38. The method of claim 31 wherein said contacting occurs in vivo.

39. The method of claim 31 wherein said contacting is part of an ex vivo procedure.

40. The method of claim 31 wherein said contacting is accomplished by intravenous administration to a subject.

41. The method of claim 31 wherein said contacting is accomplished by parenteral administration to a subject.

42. The method of claim 31 wherein said contacting is performed in situ.

43. The method of claim 31 wherein said contacting is part of a therapy directed against cancer cells.

44. The method of claim 43 wherein said cancer cells are selected from breast cancer cells and melanoma cells.

45. A pharmaceutical composition comprising the compound, tautomer, or pharmaceutically acceptable salt of any one of claims 1, 13, 19, 24, 25 and 26 and at least one other compound.

46. The pharmaceutical composition of claim 45 wherein at least one of said at least one other compound is an inhibitor of HSP90.

47. The pharmaceutical composition of claim 45 wherein at least one of said at least one other compound is an inhibitor of human HSP90.

48. Use of the compound, tautomer thereof, or pharmaceutically acceptable salt thereof of any one of claims 1, 13, 19, 24, 25 or 26 in a chemotherapy regimen.

49. The use of claim 48 wherein said regimen is part of a combinational therapy that makes use of one or more other agents selected from the group consisting of radioisotopes, antibodies, recombinant products, small molecules, antineoplastic agents, Herceptin, taxol, taxanes and taxane derivatives, gleevec, alkylating agents, anti-metabolites; epidophyllotoxin; an antineoplastic enzyme; a topoisomerase inhibitor; procarbazine; mitoxantrone; platinum coordination complexes; biological response modifiers/growth inhibitors; hormonal/anti-hormonal therapeutic agents and haematopoietic growth factors, anthracycline drugs, vinca drugs, mitomycins, bleomycins, cytotoxic nucleosides, tepothilones, discodermolide, pteridine drugs, diynenes, podophyllotoxins, carminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin, podo-phyllotoxin derivatives, etoposide, etoposide phosphate or teniposide, melphalan, vinblastine, vincristine, leurosidine, vindesine, leurosine, paclitaxel, estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

50. A compound selected from the group consisting of:
9-(tert-Butyl-dimethyl-silanyloxymethyl)-8-(2-iodo-5-methoxy-phenylsulfanyl)-9H-purin-6-ylamine; 9-(2-Chloro-ethyl)-8-(2-iodo-5-methoxy-phenylsulfanyl)-9H-purin-6-ylamine; 9-(3-chloro-propyl)-8-(2-iodo-5-methoxy-phenylsulfanyl)-9H-purin-6-ylamine; 9-(4-Chloro-butyl)-8-(2-iodo-5-methoxy-phenylsulfanyl)-9H-purin-6-ylamine; 8-(2-Iodo-5-methoxy-phenylsulfanyl)-9-[3-(4-methyl-piperazin-1-yl)-propyl]-9H-purin-6-ylamine; 9-(3-Dimethylamino-propyl)-8-(2-iodo-5-methoxy-phenylsulfanyl)-9H-purin-6-ylamine; 8-(2-Iodo-5-methoxy-phenylsulfanyl)-9-(3-piperidin-1-yl-propyl)-9H-purin-6-ylamine; 9-(3-Cyclopropylamino-propyl)-8-(2-iodo-5-methoxy-phenylsulfanyl)-9H-purin-6-ylamine; 8-(2-Iodo-5-methoxy-phenylsulfanyl)-9-(3-morpholin-4-yl-propyl)-9H-purin-6-ylamine; 8-(2-Iodo-5-methoxy-phenylsulfanyl)-9-(3-methylamino-propyl)-9H-purin-6-ylamine; 9-(3-Ethylamino-propyl)-8-(2-iodo-5-methoxy-phenylsulfanyl)-9H-purin-6-ylamine; 8-(2-Iodo-5-methoxy-phenylsulfanyl)-9-[2-(4-methyl-piperazin-1-yl)-ethyl]-9H-purin-6-ylamine; 8-(2-Iodo-5-methoxy-phenylsulfanyl)-9-(2-piperidin-1-yl-ethyl)-9H-purin-6-ylamine; 8-(2-Iodo-5-methoxy-phenylsulfanyl)-9-(2-propylamino-ethyl)-9H-purin-6-ylamine; 8-(2,5-Dimethoxy-phenylsulfanyl)-9-(3-dimethylamino-propyl)-9H-purin-6-ylamine; 8-(2-Iodo-5-methoxy-phenylsulfanyl)-9-(2-isopropylamino-ethyl)-9H-purin-6-ylamine; 9-(2-Butylamino-ethyl)-8-(2-iodo-5-methoxy-phenylsulfanyl)-9H-purin-6-ylamine; 9-(2-sec-Butylamino-ethyl)-8-(2-iodo-5-methoxy-phenylsulfanyl)-9H-purin-6-ylamine; 9-[2-(1-Ethyl-propylamino)-ethyl]-8-(2-iodo-5-methoxy-phenylsulfanyl)-9H-purin-6-ylamine; 9-(2-Cyclopropylamino-ethyl)-8-(2-iodo-5-methoxy-phenylsulfanyl)-9H-purin-6-ylamine; 8-(2-Iodo-5-methoxy-phenylsulfanyl)-9-[2-(3-methyl-butylamino)-ethyl]-9H-purin-6-ylamine; 9-[2-(3,3-Dimethyl-butylamino)-ethyl]-8-(2-iodo-5-methoxy-phenylsulfanyl)-9H-purin-6-ylamine; {2-[6-Amino-8-(2-iodo-5-methoxy-phenylsulfanyl)-purin-9-yl]-ethylamino}-acetonitrile; 2-{2-[6-Amino-8-(2-iodo-5-methoxy-phenylsulfanyl)-purin-9-yl]-ethylamino}-ethanol; 8-(2-Iodo-5-methoxy-phenylsulfanyl)-9-[2-(2-methoxy-ethylamino)-ethyl]-9H-purin-6-ylamine; 9-(2-Cyclopentylamino-ethyl)-8-(2-iodo-5-methoxy-phenylsulfanyl)-9H-purin-6-ylamine; 9-(2-Cyclohexylamino-ethyl)-8-(2-iodo-5-methoxy-phenylsulfanyl)-9H-purin-6-ylamine; 9-(2-Cycloheptylamino-ethyl)-8-(2-iodo-5-methoxy-phenylsulfanyl)-9H-purin-6-ylamine; 9-(2-Cyclooctylamino-ethyl)-8-(2-iodo-5-methoxy-phenylsulfanyl)-9H-purin-6-ylamine; 9-[2-(Cyclopropylmethyl-amino)-ethyl]-8-(2-iodo-5-methoxy-phenylsulfanyl)-9H-purin-6-ylamine; 8-(2-Iodo-5-methoxy-phenylsulfanyl)-9-[2-(2-methyl-allylamino)-ethyl]-9H-purin-6-ylamine; 9-(2-tert-Butylamino-ethyl)-8-(2-iodo-5-methoxy-phenylsulfanyl)-9H-purin-6-ylamine; 9-(3-Amino-propyl)-8-(2-iodo-5-methoxy-phenylsulfanyl)-9H-purin-6-ylamine; 9-(2-Cyclopropylamino-ethyl)-8-(2-iodo-5-methoxy-phenylsulfanyl)-9H-purin-6-ylamine; 9-(2-Allylamino-ethyl)-8-(2-iodo-5-methoxy-phenylsulfanyl)-9H-purin-6-ylamine; 8-(2-Iodo-5-methoxy-phenylsulfanyl)-9-(2-morpholin-4-yl-ethyl)-9H-purin-6-ylamine; 8-(2-Iodo-5-methoxy-phenylsulfanyl)-9-(3-propylamino-propyl)-9H-purin-6-ylamine; 9-(3-Heptylamino-propyl)-8-(2-iodo-5-methoxy-phenylsulfanyl)-9H-purin-6-ylamine; 9-(3-Cyclopentylamino-propyl)-8-(2-iodo-5-methoxy-phenylsulfanyl)-9H-purin-6-ylamine; 9-(3-Cyclooctylamino-propyl)-8-(2-iodo-5-methoxy-phenylsulfanyl)-9H-purin-6-ylamine; 8-(2-Iodo-5-methoxy-phenylsulfanyl)-9-(3-isobutylamino-propyl)-9H-purin-6-ylamine; 8-(2-Iodo-5-methoxy-phenylsulfanyl)-9-[3-(1,2,2-trimethyl-propylamino)-propyl]-9H-purin-6-ylamine; 4-{3-[6-Amino-8-(2-iodo-5-methoxy-phenylsulfanyl)-purin-9-yl]-propylamino}-piperidine-1-carboxylic acid tert-butyl ester; 9-(2-Benzylamino-ethyl)-8-(2-iodo-5-methoxy-phenylsulfanyl)-9H-purin-6-ylamine; 9-[3-(1,1-Dimethyl-propylamino)-propyl]-8-(2-iodo-5-methoxy-phenylsulfanyl)-9H-purin-6-ylamine; 9-(3-Cyclobutylamino-propyl)-8-(2-iodo-5-methoxy-phenylsulfanyl)-9H-purin-6-ylamine; 9-(3-Amino-propyl)-8-(2-iodo-5-methoxy-phenylsulfanyl)-9H-purin-6-ylamine; {2-[6-Amino-8-(2-iodo-5-methoxy-phenylsulfanyl)-purin-9-yl]-ethyl}-carbamic acid tert-butyl ester; 9-(2-Amino-ethyl)-8-(2-iodo-5-methoxy-phenylsulfanyl)-9H-purin-6-ylamine; 2-[6-Amino-8-(2-iodo-5-methoxy-phenylsulfanyl)-purin-9-yl]-acetamide; 1-[6-Amino-8-(2-iodo-5-methoxy-phenylsulfanyl)-purin-9-yl]-propan-2-one; N-{2-[6-Amino-8-(2-iodo-5-methoxy-phenylsulfanyl)-purin-9-yl]-ethyl}-acetamide; N-{2-[6-Amino-8-(2-iodo-5-methoxy-phenylsulfanyl)-purin-9-yl]-ethyl}-methanesulfonamide; N-{2-[6-Amino-8-(2-iodo-5-methoxy-phenylsulfanyl)-purin-9-yl]-ethyl}-N-isobutyl-acetamide; N-{2-[6-Amino-8-(2-iodo-5-methoxy-phenylsulfanyl)-purin-9-yl]-ethyl}-N-isobutyl-methanesulfonamide; 8-(3-Bromo-2,5-dimethoxy-phenylsulfanyl)-9-(4-methyl-pent-3-enyl)-9H-purin-6-ylamine; 8-(3-Bromo-2,5-dimethoxy-phenylsulfanyl)-9-pent-4-ynyl-9H-purin-6-ylamine; 8-(2,5-Dimethoxy-biphenyl-3-ylsulfanyl)-9-pent-4-ynyl-9H-purin-6-ylamine; 9-(4-Methyl-pent-3-enyl)-8-(thiazol-2-ylsulfanyl)-9H-purin-6-ylamine; 8-(Benzothiazol-2-ylsulfanyl)-9-(4-methyl-pent-3-enyl)-9H-purin-6-ylamine; 8-(1H-Benzoimidazol-2-ylsulfanyl)-9-(4-methyl-pent-3-enyl)-9H-purin-6-ylamine; Acetic acid 2-[6-amino-8-(naphthalen-2-ylsulfanyl)-purin-9-yl]-ethyl ester; Acetic acid 2-[6-amino-8-(naphthalen-1-ylsulfanyl)-purin-9-yl]-ethyl ester; Acetic acid 2-[6-amino-8-(quinolin-8-ylsulfanyl)-purin-9-yl]-ethyl ester; Acetic acid 2-[6-amino-8-(1H-indol-2-ylsulfanyl)-purin-9-yl]-ethyl ester; Acetic acid 2-[6-amino-8-(2,5-dimethoxy-phenylsulfanyl)-purin-9-yl]-ethyl ester; Acetic acid 2-[6-amino-8-(benzo[b]thiophen-2-ylsulfanyl)-purin-9-yl]-ethyl ester; 8-(Benzothiazol-2-ylsulfanyl)-9-pent-4-ynyl-9H-purin-6-ylamine; 9-Pent-4-ynyl-8-(quinolin-2-ylsulfanyl)-9H-purin-6-ylamine; 8-(1-Allyl-1H-benzoimidazol-2-ylsulfanyl)-9-(4-methyl-pent-3-enyl)-9H-purin-6-ylamine; 8-(1-Methyl-1H-benzoimidazol-2-ylsulfanyl)-9-(4-methyl-pent-3-enyl)-9H-purin-6-ylamine; 2-[6-Amino-8-(naphthalen-2-ylsulfanyl)-purin-9-yl]-ethanol; 2-[6-Amino-8-(naphthalen-1-ylsulfanyl)-purin-9-yl]-ethanol; 2-[6-Amino-8-(quinolin-8-ylsulfanyl)-purin-9-yl]-ethanol; Acetic acid 2-[6-amino-8-(3-chloro-1H-indol-2-ylsulfanyl)-purin-9-yl]-ethyl ester; Acetic acid 2-[6-amino-8-(3-bromo-1H-indol-2-ylsulfanyl)-purin-9-yl]-ethyl ester; Acetic acid 2-[6-amino-8-(3-iodo-1H-indol-2-ylsulfanyl)-purin-9-yl]-ethyl ester; Acetic acid 2-[6-amino-8-(1-propyl-1H-indol-2-ylsulfanyl)-purin-9-yl]-ethyl ester; Acetic acid 2-[6-amino-8-(3-iodo-1-propyl-1H-indol-2-ylsulfanyl)-purin-9-yl]-ethyl ester; Acetic acid 2-[6-amino-8-(1,4-dimethoxy-naphthalen-2-ylsulfanyl)-purin-9-yl]-ethyl ester; 3-[6-Amino-8-(benzo[1,3]dioxol-5-ylsulfanyl)-purin-9-yl]-propan-1-ol; 3-[6-Amino-8-(2,3-dihydro-benzo[1,4]dioxin-6-ylsulfanyl)-purin-9-yl]-propan-1-ol; 9-Butyl-8-(7-chloro-benzothiazol-2-ylsulfanyl)-9H-purine-6-ylamine; 9-ethyl-8-(7-chloro-benzothiazol-2-ylsulfanyl)-9H-purine-6-ylamine; 9-Propyl-8-(7-chloro-benzothiazol-2-ylsulfanyl)-9H-purine-6-ylamine; 9-Pentyl-8-(7-chloro-benzothiazol-2-ylsulfanyl)-9H-purine-6-ylamine; 8-(7-Bromo-benzothiazol-2-ylsulfanyl)-9-butyl-9H-purine-6-ylamine; 9-Butyl-8-(7-methyl-benzothiazol-2-ylsulfanyl)-9H-purine-6-ylamine; 9-Butyl-8-(7-methoxy-benzothiazol-2-ylsulfanyl)-9H-purine-6-ylamine; 9-Butyl-8-(7-ethoxy-benzothiazol-2-ylsulfanyl)-9H-purine-6-ylamine; 9-Butyl-8-(7-fluoro-benzothiazol-2-ylsulfanyl)-9H-purine-6-ylamine; 9-Butyl-8-(7-trifluoromethyl-benzothiazol-2-ylsulfanyl)-9H-purine-6-ylamine; 8-(Benzothiazol-2-ylsulfanyl)-9-butyl-9H-purine-6-ylamine; 9-Butyl-8-(6-chloro-benzothiazol-2-ylsulfanyl)-9H-purine-6-ylamine; 9-Butyl-8-(5-chloro-benzothiazol-2-ylsulfanyl)-9H-purine-6-ylamine; 9-Butyl-8-(4-chloro-benzothiazol-2-ylsulfanyl)-9H-purine-6-ylamine; 9-Butyl-8-(4-chloro-benzothiazol-2-ylsulfanyl)-9H-purine-6-ylamine; 8-(7-Chloro-benzothiazol-2-ylsulfanyl)-9-pent-4-ynyl-9H-purin-6-ylamine; 8-(7-Chloro-benzothiazol-2-ylsulfanyl)-9-(2-methoxy-ethyl)-9H-purin-6-ylamine; 8-(7-Chloro-benzothiazol-2-ylsulfanyl)-9-(2-vinyloxy-ethyl)-9H-purin-6-ylamine; 9-Butyl-8-(thiazolo[5,4-b]pyridin-2-ylsulfanyl)-9H-purine-6-ylamine; Acetic acid 4-[6-amino-8-(7-fluoro-benzothiazol-2-ylsulfanyl)-purin-9-yl]-butyl ester; 8-(4-Bromo-6,7-difluoro-benzothiazol-2-ylsulfanyl)-9-butyl-9H-purine-6-ylamine; 9-Butyl-8-(6,7-dichloro-benzothiazol-2-ylsulfanyl)-9H-purine-6-ylamine; 9-Butyl-8-(6,7-difluoro-benzothiazol-2-ylsulfanyl)-9H-purine-6-ylamine; 8-(6,7-Dichloro-benzothiazol-2-ylsulfanyl)-9-pent-4-ynyl-9H-purin-6-ylamine; Acetic acid 3-[6-amino-8-(6,7-dichloro-benzothiazol-2-ylsulfanyl)-purin-9-yl]-propyl ester; 9-But-3-enyl-8-(7-chloro-benzothoazol-2-ylsulfanyl)-9H-purine-6-ylamine; 8-(7-Chloro-benzothiazol-2-ylsulfanyl)-9-pent-4-ynyl-9H-purin-6-ylamine; 8-(7-Methoxy-benzothiazol-2-ylsulfanyl)-9-pent-4-ynyl-9H-purin-6-ylamine; 8-(7-Methyl-benzothiazol-2-ylsulfanyl)-9-pent-4-ynyl-9H-purin-6-ylamine; 9-Butyl-8-(7-methoxymethoxymethyl-benzothiazol-2-ylsulfanyl)-9H-purine-6-ylamine; Acetic acid 3-[6-amino-8-(7-methoxy-benzothiazol-2-ylsulfanyl)-purin-9-yl]-propyl ester; Acetic acid 3-[6-amino-8-(7-methyl-benzothiazol-2-ylsulfanyl)-purin-9-yl]-propyl ester; 8-(4-Amino-7-fluorol-benzothiazol-2-ylsulfanyl)-9-pent-4-ynyl-9H-purin-6-ylamine; 8-(7-Ethoxy-benzothiazol-2-ylsulfanyl)-9-pent-4-ynyl-9H-purin-6-ylamine; Acetic acid 2-[6-amino-8-(7-methoxy-benzothiazol-2-ylsulfanyl)-purin-9-yl]-ethyl ester; 8-(7-Chloro-benzothiazol-2-ylsulfanyl)-9-ethyl-9H-purine-6-ylamine; 2-Chloro-8-(7-chloro-benzothiazol-2-ylsulfanyl)-9-methyl-9H-purine-6-ylamine; 8-(7-Bromo-thiazolo[5,4-b]pyridin-2-ylsulfanyl)-9-butyl-9H-purine-6-ylamine; 8-(7-Bromo-thiazole[4,5-c]pyridin-2-ylsulfanyl)-2-chloro-9-methyl-9H-purin-6-ylamine; Acetic acid 2-[6-amino-8-(7-bromo-thiazole[4,5-c]pyridin-2-ylsulfanyl)-purin-9-yl]-ethyl ester; 8-(7-Bromo-thiazolo[5,4-b]pyridin-2-ylsulfanyl)-9-butyl-9H-purine-6-ylamine; Acetic acid 3-[6-amino-8-(7-bromo-thiazole[4,5-c]pyridin-2-ylsulfanyl)-purin-9-yl]-propyl ester; 8-(7-Bromo-thiazole[4,5-c]pyridin-2-ylsulfanyl)-9-(4-methyl-pent-3-enyl)-9H-purin-6-ylamine; Acetic acid 2-[6-amino-8-(7-bromo-thiazole[4,5-c]pyridin-2-ylsulfanyl)-purin-9-yl]-ethyl ester; 8-(7-Bromo-thiazolo[5,4-b]pyridin-2-ylsulfanyl)-2-chloro-9-methyl-9H-purine-6-ylamine; Acetic acid 3-[6-amino-8-(7-chloro-thiazolo[4,5-c]pyridin-2-ylsulfanyl)-purin-9-yl]-propyl ester; 9-Butyl-8-(7-chloro-benzooxazol-2-ylsulfanyl)-9H-purine-6-ylamine; Acetic acid 2-[6-amino-8-(7-chloro-benzooxazol-2-ylsulfanyl)-purin-9-yl]-ethyl ester; Acetic acid 3-[6-amino-8-(7-chloro-benzooxazol-2-ylsulfanyl)-purin-9-yl]-propyl ester; 9-Butyl-8-(7-fluoro-benzooxazol-2-ylsulfanyl)-9H-purine-6-ylamine; Acetic acid 2-[6-amino-8-(7-chloro-benzothiazol-2-ylsulfanyl)-purin-9-yl]-ethyl ester; Acetic acid 2-[6-amino-8-(7-fluoro-benzothiazol-2-ylsulfanyl)-purin-9-yl]-ethyl ester; Acetic acid 4-[6-amino-8-(7-chloro-benzothiazol-2-ylsulfanyl)-purin-9-yl]-butyl ester; Acetic acid 3-[6-amino-8-(7-fluoro-benzothiazol-2-ylsulfanyl)-purin-9-yl]-propyl ester; Acetic acid 3-[6-amino-8-(7-bromo-benzothiazol-2-ylsulfanyl)-purin-9-yl]-propyl ester; 2-[6-Amino-8-(7-chloro-benzothiazol-2-ylsulfanyl)-purin-9-yl]-ethanol; 2-[6-Amino-8-(7-fluoro-benzothiazol-2-ylsulfanyl)-purin-9-yl]-ethanol; 2-[6-Amino-8-(7-chloro-benzothiazol-2-ylsulfanyl)-purin-9-yl]-propan-1-ol; 4-[6-Amino-8-(7-chloro-benzothiazol-2-ylsulfanyl)-purin-9-yl]-butan-1-ol; 4-[6-amino-8-(7-fluoro-benzothiazol-2-ylsulfanyl)-purin-9-yl]-butan-1-ol; 3-[6-amino-8-(7-fluoro-benzothiazol-2-ylsulfanyl)-purin-9-yl]-propan-ol; 3-[6-Amino-8-(6,7-dichloro-benzothiazol-2-ylsulfanyl)-purin-9-yl]-propan-1-ol; 3-[6-Amino-8-(7-bromo-benzothiazol-2-ylsulfanyl)-purin-9-yl]-propan-ol; 3-[6-Amino-8-(7-methoxy-benzothiazol-2-ylsulfanyl)-purin-9-yl]-propan-ol; 2-[6-Amino-8-(7-methoxy-benzothiazol-2-ylsulfanyl)-purin-9-yl]-ethanol; 3-[6-Amino-8-(7-methyl-benzothiazol-2-ylsulfanyl)-purin-9-yl]-propan-ol; 2-[6-amino-8-(7-bromo-thiazolo[4,5-c]pyridin-2-ylsulfanyl)-purin-9-yl]-ethanol; 2-[6-Amino-8-(7-chloro-thiazolo[5,4-b]pyridin-2-ylsulfanyl)-purin-9-yl]-ethanol; 8-(7-Chloro-benzothiazol-2-ylsulfanyl)-9-[3-(1-ethyl-propylamino)-propyl]-9H-purin-6-yl amine; 9-(3-tert-Butylamino-propyl)-8-(7-chloro-benzothiazol-2-ylsulfanyl)-9H-purin-6-yl amine; 8-(7-Chloro-benzothiazol-2-ylsulfanyl)-9-(3-isobutylamino-propyl)-9H-purin-6-yl amine; 9-(3-sec-Butylamino-propyl)-8-(7-Chloro-benzothiazol-2-ylsulfanyl)-9H-purin-6-yl amine; 9-[2-(2,2-Dimethyl-propylamino)-ethyl]-8-(7-Chloro-benzothiazol-2-ylsulfanyl)-9H-purin-6-yl amine; 9-[2-Isopropylamino-ethyl]-8-(7-methoxy-benzothiazol-2-ylsulfanyl)-9H-purin-6-yl amine; 9-[2-tert-Butylamino-ethyl]-8-(7-methoxy-benzothiazol-2-ylsulfanyl)-9H-purin-6-yl amine; 9-(2-Isobutylamino-ethyl)-8-(7-methoxy-benzothiazol-2-ylsulfanyl)-9H-purin-6-yl amine; 8-(7-Chloro-benzothiazol-2-ylsulfanyl)-9-[3-(2,2-dimethyl-propylamino)-propyl]-9H-purin-6-yl amine; 8-(7-Chloro-benzothiazol-2-ylsulfanyl)-9-(2-prop-2-ynylamino-ethyl)-9H-purin-6-yl amine; 8-(7-Chloro-benzothiazol-2-ylsulfanyl)-9-(2-cyclopentylamino-ethyl)-9H-purin-6-yl amine; 8-(7-Chloro-benzothiazol-2-ylsulfanyl)-9-[2-(3-methyl-butylamino)-ethyl]-9H-purin-6-yl amine; 8-(7-Chloro-benzothiazol-2-ylsulfanyl)-9-[2-(1,1-dimethyl-propylamino)-ethyl]-9H-purin-6-yl amine; 9-(2-Allylamino-ethyl)-8-(7-chloro-benzothiazol-2-ylsulfanyl)-9H-purin-6-ylamine; 8-(7-Chloro-benzothiazol-2-ylsulfanyl)-9-(3-isopropylamino-propyl)9H-purin-6-yl amine; 8-(7-Chlorol-benzothiazol-2-ylsulfanyl)-9-(3-pyrrol-1-yl-propyl)-9H-purine-6-ylamine; 8-(7-Chloro-benzothiazol-2-ylsulfanyl)-9-[2-(3,3-dimethyl-butylamino)-ethyl]-9H-purin-6-yl amine; 8-(7-Chloro-benzothiazol-2-ylsulfanyl)-9-(3-morpholin-4-yl-propyl)-9H-purin-6-ylamine; 8-(7-Chloro-benzothiazol-2-ylsulfanyl)-9-(2-morpholin-4-yl-ethyl)-9H-purin-6-ylamine; 9-(2-Bromo-ethyl)-8-(7-chloro-benzothiazol-2-ylsulfanyl)-9H-purine-6-ylamine; 8-(7-Fluoro-benzothiazol-2-ylsulfanyl)-9-(2-vinyloxy-ethyl)-9H-purin-6-ylamine; 8-(7-Fluoro-benzothiazol-2-ylsulfanyl)-9-pent-4-ynyl-9H-purin-6-ylamine; 8-[(7-Chloro-benzothiazol-2-ylsulfanyl)-9-(2-chloro-ethyl)-9H-purine-6-ylamine; 9-(3-Bromo-propyl)-8-(7-chloro-benzothiazol-2-ylsulfanyl)-9H-purine-6-ylamine; 8-(7-Chloro-benzothoazol-2-ylsulfanyl)-9-pent-4-enyl-9H-purine-6-ylamine; 8-(7-Chloro-benzothoazol-2-ylsulfanyl)-9-hex-5-enyl-9H-purine-6-ylamine; 8-(7-Chloro-benzothiazol-2-ylsulfanyl)-9-[2,(2,5-dimethoxy-phenyl)-ethyl]-9H-purine-6-ylamine; 9-But-2-ynyl-8-(7-chloro-benzothiazol-2-ylsulfanyl)-9H-purine-6-ylamine; 8-(7-Chloro-benzothiazol-2-ylsulfanyl)-9-(3,4,4-trifluoro-but-3-enyl)-9H-purin-6-ylamine; 6-[6-Amino-8-(7-chloro-benzothiazol-2-ylsulfanyl)-purin-9-yl]-hexanenitrile; 8-(7-Chloro-benzothiazol-2-ylsulfanyl)-9-(3-methyl-but-3-enyl)-9H-purin-6-ylamine; 4-[6-Amino-8-(7-chloro-benzothiazol-2-ylsulfanyl)-purin-9-yl]-butyronitrile; 8-(7-Chloro-benzothiazol-2-ylsulfanyl)-9-hex-5-ynyl-9H-purin-6-ylamine; 8-(7-Chloro-benzothiazol-2-ylsulfanyl)-9-[3-(tetrahydro-furan-2-yl)-propyl]-9H-purin-6-ylamine; 8-(7-Chloro-benzothiazol-2-ylsulfanyl)-9-(tetrahydro-furan-2-ylmethyl)-9H-purin-6-ylamine; 8-(7-Chloro-benzothiazol-2-ylsulfanyl)-9-[2-(2-ethoxy-ethoxy)-ethyl]-9H-purin-6-ylamine; 5-[6-Amino-8-(7-chloro-benzothiazol-2-ylsulfanyl)-purin-9-yl]-pentanenitrile; 8-(7-Chlorol-benzothiazol-2-ylsulfanyl)-9-(4-methoxy-3,5-dimethyl-pyridin-2-ylmethyl)-9H-purine-6-ylamine; 8-(7-Chloro-benzothiazol-2-ylsulfanyl)-9-prop-2-ynyl-9H-purine-6-ylamine; 8-(7-Chloro-benzothiazol-2-ylsulfanyl)-9-(2-piperidin-1-yl-ethyl]-9H-purin-6-yl amine; 8-(7-Chloro-benzothiazol-2-ylsulfanyl)-9-(2-methylsulfanyl-ethyl)-9H-purin-6-ylamine; 8-(7-Chloro-benzothiazol-2-ylsulfanyl)-9-(2-ethylsulfanyl-ethyl)-9H-purin-6-ylamine; Phosphoric acid 3-[6-amino-8-(7-chloro-benzothiazol-2-ylsulfanyl)-9-yl]-propyl ester diethyl ester; Phosphoric acid 2-[6-amino-8-(7-chloro-benzothiazol-2-ylsulfanyl)-purin-9-yl]-ethyl bis-(2-chloro-ethyl)ester; {3-[6-Amino-8-(7-chloro-benzothiazol-2-ylsulfanyl)-purin-9-yl]-propenyl}-phosphonic acid diethyl ester; Phosphoric acid 2-[6-amino-8-(7-chloro-benzothiazol-2-ylsulfanyl)-9-yl]-ethyl ester diethyl ester; 8-(7-Chloro-benzothiazol-2-ylsulfanyl)-9-methyl-9H-purine-6-ylamine; 4-[6-Amino-8-(7-chloro-benzothiazol-2-ylsulfanyl)-purin-9-yl]-butan-2-one; 8-(7-Chloro-benzothiazol-2-ylsulfanyl)-9-(2-ethylsulfinyl-ethyl)-9H-purin-6-ylamine; 4-[6-Amino-8-(7-chloro-benzothiazol-2-ylsulfanyl)-purin-9-yl]-butan-2-thione; 8-(7-Chloro-benzothiazol-2-ylsulfanyl)-9-(2-ethanesulfonyl-ethyl)-9H-purin-6-ylamine; 8-(7-Chloro-benzothiazol-2-ylsulfanyl)-9-(2-methanesulfonyl-ethyl)-9H-purin-6-ylamine; (6-Amino-9-butyl-9H-purin-8-ylsulfanyl)-benzothiazol-7-yl]-methanol; 9-(2-Dimethylamino-ethyl)-8-(2-iodo-5-methoxy-phenylsulfanyl)-9H-purin-6-ylamine; 9-(2-Diethylamino-ethyl)-8-(2-iodo-5-methoxy-phenylsulfanyl)-9H-purin-6-ylamine; 8-(2-Iodo-5-methoxy-phenylsulfanyl)-9-(2-pyrrolidin-1-yl-ethyl)-9H-purin-6-ylamine; 8-(2-Iodo-5-methoxy-phenylsulfanyl)-9-(2-vinyloxy-ethyl)-9H-purin-6-ylamine; 8-(2-Iodo-5-methoxy-phenylsulfanyl)-9-(2-isopropoxy-ethyl)-9H-purin-6-ylamine; {3-[6-Amino-8-(2-iodo-5-methoxy-phenylsulfanyl)-purin-9-yl]-propyl}-methyl-carbamic acid tert-butyl ester; 8-(2-Iodo-5-methoxy-phenylsulfanyl)-9-(3-pyrrol-1-yl-propyl)-9H-purin-6-ylamine; (2,4-Diiodo-5-methoxy-phenylsulfanyl)-9-pent-4-ynyl-9H-purin-6-ylamine; {3-[6-Amino-8-(2-iodo-5-methoxy-phenylsulfanyl)-purin-9-yl]-propyl}-carbamic acid tert-butyl ester; 8-(2-Iodo-5-trifluoromethoxy-phenylsulfanyl)-9-pent-4-ynyl-9H-purin-6-ylamine and 8-(2-Iodo-5-trifluoromethyl-phenylsulfanyl)-9-pent-4-ynyl-9H-purin-6-ylamine.

51. A compound selected from the group consisting of:
8-(2-Iodo-5-methoxy-phenylsulfanyl)-9-(2-isobutylamino-ethyl)-9H-purin-6-ylamine; 9-(3-tert-Butylamino-propyl)-8-(2-iodo-5-methoxy-phenylsulfanyl)-9H-purin-6-ylamine; 9-[3-(1-Ethyl-propylamino)-propyl]-8-(2-iodo-5-methoxy-phenylsulfanyl)-9H-purin-6-ylamine; 9-(3-sec-Butylamino-propyl)-8-(2-iodo-5-methoxy-phenylsulfanyl)-9H-purin-6-ylamine (racemate); (R)-9-(3-sec-Butylamino-propyl)-8-(2-iodo-5-methoxy-phenylsulfanyl)-9H-purin-6-ylamine; (S)-9-(3-sec-Butylamino-propyl)-8-(2-iodo-5-methoxy-phenylsulfanyl)-9H-purin-6-ylamine; 8-(2-Iodo-5-methoxy-phenylsulfanyl)-9-(3-isopropylamino-propyl)-9H-purin-6-ylamine; 9-[2-(2,2-Dimethyl-propylamino)-ethyl]-8-(2-iodo-5-methoxy-phenylsulfanyl)-9H-purin-6-ylamine; {2-[6-Amino-8-(7-chloro-benzothiazol-2-ylsulfanyl)-purin-9-yl]-ethyl}-phosphonic acid diethyl ester; {2-[6-Amino-8-(7-chloro-thiazolo[4,5-c]pyridinl-2-ylsulfanyl)-purin-9-yl]-ethyl}-phosphonic acid diethyl ester; {2-[6-amino-8-(7-chloro-benzothiazol-2-ylsulfanyl)-purin-9-yl]-ethyl}-phosphonic acid diethyl ester; {2-[6-Amino-8-(7-chloro-benzothiazol-2-ylsulfanyl)-purine-9-yl]-ethyl}-phosphoramidic acid diethyl ester; {2-[6-amino-8-(7-bromo-thiazole[4,5-c]pyridin-2-ylsulfanyl)-purin-9-yl]ethyl}-phosphonic acid diethyl ester; {3-[6-Amino-8-(7-bromo-thiazole[4,5-c]pyridin-2-ylsulfanyl)-purin-9-yl]propyl}-phosphonic acid diethyl ester; 4{3-[6-Amino-8-(7-chloro-benzothiazol-2-ylsulfanyl)-9-yl]-propyl}-phosphonic acid diethyl ester; {3-[6-Amino-8-(7-chloro-benzothiazol-2-ylsulfanyl)-purin-9-yl]-propenyl}-phosphonic acid diethyl ester; 8-(7-Chloro-benzothiazol-2-ylsulfanyl)-9-[2-(2,2-dimethyl-propylamino)-ethyl]-9H-purin-6-yl amine; 8-(7-Chloro-benzothiazol-2-ylsulfanyl)-9-[2-(cyclopropylmethyl-amino)-ethyl]-9H-purin-6-yl amine; 8-(7-Chloro-benzothiazol-2-ylsulfanyl)-9-(2-cyclopropylamino)-ethyl)-9H-purin-6-yl amine; 9-(2-tert-Butylamino-ethyl)-8-(7-chloro-benzothiazole-2-ylsulfanyl)-9H-purin-6-yl amine and acetic acid 2-[6-amino-8-(7-chloro-benzothiazol-2-ylsulfanyl)-purin-9-yl]-propyl ester.

52. A pharmaceutical composition comprising a compound, tautomer, or pharmaceutically acceptable salt of claim 51 and one or more pharmaceutical carriers or excipients.

53. A method of inhibiting an HSP90, comprising:
contacting a cell having an HSP90 with a compound, tautomer or pharmaceutically acceptable salt or pharmaceutical composition according to claim 51.
